SEQUENCE LISTING (25 sequences)

SEQ ID NO: 1 - DNA, 15186 bp, Artificial Sequence
  cDNA of genomic sequence of NDV strain LaSota
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 2 - DNA, 15186 bp, Artificial Sequence
  cDNA of genomic sequence of NDV strain Hitchner B1
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 3 - protein, 553 aa, Artificial Sequence
  Amino acid sequence of F protein of NDV strain LaSota (transmembrane domain is underlined and cytoplasmic domain is in bold)
MGSRPSTKNPAPMTLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 4 - DNA, 714 bp, Artificial Sequence
  Secreted Receptor Binding Domain (RBD) of SARS-CoV-2 Spike Protein (nucleotide sequence)
atgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttctga

SEQ ID NO: 5 - protein, 237 aa, Artificial Sequence
  Secreted Receptor Binding Domain (RBD) of SARS-CoV-2 Spike Protein (amino acid sequence)
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 6 - DNA, 732 bp, Artificial Sequence
  Secreted RBD of SARS-CoV-2 Spike Protein 6xHis (nucleotide sequence)
atgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttccaccatcaccatcaccattga

SEQ ID NO: 7 - protein, 243 aa, Artificial Sequence
  Secreted RBD of SARS-CoV-2 Spike Protein 6xHis (amino acid sequence)
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 8 - DNA, 3771 bp, Artificial Sequence
  Secreted ectodomain of SARS-CoV-2 spike protein 6x His (nucleotide sequence)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaa

SEQ ID NO: 9 - protein, 1256 aa, Artificial Sequence
  Secreted ectodomain of SARS-CoV-2 spike protein 6x His (amino acid sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 10 - DNA, 3822 bp, Artificial Sequence
  Full length SARS-CoV-2 spike protein (nucleotide sequence)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccagacgggccagatctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacctga

SEQ ID NO: 11 - protein, 1273 aa, Artificial Sequence
  Full length SARS-CoV-2 spike protein (amino acid sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - DNA, 3810 bp, Artificial Sequence
  Chimeric F protein (SARS-CoV-2 spike protein ectodomain and NDV F protein transmembrane and cytoplasmic domains; nucleotide sequence)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggggggagtctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgataa

SEQ ID NO: 13 - protein, 1268 aa, Artificial Sequence
  Chimeric F protein (SARS-CoV-2 spike protein ectodomain and NDV F protein transmembrane and cytoplasmic domains; amino acid sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 14 - DNA, 3810 bp, Artificial Sequence
  S-F chimera HexaPro (Nucleotide sequence)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataa

SEQ ID NO: 15 - protein, 1268 aa, Artificial Sequence
  S-F chimera HexaPro (Amino acid sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 16 - DNA, 3798 bp, Artificial Sequence
  NDV-HXP-S (B.1.351) (Nucleotide sequence)
atgttcgtgtttctggtgctgctgcctctggtgtccagccagtgtgtgaacttcaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggcctgcctcagggcttctctgctctggaacccctggtggatctgcccatcggcatcaacatcacccggtttcagacactgcacatcagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgtggagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 17 - protein, 1265 aa, Artificial Sequence
  NDV-HXP-S (B.1.351) (Amino acid sequence)
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 18 - DNA, 3807 bp, Artificial Sequence
  NDV-HXP-S (P.1) (Nucleotide sequence)
atgttcgtgtttctggtgctgctgcctctggtgtccagccagtgtgtgaacttcaccaacagaacccagctgcctagcgcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaactaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgagcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaccatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtgaagggcttcaactgctacttcccactgcagtcctacggctttcagcccacatacggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccatcaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 19 - protein, 1268 aa, Artificial Sequence
  NDV-HXP-S (P.1) (Amino acid sequence)
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 20 - DNA, 6 bp, Artificial Sequence
  SacII Restriction Sequence
ccgcgg

SEQ ID NO: 21 - DNA, 10 bp, Artificial Sequence
  NDV Gene End Sequence
ttagaaaaaa

SEQ ID NO: 22 - DNA, 10 bp, Artificial Sequence
  NDV Gene Start Sequence
acgggtagaa

SEQ ID NO: 23 - DNA, 8 bp, Artificial Sequence
  Kozak Sequence
ccgccacc

SEQ ID NO: 24 - protein, 5 aa, Artificial Sequence
  Linker Sequence
GGGGS

SEQ ID NO: 25 - DNA, 15186 bp, Artificial Sequence
  cDNA of genomic sequence of NDV strain LaSota (L289A mutation)
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

(19) United States
(12) Patent Application Publication
(10) Pub. No.: US 2023/0310583 A1
(43) Pub. Date: Oct. 5, 2023

(54) RECOMBINANT NEWCASTLE DISEASE VIRUS EXPRESSING SARS-COV-2 SPIKE PROTEIN AND USES THEREOF

(71) Applicant: ICAHN SCHOOL OF MEDICINE AT MOUNT SINAI, New York, NY (US)

(72) Inventors: Weina SUN, New York, NY (US); Florian KRAMMER, New York, NY (US); Adolfo GARCIA-SASTRE, New York, NY (US); Peter PALESE, New York, NY (US)

(73) Assignee: ICAHN SCHOOL OF MEDICINE AT MOUNT SINAI, New York, NY (US)

(21) Appl. No.: 17/922,777

(22) PCT Filed: May 6, 2021
(86) PCT No.: PCT/US2021/031110

§ 371 Date: Nov. 1, 2022

Related U.S. Application Data
(60) Provisional application No. 63/059,924, filed on Jul. 31, 2020.
(60) Provisional application No. 63/058,435, filed on Jul. 29, 2020.
(60) Provisional application No. 63/057,267, filed on Jul. 27, 2020.
(60) Provisional application No. 63/051,858, filed on Jul. 14, 2020.
(60) Provisional application No. 63/021,677, filed on May 7, 2020.

(30) Foreign Application Priority Data
Mar. 17, 2021 (WO) PCT/US2021/022848

(51) Int. Cl. A61K 39/215; A61P 31/14; C12N 15/63

(52) U.S. Cl. CPC A61K 39/215; A61P 31/14; C12N 15/63; A61K 2039/5256

(57) ABSTRACT

Described herein are recombinant Newcastle disease viruses (“NDVs”) comprising a packaged genome, wherein the packaged genome comprises a transgene comprising a nucleotid sequence encoding a SARS-CoV-2 spike protein or nucleocapsid protein. Also described herein are recombinant NDVs comprising a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains. The recombinant NDVs and compositions thereof are useful for the immunizing against SARS-CoV-2 as well as the prevention of COVID-19.

### CROSS-REFERENCE TO RELATED APPLICATIONS

This application claims the benefit of U.S. Provisional Application No. 63/059,924, filed Jul. 31, 2020, U.S. Provisional Application No. 63/058,435, filed Jul. 29, 2020, U.S. Provisional Application No. 63/057,267, filed Jul. 27, 2020, U.S. Provisional Application No. 63/051,858, filed Jul. 14, 2020, and U.S. Provisional Application No. 63/021,677, filed May 7, 2020, and is the continuation-in-part of International Application No. PCT/US2021/022848, filed Mar. 17, 2021, the disclosure of each of which is incorporated by reference herein in its entirety.

### STATEMENT REGARDING FEDERALLY SPONSORED RESEARCH

This invention was made with government support under grant HHSN272201400008C awarded by the National Institutes of Health. The government has certain rights in the invention.

### SEQUENCE LISTING

This application incorporates by reference a Sequence Listing submitted with this application as a text file, entitled 06923-341-228_SEQ_LISTING.txt, created on May 5, 2021, and is 170,237 bytes in size.

### 1. INTRODUCTION

In one aspect, described herein are recombinant Newcastle disease virus (“NDV”) comprising a packaged genome, wherein the packaged genome comprises a transgene encoding severe acute respiratory syndrome coronavirus 2 (“SARS-CoV-2”) spike protein or a portion thereof (e.g., ectodomain or receptor binding domain of SARS-CoV-2 spike protein). In a specific embodiment, described herein are recombinant NDV comprising a packaged genome, wherein the packaged genome comprises a transgene comprising a codon optimized nucleic acid sequence encoding SARS-CoV-2 spike protein or portion thereof (e.g., ectodomain or receptor binding domain of SARS-CoV-2 spike protein). In a specific embodiment, described herein are recombinant NDV comprising a packaged genome, wherein the packaged genome comprises a transgene encoding a chimeric F protein, wherein the chimeric F protein comprises an SARS-CoV-2 spike protein ectodomain and NDV F protein transmembrane and cytoplasmic domains. In some embodiments, the ectodomain of the SARS-CoV-2 spike protein is encoded by a codon optimized nucleic acid sequence. Also described herein are compositions comprising such recombinant NDV and the use of such recombinant NDV to induce an immune response to SARS-CoV-2 spike protein, and in immunoassays to detect the presence of antibody that binds to SARS-CoV-2 spike protein.

In another aspect, described herein are recombinant NDV comprising a packaged genome, wherein the packaged genome comprises a transgene comprising a nucleotide sequence encoding SARS-CoV-2 nucleocapsid protein or a portion thereof. In a specific embodiment, described herein are recombinant NDV comprising a packaged genome, wherein the packaged genome comprises a transgene comprising a codon optimized nucleic acid sequence encoding SARS-CoV-2 nucleocapsid protein or a portion thereof. Also described herein are compositions comprising such recombinant NDV and the use of such recombinant NDV to induce an immune response to SARS-CoV-2 nucleocapsid protein, and in immunoassays to detect the presence of antibody that binds to SARS-CoV-2 nucleocapsid protein.

In another aspect, described herein are recombinant NDV comprising a packaged genome, wherein the packaged genome comprises a transgene comprising a nucleotide sequence encoding a SARS-CoV-2 nucleocapsid protein or a portion thereof, and a transgene comprising a nucleotide sequence encoding SARS-CoV-2 spike protein or a portion thereof (e.g., ectodomain or receptor binding domain of SARS-CoV-2 spike protein). In another aspect, described herein are recombinant NDV comprising a packaged genome, wherein the packaged genome comprises a transgene comprising (i) a nucleotide sequence encoding a SARS-CoV-2 nucleocapsid protein or a portion thereof and (ii) a nucleotide sequence encoding SARS-CoV-2 spike protein or a portion thereof (e.g., ectodomain or receptor binding domain of SARS-CoV-2 spike protein). Also described herein are compositions comprising such recombinant NDV and the use of such recombinant NDV to induce an immune response to SARS-CoV-2 nucleocapsid protein, and in immunoassays to detect the presence of antibody that binds to SARS-CoV-2 nucleocapsid protein.

### 2. BACKGROUND

There is an urgent need to develop therapeutics to treat COVID-19 and diagnostics to detect severe acute respiratory syndrome coronavirus-2 (SARS-CoV-2). As of May 7, 2020, more than 3,815,561 people have tested positive for SARS-CoV-2. In addition, as of May 7, 2020, more than 70,802 Americans have died from COVID-19 and globally more than 267,469 people have died from COVID-19. As of Jul. 26, 2020, SARS-CoV-2 has resulted in approximately 16.3 million infections with more than half a million deaths, and continues to pose a threat to public health. Currently, there is no vaccine to prevent COVID-19.

Newcastle disease virus (NDV) is a member of the Avulavirus genus in the Paramyxoviridae family, which has been shown to infect a number of avian species (Alexander, DJ (1988). Newcastle disease, Newcastle disease virus -- an avian paramyxovirus. Kluwer Academic Publishers: Dordrecht, The Netherlands, pp 1-22). NDV possesses a single-stranded RNA genome in negative sense and does not undergo recombination with the host genome or with other viruses (Alexander, DJ (1988). Newcastle disease, Newcastle disease virus -- an avian paramyxovirus. Kluwer Academic Publishers: Dordrecht, The Netherlands, pp 1-22). The genomic RNA contains genes in the order of 3′-NP-P-M-F-HN-L-5′, described in further detail below. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding / fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication. The V protein has been shown to inhibit interferon-alpha and to contribute to the virulence of NDV (Huang et al. (2003). Newcastle disease virus V protein is associated with viral pathogenesis and functions as an Alpha Interferon Antagonist. Journal of Virology 77: 8676-8685).

### 3. SUMMARY

In one aspect, presented herein are recombinant Newcastle disease virus (“NDV”) comprising a packaged genome, wherein the packaged genome comprises a transgene comprising a nucleotide sequence encoding a SARS-CoV-2 spike protein or a portion thereof (e.g., the ectodomain or receptor binding domain of the SARS-CoV-2 spike protein). In one embodiment, the transgene comprises a nucleotide sequence encoding full length SARS-CoV-2 spike protein. In another embodiment, the transgene comprises a nucleotide sequence encoding a protein comprising (or consisting of) the receptor binding domain of s SARS-CoV-2 spike protein. In certain embodiments, the protein further comprises a tag (e.g., a His or flag tag). In another embodiment, the transgene comprises a nucleotide sequence encoding a protein comprising (or consisting of) the ectodomain of a SARS-CoV-2 spike protein. In certain embodiments, the protein further comprises a tag (e.g., a His or flag tag). In some embodiments, the protein further comprises tetramerization domain and optionally a tag. In a specific embodiment, the transgene comprises a nucleotide sequence that encodes a SARS-CoV-2 spike protein or a portion thereof comprising the amino acid sequence set forth in SEQ ID NO:5, 7, 9 or 11. Due to the degeneracy of the nucleic acid code, multiple different nucleic acid sequences may encode for the same SARS-CoV-2 spike protein or a portion thereof (e.g., the ectodomain or receptor binding domain of the SARS-CoV-2 spike protein). In one embodiment, described herein is a recombinant NDV comprising a packaged genome comprising a transgene that comprises a nucleotide sequence encoding a SARS-CoV-2 spike protein or a portion thereof (e.g., the ectodomain or receptor binding domain of the SARS-CoV-2 spike protein), wherein the transgene comprises an RNA sequence corresponding to the negative sense of the cDNA sequence of SEQ ID NO:4, 6, 8, or 10. In another embodiment described herein is a recombinant NDV comprising a packaged genome comprising a transgene that comprises a nucleotide sequence encoding a SARS-CoV-2 spike protein or a portion thereof (e.g., the ectodomain or receptor binding domain of the SARS-CoV-2 spike protein) comprising the amino acid sequence set forth in SEQ ID NO:5, 7, 9 or 11. In another embodiment, described herein is a recombinant NDV comprising a packaged genome comprising a transgene that comprises a nucleotide sequence encoding a SARS-CoV-2 spike protein or a portion thereof (e.g., the ectodomain or receptor binding domain of the SARS-CoV-2 spike protein), wherein the transgene comprises an RNA sequence corresponding to the negative sense of the cDNA sequence of SEQ ID NO:4, 6, 8, or 10. In a preferred embodiment, a transgene comprises a codon optimized version of a nucleic acid sequence encoding a SARS-CoV-2 spike protein or a portion thereof (e.g., the ectodomain or receptor binding domain of the SARS-CoV-2 spike protein). In a specific embodiment, the SARS-CoV-2 spike protein or a portion thereof (e.g., the ectodomain or receptor binding domain of the SARS-CoV-2 spike protein) is expressed by cells infected with the recombinant NDV. In certain embodiments, the SARS-CoV-2 spike protein is incorporated into the virion of the recombinant NDV.

In a specific embodiment, described herein are recombinant NDV comprising a packaged genome, wherein the packaged genome comprises a transgene comprising a codon optimized nucleic acid sequence encoding a SARS-CoV-2 spike protein or a portion thereof (e.g., the ectodomain or receptor binding domain of the SARS-CoV-2 spike protein). In a specific embodiment, the SARS-CoV-2 spike protein or a portion thereof (e.g., the ectodomain or receptor binding domain of the SARS-CoV-2 spike protein) protein is expressed by cells infected with the recombinant NDV.

In another embodiment, described herein are recombinant NDV comprising a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains. In other words, the NDV F protein transmembrane and cytoplasmic domains replace the SARS-CoV-2 spike protein transmembrane and cytoplasmic domains so that the chimeric F protein does not include the SARS-CoV-2 spike protein transmembrane and cytoplasmic domains. In one embodiment, the transgene encodes a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In a specific embodiment, the transgene encodes a chimeric F protein comprising the amino acid sequence set forth in SEQ ID NO:13. In another embodiment, described herein are recombinant NDV comprising a packaged genome, wherein the packaged genome comprises a transgene encoding a chimeric F protein, wherein the chimeric F protein comprises a SARS-CoV-2 protein ectodomain and NDV F protein transmembrane and cytoplasmic domains, and wherein the transgene comprises an RNA sequence corresponding to the negative sense of the cDNA sequence of SEQ ID NO:12. In a preferred embodiment, a transgene comprises a codon optimized version of a nucleic acid sequence encoding the SARS-CoV-2 spike protein ectodomain. In a preferred embodiment, a transgene comprises a codon optimized version of a nucleic acid sequence encoding the SARS-CoV-2 spike protein ectodomain, wherein the SARS-CoV-2 spike protein ectodomain lacks a polybasic cleavage site. In another embodiment, a transgene comprises a codon optimized version of a nucleic acid sequence enoding a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In another embodiment, provided herein is a recombinant NDV comprising a chimeric F protein in its virion, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In a specific embodiment, the chimeric F protein comprises the amino acid sequence set forth in SEQ ID NO:13.

In another embodiment, provided herein is a recombinant NDV comprising a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In specific emboidments, the nucleic acid sequence encoding the chimeric F protein is codon optimized. In another embodiment, provided herein is a recombinant NDV comprising a packaged genome, wherein the packaged genome comprises a transgene that comprises a nucleotide sequence encoding a chimeric F protein, wherein the chimeric F protein comprises a SARS-CoV-2 spike protein ectodomain and an NDV F protein transmembrane and cytoplasmic domains, wherein the transgene comprises an RNA sequence corresponding to the negative sense of the cDNA sequence of SEQ ID NO: 14. In another embodiment, provided herein is a recombinant NDV comprising a packaged genome, wherein the packaged genome comprises a transgene that comprises a nucleotide sequence encoding a chimeric F protein, wherein the chimeric F protein comprises a SARS-CoV-2 spike protein ectodomain and an NDV F protein transmembrane and cytoplasmic domains, wherein the transgene comprises an RNA sequence corresponding to the negative sense of the cDNA sequence of SEQ ID NO: 16. In another embodiment, provided herein is a recombinant NDV comprising a packaged genome, wherein the packaged genome comprises a transgene that comprises a nucleotide sequence encoding a chimeric F protein, wherein the chimeric F protein comprises a SARS-CoV-2 spike protein ectodomain and an NDV F protein transmembrane and cytoplasmic domains, wherein the transgene comprises an RNA sequence corresponding to the negative sense of the cDNA sequence of SEQ ID NO: 18. In another embodiment, provided herein is a recombinant NDV comprising a packaged genome, wherein the packaged genome comprises a transgene that comprises a nucleotide sequence encoding a chimeric F protein, wherein the chimeric F protein comprises a SARS-CoV-2 spike protein ectodomain and an NDV F protein transmembrane and cytoplasmic domains, and wherein the transgene comprises an RNA sequence encoding the amino acid sequence set forth in SEQ ID NO: 15. In another embodiment, provided herein is a recombinant NDV comprising a packaged genome, wherein the packaged genome comprises a transgene that comprises a nucleotide sequence encoding a chimeric F protein, wherein the chimeric F protein comprises a SARS-CoV-2 spike protein ectodomain and an NDV F protein transmembrane and cytoplasmic domains, and wherein the transgene comprises an RNA sequence encoding the amino acid sequence set forth in SEQ ID NO: 17. In another embodiment, provided herein is a recombinant NDV comprising a packaged genome, wherein the packaged genome comprises a transgene that comprises a nucleotide sequence encoding a chimeric F protein, wherein the chimeric F protein comprises a SARS-CoV-2 spike protein ectodomain and an NDV F protein transmembrane and cytoplasmic domains, and wherein the transgene comprises an RNA sequence encoding the amino acid sequence set forth in SEQ ID NO: 19. In another embodiment, a transgene comprises a codon optimized version of a nucleic acid sequence enoding a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In another embodiment, provided herein is a recombinant NDV comprising a chimeric F protein in its virion, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In some embodiments, the NDV F protein transmembrane and cytoplasmic domains are fused to directly to the SARS-CoV-2 spike protein ectodomain. In a specific embodiment, the chimeric F protein comprises the amino acid sequence set forth in SEQ ID NO: 15. In another specific embodiment, the chimeric F protein comprises the amino acid sequence set forth in SEQ ID NO: 17. In another specific embodiment, the chimeric F protein comprises the amino acid sequence set forth in SEQ ID NO: 19.

In a specific embodiment, described herein are recombinant NDV comprising a packaged genome, wherein the packaged genome comprises a transgene encoding a chimeric F protein, wherein the chimeric F protein comprises a SARS-CoV-2 protein ectodomain and NDV F protein transmembrane and cytoplasmic domains, and wherein the ectodomain of the SARS-CoV-2 spike protein is encoded by a codon optimized nucleic acid sequence. In a preferred embodiment, described herein is a recombinant NDV comprising a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains, and wherein the transgene comprises an RNA sequence corresponding to the negative sense of the cDNA sequence of SEQ ID NO:12. In a specific embodiment, the chimeric F protein and NDV F protein are expressed by cells infected with the recombinant NDV. In another specific embodiment, the chimeric F protein is expressed by cells infected with the recombinant NDV and the chimeric F protein is incorporated into the NDV virion.

The recombinant NDV may have the backbone of any NDV type or strain, including, but not limited to, naturally-occurring strains, variants or mutants, mutagenized viruses, reassortants or genetically engineered viruses, or any combination thereof. In a specific embodiment, the recombinant NDV comprises an NDV backbone which is lentogenic. In another specific embodiment, the recombinant NDV comprises an NDV backbone of the NDV LaSota strain. See, .e.g., SEQ ID NO: 1 for a cDNA sequence of the genomic sequence of NDV LaSota strain. See also SEQ ID NO:25 for another cDNA sequence of the genomic sequence of NDV. In another specific embodiment, the recombinant NDV comprises an NDV backbone of the NDV Hitchner B1 strain. See, .e.g., SEQ ID NO:2 for a cDNA sequence of the genomic sequence of NDV Hitchner strain. In another specific embodiment, the recombinant NDV comprises an NDV backbone of a lentogenic strain other than the NDV Hitchner B1 strain.

The transgene encoding a SARS-CoV-2 spike protein or a chimeric F protein may be incorporated into the genome of any NDV type or strain. In a specific embodiment, the transgene is incorporated into the genome of a lentogenic NDV. In another specific embodiment, the transgene is incorporated in the genome of NDV strain LaSota. See, .e.g., SEQ ID NO: 1 for a cDNA sequence of the genomic sequence of NDV LaSota strain. See also SEQ ID NO:25 for another cDNA sequence of the genomic sequence of NDV. Another example of an NDV strain into which the transgene may be incorporated is the NDV Hitchner B1 strain. In a specific embodiment, the transgene may be incorporated into the genomic sequence of NDV Hitchner B1 strain. See, e.g., SEQ ID NO:2 for a cDNA sequence of the genomic sequence of NDV Hitchner B1 strain. In a specific embodiment, the transgene may be incorporated into the genome of a lentogenic strain other than the NDV Hitchner B1 strain. The transgene may be incorporated into the NDV genome between two transcription units (e.g., between NDV P and M genes).

In a specific embodiment, a transgene comprising a nucleotide sequence encoding a SARS-CoV-2 spike protein or portion thereof (e.g., the ectodomain or receptor binding domain of the SARS-CoV-2 spike protein) is incorporated into the genome of any NDV type or strain (e.g., NDV LaSota strain). The transgene comprising a nucleotide sequence encoding a SARS-CoV-2 spike protein may be incorporated between any two NDV transcription units (e.g., between NDV P and M genes). In certain embodiment, the genome of the recombinant NDV does not comprise a heterologous sequence encoding a heterologous protein other than the SARS-CoV-2 spike protein or a portion thereof (e.g., the ectodomain or receptor binding domain of a SARS-CoV-2 spike protein). In some embodiments, the genome of the recombinant NDV does not comprise a transgene other than a transgene comprising a nucleotide sequence encoding a SARS-CoV-2 spike protein or a portion thereof (e.g., the ectodomain or receptor binding domain of a SARS-CoV-2 spike protein). In certain embodiments, the genome of the recombinant NDV comprises a transgene comprising a nucleotide sequence encoding a SARS-CoV-2 spike protein or a portion thereof (e.g., the ectodomain or receptor binding domain of a SARS-CoV-2 spike protein) or chimeric F protein, and a transgene comprising a nucleotide sequence encoding a SARS-CoV-2 nucleocapsid protein, wherein the chimeric F protein comprises a SARS-CoV-2 spike protein ectodomain and NDV F protein transmembrane and cytoplasmic domains. In such embodiments, the genome of the recombinant NDV may not comprise any additional transgenes.

In a specific embodiment, a transgene comprising a nucleotide sequence encoding a chimeric F protein is incorporated into the genome of any NDV type or strain (e.g., NDV LaSota strain), wherein the chimeric F protein comprises a SARS-CoV-2 spike protein ectodomain and NDV F protein transmembrane and cytoplasmic domains. The transgene comprising a nucleotide sequence encoding a chimeric F protein may be incorporated between any two NDV transcription units (e.g., between NDV P and M genes). In certain embodiment, the genome of the recombinant NDV does not comprise a heterologous sequence encoding a heterologous protein other than the chimeric F protein, wherein the chimeric F protein comprises a SARS-CoV-2 spike protein ectodomain and NDV F protein transmembrane and cytoplasmic domains. In some embodiments, the genome of the recombinant NDV does not comprise a transgene other than a transgene comprising a nucleotide sequence encoding a chimeric F protein, wherein the chimeric F protein comprises a SARS-CoV-2 spike protein ectodomain and NDV F protein transmembrane and cytoplasmic domains.

In a specific embodiment, a transgene comprising a nucleotide sequence encoding a chimeric F protein is incorporated into the genome of any NDV type or strain (e.g., NDV LaSota strain), wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. The transgene comprising a nucleotide sequence encoding a chimeric F protein may be incorporated between any two NDV transcription units (e.g., between NDV P and M genes). In certain embodiment, the genome of the recombinant NDV does not comprise a heterologous sequence encoding a heterologous protein other than the chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site. In some embodiments, the genome of the recombinant NDV does not comprise a transgene other than a transgene comprising a nucleotide sequence encoding a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site.

In a specific embodiment, a transgene comprising a nucleotide sequence encoding a chimeric F protein is incorporated into the genome of any NDV type or strain (e.g., NDV LaSota strain), wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. The transgene comprising a nucleotide sequence encoding a chimeric F protein may be incorporated between any two NDV transcription units (e.g., between NDV P and M genes). In certain embodiment, the genome of the recombinant NDV does not comprise a heterologous sequence encoding a heterologous protein other than the chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. In some embodiments, the genome of the recombinant NDV does not comprise a transgene other than a transgene comprising a nucleotide sequence encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.

In another aspect, provided herein are compositions (e.g., immunogenic compositions) comprising a recombinant NDV described herein. In some embodiments, the recombinant NDV is a live virus. In other embodiments, the recombinant NDV is inactivated. The recombinant NDV may be inactivated using techniques knowns to one of skill in the art or described herein (see, e.g., Section 10, infra). A composition (e.g., immunogenic compositions) may further comprise pharmaceutically acceptable carrier. In certain embodiments, a composition (e.g., immunogenic compositions) may further comprise an adjuvant known to one of skill in the art or described herein (see, e.g., Section 10 or 11, infra). The compositions may be used in a method to induce an immune response to SARS-CoV-2 spike protein, to immunize against SARS-CoV-2, and/or to prevent COVID-19.

In another aspect, presented herein are methods for inducing an immune response to a SARS-CoV-2 spike protein comprising administering to a subject (e.g., a human subject) a recombinant NDV described herein or a composition comprising a recombinant NDV described herein. The composition may comprise an inactivated NDV, such as described in Section 10 or 11. Alternatively, the composition may comprise live NDV. See, e.g., Section 5.4 regarding compositions.

In another aspect, presented herein are methods for inducing an immune response to a SARS-CoV-2 spike protein comprising administering to a subject (e.g., a human subject) a recombinant NDV, wherein the recombinant NDV comprises a packaged genome comprising a transgene that comprises a nucleotide sequence encoding a SARS-CoV-2 spike protein or portion thereof (e.g., the ectodomain or receptor binding domain of the SARS-CoV-2 spike protein). In one embodiment, the SARS-CoV-2 spike protein or portion thereof (e.g., the ectodomain or receptor binding domain of the SARS-CoV-2 spike protein) comprises the amino acid sequence set forth in SEQ ID NO:5, 7, 9, or 11. Due to the degeneracy of the nucleic acid code, a number of different nucleic acid sequences may encode for the same SARS-CoV-2 spike protein or portion thereof (e.g., the ectodomain or receptor binding domain of the SARS-CoV-2 spike protein). In some embodiments, the SARS-CoV-2 spike protein or portion thereof (e.g., the ectodomain or receptor binding domain of the SARS-CoV-2 spike protein) is encoded a nucleotide sequence comprising the nucleotide sequence set forth SEQ ID NO: 4, 6, 8, or 10. In a specific embodiment, presented herein are methods for inducing an immune response to a SARS-CoV-2 spike protein comprising administering to a subject (e.g., a human subject) a recombinant NDV, wherein the recombinant NDV comprises a packaged genome comprising a transgene, and wherein the transgene comprises a codon optimized nucleic acid sequence encoding a SARS-CoV-2 spike protein or portion thereof (e.g., the ectodomain or receptor binding domain of the SARS-CoV-2 spike protein). In a specific embodiment, the SARS-CoV-2 spike protein or portion thereof (e.g., the ectodomain or receptor binding domain of the SARS-CoV-2 spike protein) is expressed by cells infected with the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for inducing an immune response to a SARS-CoV-2 spike protein or portion thereof (e.g., the ectodomain or receptor binding domain of the SARS-CoV-2 spike protein) comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains. In a specific embodiment, presented herein are methods for inducing an immune response to a SARS-CoV-2 spike protein or portion thereof (e.g., the ectodomain or receptor binding domain of the SARS-CoV-2 spike protein) comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In one embodiment, the chimeric F protein comprises the amino acid sequence set forth in SEQ ID NO: 13. Due to the degeneracy of the nucleic acid code, a number of different nucleic acid sequences may encode for the same chimeric F protein. For example, the chimeric F protein may be encoded by a sequence comprising the nucleotide sequence set forth in SEQ ID NO:12. In a specific embodiment, presented herein are methods for inducing an immune response to a SARS-CoV-2 protein comprising administering to a subject (e.g., a human subject) a recombinant NDV, wherein the recombinant NDV comprises a packaged genome comprising a transgene that comprises a nucleotide sequence encoding a chimeric F protein, wherein the chimeric F protein comprise a SARS-CoV-2 spike protein ectodomain and NDV F protein transmembrane and cytoplasmic domains, and wherein the transgene comprises a codon optimized nucleic acid sequence encoding the SARS-CoV-2 spike protein ectodomain. In a specific embodiment, presented herein are methods for inducing an immune response to a SARS-CoV-2 protein comprising administering to a subject (e.g., a human subject) a recombinant NDV, wherein the recombinant NDV comprises a packaged genome comprising a transgene that comprises a nucleotide sequence encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In a specific embodiment, the chimeric F protein is expressed by cells infected with the recombinant NDV. In another specific embodiment, the chimeric F protein is incorporated into the virion of the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for inducing an immune response to a SARS-CoV-2 spike protein, comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In one embodiment, the chimeric F protein comprises the amino acid sequence set forth in SEQ ID NO: 15. In another embodiment, the chimeric F protein comprises the amino acid sequence set forth in SEQ ID NO: 17. In another embodiment, the chimeric F protein comprises the amino acid sequence set forth in SEQ ID NO: 19. Due to the degeneracy of the nucleic acid code, a number of different nucleic acid sequences may encode for the same chimeric F protein. For example, the chimeric F protein may be encoded by a sequence comprising the nucleotide sequence set forth in SEQ ID NO:14. In another example, the chimeric F protein may be encoded by a sequence comprising the nucleotide sequence of SEQ ID NO:16. In another example, the chimeric F protein may be encoded by a sequence comprising the nucleotide sequence of SEQ ID NO:18. In a specific embodiment, the chimeric F protein is expressed by cells infected with the recombinant NDV. In another specific embodiment, the chimeric F protein is incorporated into the virion of the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for immuniziang against SARS-CoV-2 comprising administering to a subject (e.g., a human subject) a recombinant NDV described herein or a composition comprising a recombinant NDV described herein. The composition may comprise an inactivated NDV, such as described in Section 10 or 11. Alternatively, the composition may comprise live NDV. See, e.g., Section 5.4 regarding compositions.

In another aspect, presented herein are methods for immunizing against SARS-CoV-2 comprising administering to a subject (e.g., a human subject) a recombinant NDV, wherein the recombinant NDV comprises a packaged genome comprising a transgene that comprises a nucleotide sequence encoding a SARS-CoV-2 spike protein or portion thereof (e.g., the ectodomain or receptor binding domain of the SARS-CoV-2 spike protein). In one embodiment, the SARS-CoV-2 spike protein or portion thereof (e.g., the ectodomain or receptor binding domain of the SARS-CoV-2 spike protein) comprises the amino acid sequence set forth in SEQ ID NO: 5, 7, 9, or 11. Due to the degeneracy of the nucleic acid code, a number of different nucleic acid sequences may encode for the same SARS-CoV-2 spike protein or portion thereof (e.g., the ectodomain or receptor binding domain of the SARS-CoV-2 spike protein). In some embodiments, the SARS-CoV-2 spike protein or portion thereof (e.g., the ectodomain or receptor binding domain of the SARS-CoV-2 spike protein) is encoded by a nucleic acid sequence comprising the sequence of SEQ ID NO:4, 6, 8 or 10. In a specific embodiment, presented herein are methods for immunizing against SARS-CoV-2 comprising administering to a subject (e.g., a human subject) a recombinant NDV, wherein the recombinant NDV comprises a packaged genome comprising a transgene, and wherein the transgene comprises a codon optimized nucleic acid sequence encoding a SARS-CoV-2 spike protein or portion thereof (e.g., the ectodomain or receptor binding domain of the SARS-CoV-2 spike protein). In a specific embodiment, the SARS-CoV-2 spike protein or portion thereof (e.g., the ectodomain or receptor binding domain of the SARS-CoV-2 spike protein) is expressed by cells infected with the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for immunizing against SARS-CoV-2 comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains. In a specific embodiment, presented herein are methods for immunizing against SARS-CoV-2, comprising administering to a subject (e.g., a human subject) a recombinant NDV, wherein the recombinant NDV comprises a packaged genome comprising a transgene that comprises a nucleotide sequence encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In one embodiment, the chimeric F protein comprises the amino acid sequence set forth in SEQ ID NO:13. Due to the degeneracy of the nucleic acid code, a number of different nucleic acid sequences may encode for the same chimeric F protein. For example, the chimeric F protein may be encoded by a nucleotide sequence comprising the sequence of SEQ ID NO:12. In a specific embodiment, presented herein are methods for immunizing against SARS-CoV-2 comprising administering to a subject (e.g., a human subject) a recombinant NDV, wherein the recombinant NDV comprises a packaged genome comprising a transgene encoding a chimeric F protein, wherein the chimeric F protein comprise a SARS-CoV-2 spike protein ectodomain and NDV F protein transmembrane and cytoplasmic domains, and wherein the transgene comprises a codon optimized nucleic acid sequence encoding the SARS-CoV-2 spike protein ectodomain. In a specific embodiment, the chimeric F protein is expressed by cells infected with the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods of immunizing against SARS-CoV-2, comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In one embodiment, the chimeric F protein comprises the amino acid sequence set forth in SEQ ID NO: 15. In another embodiment, the chimeric F protein comprises the amino acid sequence set forth in SEQ ID NO: 17. In another embodiment, the chimeric F protein comprises the amino acid sequence set forth in SEQ ID NO: 19. Due to the degeneracy of the nucleic acid code, a number of different nucleic acid sequences may encode for the same chimeric F protein. For example, the chimeric F protein may be encoded by a sequence comprising the nucleotide sequence set forth in SEQ ID NO:14. In another example, the chimeric F protein may be encoded by a sequence comprising the nucleotide sequence set forth in SEQ ID NO:16. In another example, the chimeric F protein may be encoded by a sequence comprising the nucleotide sequence set forth in SEQ ID NO: 18. In a specific embodiment, the chimeric F protein is expressed by cells infected with the recombinant NDV. In another specific embodiment, the chimeric F protein is incorporated into the virion of the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for preventing COVID-19 comprising administering to a subject (e.g., a human subject) a recombinant NDV described herein or a composition comprising a recombinant NDV described herein. The composition may comprise an inactivated NDV, such as described in Section 10 or 11. Alternatively, the composition may comprise live NDV. See, e.g., Section 5.4 regarding compositions.

In another aspect, presented herein are methods for the prevention of COVID-19 comprising administering to a subject (e.g., a human subject) a recombinant NDV, wherein the recombinant NDV comprises a packaged genome comprising a transgene that comprises a nucleotide sequence encoding a SARS-CoV-2 spike protein or portion thereof (e.g., the ectodomain or receptor binding domain of the SARS-CoV-2 spike protein). In one embodiment, the SARS-CoV-2 spike protein or portion thereof (e.g., the ectodomain or receptor binding domain of the SARS-CoV-2 spike protein) comprises the amino acid sequence set forth in SEQ ID NO: 5, 7, 9, or 11. Due to the degeneracy of the nucleic acid code, a number of different nucleic acid sequences may encode for the same SARS-CoV-2 spike protein or portion thereof (e.g., the ectodomain or receptor binding domain of the SARS-CoV-2 spike protein). In some embodiments, the SARS-CoV-2 spike protein or portion thereof (e.g., the ectodomain or receptor binding domain of the SARS-CoV-2 spike protein) is encoded by a nucleic acid sequence comprising the sequence of SEQ ID NO:4, 6, 8 or 10. In a specific embodiment, presented herein are methods for the prevention of COVID-19 comprising administering to a subject (e.g., a human subject) a recombinant NDV, wherein the recombinant NDV comprises a packaged genome comprising a transgene, and wherein the transgene comprises a codon optimized nucleic acid sequence encoding a SARS-CoV-2 spike protein or portion thereof (e.g., the ectodomain or receptor binding domain of the SARS-CoV-2 spike protein). In a specific embodiment, the SARS-CoV-2 spike protein or portion thereof (e.g., the ectodomain or receptor binding domain of the SARS-CoV-2 spike protein)is expressed by cells infected with the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for the prevention of COVID-19 comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains. In a specific embodiment, presented herein are methods for the prevention of COVID-19, comprising administering to a subject (e.g., a human subject) a recombinant NDV, wherein the recombinant NDV comprises a packaged genome comprising a transgene that comprises a nucleotide sequence encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In one embodiment, the chimeric F protein comprises the amino acid sequence set forth in SEQ ID NO: 13. Due to the degeneracy of the nucleic acid code, a number of different nucleic acid sequences may encode for the same chimeric F protein. For example, the chimeric F protein may be encoded by a nucleotide sequence comprising the sequence of SEQ ID NO:12. In a specific embodiment, presented herein are methods for the prevention of COVID-19 comprising administering to a subject (e.g., a human subject) a recombinant NDV, wherein the recombinant NDV comprises a packaged genome comprising a transgene encoding a chimeric F protein, wherein the chimeric F protein comprise a SARS-CoV-2 spike protein ectodomain and NDV F protein transmembrane and cytoplasmic domains, and wherein the transgene comprises a codon optimized nucleic acid sequence encoding the SARS-CoV-2 spike protein ectodomain. In a specific embodiment, the chimeric F protein is expressed by cells infected with the recombinant NDV. In another specific embodiment, the chimeric F protein is incorporated into the virion of the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, presented herein are methods for the prevention of COVID-19, comprising administering to a subject (e.g., a human subject) a recombinant NDV, wherein the recombinant NDV comprises a packaged genome comprising a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In some embodiments, the NDV F protein transmembrane and cytoplasmic domains are fused to the SARS-CoV-2 spike protein ectodomain via a linker. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In one embodiment, the chimeric F protein comprises the amino acid sequence set forth in SEQ ID NO: 15. In another embodiment, the chimeric F protein comprises the amino acid sequence set forth in SEQ ID NO: 17. In another embodiment, the chimeric F protein comprises the amino acid sequence set forth in SEQ ID NO: 19. Due to the degeneracy of the nucleic acid code, a number of different nucleic acid sequences may encode for the same chimeric F protein. For example, the chimeric F protein may be encoded by a sequence comprising the nucleotide sequence set forth in SEQ ID NO:14. In another example, the chimeric F protein may be encoded by a sequence comprising the nucleotide sequence set forth in SEQ ID NO:16. In another example, the chimeric F protein may be encoded by a sequence comprising the nucleotide sequence set forth in SEQ ID NO: 18. In a specific embodiment, the chimeric F protein is expressed by cells infected with the recombinant NDV. In another specific embodiment, the chimeric F protein is incorporated into the recombinant NDV. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

In another aspect, provided herein is a recombinant NDV comprising a packaged genome that comprises a transgene comprising a nucleotide sequence encoding a SARS-CoV-2 nucleocapid. In a specific embodiment, the recombinant NDV is a composition, such as described in Section 5.4. In another aspect, provided herein is a method for inducing an immune response to SARS-CoV-2 nucleocapsid comprising administering to a subject (e.g., a human subject) a recombinant NDV comprising a packaged genome that comprises a transgene comprising a nucleotide sequence encoding a SARS-CoV-2 nucleocapid. In another aspect, provided herein is a method for immunizing a subject (e.g., a human subject) against SARS-CoV-2 comprising administering to the subject (e.g., a human subject) a recombinant NDV comprising a packaged genome that comprises a transgene comprising a nucleotide sequence encoding a SARS-CoV-2 nucleocapid. In another aspect, provided herein is a method for preventing COVID-19 in a subject (e.g., a human subject) SARS-CoV-2 nucleocapsid comprising administering to the subject (e.g., a human subject) a recombinant NDV comprising a packaged genome that comprises a transgene comprising a nucleotide sequence encoding a SARS-CoV-2 nucleocapid. In another specific embodiment, the recombinant NDV is administered to a subject intranasally or intramuscularly. In another specific embodiment, the subject is a human infant. In another specific embodiment, the subject is a human infant six months old or older. In another specific embodiment, the subject is a human toddler. In another specific embodiment, the subject is a human child. In another specific embodiment, the subject is a human adult. In another specific embodiment, the subject is an elderly human.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally or intramusularly. See, e.g., Sections 5.1, and 6-12, infra for information regarding recombinant NDV, Section 5.5.3 for information regarding other therapies, Section 5.4, infra, for information regarding compositions and routes of administration, and Sections 5.5.1 and 6, 7, 8, 10 and 11, infra, for information regarding methods of immunizing against SARS-CoV-2.

In another aspect, provided herein is a nucleotide sequence comprising an NDV genome and a transgene described herein. The nucleotide sequence may comprise a nucleic acid sequence of an NDV genome known in the art or described (see, e.g., Section 5.1 or the Examples below; see also SEQ ID NO: 1, 2 or 25) and a nucleic acid sequence of a transgene described herein. In a specific embodiment, the nucleotide sequence is isolated.

In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

In one embodiment, provided herein is a nucleotide sequence comprising an NDV genome and a transgene, wherein the transgene comprises a codon-optimized nucleotide sequence of a SARS-CoV-2 spike protein or a portion thereof (e.g., the receptor binding domain or ectodomain of the SARS-CoV-2 spike protein), and a gene end sequence, a gene start sequence and a Kozak sequence at the 5′ end. See, e.g., SEQ ID NOS: 21-23 for examples of a gene end sequence, a gene start sequence and a Kozak sequence that may be used. In certain embodiments, the additional nucleotides are present at the 3′ end in order to follow the “rule of six.” In a specific embodiment, the SARS-CoV-2 spike protein or a portion thereof comprises the amino acid sequence of SEQ ID NO:5, 7, 9 or 11. In some embodiments, the transgene is between the NDV P and M genes. In a specific embodiment, the nucleotide sequence is isolated.

In another embodiment, provided herein is a nucleotide sequence comprising an NDV genome and a transgene, wherein the transgene comprises a codon-optimized comprises a nucleotide sequence encoding a chimeric F protein and a gene end sequence, a gene start sequence and a Kozak sequence at the 5′ end, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the additional nucleotides are present at the 3′ end in order to follow the “rule of six.” In a specific embodiment, the chimeric F protein comprises the amino acid sequence of SEQ ID NO:13. In some embodiments, the transgene is between the NDV P and M genes. In a specific embodiment, the nucleotide sequence is isolated.

In another embodiment, provided herein is a nucleotide sequence comprising an NDV genome and a transgene, wherein the transgene comprises a codon-optimized comprises a nucleotide sequence encoding a chimeric F protein and a gene end sequence, a gene start sequence and a Kozak sequence at the 5′ end, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the additional nucleotides are present at the 3′ end in order to follow the “rule of six.” In a specific embodiment, the chimeric F protein comprises the amino acid sequence of SEQ ID NO:15. In another specific embodiment, the chimeric F protein comprises the amino acid sequence of SEQ ID NO:17. In another specific embodiment, the chimeric F protein comprises the amino acid sequence of SEQ ID NO:19. In some embodiments, the transgene is between the NDV P and M genes. In a specific embodiment, the nucleotide sequence is isolated.

### 3.1 Terminology

As used herein, the term “about” or “approximately” when used in conjunction with a number refers to any number within 1, 5 or 10% of the referenced number.

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “heterologous” in the context of NDV refers an entity not found in nature to be associated with (e.g., encoded by, expressed by the genome of, or both) a naturally occurring NDV. In a specific embodiment, a heterologous sequence encodes a protein that is not found associated with naturally occurring NDV.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, are deficient in the activity of antiviral genes induced by one, two or more types of IFN, or induced by any type of IFN, or any combination thereof.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to, concomitantly with, or subsequent to the administration of a second therapy to a subject.

As used herein, the terms “SARS-CoV-2 nucleocapsid” refers to a SARS-CoV-2 nucleocapsid known to those of skill in the art. In certain embodiments, the nucleocapsid protein comprises the amino acid or nucleic acid sequence found at GenBank Accession No. MT081068.1, MT081066.1 or MN908947.3. See also, e.g., GenBank Accession Nos. MN908947.3, MT447160, MT44636, MT446360, MT444593, MT444529, MT370887, and MT334558 for examples of amino acid sequences of SARS-CoV-2 nucleocapsid protein and nucleotide sequences encoding SARS-CoV-2 nucleocapsid protein.

As used herein, the terms “SARS-CoV-2 spike protein” and “spike protein of SARS-CoV-2” refer to a SARS-CoV-2 spike protein known to those of skill in the art. See, e.g., GenBank Accession Nos. MN908947.3, MT447160, MT44636, MT446360, MT444593, MT444529, MT370887, and MT334558 for examples of amino acid sequences of SARS-CoV-2 spike protein and nucleotide sequences encoding SARS-CoV-2 spike protein. In certain embodiments, the spike protein comprises the amino acid or nucleic acid sequence found at GenBank Accession No. MN908947.3. A typical spike protein comprises domains known to those of skill in the art including an S1 domain, a receptor binding domain, an S2 domain, a transmembrane domain and a cytoplasmic domain. See, e.g., Wrapp et al., 2020, Science 367: 1260-1263 for a description of SARS-CoV-2 spike protein (in particular, the structure of such protein). The spike protein may be characterized has having a signal peptide (e.,g a signal peptide of 1-14 amino acid residues of the amino acid sequence of GenBank Accession No. MN908947.3), a receptor binding domain (e.g., a receptor binding domain of 319-541 amino acid residues of GenBank Accession No. MN908947.3), an ectodomain (e.g., an ectodomain of 15-1213 amino acid residues of GenBank Accession No. MN908947.3), and a transmembrane and endodomain (e.g., a transmembrane and endodomain of 1214-1273 amino acid residues of GenBank Accession No. MN908947.3).

As used herein, the terms “therapies” and “therapy” can refer to any protocol(s), method(s), agent(s) or a combination thereof that can be used in the treatment or prevention of COVID-19, or vaccination. In certain embodiments, the term “therapy” refers to a recombinant NDV described herein. In other embodiments, the term “therapy” refers to an agent that is not a recombinant NDV described herein.

As used herein, the term “wild-type” in the context of nucleotide and amino acid sequences refers to the nucleotide and amino acid sequences of viral strains found in nature. In particular, the sequences described as wild-type herein are sequences that have been reported in public databases as sequences from natural viral isolates.

### 4. BRIEF DESCRIPTION OF THE FIGURES

FIG. 1. Depiction of construction of NDV LaSota (LS) rescue plasmids. SEQ ID NOs. 20-23 provide the sequences of SacII restriction sequence, the gene end sequence (GE), the gene start sequence (GS) and a Kozak sequence. Adapted from Gayathri Vijayakumar and Dmitriy Zamarin, Christine E. Engeland (ed.), Oncolytic Viruses, Methods in Molecular Biology, vol. 2058.

FIG. 2. Depiction of the methodology used to rescue NDV expressing 1) the secreted RBD (S_RBD 6 × His), 2) the ectodomain of the spike (S_Ecto 6 × His), 3) the secreted RBD (S_RBD); 4) full-length spike (S); or 5) a modified chimeric spike (S-F), in which the ectodomain of the spike is fused to the transmembrane domain and cytoplasmic tail of the F protein of NDV. Adapted from Gayathri Vijayakumar and Dmitriy Zamarin, Christine E. Engeland (ed.), Oncolytic Viruses, Methods in Molecular Biology, vol. 2058.

FIGS. 3A-3B. FIG. 3A. The RNA of HA positive NDV_LS_RBD and NDV_LS_S_RBD 6×His samples were extracted and RT-PCR was performed using primers flanking the insertion to amplify the transgene. The results of amplification are shown on the gels. One example is shown for each construct. FIG. 3B. Allantoic fluid from eggs containing indicated viruses were coated onto ELISA plates. Binding assay was performed using anti-S_RBD monoclonal antibody CR3022 or anti-His tag monoclonal.

FIGS. 4A-4C. FIG. 4A. The RNA of HA positive samples were extracted. RT-PCR was performed using primers flanking the insertion site to amplify the transgene. The results of the amplification are shown on the gel. One example is shown. FIG. 4B. Vero E6 cells were infected with indicated viruses, the cells were fixed and stained with indicated antibody/antisera. FIG. 4C. CEF cells were infected with the indicated viruses, cell lysates were resolved onto SDS-PAGE, and protein expression was determined by Western blot using anti-spike antibody 2B3E5. Anti-NDV NP was used as control.

FIGS. 5A-5B. FIG. 5A. The RNA of HA positive NDV_LS_S_ecto 6× His samples were extracted and RT-PCR was performed using primers flanking the insertion site to amplify the transgene. The results of the amplification are shown on the gel. FIG. 5B. Allantoic fluid of NDV_LS_S_ecto_6×His were coated onto ELISA plates for screening for protein expression and the binding assay was performed using anti-S_RBD monoclonal antibody CR3022.

FIG. 6. Six well plates of CEF cells were infected with indicated viruses. Protein expression in cell lysates from such infected cells was determined Western blot. Four HA positive samples were tested for NDV_LS/L289A_S_ecto 6×His and NDV_LS/L289A_S-F.

FIG. 7. Depicts the use of NDV vectors expressing a SARS-CoV-2 spike protein or portion thereof (e.g., the ectodomain or receptor binding domain of SARS-CoV-2 spike protein) as vaccines.

FIGS. 8A-8C. NDV vectors expressing the spike protein of SARS-CoV-2. FIG. 8A. Two forms of spike proteins expressed by NDV. Spike (S) has the wild type amino acid sequence. The Spike-F chimera (S-F) consists of the ectodomain of S without the polybasic cleavage site and the transmembrane domain (TM) and cytoplasmic tail (CT) of the F protein from NDV. FIG. 8B. Illustration of genome structures of wild type NDV LaSota (WT NDV_LS), NDV expressing the S or S-F in the wild type LaSota backbone (NDV_LS_S or NDV_LS_S-F) or NDV expressing the S-F in the L289A mutant backbone (NDV_LS/L289A_S-F). The L289A mutation supports the HN-independent fusion of the F protein. FIG. 8C. Titers of NDV vectors grown in embryonated chicken eggs. The rescued viruses were grown in 10-day old embryonated chicken eggs for 2 or 3 days at 37° C. at limiting dilutions. The peak titers of each virus were determined by immunofluorescence assay (IFA).

FIGS. 9A-9B. Expression of spike protein in infected cells and NDV particles. FIG. 9A. Expression of the S and S-F protein in infected cells. Vero E6 cells were infected with three NDV vectors encoding the S or S-F for 16 to 18 hours. A WT NDV control was included. The next day, cells were fixed with methanol-free paraformaldehyde. Surface proteins were stained with anti-NDV rabbit serum or a spike receptor-binding domain (RBD)-specific monoclonal antibody CR3022. FIG. 9B. Incorporation of S and S-F into NDV particles. Three NDV vectors expressing the S or S-F including the NDV_LS_S (green), NDV_LS_S-F (red) and NDV_LS/L289A_S-F (blue) were concentrated through a 20% sucrose cushion. Two clones were shown for NDV_LS_S and NDV_LS_S-F. The concentrated WT NDV expressing no transgenes was used as a control. Two micrograms of each concentrated virus were resolved on a 4-20% SDS-PAGE, the spike protein and NDV HN protein were detected by western blot using an anti-spike 2B3E5 mouse monoclonal antibody and an anti-HN 8H2 mouse monoclonal antibody.

FIGS. 10A-10C. NDV vector vaccines elicit high titers of binding and neutralizing antibodies in mice. FIG. 10A. Vaccination groups and regimen. A prime-boost vaccination regimen was used with a three-week interval. Mice were bled pre-boost and 8 days after the boost. Mice were challenged with a mouse adapted SARS-CoV-2 MA strain 11 days after the boost. A total of ten groups of mice were used in a vaccination and challenge study. Group 1 (10 µg) and 2 (50 µg) received the WT NDV; Group 3 (10 µg) and 4 (50 µg) received the NDV_LS_S; Group 5 (10 µg) and 6 (50 µg) received NDV_LS_S-F; Group 7 (10 µg) and 8 (50 µg) received NDV_LS/L289A_S-F; Group 9 received PBS as negative controls. An age-matched healthy control group 10 was provided upon challenge. FIG. 10B. Spike-specific serum IgG titers measured by ELISAs. Sera from animals at 3 weeks after-prime (patterned bars) and 8 days after-boost (solid bars) were isolated. Serum IgG was measured against a recombinant trimeric spike protein by ELISAs. The endpoint titers were calculated as the readout for ELISAs. FIG. 10C. Neutralization titers of serum antibodies. Sera from 3 weeks after-prime and 8 days after-boost were pooled within each group. Technical duplicates were performed to measure neutralization activities of serum antibodies using a USA-WA1/2020 SARS-CoV-2 strain. The ID50 value was calculated as the readout of the neutralization assay. For the samples (WT NDV and PBS groups) showing no neutralizing activity in the assay, an ID50 of 10 was given as the starting dilution of the sera is 1:20 (LoD: limit of detection).

FIGS. 11A-11B. NDV vector vaccines protected mice from the SARS-CoV-2 challenge. FIG. 11A. Viral titers in the lungs. All mice were infected intranasally with 10⁴ PFU SARS-CoV-2 MA strain except the healthy control group, which was mock infected with PBS. At day 4 post-challenge, lungs were collected and homogenized in PBS. Viral titers in the lung homogenates were determined by plaque assay. Plaque-forming units (PFU) per lung lobe was calculated. Geometric mean titer was shown for all the groups. LoD: limit of detection. FIG. 11B. Immunohistochemistry (IHC) staining of lungs. A SARS-CoV-2 NP specific antibody was used for IHC to detect viral antigens. Slides were counterstained with hematoxylin. A presentative image was shown for each group. The brown staining indicates the presence of NP protein of SARS-CoV-2.

FIGS. 12A-12B. FIG. 12A. Immunization groups and regimen. C57BL/6 mice were vaccinated with 10⁵ ffu/mouse of NDV_LS_S, NDV_LS_S-F, NDV_LS/L289A_S-F or NDV_LS_RBD (secreted RBD was expressed as the transgene) intranasally (i.n.). Wild type NDV_LS was given to a group of mice at 10⁵ ffu/mouse as negative controls. Six weeks after the prime, each group of mice were bled and then boosted with the same virus at the same dose (10⁵ ffu/mouse). FIG. 12B. Serum IgG titers. Pre-boost (6 weeks after prime) serum IgG toward the full-length spike was measured by ELISAs.

FIG. 13. Viruses (WT NDV-LS, NDV_LS/L289A_S-F, NDV_LS/L289A_S-F HexaPro) and were concentrated through a 20% sucrose cushion. Protein content was determined by BCA. Five or ten micrograms of each virus was resolved on a 4-20% SDS-PAGE. The gel was stained with Coomassie G-250.

FIGS. 14A-14B. Design and concept of an inactivated NDV-based SARS-CoV-2 vaccine. FIG. 14A. Design of the NDV-S vaccine. The sequence of the S-F chimera (green: ectodomain of S; black: the transmembrane domain and cytoplasmic tail of NDV F protein) was inserted between the P and M gene of the NDV LaSota (NDV_LS) strain L289A mutant (NDV_LS/L289A). NDV-S: NDV_LS/L289A_S-F. The polybasic cleavage site of the S was removed (⁶⁸²RRAR⁶⁸⁵ to A). FIG. 14B The concept overview of a inactivated NDV-based SARS-CoV-2 vaccine. The NDV-S vaccine could be produced using current global influenza virus vaccine production capacity. Such an NDV-S vaccine displays abundant S protein on the surface of the virions. The NDV-S vaccine will be inactivated by betapropiolactone (BPL). The NDV-S vaccine will be administered intramuscularly (i.m.) to elicit protective antibody responses in humans.

FIGS. 15A-15C. The antigenicity of the S-F chimera is stable. FIG. 15A Stability of the S-F chimera. Allantoic fluid containing the NDV-S virus was aliquoted into equal amounts (15 ml) and stored at 4° C. Virus from each aliquot was concentrated through a 20% sucrose cushion, re-suspended in equal amount of PBS, and then stored at - 80° C. for several weeks (wk 0, wk 1, wk 2, wk 3). One microgram of each concentrated virus was resolved onto 4-20% SDS-PAGE. Protein degradation was evaluated by western blot using a S-specific mouse monoclonal antibody 2B3E5. HN protein of NDV was used as an NDV protein control. FIG. 15B. Antigenicity of the S-F before and after BPL inactivation. Live or inactivated (using 0.05% BPL) NDV-S virus was concentrated through a 20% sucrose cushion as described previously. Two micrograms of live or BPL inactivated virus were loaded onto 4-20% SDS-PAGE. Antigenicity loss of the S-F was evaluated by western blot as described in FIG. 15A. FIG. 15C. Inactivation of the virus by betapropiolactone (BPL). Viruses in the allantoic fluid were inactivated by 0.05% BPL, as described previously. Clarified allantoic fluids with live and inactivated viruses were diluted in PBS (at 1000-fold dilution) and inoculated into 10-day-old embryonated chicken eggs. The eggs were incubated at 37° C. for 3 days. The loss of infectivity of the inactivated virus was confirmed by the lack of growth of the virus determined by a hemagglutination (HA) assay.

FIGS. 16A-16C. Inactivated NDV-S vaccine elicited high antibody responses in mice. FIG. 16A. Immunization regimen of inactivated NDV-S vaccine in mice. BALB/c mice were given two immunizations via intramuscular administration route with a 2-week interval. Mice were bled pre-boost and 11 days after the boost for in vitro serological assays. Mice were challenged with a mouse-adapted SARS-CoV-2 strain 19 days after the boost. FIG. 16B. Spike-specific serum IgG titers. Serum IgG titers from animals after prime (pattern bars) and boost (solid bars) toward the recombinant trimeric spike protein was measured by ELISAs. Endpoint titers were shown as the readout for ELISAs. FIG. 16C. Neutralization titers of serum antibodies. Microneutralization assays were performed to determine the neutralizing activities of serum antibodies from animals after the boost (D26) using the USA-WA1/2020 SARS-CoV-2 strain. The ID₅₀ of serum samples showing no neutralizing activity (WT NDV) is set as 10. (LoD: limited of detection).

FIGS. 17A-17B. Inactivated NDV-S vaccine protects mice from SARS-CoV-2 infection. FIG. 17A. Weight loss of mice infected with SARS-CoV-2. Weight loss of mice challenged with a mouse-adapted SARS-CoV-2 strain were monitored for 4 days. FIG. 17B. Viral titers in the lung. Lungs of mice were harvested at day 4 post infection. Viral titers of the lung homogenates were determined by plaque assay. Geometric mean titer (PFU/lobe) was shown. (LoD: limit of detection)

FIGS. 18A-18D. Inactivated NDV-S vaccine attenuates SARS-CoV-2 induced diseases in hamsters. FIG. 18A. Immunization groups and regimen. Golden Syrian hamsters were vaccinated with inactivated NDV-S following a prime-boost regimen with a 2-week interval. Hamsters were challenge 24 days after the boost with the USA-WA1/2020 SARS-CoV-2 strain. Four groups of hamsters (n=8) were included in this study. Group 1 received 10 µg of inactivated NDV-S vaccine without any adjuvants. Group 2 received 5 µg of inactivated NDV-S vaccine adjuvanted with AddaVax. Group 3 receiving the 10 µg of inactivated WT NDV was included as vector-only (negative) control. Group 4 receiving no vaccine were mock challenged with PBS as healthy controls. FIG. 18 B. Spike-specific serum IgG titers. Hamsters were bled pre-boost and a subset of hamsters were terminally bled at 2 dpi. Vaccine-induced serum IgG titers towards the trimeric spike protein were determined by ELISAs. Endpoint titers were shown as the readout for ELISAs. FIG. 18C. Weight loss of hamsters challenged with SARS-CoV-2. Weight loss of SARS-CoV-2 infected hamsters were monitored for 5 days. FIG. 18D. Viral titers in the lungs. Viral titers in the upper right (UR) and lower right (LR) lung lobes of the animals at 2 and 5 dpi were measured by a plaque assay (LoD: limit of detection). Statistical analysis was performed using the Kruskal-Wallis test with Dunn’s correction for multiple comparisons. P-values between groups were shown.

FIGS. 19A-19B. Design of the NDV-HXP-S variants. FIG. 19A. Schematic illustration of the design of NDV-HXP-S construct. FIG. 19B. Mutations introduced into NDV-HXP-S (B.1.351) and NDV-HXP-S (P.1). The mutations were introduced into the hexaPro spike, in which the polybasic cleavage site was deleted (⁶⁸²RRAR⁶⁸⁵) and the transmembrane/cytoplasmic domains were replaced with those from NDV fusion protein. Deletions instead of amino acid substitutions are underlined.

FIGS. 20A-20B. Characterization of the NDV-HXP-S variants. FIG. 20A. SDS-PAGE of the concentrated NDV-HXP-S variants to identify the expression of the spike protein (Psg 3: one passage from the pre-MVS; psg 4: one passage from psg 3). One (psg 3) and/ or two passages (psg 4) of the B.1.351 clone 7-6 and P.1 clone 7-6 were harvested and concentrated through a 20% sucrose cushion via ultracentrifugation. Fifteen (15) micrograms of each virus was loaded. The gels were stained with Coomassie blue. FIG. 20B. Binding of monoclonal antibodies to the spikes expressed by the NDV-HXP-S variants by ELISAs. One passage (10^-6 dilution) of the B.1.351 clone 7-6 and P.1 clone 7-6 were harvested and concentrated through a 20% sucrose cushion via ultracentrifugation. WT and B.1.351 cross-reactive human mAbs 1D07 (RBD), 2B12 (NTD), and CR3022 (RBD), and a mouse mAb 3A7 (RBD) were tested against both purified NDV-HXP-S variants.

FIGS. 21A-21B. NDV-HXP-S variant with different mutation profiles. To explore mutations that contribute to the expression, stability, and integrity of the spike additional variants are rescued. FIG. 21A. Mutant NDV-HXP-S variants that have been rescued. FIG. 21B. Other NDV-HXP-S variants for rescue. Amino acid substitutions that are different from sequences in FIG. 19B are in bold. Deletions instead of amino acid substitutions are underlined.

FIG. 22. Viral titers in the lung. Lungs of mice were harvested at day 4 post-infection. Viral titers of the lung homogenates were determined by a plaque assay. Geometric mean titer (PFU/lobe) is shown (LoD: limit of detection). Statistical analysis was performed using the Kruskal-Wallis test with Dunn’s correction for multiple comparisons. P-values between groups were shown.

### 5. DETAILED DESCRIPTION

### 5.1 Recombinant Newcastle Disease Virus

In one aspect, provided herein are recombinant NDV described heren that may be used to immunize a subject (e.g., a human subject) against SARS-CoV-2. The recombinant NDV may be administered as a live virus or an inactivated virus. The data provided in the Examples demonstrates that utility of recombinant NDV described herein to immunize against SARS-CoV-2. For example, the data in Section 7, infra, demonstrates that high levels of neutralizing antibodies is achieved when recombinant NDV vector COVID-19 vaccines are administered to mice. In addition, when recombinant NDV COVID-19 vaccines are administered to mice, they protect the mice from mouse-adapted SARS-CoV-2 challenge with no detectable viral titer and viral antigen in the lungs. The data in Section 10, infra, demonstrates, e.g., that inactivated NDV chimera stably expressing the membrane-anchored form of the spike protein (NDV-S) as a potent COVID-19 vaccine in mice and hamsters. The inactivated NDV-S vaccine was immunogenic, inducing strong binding and/or neutralizing antibodies in both anmals. The inactivated NDV-S vaccine protected animals from SARS-CoV-2 infections. In the presence of an adjuvant, antigen-sparing could be achieved, which would potentially further ensure the low-cost of the vaccine when produced using the existing influenza virus vaccine capacity.

### 5.1.1 NDV

Any NDV type or strain may be serve as the “backbone” that is engineered to encode a transgene described herein, including, but not limited to, naturally-occurring strains, variants or mutants, mutagenized viruses, reassortants and/or genetically engineered viruses. See, e.g., Section 5.1.2 and Examples 6, 7, 9, 10 and 12 for examples of transgenes. In a specific embodiment, the nucleotide sequence is incorporated into the genome of a lentogenic NDV. In another specific embodiment, the nucleotide sequence is incorporated in the genome of NDV strain LaSota. In another example of an NDV strain into which the nucleotide sequence may be incorporated is the NDV Hitchner B1 strain. In some embodiments, a lentogenic strain other than NDV Hitchner B1 strain is used as the backbone into which a nucleotide sequence may be incorporated. The nucleotide sequence may be incorporated into the NDV genome between two transcription units (e.g., between the M and P transcription units or between the HN and L transcription units).

In a specific embodiment, the NDV that is engineered to encode a transgene described herein is a naturally-occurring strain. Specific examples of NDV strains include, but are not limited to, Hitchner B1 strain (see, e.g., GenBank No. AF309418 or NC_002617) and La Sota strain (see, e.g., GenBank Nos. AY845400, AF07761.1 and JF950510.1and GI No. 56799463). In a specific embodiment, the NDV that is engineered to encode a transgene described herein is the Hitchner B1 strain. In another embodiment, the NDV that is engineered to encode a transgene described herein is a B1 strain as identified by GenBank No. AF309418 or NC_002617. In a specific embodiment, the nucleotide sequence of the Hitchner B1 genome comprises an RNA sequence corresponding to the negative sense of the cDNA sequence set forth in SEQ ID NO:2. In another specific embodiment, the NDV that is engineered to encode a transgene described herein is the La Sota strain. In another embodiment, the NDV that is engineered to encode a transgene described herein is a LaSota strain as identified by AY845400, AF07761.1 or JF950510.1. In a specific embodiment, the nucleotide sequence of the La Sota genome comprises an RNA sequence corresponding to the negative sense of the cDNA sequence set forth in SEQ ID NO:1. In another specific embodiment, the nucleotide sequence of the La Sota genome comprises an RNA sequence corresponding to the negative sense of the cDNA sequence set forth in SEQ ID NO:25. One skilled in the art will understand that the NDV genomic RNA sequence is an RNA sequence corresponding to the negative sense of a cDNA sequence encoding the NDV genome. Thus, any program that generates converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Tables 1 and 2, infra, may be readily converted to the negative-sense RNA sequence of the NDV genome by one of skill in the art.

In a specific embodiment, the NDV that is engineered to encode a transgene described herein comprises a genome encoding an NDV F protein in which a leucine amino acid residue at amino acid position 289 of NDV F protein is substituted for alanine (as described by, e.g., Sergel et al., 2000, Journal of Virology 74: 5101-5107). In another specific embodiment, the NDV that is engineered to encode a transgene described herein comprises a genome encoding an NDV F protein in which a leucine amino acid residue at amino acid position 289 of NDV F protein (as counted by the LaSota strain F protein) is substituted for alanine. In another specific embodiment, the NDV that is engineered to encode a transgene described herein comprises a genome comprises a nucleotide sequence encoding an NDV F protein in which leucine at the amino acid position corresponding to amino acid residue 289 of LaSota NDV F protein is substituted for alanine. In another specific embodiment, the NDV that is engineered to encode a transgene described herein comprises a genome comprises a nucleotide sequence encoding an NDV F protein in which leucine at the amino acid residue 289 of LaSota NDV F protein is substituted for alanine. In another specific embodiment, the NDV that is engineered to encode a transgene described herein is the LaSota strain (e.g., GenBank Accession Nos. AY845400, AF07761.1 or JF950510.1) and the genome of the LaSota strain encodes an NDV F protein in which a leucine amino acid residue at amino acid position 289 of NDV F protein is substituted for alanine. In another specific embodiment, the NDV that is engineered to encode a transgene described herein is the LaSota strain (e.g., GenBank Accession Nos. AY845400, AF07761.1 or JF950510.1) and the genome of the LaSota strain comprises a nucleotide sequence encoding LaSota NDV F protein in which leucine at amino acid residue 289 of the NDV F protein is substituted for alanine. In another specific embodiment, the NDV that is engineered to encode a transgene described herein is the Hitchner B1 strain (e.g., GenBank No. AF309418 or NC_002617) and the genome of the Hitchner B1 strain encodes an NDV F protein in which a leucine amino acid residue at amino acid position 289 of NDV F protein is substituted for alanine.

In specific embodiments, the NDV that is engineered to encode a transgene described herein is not pathogenic in birds as assessed by a technique known to one of skill. In certain specific embodiments, the NDV that is engineered to encode a transgene described herein is not pathogenic as assessed by intracranial injection of 1-day-old chicks with the virus, and disease development and death as scored for 8 days. In some embodiments, the NDV that is engineered to encode a transgene described herein has an intracranial pathogenicity index of less than 0.7, less than 0.6, less than 0.5, less than 0.4, less than 0.3, less than 0.2 or less than 0.1. In certain embodiments, the NDV that is engineered to encode a transgene described herein has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14_NEWCASTLE_DIS.pdf, which is incorporated herein by reference in its entirety.

In certain embodiments, the NDV that is engineered to encode a transgene described herein is a mesogenic strain that has been genetically engineered so as not be a considered pathogenic in birds as assessed by techniques known to one skilled in the art.

In preferred embodiments, the NDV that is engineered to encode a transgene described herein is non-pathogenic in humans. In preferred embodiments, the NDV that is engineered to encode a transgene described herein is non-pathogenic in human and avians. In certain embodiments, the NDV that is engineered to encode a transgene described herein is attenuated such that the NDV remains, at least partially, infectious and can replicate in vivo, but only generate low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the NDV genome comprises sequences necessary for infection and replication of the virus such that progeny is produced and the infection level is subclinical.

In a specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein. In certain embodiments, the NDV transcription units are LaSota NDV transcription units. In a specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at the amino acid residue corresponding to amino acid position 289 of LaSota NDV F protein. In another specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at amino acid position 289 of LaSota NDV F protein. In certain embodiments, the NDV transcription units are LaSota NDV transcription units. In certain embodiments, the nucleic acid sequence is part of a vector (e.g., a plasmid, such as described in the Examples below). In specific embodiments, the nucleic acid sequence is isolated.

In a specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein. In another specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position corresponding to amino acid residue 289 of LaSota NDV F. In another specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position 289 of LaSota NDV F. In certain embodiments, the NDV proteins are LaSota NDV proteins. In another specific embodiment, provided herein is a nucleic acid sequence comprising a nucleotide sequence of an NDV genome known in the art or described (see, e.g., Section 5.1 or the Examples below; see also SEQ ID NO: 1, 2 or 25) and a transgene described herein. In certain embodiments, the nucleic acid sequence is part of a vector (e.g., a plasmid, such as described in the Examples below). In a specific embodiment, the nucleotide sequence is isolated.

In specific embodiments, a nucleic acid sequence or nucleotide sequence described herein is a recombinant nucleic acid sequence or recombinant nucleotide sequence. In certain embodiments, a nucleotide sequence or nucleic acid sequence described herein may be a DNA molecule (e.g., cDNA), an RNA molecule, or a combination of a DNA and RNA molecule. In some embodiments, a nucleotide sequence or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The nucleic acid or nucleotide sequences can be single-stranded, double-stranded, may contain both single- stranded and double-stranded portions, and may contain triple-stranded portions. In a specific embodiment, a nucleotide sequence or nucleic acid sequence described herein is a negative sense single-stranded RNA. In another specific embodiment, a nucleotide sequence or nucleic acid sequence described herein is a positive sense single-stranded RNA. In another specific embodiment, a nucleotide sequence or nucleic acid sequence described herein is a cDNA.

### 5.1.2 Sars-Cov-2 Spike Protein/Chimeric F Protein With The Sars-Coov-2 Spike Protein Ectodomain

In a specific embodiment, a transgene comprising a nucleotide sequence encoding a SARS-CoV-2 spike protein or portion thereof (e.g., ectodomain or receptor binding domain of the SARS-CoV-2 spike protein) is incorporated into the genome of any NDV type or strain. (e.g., NDV LaSota strain) See, e.g., Section 5.1.1, supra, for types and strains of NDV that may be used. The transgene encoding a SARS-CoV-2 spike protein or portion thereof (e.g., ectodomain or receptor binding domain of the SARS-CoV-2 spike protein) may inserted into any NDV type or strain (e.g., NDV LaSota strain). In a specific embodiment, a transgene encoding a SARS-CoV-2 spike protein or portion thereof (e.g., ectodomain or receptor binding domain of the SARS-CoV-2 spike protein) is incorporated into the genome of any NDV type or strain (e.g., NDV LaSota strain). See, e.g., Section 3.1 and Table 3 in Section 5.8 for exemplary sequences for SARS-CoV-2 spike proteins or portion thereof (e.g., ectodomain or receptor binding domain of the SARS-CoV-2 spike protein) and exemplary nucleic acid sequences encoding SARS-CoV-2 spike protein or portion thereof (e.g., ectodomain or receptor binding domain of the SARS-CoV-2 spike protein). One of skill in the art would be able to use such sequence information to produce a transgene for incorporation into the genome of any NDV type or strain. Given the degeneracy of the nucleic acid code, there are a number of different nucleic acid sequences that may encode the same SARS-CoV-2 spike protein or portion thereof (e.g., ectodomain or receptor binding domain of the SARS-CoV-2 spike protein). In a specific embodiment, a transgene encoding a SARS-CoV-2 spike protein or portion thereof (e.g., ectodomain or receptor binding domain of the SARS-CoV-2 spike protein) is codon optimized. See, e.g., Section 5.1.5, infra, for a discussion regarding codon optimization. In some embodiments, the transgene encoding a SARS-CoV-2 spike protein or portion thereof (e.g., ectodomain or receptor binding domain of the SARS-CoV-2 spike protein) comprises the nucleic acid sequence comprising the sequence set forth in SEQ ID NO: 4, 6, 8, or 10. In some embodiments, the transgene encoding a SARS-CoV-2 spike protein or portion thereof (e.g., ectodomain or receptor binding domain of the SARS-CoV-2 spike protein) comprises a nucleic acid sequence encoding the amino acid sequence comprising the sequence set forth in SEQ ID NO: 5, 7, 9, or 11. In certain embodiments, the transgene encoding a SARS-CoV-2 spike protein or portion thereof (e.g., ectodomain or receptor binding domain of the SARS-CoV-2 spike protein) comprises a nucleic acid sequence encoding an amino acid sequence comprising the SARS-CoV-2 spike protein portion of the sequence set forth in SEQ ID NO: 7 or 9. In certain embodiments, the transgene encoding a SARS-CoV-2 spike protein comprises a nucleic acid sequence encoding an amino acid sequence comprising the sequence set forth in SEQ ID NO:11. In certain embodiments, the transgene encoding a SARS-CoV-2 spike protein comprises a nucleic acid sequence encoding an amino acid sequence comprising the sequence set forth in SEQ ID NO:11 minus the signal peptide. The transgene encoding a SARS-CoV-2 spike protein or portion thereof (e.g., ectodomain or receptor binding domain of the SARS-CoV-2 spike protein) may be incorporated between any two NDV transcription units (e.g., between the NDV P and M transcription units, or between the HN and L transcription units).

In certain embodiments, a portion of a SARS-CoV-2 spike protein comprises the receptor binding domain of the SARS-CoV-2 spike protein. In some embodiments, a portion of a SARS-CoV-2 spike protein comprises the receptor binding domain of the SARS-CoV-2 spike protein and 5, 10, 15, 20, 30, 40, 50, 75 or more N-terminus amino acid residues of the SARS-CoV-2 protein 5, 10, 15, 20, 30, 40, 50, 75 or more C-terminus amino acid residues of the SARS-CoV-2 protein, or 5, 10, 15, 20, 30, 40, 50, 75 or more N-terminus amino acid residues and 5, 10, 15, 20, 30, 40, 50, 75 or more C-terminus amino acid residues of the SARS-CoV-2 protein. In some embodiments, a portion of a SARS-CoV-2 spike protein comprises the receptor binding domain of the SARS-CoV-2 spike protein and 5 to 25, 5 to 50, 25 to 50, 25 to 75, or 50 to 75 N-terminus amino acid residues of the SARS-CoV-2 protein, 5 to 25, 5 to 50, 25 to 50, 25 to 75, or 50 to 75 C-terminus amino acid residues of the SARS-CoV-2 protein, or 5 to 25, 5 to 50, 25 to 50, 25 to 75, or 50 to 75 N-terminus amino acid residues and 5 to 25, 5 to 50, 25 to 50, 25 to 75, or 50 to 75 C-terminus amino acid residues of the SARS-CoV-2 protein.

In certain embodiments, a portion of a SARS-CoV-2 spike protein comprises the S1 domain of the SARS-CoV-2 spike protein. In some embodiments, a portion of a SARS-CoV-2 spike protein comprises the S1 domain of the SARS-CoV-2 spike protein and 5, 10, 15 or more N-terminus amino acid residues of the SARS-CoV-2 protein 5, 10, 15, 20, 30, 40, 50, 75 or more C-terminus amino acid residues of the SARS-CoV-2 protein, or 5, 10, 15 or more N-terminus amino acid residues and 5, 10, 15, 20, 30, 40, 50, 75 or more C-terminus amino acid residues of the SARS-CoV-2 protein. In some embodiments, a portion of a SARS-CoV-2 spike protein comprises the S1 domain of the SARS-CoV-2 spike protein and 5 to 15 N-terminus amino acid residues of the SARS-CoV-2 protein, 5 to 25, 5 to 50, 25 to 50, 25 to 75, or 50 to 75 C-terminus amino acid residues of the SARS-CoV-2 protein, or 5 to 15 N-terminus amino acid residues and 5 to 25, 5 to 50, 25 to 50, 25 to 75, or 50 to 75 C-terminus amino acid residues of the SARS-CoV-2 protein.

In certain embodiments, a portion of a SARS-CoV-2 spike protein comprises the S2 domain of the SARS-CoV-2 spike protein. In some embodiments, a portion of a SARS-CoV-2 spike protein comprises the S2 domain of the SARS-CoV-2 spike protein and 5, 10, 15, 20, 30, 40, 50, 75 or more N-terminus amino acid residues of the SARS-CoV-2 protein 5, 10, 15, 20, 30, 40, 50, 75 or more C-terminus amino acid residues of the SARS-CoV-2 protein, or 5, 10, 15, 20, 30, 40, 50, 75 or more N-terminus amino acid residues and 5, 10, 15, 20, 30, 40, 50, 75 or more C-terminus amino acid residues of the SARS-CoV-2 protein. In some embodiments, a portion of a SARS-CoV-2 spike protein comprises the S2 domain of the SARS-CoV-2 spike protein and 5 to 25, 5 to 50, 25 to 50, 25 to 75, or 50 to 75 N-terminus amino acid residues of the SARS-CoV-2 protein, 5 to 25, 5 to 50, 25 to 50, 25 to 75, or 50 to 75 C-terminus amino acid residues of the SARS-CoV-2 protein, or 5 to 25, 5 to 50, 25 to 50, 25 to 75, or 50 to 75 N-terminus amino acid residues and 5 to 25, 5 to 50, 25 to 50, 25 to 75, or 50 to 75 C-terminus amino acid residues of the SARS-CoV-2 protein.

In certain embodiments, a portion of a SARS-CoV-2 spike protein comprises the S1 domain and S2 domain of the SARS-CoV-2 spike protein. In some embodiments, a portion of a SARS-CoV-2 spike protein comprises the S1 domain and S2 domain of the SARS-CoV-2 spike protein and 5, 10, 15 or more N-terminus amino acid residues of the SARS-CoV-2 protein, 5, 10, 15, 20, 30, 40, 50, 75 or more C-terminus amino acid residues of the SARS-CoV-2 protein, or 5, 10, 15 or more N-terminus amino acid residues and 5, 10, 15, 20, 30, 40, 50, 75 or more C-terminus amino acid residues of the SARS-CoV-2 protein. In some embodiments, a portion of a SARS-CoV-2 spike protein comprises the S1 domain and S2 domain of the SARS-CoV-2 spike protein and 5 to 15 N-terminus amino acid residues of the SARS-CoV-2 protein, 5 to 25, 5 to 50, 25 to 50, 25 to 75, or 50 to 75 C-terminus amino acid residues of the SARS-CoV-2 protein, or 5 to 15 N-terminus amino acid residues and 5 to 25, 5 to 50, 25 to 50, 25 to 75, or 50 to 75 C-terminus amino acid residues of the SARS-CoV-2 protein.

In certain embodiments, a portion of a SARS-CoV-2 spike protein comprises the ectodomain of the SARS-CoV-2 spike protein. In some embodiments, a portion of a SARS-CoV-2 spike protein comprises the ectodomain of the SARS-CoV-2 spike protein and 5, 10, 15 or more N-terminus amino acid residues of the SARS-CoV-2 protein 5, 10, 15, 20, 30, 40, 50, 75 or more C-terminus amino acid residues of the SARS-CoV-2 protein, or 5, 10, 15 or more N-terminus amino acid residues and 5, 10, 15, 20, 30, 40, 50, 75 or more C-terminus amino acid residues of the SARS-CoV-2 protein. In some embodiments, a portion of a SARS-CoV-2 spike protein comprises ectodomain of the SARS-CoV-2 spike protein and 5 to 15 N-terminus amino acid residues of the SARS-CoV-2 protein, 5 to 25, 5 to 50, 25 to 50, 25 to 75, or 50 to 75 C-terminus amino acid residues of the SARS-CoV-2 protein, or 5 to 15 N-terminus amino acid residues and 5 to 25, 5 to 50, 25 to 50, 25 to 75, or 50 to 75 C-terminus amino acid residues of the SARS-CoV-2 protein.

In certain embodiments, a portion of a SARS-CoV-2 spike protein comprises 200, 220, 222, 250, 300, 350, 400, or more amino acid residues. In some embodiments, a portion of a SARS-CoV-2 spike protein comprises 450, 500, 550, 600, 650, 700, 750, 800, 850, 900, 950, 1000, 1100, 1200 or more.

In another embodiment, described herein is a transgene comprising a nucleotide sequence encoding a full length SARS-CoV-2 spike protein or a fragment thereof. In certain embodiments, the protein further comprises a domain(s) that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In certain embodiments, a fragment of the SARS-CoV-2 spike protein is at least 1000, 1025, 1075, 1100, 1125, 1150, 1200 or 1215 amino acid residues in length. In a specific embodiment, described herein is a transgene comprising a nucleotide sequence encoding the amino acid sequence set forth in SEQ ID NO:11.

In another embodiment, provided herein is a transgene comprising a nucleotide sequence that is at least 85%, 90%, 95%, 96%, 97%, 98% or 99% identical to the nucleotide sequence of SEQ ID NO:10. In another embodiment, provided herein is a transgene comprising a nucleotide sequence that is at least 95%, 96%, 97%, 98% or 99% identical to the nucleotide sequence of SEQ ID NO: 10. In another embodiment, provided herein is a transgene comprising a nucleotide sequence that is at least 96%, 97%, 98% or 99% identical to the nucleotide sequence of SEQ ID NO:10. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO:11. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO:11. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO:11. Methods/techniques known in the art may be used to determine sequence identity (see, e.g., “Best Fit” or “Gap” program of the Sequence Analysis Software Package, version 10; Genetics Computer Group, Inc.). In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His- His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids substituted with another amino acid (e.g., a conservative amino acid substitution). In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids at the C-terminus substituted with another amino acid (e.g., a conservative amino acid substitution). In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids at the N-terminus substituted with another amino acid (e.g., a conservative amino acid substitution). In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution). In specific embodiments, the SARS-CoV-2 spike protein is the mature form of the protein. In other embodiments, the SARS-CoV-2 spike protein is the immature form of the protein. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprise one or more polypeptide domains. The one or more polypeptide domains may be at the C-terminus or N-terminus. In a specific embodiment, the one or more polypeptide domains are at the C-terminus Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids deleted. In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids deleted from the C-terminus. In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids deleted from the N-terminus. In specific embodiments, the SARS-CoV-2 spike protein is the mature form of the protein. In other embodiments, the SARS-CoV-2 spike protein is the immature form of the protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, 12, 13, 14, or 15 mutations (e.g., amino acid substitutions, amino acid deletions, amino acid additions, or a combination thereof). In another embodiment, provided herein is a transgene comprises a nucleotide sequence encoding a protein comprising (or consisting of) a SARS-CoV-2 spike protein with 1, 2, 3, 4, 5, 6, 7, 8, 9, 10, 11, or 12 amino acids deleted and 1, 2, 3, 4, 5, 6, 7, 8, 9, 10, 11 or 12 amino acid substitutions. In specific embodiments, the SARS-CoV-2 spike protein is the mature form of the protein. In other embodiments, the SARS-CoV-2 spike protein is the immature form of the protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, described herein is a transgene comprising a nucleotide sequence encoding a protein comprising (or consisting of) the receptor binding domain of a SARS-CoV-2 spike protein. In certain embodiments, protein further comprise one or more polypeptide domains. Useful polypeptide domains include domains that facilitate purification, folding and cleavage of portions of a polypeptide. For example, a His tag (His- His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the receptor binding domain of a SARS-CoV-2 spike protein and a His tag (e.g., a (His)n, where n is 6). In certain embodiments, a protein comprising (or consisting) of the receptor binding domain of a SARS-CoV-2 spike polypeptide is a secreted polypeptide. In a specific embodiment, described herein is a transgene comprising a nucleotide sequence encoding the amino acid sequence set forth in SEQ ID NO:5 or 7. In a specific embodiment, when designing a protein comprising SARS-CoV-2 spike polypeptide receptor binding domain, care is taken to maintain the stability of the resulting protein.

In another embodiment, provided herein is a transgene comprising a nucleotide sequence that is at least 85%, 90%, 95%, 96%, 97%, 98% or 99% identical to the nucleotide sequence of SEQ ID NO:4 or 6. In another embodiment, provided herein is a transgene comprising a nucleotide sequence that is at least 85%, 90%, 95%, 96%, 97%, 98% or 99% identical to the nucleotide sequence of SEQ ID NO:4 minus the nucleotide sequence encoding the signal sequence. In another embodiment, provided herein is a transgene comprising a nucleotide sequence that is at least 95%, 96%, 97%, 98% or 99% identical to the nucleotide sequence of SEQ ID NO:4 or 6. In another embodiment, provided herein is a transgene comprising a nucleotide sequence that is at least 96%, 97%, 98% or 99% identical to the nucleotide sequence of SEQ ID NO:4 or 6. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO: 5 or 7. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO: 5 minus the signal sequence. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO: 5 or 7. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO: 5 or 7. Methods/techniques known in the art may be used to determine sequence identity (see, e.g., “Best Fit” or “Gap” program of the Sequence Analysis Software Package, version 10; Genetics Computer Group, Inc.). In certain embodiments, the protein further comprises one or more polypeptide domains. The one or more polypeptide domains may be at the C-terminus or N-terminus. In a specific embodiment, the one or more polypeptide domains are at the C-terminus Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids substituted with another amino acid (e.g., a conservative amino acid substitution). In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids at the C-terminus substituted with another amino acid (e.g., a conservative amino acid substitution). In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids at the N-terminus substituted with another amino acid (e.g., a conservative amino acid substitution). In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution). Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprises one or more polypeptide domains. The one or more polypeptide domains may be at the C-terminus or N-terminus. In a specific embodiment, the one or more polypeptide domains are at the C-terminus Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids deleted from the C-terminus. In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids deleted from the N-terminus. In another embodiment, provided herein is a transgene comprises a nucleotide sequence encoding a protein comprising (or consisting of) a SARS-CoV-2 spike protein receptor binding domain with 1, 2, 3, 4, 5, 6, 7, 8, 9, 10, 11, or 12 amino acids deleted from the N-terminus and 1, 2, 3, 4, 5, 6, 7, 8, 9, 10, 11, or 12 amino acids deleted from the C-terminus. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6.

In another embodiment, described herein is a transgene comprising a nucleotide sequence encoding a protein comprising (or consisting of) the ectodomain of a SARS-CoV-2 spike protein. In some embodiments, the SARS-CoV-2 spike protein ectodomain lacks the polybasic cleavage site (e.g., amino acid residues 682 to 685 (RRAR) are substituted with a single alanine). In certain embodiments, protein further comprises one or more polypeptide domains. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the ectodomain of a SARS-CoV-2 spike protein and a His tag (e.g., a (His)n, where n is 6). In a specific embodiment, described herein is a transgene comprising a nucleotide sequence encoding the amino acid sequence set forth in SEQ ID NO:9 or SEQ ID NO:9 minus the histidine tag. In certain embodiments, a protein comprising (or consisting) of the ectodomain of a SARS-CoV-2 spike polypeptide) is a secreted polypeptide. In a specific embodiment, when designing a protein comprising SARS-CoV-2 spike polypeptide ectodomain, care is taken to maintain the stability of the resulting protein.

In another embodiment, described herein is a transgene comprising a nucleotide sequence encoding a protein comprising (or consisting of) the ectodomain of a SARS-CoV-2 spike protein. In certain embodiments, the protein further comprise one or more tetramerization domains (e.g., human tetramerization domains) known to one of skill in the art. In some embodiments, such a protein further comprises a domain(s) that facilitate purification, folding and cleavage of portions of a polypeptide. For example, a His tag (His- His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the ectodomain of a SARS-CoV-2 spike protein and a tetramerization domain, and optionally a His tag (e.g., a (His)n, where n is 6). In certain embodiments, such a protein is a secreted polypeptide. In a specific embodiment, when designing a protein comprising SARS-CoV-2 spike polypeptide ectodomain, care is taken to maintain the stability of the resulting protein.

In another embodiment, provided herein is a transgene comprising a nucleotide sequence that is at least 85%, 90%, 95%, 96%, 97%, 98% or 99% identical to the nucleotide sequence of SEQ ID NO:8 or SEQ ID NO:8 minus the nucleotide sequence encoding the histidine tag. In another embodiment, provided herein is a transgene comprising a nucleotide sequence that is at least 95%, 96%, 97%, 98% or 99% identical to the nucleotide sequence of SEQ ID NO: 8 or SEQ ID NO:8 minus the nucleotide sequence encoding the histidine tag. In another embodiment, provided herein is a transgene comprising a nucleotide sequence that is at least 96%, 97%, 98% or 99% identical to the nucleotide sequence of SEQ ID NO: 8 or SEQ ID NO:8 minus the nucleotide sequence encoding the histidine tag. In another embodiment, provided herein is a transgene comprising a nucleotide sequence that is at least 85%, 90%, 95%, 96%, 97%, 98% or 99% identical to the nucleotide sequence of SEQ ID NO:8 or SEQ ID NO:8 minus the histidine tag. In another embodiment, provided herein is a transgene comprising a nucleotide sequence that is at least 85%, 90%, 95%, 96%, 97%, 98% or 99% identical to the nucleotide sequence of SEQ ID NO:8 minus the nucleotide sequence encoding the histidine tag and minus the nucleotide sequence encoding the signal sequence. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO: 9 or SEQ ID NO:9 minus the histidine tag. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO: 9 or SEQ ID NO:9 minus the histidine tag. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO:9 or SEQ ID NO:9 minus the histidine tag. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO:9 minus the histidine tag and signal sequence. Methods/techniques known in the art may be used to determine sequence identity (see, e.g., “Best Fit” or “Gap” program of the Sequence Analysis Software Package, version 10; Genetics Computer Group, Inc.). In certain embodiments, the protein further comprise one or more tetramerization domains (e.g., human tetramerization domains) known to one of skill in the art.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity = number of identical overlapping positions/total number of positions X 100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared (e.g., the full-length of a gene coding sequence, or a fragment thereof). In some embodiments, a fragment of a nucleotide sequence is at least 25, at least 50, at least 75, or at least 100 nucleotides. Similarly, “percent sequence identity” may be readily determined for amino acid sequences, over the full-length of a protein, or a fragment thereof. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids substituted with another amino acid (e.g., a conservative amino acid substitution). In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids at the C-terminus substituted with another amino acid (e.g., a conservative amino acid substitution). In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids at the N-terminus substituted with another amino acid (e.g., a conservative amino acid substitution). In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution). Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the SARS-CoV-2 spike protein ectodomain lacks the polybasic cleavage site (e.g., amino acid residues 682 to 685 (RRAR) are substituted with a single alanine). In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6. In certain embodiments, the protein comprises one or more tetramerization domains (e.g., human tetramerization domains) known to one of skill in the art.

In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids deleted from the C-terminus. In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids deleted from the N-terminus. In certain embodiments, the SARS-CoV-2 spike protein ectodomain lacks the polybasic cleavage site (e.g., amino acid residues 682 to 685 (RRAR) are substituted with a single alanine). In certain embodiments, the protein further comprise one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6. In certain embodiments, the protein comprises one or more tetramerization domains (e.g., human tetramerization domains) known to one of skill in the art.

In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids deleted. In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids deleted from the C-terminus. In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acids deleted from the N-terminus. In certain embodiments, the SARS-CoV-2 spike protein ectodomain lacks the polybasic cleavage site (e.g., amino acid residues 682 to 685 (RRAR) are substituted with a single alanine). In certain embodiments, the protein further comprise one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6. In certain embodiments, the protein comprises one or more tetramerization domains (e.g., human tetramerization domains) known to one of skill in the art.

In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof). In another embodiment, provided herein is a transgene comprises a nucleotide sequence encoding a protein comprising (or consisting of) a SARS-CoV-2 spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In certain embodiments, the SARS-CoV-2 spike protein ectodomain lacks the polybasic cleavage site (e.g., amino acid residues 682 to 685 (RRAR) are substituted with a single alanine). In certain embodiments, the protein further comprise one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n, wherein n is 6. In certain embodiments, the protein comprises one or more tetramerization domains (e.g., human tetramerization domains) known to one of skill in the art.

In another embodiment, described herein are transgenes comprising a nucleotide sequence encoding a chimeric F protein, wherein the chimeric F protein comprises a SARS-CoV-2 spike protein ectodomain and NDV F protein transmembrane and cytoplasmic domains. In other words, the NDV F protein transmembrane and cytoplasmic domains replace the SARS-CoV-2 spike protein transmembrane and cytoplasmic domains so that the chimeric F protein does not include the SARS-CoV-2 spike protein transmembrane and cytoplasmic domains. In specific embodiments, the SARS-CoV-2 spike protein ectodomain lacks the polybasic cleavage site (e.g., amino acid residues of the polybasic cleavage site (RRAR) are substituted with a single alainine). The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determined the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the transgene encoding the chimeric F protein is codon optimized. See, e.g., Section 5.1.5, infra, for a discussion regarding codon optimization. In specific embodiment, described herein is a transgene comprising a nucleotide sequence encoding a chimeric F protein, wherein the chimeric F protein comprises a SARS-CoV-2 spike protein ectodomain and NDV F protein transmembrane and cytoplasmic domains, and wherein the transgene comprises an RNA sequence corresponding to the negative sense of the cDNA sequence of SEQ ID NO: 12. In a preferred embodiment, a transgene comprises a codon optimized version of a nucleic acid sequence encoding the chimeric F protein. In a specific embodiment, a transgene described herein comprises a nucleotide sequence encoding the amino acid sequence set forth in SEQ ID NO:13. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, described herein are transgenes comprising a nucleotide sequence encoding a chimeric F protein, wherein the chimeric F protein comprises a SARS-CoV-2 spike protein ectodomain plus or minus 1, 2, 3, 4, 5, 6, 7, 8 or more amino acid residues at C-terminus and NDV F protein transmembrane and cytoplasmic domains. In other words, the portion of the SARS-CoV-2 spike protein encoded by the chimeric F protein does not include the full length SARS-CoV-2 spike protein transmembrane and cytoplasmic domains. In specific embodiments, the SARS-CoV-2 spike protein ectodomain lacks the polybasic cleavage site (e.g., amino acid residues of the polybasic cleavage site (RRAR) are substituted with a single alainine). The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determined the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the transgene encoding the chimeric F protein is codon optimized. See, e.g., Section 5.1.5, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprising a nucleotide sequence that is at least 85%, 90%, 95%, 96%, 97%, 98% or 99% identical to the nucleotide sequence of SEQ ID NO:12. In another embodiment, provided herein is a transgene comprising a nucleotide sequence that is at least 95%, 96%, 97%, 98% or 99% identical to the nucleotide sequence of SEQ ID NO:12. In another embodiment, provided herein is a transgene comprising a nucleotide sequence that is at least 96%, 97%, 98% or 99% identical to the nucleotide sequence of SEQ ID NO:12. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO:13. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO:13. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO:13. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In a specific embodiment the NDV genome is of the LaSota strain.

In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids substituted with another amino acid (e.g., a conservative amino acid substitution) and NDV F protein transmembrane and cytoplasmic domains. In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids at the C-terminus substituted with another amino acid (e.g., a conservative amino acid substitution) and NDV F protein transmembrane and cytoplasmic domains. In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids at the N-terminus substituted with another amino acid (e.g., a conservative amino acid substitution) and NDV F protein transmembrane and cytoplasmic domains. In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution), and NDV F protein transmembrane and cytoplasmic domains. In specific embodiments, the SARS-CoV-2 spike protein ectodomain lacks the polybasic cleavage site (e.g., amino acid residues 682 to 685 (RRAR) to a single alanine. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein ectodomain is fused directly to the NDV F protein transmembrane and cytoplasmic domains. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the SARS-CoV-2 spike protein ectodomain lacks the polybasic cleavage site (e.g., amino acid residues 682 to 685 (RRAR) are substituted with a single alanine). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acids deleted from the N-terminus, and NDV F protein transmembrane and cytoplasmic domains. In certain embodiments, the SARS-CoV-2 spike protein ectodomain lacks the polybasic cleavage site (e.g., amino acid residues 682 to 685 (RRAR) are substituted with a single alanine). In specific embodiments, the SARS-CoV-2 spike protein ectodomain lacks the polybasic cleavage site (e.g., amino acid residues 682 to 685 (RRAR) to a single alanine. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof), and NDV F protein transmembrane and cytoplasmic domains. In another embodiment, provided herein is a transgene comprises a nucleotide sequence encoding a chimeric F protein comprising (or consisting of) a SARS-CoV-2 spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In certain embodiments, the SARS-CoV-2 spike protein ectodomain lacks the polybasic cleavage site (e.g., amino acid residues 682 to 685 (RRAR) are substituted with a single alanine). In specific embodiments, the SARS-CoV-2 spike protein ectodomain lacks the polybasic cleavage site (e.g., amino acid residues 682 to 685 (RRAR) to a single alanine. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, described herein are transgenes comprising a nucleotide sequence encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determined the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the transgene encoding the chimeric F protein is codon optimized. See, e.g., Section 5.1.5, infra, for a discussion regarding codon optimization. In a specific embodiment, described herein is a transgene comprising a nucleotide sequence encoding a chimeric F protein, wherein the chimeric F protein comprises a SARS-CoV-2 spike protein ectodomain and an NDV F protein transmembrane and cytoplasmic domains, wherein the transgene comprises an RNA sequence corresponding to the negative sense of the cDNA sequence of SEQ ID NO: 14. In another specific embodiment, described herein is a transgene comprising a nucleotide sequence encoding a chimeric F protein, wherein the chimeric F protein comprises a SARS-CoV-2 spike protein ectodomain and an NDV F protein transmembrane and cytoplasmic domains, wherein the transgene comprises an RNA sequence corresponding to the negative sense of the cDNA sequence of SEQ ID NO: 16. In another specific embodiment, described herein is a transgene comprising a nucleotide sequence encoding a chimeric F protein, wherein the chimeric F protein comprises a SARS-CoV-2 spike protein ectodomain and an NDV F protein transmembrane and cytoplasmic domains, wherein the transgene comprises an RNA sequence corresponding to the negative sense of the cDNA sequence of SEQ ID NO: 18. In a preferred embodiment, a transgene comprises a codon optimized version of a nucleic acid sequence encoding the chimeric F protein. In a specific embodiment, a transgene described herein comprises a nucleotide sequence encoding the amino acid sequence set forth in SEQ ID NO:15. In another specific embodiment, a transgene described herein comprises a nucleotide sequence encoding the amino acid sequence set forth in SEQ ID NO:17. In a specific embodiment, a transgene described herein comprises a nucleotide sequence encoding the amino acid sequence set forth in SEQ ID NO:19. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units).

In another embodiment, provided herein is a transgene comprising a nucleotide sequence that is at least 85%, 90%, 95%, 96%, 97%, 98% or 99% identical to the nucleotide sequence of SEQ ID NO:14. In another embodiment, provided herein is a transgene comprising a nucleotide sequence that is at least 95%, 96%, 97%, 98% or 99% identical to the nucleotide sequence of SEQ ID NO:14. In another embodiment, provided herein is a transgene comprising a nucleotide sequence that is at least 96%, 97%, 98% or 99% identical to the nucleotide sequence of SEQ ID NO:14. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO:15. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO:15. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO:15. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprising a nucleotide sequence that is at least 85%, 90%, 95%, 96%, 97%, 98% or 99% identical to the nucleotide sequence of SEQ ID NO:16. In another embodiment, provided herein is a transgene comprising a nucleotide sequence that is at least 95%, 96%, 97%, 98% or 99% identical to the nucleotide sequence of SEQ ID NO:16. In another embodiment, provided herein is a transgene comprising a nucleotide sequence that is at least 96%, 97%, 98% or 99% identical to the nucleotide sequence of SEQ ID NO:16. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO:17. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO:17. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO:17. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprising a nucleotide sequence that is at least 85%, 90%, 95%, 96%, 97%, 98% or 99% identical to the nucleotide sequence of SEQ ID NO:18. In another embodiment, provided herein is a transgene comprising a nucleotide sequence that is at least 95%, 96%, 97%, 98% or 99% identical to the nucleotide sequence of SEQ ID NO:18. In another embodiment, provided herein is a transgene comprising a nucleotide sequence that is at least 96%, 97%, 98% or 99% identical to the nucleotide sequence of SEQ ID NO:18. In another embodiment, provided herein is a transgene comprising a nucleotide sequence encoding a protein comprising (or consisting of) an amino acid sequence that is at least 85%, 90%, 95%, 96%, 97%, 98% or 99% identical to the amino acid sequence of SEQ ID NO:19. In another embodiment, provided herein is a transgene comprising a nucleotide sequence encoding a protein comprising (or consisting of) an amino acid sequence that is at least 95%, 96%, 97%, 98% or 99% identical to the amino acid sequence of SEQ ID NO:19. In another embodiment, provided herein is a transgene comprising a nucleotide sequence encoding a protein comprising (or consisting of) an amino acid sequence that is at least 96%, 97%, 98% or 99% identical to the amino acid sequence of SEQ ID NO:19. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprising a nucleotide sequence encoding a chimeric F protein, wherein the chimeric F protein comprises (or consists of) a SARS-CoV-2 spike protein ectdomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the nucleotide sequence of SEQ ID NO:9, SEQ ID NO:9 without the His tag, or SEQ ID NO:9 without the His tag and signal sequence. In another embodiment, provided herein is a transgene comprising a nucleotide sequence encoding a chimeric F protein, wherein the chimeric F protein comprises (or consists of) a SARS-CoV-2 spike protein ectdomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the nucleotide sequence of SEQ ID NO:13 without the NDV F protein transmembrane and cytoplasmic domains. In another embodiment, provided herein is a transgene comprising a nucleotide sequence encoding a chimeric F protein, wherein the chimeric F protein comprises (or consists of) a SARS-CoV-2 spike protein ectdomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the nucleotide sequence of SEQ ID NO:15 without the NDV F protein transmembrane and cytoplasmic domains. In another embodiment, provided herein is a transgene comprising a nucleotide sequence encoding a chimeric F protein, wherein the chimeric F protein comprises (or consists of) a SARS-CoV-2 spike protein ectdomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the nucleotide sequence of SEQ ID NO: 17 without the NDV F protein transmembrane and cytoplasmic domains. In another embodiment, provided herein is a transgene comprising a nucleotide sequence encoding a chimeric F protein, wherein the chimeric F protein comprises (or consists of) a SARS-CoV-2 spike protein ectdomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the nucleotide sequence of SEQ ID NO: 19 without the NDV F protein transmembrane and cytoplasmic domains. In certain embodiments, the transgene encoding the chimeric F protein is codon optimized. See, e.g., Section 5.1.5, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein SARS-CoV-2 spike protein ectodomain has 1, 2, 3, 4, 5, 6, 7, 8, 9, 10, 11, or 12 amino acids substituted with another amino acid (e.g., a conservative amino acid substitution) and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein SARS-CoV-2 spike protein ectodomain has 1, 2, 3, 4, 5, 6, 7, 8, 9, 10, 11, or 12 amino acids at the C-terminus substituted with another amino acid (e.g., a conservative amino acid substitution) and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein SARS-CoV-2 spike protein ectodomain has 1, 2, 3, 4, 5, 6, 7, 8, 9, 10, 11, or 12 amino acids at the N-terminus substituted with another amino acid (e.g., a conservative amino acid substitution) and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein SARS-CoV-2 spike protein ectodomain has 1, 2, 3, 4, 5, 6, 7, 8, 9, 10, 11, or 12 amino acids at the C-terminus substituted with another amino acid (e.g., a conservative amino acid substitution), has 1, 2, 3, 4, 5, 6, 7, 8, 9, 10, 11, or 12 amino acids at the N-terminus substituted with another amino acid (e.g., a conservative amino acid substitution) and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein ectodomain is fused directly to the NDV F protein transmembrane and cytoplasmic domains. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophylic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the SARS-CoV-2 spike protein ectodomain lacks the polybasic cleavage site (e.g., amino acid residues 682 to 685 (RRAR) are substituted with a single alanine). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein the SARS-CoV-2 spike protein ectodomain has 1, 2, 3, 4, 5, 6, 7, 8, 9, 10, 11, or 12 amino acids deleted and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein the SARS-CoV-2 spike protein ectodomain has 1, 2, 3, 4, 5, 6, 7, 8, 9, 10, 11, or 12 amino acids at the C-terminus deleted and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein the SARS-CoV-2 spike protein ectodomain has 1, 2, 3, 4, 5, 6, 7, 8, 9, 10, 11, or 12 amino acids at the N-terminus deleted and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein the SARS-CoV-2 spike protein ectodomain has 1, 2, 3, 4, 5, 6, 7, 8, 9, 10, 11, 12, 13, 14 or 15 mutations (e.g. amino acid substitutions, amino acid additions, amino acid deletions or a combination thereof) and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In another embodiment, provided herein is a transgene comprises a nucleotide sequence encoding a chimeric F protein comprising (or consisting of) a SARS-CoV-2 spike protein ectodomain and NDV F protein transmembrane and cytoplasmic domains, wherein the SARS-CoV-2 spike protein ectodomain has 1, 2, 3, 4, 5, 6, 7, 8, 9, 10, 11, or 12 amino acid substitutions, 1, 2, 3, 4, 5, 6, 7, 8, 9, 10, 11, or 12 amino acids deleted and lacks a polybasic cleavage site (e.g., amino acid residues of the polybasic domain (RRAR) substituted with a single alanine), and wherein amino acid residues corresponding to amino acid residues 817, 892, 899, 942, 986, and 987 of the spike protein found at GenBank Accession No. MN908947 are substituted with prolines. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another specific embodiment, provided herein is a transgene comprising a nucleotide sequence that can hybridize under high, moderate or typical stringency hybridization conditions to a nucleic acid sequence set forth in SEQ ID NO: 4, 6, 8, 10, 12 or 14. In another specific embodiment, provided herein is a transgene comprising a nucleotide sequence tht can hybridize under high, moderate to typical stringency hybridization conditions to a nucleic acid sequence encoding the protein set forth in SEQ ID NO: 5, 7, 9, 11, 13, or 15. Hybridization conditions are known to one of skill in the art (see, e.g., U.S. Pat. Application No. 2005/0048549 at, e.g., paragraphs 72 and 73). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another specific embodiment, provided herein is a transgene comprising a nucleotide sequence that can hybridize under high, moderate or typical stringency hybridization conditions to a nucleic acid sequence set forth in SEQ ID NO: 16 or 18. In another specific embodiment, provided herein is a transgene comprising a nucleotide sequence tht can hybridize under high, moderate to typical stringency hybridization conditions to a nucleic acid sequence encoding the protein set forth in SEQ ID NO: 17 or 19. Hybridization conditions are known to one of skill in the art (see, e.g., U.S. Pat. Application No. 2005/0048549 at, e.g., paragraphs 72 and 73).

In another specific embodiment, provided herein is a transgene comprising a nucleotide sequence that can hybridize under high, moderate or typical stringency hybridization conditions to a nucleic acid sequence set forth in SEQ ID NO: 8 minus the His tag. In another specific embodiment, provided herein is a transgene comprising a nucleotide sequence tht can hybridize under high, moderate to typical stringency hybridization conditions to a nucleic acid sequence encoding the protein set forth in SEQ ID NO: 9 minus the His tag. Hybridization conditions are known to one of skill in the art (see, e.g., U.S. Pat. Application No. 2005/0048549 at, e.g., paragraphs 72 and 73). In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, provided herein is a transgene that comprises a nucleotide sequence encoding a chimeric F protein, wherein the chimeric F protein comprises a SARS-CoV-2 spike protein ectodomain and an NDV F protein transmembrane and cytoplasmic domains, wherein the SARS-CoV-2 ectodomain is the SARS-CoV-2 ectodomain of the amino acid sequence set forth in SEQ ID NO:13, 15, 17 or 19. In another embodiment, provided herein is a transgene that comprises a nucleotide sequence encoding a chimeric F protein, wherein the chimeric F protein comprises a SARS-CoV-2 spike protein ectodomain and an NDV F protein transmembrane and cytoplasmic domains, wherein the SARS-CoV-2 ectodomain is at least 85%, at least 90%, or at least 95%, identical to the SARS-CoV-2 ectodomain of the amino acid sequence set forth in SEQ ID NO:13, 15, 17 or 19. In another embodiment, provided herein is a transgene that comprises a nucleotide sequence encoding a chimeric F protein, wherein the chimeric F protein comprises a SARS-CoV-2 spike protein ectodomain and an NDV F protein transmembrane and cytoplasmic domains, wherein the SARS-CoV-2 ectodomain is at least 95%, at least 98% or at least 99% identical to the SARS-CoV-2 ectodomain of the amino acid sequence set forth in SEQ ID NO:13, 15, 17 or 19. The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determined the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the transgene encoding the chimeric F protein is codon optimized. See, e.g., Section 5.1.5, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units).

In certain embodiments, provided herein is a transgene comprising a nucleotide sequence encoding a SARS-CoV-2 spike protein or portion thereof thereof (e.g., the ectodomain or receptor binding domain of SARS-CoV-2 spike protein), wherein the SARS-CoV-2 spike protein or portion thereof is the SARS-CoV-2 spike protein or portion thereof of a SARS-CoV-2 variant, such as disclosed in GISAID. In specific embodiments, the SARS-CoV-2 spike protein or a portion thereof of one of the SARS-CoV-2 variants found in Table 6 or Table 7 below. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, described herein are transgenes comprising a nucleotide sequence encoding a chimeric F protein, wherein the chimeric F protein comprises the ectodomain of a spike protein of a SARS-CoV-2 variant and NDV F protein transmembrane and cytoplasmic domains, wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determined the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the transgene encoding the chimeric F protein is codon optimized. See, e.g., Section 5.1.5, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units). In specific embodiments, the NDV F protein transmembrane and cytoplasmic domains are from the same NDV strain as the transcription units of the NDV genome. In a specific embodiment the NDV genome is of the LaSota strain.

In another embodiment, described herein are transgenes comprising a nucleotide sequence encoding a chimeric F protein, wherein the chimeric F protein comprises the ectodomain of a spike protein of a SARS-CoV-2 variant and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. SARS-CoV-2 variants include those found in the GISAID database or described herein. The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determined the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In certain embodiments, the transgene encoding the chimeric F protein is codon optimized. See, e.g., Section 5.1.5, infra, for a discussion regarding codon optimization. In a specific embodiment, a transgene encoding a chimeric F protein is incorporated into the genome of any NDV type or strain (e.g., NDV LaSota strain). See., e.g., Section 5.1.1, supra, for types and strains of NDV that may be used. The transgene encoding a chimeric F protein may be incorporated between any two NDV transcription units (e.g., between the NDV P and M transcription units, or between the HN and L transcription units).

In certain embodiments, a SARS-CoV-2 variant is a B.1.526, B.1.526.1, B.1.525, or P.2 variant. In some embodiments, a SARS-CoV2 variant is a B.1.1.7, B.1.351, P.1, B.1.427, or B.1.429 variant. In some embodiments, the spike protein of a SARS-CoV-2 variant comprises one, two or more, or all of the following amino acid substitutions: L5F, T95I, D253G, S477N, E484K, D614G, and A701V. In certain embodiments, the spike protein of a SARS-CoV-2 varian comprises amino acid substitutions: T95I, D253G,and D614G. In some embodiments, the spike protein of a SARS-CoV-2 variant comprises one, two or more, or all of the following mutations: D80G, Δ144, F157S, L452R, D614G, T791I, T859N*, and D950H. In certain embodiments, the spike protein of a SARS-CoV-2 variant comprises the following mutations: D80G, Δ144, F157S, L452R, D614G, and D950H. In some embodiments, the spike protein of a SARS-CoV-2 variant comprises one, two or more, or all of the following mutations: A67V, Δ69/70, Δ144, E484K, D614G, Q677H, and F888.L. In certain embodiments, the spike protein of a SARS-CoV-2 variant comprises the following mutations: A67V, Δ69/70, Δ144, E484K, D614G, Q677H, and F888.L. In some embodiments, the spike protein of a SARS-CoV-2 variant comprises one, two or more, or all of the following mutations: E484K, F565L, D614G, and V1176F. In certain embodiments, the spike protein of a SARS-CoV-2 variant comprises the following mutations: E484K, D614G, and V1176F. In some embodiments, the spike protein of a SARS-CoV-2 variant comprises one, two or more, or all of the following mutations: Δ69/70, Δ144, E484K* S494P, N501Y, A570D, D614G, P681H, T716I, S982A, D1118H, and K1191N. In certain embodiments, the spike protein of a SARS-CoV-2 variant comprises the following mutations: Δ69/70, Δ144, N501Y, A570D, D614G, P681H, T716I, S982A, and D1118H. In some embodiments, the spike protein of a SARS-CoV-2 variant comprises one, two or more, or all of the following mutations: L18F, T20N, P26S, D138Y, R190S, K417T, E484K, N501Y, D614G, H655Y, and T1027I. In certain embodiments, the spike protein of a SARS-CoV-2 variant comprises the following mutations: L18F, T20N, P26S, D138Y, R190S, K417T, E484K, N501Y, D614G, H655Y, and T1027I. In some embodiments, the spike protein of a SARS-CoV-2 variant comprises one, two or more, or all of the following mutations: D80A, D215G, Δ241/242/243, K417N, E484K, N501Y, D614G, and A701V. In certain embodiments, the spike protein of a SARS-CoV-2 variant comprises the following mutations: D80A, D215G, Δ241/242/243, K417N, E484K, N501Y, D614G, and A701V. In some embodiments, the spike protein of a SARS-CoV-2 variant comprises one or both of the following mutations: L452R and D614G. In certain embodiments, the spike protein of a SARS-CoV-2 variant comprises one, two or more, or all of the following mutations: S13I, W152C, L452R, and D614G. In some embodiments, the spike protein of a SARS-CoV-2 variant comprises the following mutations: S13I, W152C, L452R, and D614G. In certain embodiments, the spike protein of a SARS-CoV-2 variant comprises the amino acid substitution L452R. In certain embodiments, the spike protein of a SARS-CoV-2 variant comprises the amino acid substitution E484K.

TABLE 6

SARS-CoV-2 Variants
GR/501Y.V3(P.1)

| | |
|---|---|
| Strain Name | GISAID Accession Number |
| hCoV-19/England/CAMC-151FD4B/2021 | EPI_ISL_1740541 |
| hCoV-19/USA/NY-PRL-2021_0423_00N21/2021 | EPI_ISL_1717950 |
| hCoV-19/Brazil/SP-BT19771/2021 | EPI_ISL_1734866 |

GH/501.Y.V2 (B.1.351)

| Strain Name | GISAID Accession Number |
|---|---|
| hCoV-19/USA/KS-KHEL-1005/2021 | EPI_ISL_1700765 |
| hCoV-19/England/RAND-1520521/2021 | EPI_ISL_1740535 |
| hCoV-19/Brazil/SP-899592/2021 | EPI_ISL_1732275 |
| hCoV-19/India/WB-1931501021109/2021 | EPI_ISL_1589849 |
| hCoV-19/South Africa/Tygerberg 739/2021 | EPI_ISL_1502185 |
| VUI202012/01 GR501Y.V1 (B.1.1.7) | |
| Strain Name | GISAID Accession Number |
| hCoV-19/England/CAMC-151FDA5/2021 | EPI_ISL_1740737 |
| hCoV-19/USA/NY-PRL-2021 0423 00G09/2021 | EPI_ISL_1718128 |
| hCoV-19/Brazil/SP-2603/2021 | EPI_ISL_1707692 |
| hCoV-19/India/ILSGS00920/2021 | EPI_ISL_1663496 |
| hCoV-19/South Africa/KRISP-K011005/2021 | EPI_ISL_1550960 |

GH/452R.V1 (B.1.429+B.1.427)

| | |
|---|---|
| Strain Name | GISAID Accession Number |
| hCoV-19/USA/NY-PRL-2021_0423_00L18/2021 | EPI_ISL_1718148 |
| hCoV-19/England/ALDP-14CC0BE/2021 | EPI_ISL_1535254 |
| hCoV-19/India/MH-ICMR-NIV-INSACOG-GSEQ-192/2021 | EPI_ISL_1703805 |
| hCoV-19/South Africa/N00859/2020 | EPI_ISL_1239269 |

G/484K.V3 (B.1.525)

| | |
|---|---|
| Strain Name | GISAID Accession Number |
| hCoV-19/USA/NY-PRL-2021 0423 00K24/2021 | EPI_ISL_1717990 |
| hCoV-19/Scotland/QEUH-150C321/2021 | EPI_ISL_1741746 |
| hCoV-19/India/ILSGS00918/2021 | EPI_ISL_1663494 |
| hCoV-19/Brazil/BA-LACEN-125/2021 | EPI_ISL_1583653 |

G/452R.V3 (B.1.617+)

| | |
|---|---|
| Strain Name | GISAID Accession Number |
| hCoV-19/England/CAMC-151FDF0/2021 | EPI_ISL_1740580 |
| hCoV-19/USA/CA-CDC-FG-021941/2021 | EPI_ISL_1733902 |
| hCoV-19/India/CG-AIIMS-Raipur-L15928/2021 | EPI_ISL_1731755 |

TABLE 7

SARS-CoV-2 Variants (adapted from Sarkar et al., 2021, Arch Virol. 19: 1-12)

| | | |
|---|---|---|
| Region | State | Accession No. |
| East India | West Bengal | EPI-ISL-455640 - EPI-ISL-455641, EPI-ISL-455644 - EPI-ISL-455676, EPI-ISL-455678 - EPI-ISL-455679, EPI_ISL_511906, EPI_ISL_511902, EPI_ISL_508483, EPI_ISL_508475, EPI_ISL_508468, EPI_ISL_508457, EPI_ISL_508452, EPI_ISL_508448, EPI_ISL_508446, EPI_ISL_508413, EPI_ISL_508410, EPI_ISL_508406, EPI_ISL_508402, EPI_ISL_508399, EPI_ISL_508396, EPI_ISL_508389, EPI_ISL_508385, EPI_ISL_508382, EPI_ISL_508378, EPI_ISL_508375, EPI_ISL_508370, EPI_ISL_508367, EPI_ISL_508365, EPI_ISL_508362, EPI_ISL_508359, EPI_ISL_508356, EPI_ISL_508353, EPI_ISL_508349, EPI_ISL_508347, EPI_ISL_508344, EPI_ISL_508341, EPI_ISL_508338, EPI_ISL_455676, EPI_ISL_455673, EPI_ISL_455669, EPI_ISL_455666, EPI_ISL_455662, EPI_ISL_455658, EPI_ISL_455653, EPI_ISL_455650, EPI_ISL_455646, EPI_ISL_455641 |
| | Odisha | EPI-ISL-435088, EPI-ISL-455478, EPI-ISL-455749, EPI-ISL-455751 - EPI-ISL-455752, EPI-ISL-455754 - EPI-ISL-455755, EPI-ISL-455757 - EPI-ISL-455758, EPI-ISL-455760 - EPI-ISL-455761, EPI-ISL-455763 - EPI-ISL-455766, EPI-ISL-455767 - EPI-ISL-455768, EPI-ISL-455770 - EPI-ISL-455771, EPI-ISL-455775 - EPI-ISL-455780, EPI-ISL-455782 - EPI-ISL-455784, EPI-ISL-455786 - EPI-ISL-455787, EPI-ISL-508434, EPI-ISL-481204, EPI-ISL-481206, EPI-ISL-481199, EPI-ISL-481198, EPI-ISL-481196, EPI-ISL-481195, EPI-ISL-481194, EPI-ISL-481192, EPI-ISL-481191, EPI-ISL-463014, EPI-ISL-463017, EPI-ISL-463019, EPI-ISL-463026, EPI-ISL-463029, EPI-ISL-463037, EPI-ISL-463049, EPI-ISL-463052, EPI-ISL-463056, EPI-ISL-463061, EPI-ISL-463067, EPI-ISL-463074, EPI-ISL-463081, EPI-ISL-463088, EPI-ISL-481113, EPI-ISL-481116, EPI-ISL-481119, EPI-ISL-481123, EPI-ISL-481126, EPI-ISL-481132, EPI-ISL-481135, EPI-ISL-481140, EPI-ISL-481147, EPI-ISL-481149, EPI-ISL-481152, EPI-ISL-481159, EPI-ISL-481164, EPI-ISL-481167, EPI-ISL-481173, EPI-ISL-481176, EPI-ISL-481179, EPI-ISL-481185, EPI-ISL-481190, EPI-ISL-481193, EPI-ISL-481197, EPI-ISL-4811200, EPI-ISL-481203, EPI-ISL-481205 |
| | Bihar | EPI-ISL-435112, EPI-ISL-436417, EPI-ISL-436419, EPI-ISL-436439, EPI-ISL-436441, EPI-ISL-436449 |
| Western India | Gujarat | EPI-ISL-426414 - EPI-ISL-426415, EPI-ISL-435050 - EPI-ISL-435056, EPI-ISL-437438, EPI-ISL-437441- EPI-ISL-437442, EPI-ISL-437444 - EPI-ISL-437454, EPI-ISL-44456 - EPI-ISL-444486, EPI-ISL-447030 - EPI-ISL-447035, EPI-ISL-447037- EPI-ISL-447053, EPI-ISL-447534 - EPI-ISL-447555, EPI-ISL-450781 - EPI-ISL-450791, EPI-ISL-451149 - EPI-ISL-451156, EPI-ISL-451158 - EPI-ISL-451163, EPI-ISL-455015 - EPI-ISL-455027, EPI_ISL_458088, EPI _ISL__458093, EPI_ISL_458097, EPI_ISL_458099, EPI_ISL_458112, EPI_ISL_458108, EPI_ISL_458113, EPI_ISL_461480, EPI_ISL_461486, EPI_ISL_461490, EPI_ISL_461493, EPI_ISL_461498, EPI_ISL_461503, EPI_ISL_461506, EPI_ISL_467029, EPI_ISL_467032, EPI_ISL_467035, EPI_ISL_467038, EPI_ISL_467041, EPI_ISL_467045, EPI_ISL_467050, EPI_ISL_467052, EPI_ISL_467054, EPI_ISL_469026, EPI_ISL_469029, EPI_ISL_469033, EPI_ISL_469038, EPI_ISL_469040, EPI_ISL_469044, EPI_ISL_469048, EPI_ISL_475027, EPI_ISL_475031, EPI_ISL_475036, EPI_ISL_475040, EPI_ISL_475047, EPI_ISL- 475055, EPI_ISL_476855, EPI_ISL_476867, EPI_ISL_476873, EPI_ISL_476877, EPI_ISL_476880, EPI_ISL_483825, EPI_ISL_483828, EPI_ISL_483833, EPI_ISL_483839, EPI_ISL_483845, EPI_ISL_483849, EPI_ISL_483852, EPI_ISL_483858, EPI_ISL_483862, EPI_ISL_483868, EPI_ISL_483871, EPI_ISL_483877, EPI_ISL_495017, EPI_ISL_495025, EPI_ISL_495028, EPI_ISL_495042, EPI_ISL_495047, EPI_ISL_495052, EPI_ISL_495059, EPI_ISL_495076, EPI_ISL_500947, EPI_ISL_512060, EPI_ISL_512066, EPI_ISL_512072, EPI_ISL_512076, EPI_ISL_514584, EPI_ISL_514591, EPI_ISL_514602, EPI_ISL_524713, EPI_ISL_524719, EPI_ISL_524728, EPI_ISL_524736, EPI_ISL_524748, EPI_ISL_524757, EPI_ISL_524763, EPI_ISL_525420, EPI_ISL_525421, EPI_ISL_525422 |
| | Maharastra | EPI-ISL-436444, EPI-ISL-450321 - EPI-ISL-450325, EPI-ISL-452192 - EPI-ISL-452198, EPI-ISL-452201 - EPI-ISL-452203, EPI-ISL-452205, EPI-ISL-452207 - EPI-ISL-452217, EPI-ISL-454524 - EPI-ISL-454529, EPI-ISL-454531 - EPI-ISL-454534, EPI-ISL-454536 - EPI-ISL-454537, EPI-ISL-454540, EPI-ISL-454542- EPI-ISL-454543, EPI-ISL-454546 - EPI-ISL-454547, EPI-ISL-454549, EPI-ISL-454551 - EPI-ISL-454552, EPI-ISL-454556 - EPI-ISL-454557, EPI-ISL-454560, EPI-ISL-454563 - EPI-ISL-454570, EPI-ISL-479495, EPI-ISL-479498, EPI-ISL-479503, EPI-ISL-479501, EPI-ISL-479505, EPI-ISL-479508, EPI-ISL-479510, EPI-ISL-479513, EPI-ISL-479515, EPI-ISL-479518, EPI-ISL-479527, EPI-ISL-479529, EPI-ISL-479534, EPI-ISL-479538, EPI-ISL-479542, EPI-ISL-479546, EPI-ISL-479550, EPI-ISL-479553, EPI-ISL-479558, EPI-ISL-479563, EPI-ISL-479567, EPI-ISL-479572, EPI-ISL-496534, EPI-ISL-496542, EPI-ISL-496546, EPI-ISL-496548, EPI-ISL-496551, EPI-ISL-496557, EPI-ISL-496561, EPI-ISL-496565, EPI-ISL-496569, EPI-ISL-496576, EPI-ISL-496580, EPI-ISL-496583, EPI-ISL-496602, EPI-ISL-508209, EPI-ISL-508217, EPI-ISL-508222, EPI-ISL-508226, EPI-ISL-508232, EPI-ISL-508239, EPI-ISL-508249, EPI-ISL-508252, EPI-ISL-508257, EPI-ISL-508264, EPI-ISL-508271, EPI-ISL-508275, EPI-ISL-508278, EPI-ISL-508284, EPI-ISL-508425, EPI-ISL-508431, EPI-ISL-508436, EPI-ISL-508440, EPI-ISL-508926, EPI-ISL-508934, EPI-ISL-508939 |
| South India | Tamilnadu | EPI-ISL-435075, EPI-ISL-435078 - EPI-ISL-435080, EPI-ISL-435083 - EPI-ISL-435084, EPI-ISL-435087, EPI-ISL-435091, EPI-ISL-435093, EPI-ISL-435094 - EPI-ISL-435096, EPI-ISL-436418, EPI-ISL-447584 - EPI-ISL-447587, EPI-ISL-481113, EPI-ISL-471584, EPI-ISL-458044, EPI-ISL-458042, EPI-ISL-458040, EPI-ISL-458038, EPI-ISL-458036, EPI-ISL-458033, EPI-ISL-458031, EPI-ISL-458030 |
| | Telengana | EPI-ISL-437626, EPI-ISL-438138, EPI-ISL-447847 - EPI-ISL-447866, EPI-ISL-447556 - EPI-ISL-447583, EPI-ISL-450326 - EPI-ISL-450331, EPI-ISL-450331, EPI_ISL_495297, EPI_ISL_495295, EPI_ISL_495290, EPI_ISL_495288, EPI_ISL_495285, EPI_ISL_495282, EPI_ISL_495280, EPI_ISL_495276, EPIISL_495272, EPI_ISL_495270, EPI_ISL_495267, EPI_ISL_495262, EPI_ISL_495258, EPI_ISL_495253, EPI_ISL_495249, EPIISL_495245, EPI_ISL_495240, EPI_ISL_495236, EPI_ISL_495232, EPI_ISL_495229, EPI_ISL_495226, EPI_ISL_495223, EPI_ISL_495219, EPIISL_495215, EPI_ISL_495211, EPI_ISL_495208, EPI_ISL_495205, EPIISL_495201, EPI_ISL_495198, EPI_ISL_495195, EPI_ISL_495192, EPI_ISL_495190, EPI_ISL_495188, EPI_ISL_495184, EPI_ISL_495180, EPIISL_495175, EPI_ISL_495169, EPI_ISL_495165, EPI_ISL_495163, EPIISL_471644, EPI_ISL_471641, EPI_ISL_471636, EPI_ISL_471631, EPIISL_471627, EPI_ISL_471623, EPI_ISL_471619, EPI_ISL_471616, EPIISL_471608, EPI_ISL_471603, EPI_ISL_471597, EPI_ISL_471591, EPIISL_471587, EPI_ISL_458077, EPI_ISL_458073, EPI_ISL_458068, EPIISL_458062, EPI_ISL_458058, EPI_ISL_458050, EPI_ISL_458046, EPI_ISL_458045 |
| | Karnataka | EPI-ISL-428479, EPI-ISL-428481 - EPI-ISL-428484, EPI-ISL-428486, EPI-ISL-428487, EPI-ISL-436137 - EPI-ISL-436141, EPI-ISL-436156, EPI-ISL-436157, EPI-ISL-436447, EPI_ISL_515971, EPI_ISL_515967, EPI_ISL_515942, EPI_ISL_508336, EPI_ISL_508331, EPI_ISL_508327, EPI_ISL_508323, EPI_ISL_508319, EPI_ISL_508311, EPI_ISL_508304, EPI_ISL_508299, EPI_ISL_508293, EPI_ISL_508288, EPI_ISL_486408, EPI_ISL_486399, EPI_ISL_486394, EPI_ISL_486383, EPI_ISL_477256, EPI_ISL_477242, EPI_ISL_477239, EPI_ISL_477210, EPI_ISL_477205 |
| | Kerala | EPI-ISL-413522, EPI-ISL-413523 |
| Central India | Madhya Pradesh | EPI-ISL-436453, EPI-ISL-436456, EPI-ISL-436457 - EPI-ISL-436463, EPI-ISL-452790 - EPI-ISL-452795, EPI-ISL-476023, EPI-ISL-476840, EPI-ISL-476842, EPI-ISL-476844, EPI-ISL-476846, EPI-ISL-476849, EPI-ISL-476852, EPI-ISL-476854, EPI-ISL-476883, EPI-ISL-476884, EPI-ISL-476886, EPI-ISL-476889, EPI-ISL-476891, EPI-ISL-476893, EPI-ISL-476894, EPI-ISL-476895, EPI-ISL-476896 |
| North India | Delhi | EPI-ISL-435061, EPI-ISL-435063 - EPI-ISL-435072, EPI-ISL-435108 - EPI-ISL-435110, EPI-ISL-436415, EPI-ISL-436424 - EPI-ISL-436426, EPI-ISL-436428 - EPI-ISL-436437, EPI-ISL-436445, EPI-ISL-436448, EPI-ISL-436450 - EPI-ISL-436452, EPI-ISL-436454 - EPI-ISL-436455, EPI_ISL_459911, EPI_ISL_459913, EPI_ISL_459919, EPI_ISL_459923, EPI_ISL_459933, EPI_ISL_459940, EPI_ISL_459943, EPI_ISL_482498, EPI_ISL_482512, EPI_ISL_482547, EPI_ISL_482555, EPI_ISL_482587, EPI_ISL_482612, EPI_ISL_482630, EPI_ISL_482635, EPI_ISL_482664, EPI_ISL_508417, EPI_ISL_508421, EPI_ISL_508422, EPI_ISL_508495 |
| | Haryana | EPI-ISL-435076, EPI-ISL-454858 - EPI-ISL-454867 |
| | Ladakh | EPI-ISL-435101 - EPI-ISL-435106 |
| | Jammu/ Kargil | EPI-ISL-435090, EPI-ISL-435107 |
| | Punjab | EPI-ISL-435062 |
| | Rajasthan | EPI-ISL-436420, EPI-ISL-454830 - EPI-ISL-454833, EPI-ISL-455655 |
| | Uttar Pradesh | EPI-ISL-435060, EPI-ISL-435082, EPI-ISL-435099, EPI-ISL-435100, EPI-ISL-436413, EPI-ISL-508202, EPI-ISL-508203, EPI-ISL-508419, EPI-ISL-508428, EPI-ISL-516940, EPI-ISL-516942, EPI-ISL-516946, EPI-ISL-516948, EPI-ISL-516949, EPI-ISL-516969, EPI-ISL-516974, EPI-ISL-516976, EPI-ISL-516977, EPI-ISL-516981, EPI-ISL-516983, EPI-ISL-516986 |
| | Uttarakhand | EPI-ISL-508156, EPI-ISL-508157, EPI-ISL-508159, EPI-ISL-508160, EPI-ISL-508160, EPI-ISL-508162, EPI-ISL-508164, EPI-ISL-508165, EPI-ISL-508169, EPI-ISL-508170, EPI-ISL-508172, EPI-ISL-508174, EPI-ISL-508175, EPI-ISL-508178, EPI-ISL-508180, EPI-ISL-508181, EPI-ISL-508182, EPI-ISL-508185, EPI-ISL-508187, EPI-ISL-508197, EPI-ISL-508201, EPI-ISL-508205, EPI-ISL-511908, EPI-ISL-511910, EPI-ISL-5011922 |

In a specific embodiment, a transgene encoding a SARS-CoV-2 spike protein or portion thereof (e.g., the ectodomain or receptor binding domain of SARS-CoV-2 spike protein) or a chimeric F protein is as described in the Examples (Sections 6-10), infra. In another specific embodiment, a transgene encoding a SARS-CoV-2 spike protein or portion thereof (e.g., the ectodomain or receptor binding domain of SARS-CoV-2 spike protein) or a chimeric F protein is one described in Section 6, 7, 8, 9, 10, 11 or 12, infra.

In certain embodiments, a transgene encoding a SARS-CoV-2 spike protein or portion thereof (e.g., the ectodomain or receptor binding domain of SARS-CoV-2 spike protein), or a chimeric F protein comprises NDV regulatory signals (e.g., gene end, intergenic, and gene start sequences) and Kozak sequences. In some embodiments, a transgene encoding a SARS-CoV-2 spike protein or portion thereof (e.g., the ectodomain or receptor binding domain of SARS-CoV-2 spike protein), or a chimeric F protein comprises NDV regulatory signals (e.g., gene end, intergenic, and gene start sequences), Kozak sequences and restriction sites to facilitate cloning. In certain embodiments, a transgene encoding a SARS-CoV-2 spike protein or portion thereof (e.g., the ectodomain or receptor binding domain of SARS-CoV-2 spike protein), or a chimeric F protein comprises NDV regulatory signals (gene end, intergenic and gene start sequences), Kozak sequences, restriction sites to facilitate cloning, and additional nucleotides in the non-coding region to ensure compliance with the rule of six. See, e.g., SEQ ID NOS: 20-23 for examples of a restriction sequence (SacII), a gene end sequence, a gene start sequence and a Kozak sequence that may be used. In a preferred embodiment, the transgene complies with the rule of six.

In a specific embodiment, a transgene described herein is isolated.

### 5.1.3 Recombinant NDV Encoding A Sars-Cov-2 Spike Protein or A Chimeric F Protein With A Sars-Cov-2 Spike Protein Ectodomain

In one aspect, presented herein are recombinant Newcastle disease virus (“NDV”) comprising a packaged genome, wherein the packaged genome comprises a transgene encoding a SARS-CoV-2 spike protein or portion thereof (e.g., the ectodomain or receptor binding domain of SARS-CoV-2 spike protein). See, e.g., Sections 5.1.2 and 6-12 for transgenes encoding a SARS-CoV-2 spike protein or portion thereof (e.g., the ectodomain or receptor binding domain of SARS-CoV-2 spike protein) which the packaged genome may comprise. In a specific embodiment, the SARS-CoV-2 spike protein or portion thereof (e.g., the ectodomain or receptor binding domain of SARS-CoV-2 spike protein) is expressed by cells infected with the recombinant NDV. In certain embodiments, the SARS-CoV-2 spike protein or portion thereof (e.g., the ectodomain or receptor binding domain of SARS-CoV-2 spike protein) is incorporated into the NDV virion. In another embodiment, described herein are recombinant NDV comprising a packaged genome, wherein the packaged genome comprises a transgene encoding a chimeric F protein, wherein the chimeric F protein comprises a SAR-CoV-2 spike protein ectodomain and NDV F protein transmembrane and cytoplasmic domains. In another embodiment, provided herein is a recombinant NDV comprising a packaged genome comprising a transgene that comprises a nucleotide sequence encoding a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site (e.g., amino acid residues 682 to 685 of the polybasic cleavage site are substituted for a single alanine). In another embodiment, provided herein is a recombinant NDV comprising a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site (e.g., amino acid residues 682 to 685 of the polybasic cleavage site are substituted for a single alanine). In other words, the NDV F protein transmembrane and cytoplasmic domains replace the SARS-CoV-2 spike protein transmembrane and cytoplasmic domains so that the chimeric F protein does not include the SARS-CoV-2 spike protein transmembrane and cytoplasmic domains. In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein ectodomain is fused directly to the NDV F protein transmembrane and cytoplasmic domains. In a specific embodiment, the NDV F protein transmembrane and cytoplasmic domains are from the same strain of NDV as the NDV backbone. For example, if the NDV backbone is NDV LaSota, then the transmembrane and cytoplasmic domains of the chimeric F protein are NDV LaSota transmembrane and cytoplasmic domains. See, e.g., Sections 5.1.2 and 6-12 for transgenes encoding a chimeric F protein which the packaged genome may comprise. In a specific embodiment, the chimeric F protein is expressed by cells infected with the recombinant NDV. In another specific embodiment, the chimeric F protein is incorporated into the NDV virion. In another specific embodiment, the chimeric F protein is expressed by cells infected with the recombinant NDV and the chimeric F protein is incorporated into the NDV virion.

In a specific embodiment, a recombinant NDV is as described in the Examples (Sections 6-10), infra. In a specific embodiment, a recombinant NDV one of the NDVs described Section 6, 7, 8, 9, 10, 11 or 12, infra.

In specific embodiments, a recombinant NDV described herein is replication competent. In other embodiments, a recombinant NDV described herein has been inactivated, such as described in Section 10.

In certain embodiments, the genome of the recombinant NDV does not comprise a heterologous sequence encoding a heterologous protein other than a SARS-CoV-2 spike protein or portion thereof (e.g., the ectodomain or receptor binding domain of SARS-CoV-2 spike protein). In some embodiments, the genome of the recombinant NDV does not comprise a transgene other than a transgene encoding a SARS-CoV-2 spike protein or portion thereof (e.g., the ectodomain or receptor binding domain of SARS-CoV-2 spike protein). In certain embodiments, a recombinant NDV described herein comprises a packaged genome, wherein the genome comprises the genes found in NDV and a transgene encoding a SARS-CoV-2 spike protein or portion thereof (e.g., the ectodomain or receptor binding domain of SARS-CoV-2 spike protein). In other words, the recombinant NDV encodes for both NDV F protein and the SARS-CoV-2 spike protein or portion thereof (e.g., the ectodomain or receptor binding domain of SARS-CoV-2 spike protein). In certain embodiments, a recombinant NDV described herein comprises a packaged genome, wherein the genome comprises the genes found in NDV, a transgene encoding a SARS-CoV-2 spike protein or portion thereof (e.g., the ectodomain or receptor binding domain of SARS-CoV-2 spike protein), a transgene encoding a SARS-CoV-2 nucleocapsid protein (see, e.g., in Section 5.1.4), but does not include another other transgenes. In some embodiments, a recombinant NDV described herein comprises a packaged genome, wherein the genome comprises the genes found in NDV and a transgene encoding a SARS-CoV-2 spike protein or portion thereof (e.g., the ectodomain or receptor binding domain of SARS-CoV-2 spike protein) but does not include any other transgenes.

In some embodiments, the packaged genome of NDV encodes a chimeric F protein, wherein the chimeric F protein comprises a SARS-CoV-2 spike protein ectodomain and NDV F protein transmembrane and cytoplasmic domains. In certain embodiments, the packaged genome of NDV comprises a transgene encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site (e.g., amino acid residues 682 to 685 of the polybasic cleavage site are substituted for a single alanine). In some embodiments, the packaged genome of NDV comprises a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site (e.g., amino acid residues 682 to 685 of the polybasic cleavage site are substituted for a single alanine). In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 spike protein ectodomain is fused directly to the NDV F protein transmembrane and cytoplasmic domains. In certain embodiment, the genome of the recombinant NDV does not comprise a heterologous sequence encoding a heterologous protein other than the chimeric F protein. In some embodiments, the genome of the recombinant NDV does not comprise a transgene other than a transgene encoding a chimeric F protein described herein. In preferred embodiments, a recombinant NDV described herein comprises a packaged genome, wherein the genome comprises the genes found in NDV and a transgene encoding a chimeric F protein. In other words, the recombinant NDV encodes for both NDV F protein and the chimeric F protein.

In a specific embodiment, provided herein is a NDV virion comprising a chimeric F protein described herein. See, e.g., Section 5.1.2 and the Examples (e.g., Section 10 or 12) for examples of a chimeric F protein that may incorporated into the virion of a recombinant NDV. In specific embodiments, the NDV virion is recombinantly produced.

In another embodiment, provided herein is a recombinant NDV comprising a chimeric F protein in its virion, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In a specific embodiment, the chimeric F protein comprises the amino acid sequence set forth in SEQ ID NO:13. In another specific embodiment, the chimeric F protein comprises an amino acid sequence that is at least 85%, at least 90%, at least 95%, at least 96%, at least 98% or at least 99% identical to the amino acid sequence set forth in SEQ ID NO:13.

In another embodiment, provided herein is a recombinant NDV comprising a chimeric F protein in its virion, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site. The SARS-CoV-2 spike protein ectodomain may lack the polybasic cleavage site as a result of amino acid residues 682 to 685 of the polybasic cleavage site being substituted with a single alanine. In certain embodiments, the NDV F protein transmembrane and cytoplasmic domains are fused to the SARS-CoV-2 spike protein ectodomain through a linker sequence (e.g., GGGGS (SEQ ID NO:24)). In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In a specific embodiment, the chimeric F protein comprises the amino acid sequence set forth in SEQ ID NO: 15. In another specific embodiment, the chimeric F protein comprises an amino acid sequence that is at least 85%, at least 90%, at least 95%, at least 96%, at least 98% or at least 99% identical to the amino acid sequence set forth in SEQ ID NO:15. In another specific embodiment, the chimeric F protein comprises the amino acid sequence set forth in SEQ ID NO:17. In another specific embodiment, the chimeric F protein comprises an amino acid sequence that is at least 85%, at least 90%, at least 95%, at least 96%, at least 98% or at least 99% identical to the amino acid sequence set forth in SEQ ID NO:17. In another specific embodiment, the chimeric F protein comprises the amino acid sequence set forth in SEQ ID NO:19. In another specific embodiment, the chimeric F protein comprises an amino acid sequence that is at least 85%, at least 90%, at least 95%, at least 96%, at least 98% or at least 99% identical to the amino acid sequence set forth in SEQ ID NO: 19.

### 5.1.4 Sars-Cov-2 Nucleocapsid Protein

In a specific embodiment, a transgene encoding a SARS-CoV-2 protein is incorporated into the genome of any NDV type or strain. See, e.g.,Section 5.1.1, supra, for types and strains of NDV that may be used. The transgene encoding any SARS-CoV-2 nucleocapsid protein may inserted into any NDV type or strain (e.g., NDV LaSota strain). One of skill in the art would be able to use such sequence information to produce a transgene for incorporation into the genome of any NDV type or strain. In a specific embodiment, a transgene encoding a SARS-CoV-2 nucleocapsid protein is codon optimized. See, e.g., Section 5.1.5, infra, for a discussion regarding codon optimization. The transgene encoding a SARS-CoV-2 nucleocapsid protein may be incorporated between any two NDV transcription units (e.g., between the NDV P and M transcription units, or between the HN and L transcription units).

In certain embodiments, a transgene encoding a SARS-CoV-2 nucleocapsid protein comprises NDV regulatory signals (e.g., gene end, intergenic, and gene start sequences) and Kozak sequences. In some embodiments, a transgene encoding a SARS-CoV-2 nucleocapsid comprises NDV regulatory signals (e.g., gene end, intergenic, and gene start sequences), Kozak sequences and restriction sites to facilitate cloning. In certain embodiments, a transgene encoding a SARS-CoV-2 nucleocapsid comprises NDV regulatory signals (gene end, intergenic and gene start sequences), Kozak sequences, restriction sites to facilitate cloning, and additional nucleotides in the non-coding region to ensure compliance with the rule of six. In a preferred embodiment, the transgene complies with the rule of six.

In a specific embodiment, a transgene described herein is isolated.

In a specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein. In certain embodiments, the NDV transcription units are LaSota NDV transcription units. In a specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at the amino acid residue corresponding to amino acid position 289 of LaSota NDV F protein. In another specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at amino acid position 289 of LaSota NDV F protein. In certain embodiments, the NDV transcription units are LaSota NDV transcription units. In certain embodiments, the nucleic acid sequence is part of a vector (e.g., a plasmid, such as described in the Examples below). In specific embodiments, the nucleic acid sequence is isolated.

In a specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein. In another specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position corresponding to amino acid residue 289 of LaSota NDV F. In another specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position 289 of LaSota NDV F. In certain embodiments, the NDV proteins are LaSota NDV proteins. In another specific embodiment, provided herein is a nucleic acid sequence comprising a nucleotide sequence of an NDV genome known in the art or described (see, e.g., Section 5.1 or the Examples below; see also SEQ ID NO: 1, 2 or 25) and a transgene described herein. In certain embodiments, the nucleic acid sequence is part of a vector (e.g., a plasmid, such as described in the Examples below). In a specific embodiment, the nucleotide sequence is isolated.

In specific embodiments, a nucleic acid sequence or nucleotide sequence described herein is a recombinant nucleic acid sequence or recombinant nucleotide sequence. In certain embodiments, a nucleotide sequence or nucleic acid sequence described herein may be a DNA molecule (e.g., cDNA), an RNA molecule, or a combination of a DNA and RNA molecule. In some embodiments, a nucleotide sequence or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The nucleic acid or nucleotide sequences can be single-stranded, double-stranded, may contain both single- stranded and double-stranded portions, and may contain triple-stranded portions. In a specific embodiment, a nucleotide sequence or nucleic acid sequence described herein is a negative sense single-stranded RNA. In another specific embodiment, a nucleotide sequence or nucleic acid sequence described herein is a positive sense single-stranded RNA. In another specific embodiment, a nucleotide sequence or nucleic acid sequence described herein is a cDNA.

In a specific embodiment, provided herein is a recombinant NDV comprising a packaged genome that comprises a transgene comprising a nucleotide sequence encoding a SARS-CoV-2 nucleocapid. In another specific embodiment, provided herein is recombinant NDV comprising a SARS-CoV-2 nucleocapsid in its virion.

### 5.1.5 Codon Optimization

Any codon optimization technique known to one of skill in the art may be used to codon optimize a nucleic acid sequence encoding a SARS-CoV-2 spike protein or a domain thereof (e.g., the ectodomain or receptor binding domain thereof). Similarly, any codon optimization technique may be used to codon optimize a nucleic acid sequence encoding a SARS-CoV-2 nucleocapsid protein. Methods of codon optimization are known in the art, e.g, the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence encoding a SARS-CoV-2 spike protein or a domain thereof (e.g., the ectodomain or receptor binding protein thereof), or a SARS-CoV-2 nucleocapsid protein is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. This nucleic acid sequence optimized for mammalian expression may be inspected for: (1) the presence of stretches of 5×A or more that may act as transcription terminators; (2) the presence of restriction sites that may interfere with subcloning; (3) compliance with the rule of six. Following inspection, (1) stretches of 5×A or more that may act as transcription terminators may be replaced by synonymous mutations; (2) restriction sites that may interfere with subcloning may be replaced by synonymous mutations; (3) NDV regulatory signals (gene end, intergenic and gene start sequences), and Kozak sequences for optimal protein expression may be added; and (4) nucleotides may be added in the non-coding region to ensure compliance with the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.2 Construction of NDVS

The recombinant NDVs described herein (see, e.g., Sections 5.1 and 6, 7, 9, 10, 11, and 12) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. Pat. Application Serial No. 09/152,845; in International Patent Publications PCT WO97/12032 published Apr. 3, 1997; WO96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain or LaSota strain) is constructed, inserted into a plasmid vector and engineered to contain a unique restriction site between two transcription units (e.g., the NDV P and M genes; or the NDV HN and L genes). A nucleotide sequence encoding a heterologous amino acid sequence (e.g., a transgene or other sequence described herein such as, e.g., a nucleotide sequence encoding a SARS-CoV-2 spike protein or portion thereof (e.g., ectodomain or receptor binding domain of the SARS-CoV-2 spike protein), a chimeric F protein, SARS-CoV-2 nucleocapsid protein) may be inserted into the viral genome at the unique restriction site. Alternatively, a nucleotide sequence encoding a heterologous amino acid sequence (e.g., a transgene or other sequence described herein such as, e.g., a nucleotide sequence encoding SARS-CoV-2 spike protein or portion thereof (e.g., ectodomain or receptor binding domain of the SARS-CoV-2 spike protein) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribozomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., García-Sastre et al., 1994, J. Virol. 68:6254-6261 and García-Sastre et al., 1994 Dev. Biol. Stand. 82:237-246, each of which are incorporated by reference herein in their entirety).

Methods for cloning recombinant NDV to encode a transgene and express a heterologous protein encoded by the transgene (e.g., a trangene comprises a nucleotide sequence encoding SARS-CoV-2 spike protein or portion thereof (e.g., ectodomain or receptor binding domain of the SARS-CoV-2 spike protein), a chimeric F protein or SARS-CoV-2 nucleocapsid) are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. Regarding the rule of six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of NDV (e.g., recombinant NDV), which is incorporated by reference herein in its entirety.

In a specific embodiment, an NDV described herein (see, e.g., Sections 5.1, and 6-12) may be generated according to a method described in Sections 6-10 and 12, infra.

In a specific embodiment, a recombinant NDV comprising a packaged genome comprising a transgene that comprises a nucleotide sequence encoding SARS-CoV-2 spike protein or portion thereof (e.g., ectodomain or receptor binding domain of the SARS-CoV-2 spike protein) described herein comprises a LaSota strain backbone. In another specific embodiment, a recombinant NDV comprising a packaged genome comprising a transgene that comprises a nucleotide sequence encoding SARS-CoV-2 spike protein or portion thereof (e.g., ectodomain or receptor binding domain of the SARS-CoV-2 spike protein) described herein comprises a LaSota strain backbone such as described in Section 6, 7, 9, 10, or 12. In a specific embodiment, the genomic sequence of the LaSota strain backbone (i.e., without the transgene) is as set forth in SEQ ID NO:1. In a specific embodiment, the genomic sequence of the La Sota strain backbone (i.e., without the transgene) is as set forth in SEQ ID NO:25. As the skilled person will appreciate the genome of NDV is negative-sense and single stranded. SEQ ID NOS:1 and 25 provide cDNA sequences.

In a specific embodiment, a recombinant NDV comprising a packaged genome comprising a transgene encoding a SARS-CoV-2 nucleocapsid protein described herein comprises a LaSota strain backbone. In another specific embodiment, a recombinant NDV comprising a packaged genome comprising a transgene encoding a SARS-CoV-2 nucleocapsid protein described herein comprises a LaSota strain backbone such as described in Section 6, 7, 9, 10 or 12. In a specific embodiment, the genomic sequence of the LaSota strain backbone (i.e., without the transgene) is as set forth in SEQ ID NO:1. In a specific embodiment, the genomic sequence of the La Sota strain backbone (i.e., without the transgene) is as set forth in SEQ ID NO:25. As the skilled person will appreciate the genome of NDV is negative-sense and single stranded. SEQ ID NOS:1 and 25 provide cDNA sequences.

In a specific embodiment, a recombinant NDV comprising a packaged genome comprising a transgene encoding a chimeric F protein described herein comprises a LaSota strain backbone. In a specific embodiment, a recombinant NDV comprising a packaged genome comprising a transgene encoding a chimeric F protein described herein comprises a LaSota strain backbone such as described in Section 6, 7, 9, 10 or 12. In a specific embodiment, the genomic sequence of the LaSota strain backbone (i.e., without the transgene) is as set forth in SEQ ID NO:1. In another specific embodiment, the genomic sequence of the LaSota strain backbone (i.e., without the transgene) is as set forth in SEQ ID NO:25. As the skilled person will appreciate the genome of NDV is negative-sense and single stranded. SEQ ID NOS:1 and 25 provide cDNA sequences.

### 5.3 Propagation of NDVS

The recombinant NDVs described herein (e.g., Sections 5.1 and 6-12) can be propagated in any substrate that allows the virus to grow to titers that permit the uses of the viruses described herein. In one embodiment, the substrate allows the recombinant NDVs described herein to grow to titers comparable to those determined for the corresponding wild-type viruses.

The recombinant NDVs described herein (e.g., Sections 5.1 and 6-12) may be grown in cells (e.g., avian cells, chicken cells, etc.) that are susceptible to infection by the viruses, embryonated eggs (e.g., chicken eggs or quail eggs) or animals (e.g., birds). Such methods are well-known to those skilled in the art. In a specific embodiment, the recombinant NDVs described herein may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the recombinant NDVs described herein may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained, derived, or obtained and derived from a human(s). In another embodiment, the recombinant NDVs described herein are propagated in interferon deficient systems or interferon (IFN) deficient substrates, such as, e.g., IFN deficient cells (e.g., IFN deficient cell lines) or IFN deficient embyronated eggs. In another embodiment, the recombinant NDVs described herein are propagated in chicken cells or embryonated chicken eggs. Representative chicken cells include, but are not limited to, chicken embryo fibroblasts and chicken embryo kidney cells. In a specific embodiment, the recombinant NDVs described herein are propagated in Vero cells. In another specific embodiment, the recombinant NDVs described herein are propagated in chicken eggs or quail eggs. In certain embodiments, a recombinant NDV virus described herein is first propagated in embryonated eggs and then propagated in cells (e.g., a cell line).

The recombinant NDVs described herein may be propagated in embryonated eggs (e.g. chicken embryonated eggs), e.g., from 6 to 14 days old, 6 to 12 days old, 6 to 10 days old, 6 to 9 days old, 6 to 8 days old, 8 to 10 day old, 9 to 11 days old, or 10 to 12 days old. In a specific embodiment, 10 day old embryonated chicken eggs are used to propagate the recombinant NDVs described herein. Young or immature embryonated eggs (e.g. chicken embryonated eggs) can be used to propagate the recombinant NDVs described herein. Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. The recombinant NDVs described herein can be propagated in different locations of the embryonated egg, e.g., the allantoic cavity (such as, e.g., the allantoic cavity of chicken embryonated eggs). For a detailed discussion on the growth and propagation viruses, see, e.g., U.S. Pat. No. 6,852,522 and U.S. Pat. No. 7,494,808, both of which are hereby incorporated by reference in their entireties.

In a specific embodiment, a virus is propagated as described one of the Examples below (e.g., Section 6, 7, 8, 9, 10, or 12).

For virus isolation, the recombinant NDVs described herein can be removed from embryonated eggs or cell culture and separated from cellular components, typically by well known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography. In a specific embodiment, a virus is isolated as described one of the Examples below (e.g., Section 6, 7, 8, 9, 10, or 12).

In a specific embodiment, virus isolation from allantoic fluid of an infected egg (e.g., a chicken egg) begins with harvesting allantoic fluid, which is clarified using a filtration system to remove cells and other large debris.

In a specific embodiment, provided herein is a cell (e.g., a cell line) or embryonated egg (e.g., a chicken embryonated egg) comprising a recombinant NDV described herein. In another specific embodiment, provided herein is a method for propagating a recombinant NDV described herein, the method comprising culturing a cell (e.g., a cell line) or embryonated egg (e.g., a chicken embryonated egg) infected with the recombinant NDV. In some embodiments, the method may further comprise isolating or purifying the recombinant NDV from the cell or embryonated egg. In a specific embodiment, provided herein is a method for propagating a recombinant NDV described herein, the method comprising (a) culturing a cell (e.g., a cell line) or embyronated egg infected with a recombinant NDV described herein; and (b) isolating the recombinant NDV from the cell or embyronated egg. The cell or embyronated egg may be one described herein or known to one of skill in the art. In some embodiments, the cell or embyronated egg is IFN deficient.

In a specific embodiment, provided herein is a method for producing a pharmaceutical composition (e.g., an immunogenic composition) comprising a recombinant NDV described herein, the method comprising (a) propagating a recombinant NDV described herein a cell (e.g., a cell line) or embyronated egg; and (b) isolating the recombinant NDV from the cell or embyronated egg. The method may further comprise adding the recombinant NDV to a container along with a pharmaceutically acceptable carrier.

### 5.4 Compositions and Routes of Administration

Provided herein are compositions comprising a recombinant NDV described herein (e.g., Section 5.1, 6, 7, 8, 9, 10, 11, or 12). In a specific embodiment, the compositions are pharmaceutical compositions, such as immunogenic compositions (e.g., vaccine compositions). In a specific embodiment, provided herein are immunogenic compositions comprising a recombinant NDV described herein (e.g., Section 5.1, 6, 7, 8, 9, 10, 11 or 12). The compositions may be used in methods of inducing an immune response to SARS-CoV-2 spike protein or nucleocapsid protein. The compositions may be used in methods for inducing an immune response to SARS-CoV-2 or immunizing against SARS-CoV-2. The compositions may be used in methods for immunizing against COVID-19. The compositions may be used in methods for preventing COVID-19.

In one embodiments, a pharmaceutical composition comprises a recombinant NDV described herein (e.g., Section 5.1, 6, 7, 8, 9, 10, 11 or 12), in an admixture with a pharmaceutically acceptable carrier. In some embodiments, the pharmaceutical composition further comprises one or more additional prophylactic or therapeutic agents. In a specific embodiment, a pharmaceutical composition comprises an effective amount of a recombinant NDV described herein (e.g., Section 5.1, 6, 7, 8, 9, 10, 11, or 12), and optionally one or more additional prophylactic or therapeutic agents, in a pharmaceutically acceptable carrier. In some embodiments, the recombinant NDV (e.g., Section 5.1, 6, 7, 8, 9, 10, 11 or 12) is the only active ingredient included in the pharmaceutical composition. In specific embodiments, two or more recombinant NDV are included in the pharmaceutical composition. In a particular embodiment, the pharmaceutical composition is an immunogenic composition. In a specific embodiment, administration of an immunogenic composition described herein to a subject (e.g., a human) generates neutralizing antibody (e.g., anti-SARS-CoV-2 spike protein IgG). In certain embodiments, administration of an immunogenic composition described herein to a subject (e.g., a human) generates an immune response that provides some level of protection against developing COVID-19. In some embodiments, administration of an immunogenic composition to a subject (e.g., human) generates an immune response in the subject that reduces the likelihood of developing COVID-19 by at least 25%, at least 50%, at least 75%, at least 80%, at least 85%, at least 90%, or at least 95% relative a subject of the same species not administered the immunogenic composition.

In a specific embodiment, a pharmaceutical composition comprises a first recombinant NDV and a second recombinant NDV, in an admixture with a pharmaceutically acceptable carrier, wherein the first recombinant NDV comprises a packaged genome comprising a first transgene, wherein the first transgene comprises a nucleotide sequence encoding a SARS-CoV-2 spike protein or portion thereof (e.g., ectodomain or receptor binding domain of the SARS-CoV-2 spike protein), and wherein the second recombinant NDV comprises a packaged genome comprising a second transgene, wherein the second transgene comprises a nucleotide sequence encoding a SARS-CoV-2 nucleocapsid protein. In another specific embodiment, a pharmaceutical composition comprises a first recombinant NDV and a second recombinant NDV, in an admixture with a pharmaceutically acceptable carrier, wherein the first recombinant NDV comprises a packaged genome comprising a first transgene, wherein the first transgene comprises a nucleotide sequence encoding a SARS-CoV-2 nucleocapsid protein, wherein the second recombinant NDV comprises a packaged genome comprising a second transgene, and wherein the second transgene comprises a nucleotide sequence encoding a chimeric F protein described herein. In another specific embodiment, a pharmaceutical composition comprises a first recombinant NDV and a second recombinant NDV, in an admixture with a pharmaceutically acceptable carrier, wherein the first recombinant NDV comprises a packaged genome comprising a first transgene, wherein the first transgene comprises a nucleotide sequence encoding a SARS-CoV-2 nucleocapsid protein, wherein the second recombinant NDV comprises a packaged genome comprising a second transgene, and wherein the second transgene comprises a nucleotide sequence encoding a chimeric F protein comprising a SARS-CoV-2 spike protein or portion thereof (e.g., ectodomain or receptor binding domain of the SARS-CoV-2 spike protein) and NDV F protein transmembrane and cytoplasmic domains. See, e.g., Section 5.1, 6, 7, 8, 9, 10, 11 or 12 for nucleic acid sequences encoding such transgenes. In a particular embodiment, the pharmaceutical composition is an immunogenic composition.

In a specific embodiment, the recombinant NDV included in a pharmaceutical composition described herein is a live virus. In particular, embodiment, the recombinant NDV included in a pharmaceutical composition described herein is an attenuated live virus. In some embodiments, the recombinant NDV included in a pharmaceutical composition described herein is inactivated. Any technique known to one of skill in the art may be used to inactivate a recombinant NDV described herein. For example, formalin or beta-propiolactone may be used to inactivate a recombinant NDV described herein. In a specific embodiment, the recombinant NDV included in a pharmaceutical described herein is inactivated using 2% beta-Propiolactone, such as described in Section 10, infra, or another technique known to one of skill in the art. For example, in certain embodiments, to prepare inactivated concentrated recombinant NDV, 1 part of 0.5 M disodium phosphate (DSP) may be mixed with 38 parts of the allantoic fluid of an embryonated egg infected with the virus to stabilize the pH, one part of 2% beta-Propiolactone (BPL) is added dropwise to the mixture during shaking, and incubated on ice for 30 min, the mixture is then placed in a 37° C. water bath for approximately 1 to 3 hours shaken every 5-30 min. The inactivated allantoic fluid is clarified by centrifugation at 4,000 rpm for 20-40 minutes. In a specific embodiment, a chimeric F protein is stable in an inactivated recombinant NDV described herein for a period of time as assessed using the methodology described in Section 10, infra.

The pharmaceutical compositions provided herein can be in any form that allows for the composition to be administered to a subject. In a specific embodiment, the pharmaceutical compositions are suitable for veterinary administration, human administration, or both. As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington’s Pharmaceutical Sciences” by E.W. Martin. The formulation should suit the mode of administration.

In a specific embodiment, the pharmaceutical compositions are formulated to be suitable for the intended route of administration to a subject. For example, the pharmaceutical composition may be formulated to be suitable for parenteral, intravenous, intraarterial, intrapleural, inhalation, intranasal, intraperitoneal, oral, intradermal, colorectal, intraperitoneal, intracranial, and intratumoral administration. In one embodiment, the pharmaceutical composition may be formulated for intravenous, intraarterial, oral, intraperitoneal, intranasal, intratracheal, intrapleural, intracranial, subcutaneous, intramuscular, topical, pulmonary, or intratumoral administration. In a specific embodiment, the pharmaceutical composition may be formulated for intranasal administration. In another embodiment, the pharmaceutical composition may be formulated for intramuscular administration.

In a specific embodiment, the pharmaceutical composition comprising a recombinant NDV described herein (see, e.g., Sections 5.1 and 6-12) is formulated to be suitable for intranasal administration to the subject (e.g., human subject). In a particular embodiment, the pharmaceutical composition is an immunogenic composition.

In a specific embodiment, a pharmaceutical composition described herein (e.g., a pharmaceutical composition comprising an inactivated recombinant NDV described herein) may comprise an adjuvant. In certain embodiments, the compositions described herein comprise, or are administered in combination with, an adjuvant. The adjuvant for administration in combination with a composition described herein may be administered before, concommitantly with, or after administration of the composition. In specific embodiments, an inactivated virus immunogenic composition described herein comprises one or more adjuvants. In some embodiments, the term “adjuvant” refers to a compound that when administered in conjunction with or as part of a composition described herein augments, enhances and/or boosts the immune response to a recombinant NDV, but when the compound is administered alone does not generate an immune response to the virus. In some embodiments, the adjuvant generates an immune response to a recombinant NDV and does not produce an allergy or other adverse reaction. Adjuvants can enhance an immune response by several mechanisms including, e.g., lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund’s adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as poly glutamic acid or polylysine. In a specific embodiment, the adjuvant is an adjuvant described in Section 10, infra. In certain embodiments, the adjuvant is a liposomal suspension adjuvant (R-enantiomer of the cationic lipid DOTAP, R-DOTAP) or an MF-59 like oil-in-water emulsion adjuvant (AddaVax). In some embodiments, the adjuvant is a toll-like receptor 9 (TLR9) agonist adjuvant. In certain embodiments, the adjuvant is CpG 1018, such as described in Section 11, infra. In some embodiments, a composition described herein (e.g., a live recombinant NDV composition) does not contain an adjuvant.

In a specific embodiment, a pharmaceutical composition (e.g., an immunogenic composition) is one described in the Examples (e.g., Section 7, 8, 9, or 10). In another specific embodiment, a pharmaceutical composition (e.g., an immunogenic composition) is one described in the Section 6, 7, 8, 9, 10, 11 or 12).

In certain embodiments, a pharmaceutical composition (e.g., an immunogenic composition) described herein comprises 10⁴ to 10¹² EID50 of a recombinant NDV described herein. In some embodiments, pharmaceutical composition (e.g., an immunogenic composition) described herein comprises 1 to 15 micrograms of SARS-CoV-2 spike protein or a portion or a chimeric F protein expressed by a recombinant NDV described herein. In some embodiments, pharmaceutical composition (e.g., an immunogenic composition) described herein comprises 1 to 15 micrograms per ml of SARS-CoV-2 spike protein or a portion or a chimeric F protein expressed by a recombinant NDV described herein.

In a specific embodiment, a pharmaceutical composition described herein may be stored at 2 ° to 8° C. In certain embodiments, a pharmaceutical composition described herein is stable for at least 1 month, at least 2 months, at least 3 months, at least 4 months, at least 5 months, at least 6 months, at least 9 months or at least 1 year at 2° to 8° C. In some embodiments, a pharmaceutical composition described herein is stable for 3-6 months, 3-9 months, 6-12 months, or 9-12 months at 2° to 8° C. In certain embodiments, the stability is assessed by protein denaturation assays, immunoassays or a combination thereof.

### 5.5 Uses of a Recombinant NDV

### 5.5.1 Prevention of Covid-19

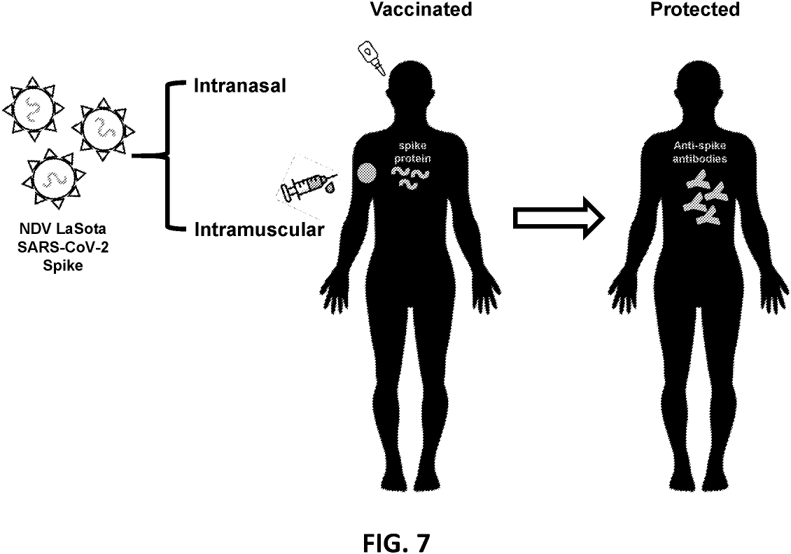

The recombinant NDV described herein may be used to immunize a subject against SARS-CoV-2, induce an immune response to a SARS-CoV-2 spike protein or nucleocapsid protein, or prevent COVID-19. See, e.g., FIG. 7 for uses of the recombinant NDV described herein. In one aspect, presented herein are methods for inducing an immune response in a subject (e.g., a human subject) comprising administering the subject (e.g., a human subject) a recombinant NDV described herein or a composition comprising a recombinant NDV described herein. See, e.g., Section 5.1 and the Examples for recombinant NDV and Section 5.4 as well as the Examples (e.g., Sections 10 and 11) for compositions. In another aspect, presented herein are methods for inducing an immune response in a subject (e.g., a human subject) comprising administering the subject (e.g., a human subject) a recombinant NDV, wherein the recombinant NDV comprises a packaged genome comprising a transgene that comprises a nucleotide sequence encoding a SARS-CoV-2 spike protein or portion thereof (e.g., the ectodomain or receptor binding domain of a SARS-CoV-2 spike protein). See, e.g., Sections 5.1.2 and 6-12 for transgenes encoding a SARS-CoV-2 spike protein or portion thereof (e.g., the ectodomain or receptor binding domain of a SARS-CoV-2 spike protein) which the packaged genome may comprise. See also Sections 5.1.3 and 6-12 for examples of recombinant NDV that may be used in the methods. In a specific embodiment, the transgene comprises a codon optimized nucleic acid sequence encoding the SARS-CoV-2 spike protein or portion thereof (e.g., the ectodomain or receptor binding domain of a SARS-CoV-2 spike protein). In another aspect, presented herein are methods for inducing an immune response in a subject (e.g., a human subject) comprising administering the subject (e.g., a human subject) a recombinant NDV, wherein the recombinant NDV comprises a packaged genome comprising a transgene that comprises a nucleotide sequence encoding a chimeric F protein, wherein the chimeric F protein comprises the ectodomain of a SARS-CoV-2 spike protein and the transmembrane and cytoplasmic domains of NDV F protein. In specific embodiments, the SARS-CoV-2 spike protein ectodomain lacks the polybasic cleavage site (e.g., amino acid residues of the polybasic cleavage site (RRAR) are substituted with a single alainine). In another embodiment, presented herein are methods for inducing an immune response against SARS-CoV-2 spike protein in a subject (e.g., a human subject) against SARS-CoV-2 comprising administering the subject (e.g., a human subject) a recombinant NDV, wherein the recombinant NDV comprises a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site (e.g., amino acid residues 682 to 685 of the polybasic cleavage site are substituted for a single alanine). In specific embodiments, the SARS-CoV-2 spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. See, e.g., Sections 5.1.2 and 6-10 for transgenes encoding a chimeric F protein which the packaged genome may comprise. See also Sections 5.1.3 and 6-12 for examples of recombinant NDV that may be used in the methods. In a specific embodiment, the ectodomain of the SARS-CoV-2 spike protein is encoded by a codon optimized nucleic acid sequence. In certain embodiments, the method further comprises administering to the subject a second recombinant NDV, wherein the second recombinant NDV comprises a packaged genome comprising a transgene that comprises a nucleotide sequence encoding a SARS-CoV-2 nucleocapsid protein.

In another aspect, presented herein are methods for inducing an immune response in a subject (e.g., a human subject) comprising administering the subject (e.g., a human subject) a recombinant NDV, wherein the recombinant NDV comprises a packaged genome comprising a transgene that comprises a nucleotide sequence encoding a SARS-CoV-2 nucleocapsid . See, e.g., Sections 5.1.2 and 6-10 for transgenes encoding a SARS-CoV-2 nucleocapsid protein which the packaged genome may comprise. See also Sections 5.1.4 and 6-12 for examples of recombinant NDV that may be used in the methods. In a specific embodiment, the transgene comprises a codon optimized nucleic acid sequence encoding the SARS-CoV-2 nucleocapsid protein. See also Sections 5.1.3 and 6-12 for examples of recombinant NDV that may be used in the methods.

In another aspect, presented herein are methods for immunizing a subject (e.g., a human subject) against SARS-CoV-2 comprising administering the subject (e.g., a human subject) a recombinant NDV described herein or a composition comprising a recombinant NDV described herein. See, e.g., Section 5.1 and the Examples for recombinant NDV and Section 5.4 as well as the Examples (e.g., Sections 10 and 11) for compositions. In another aspect, presented herein are methods for immunizing a subject (e.g., a human subject) against SARS-CoV-2 comprising administering the subject (e.g., a human subject) a recombinant NDV, wherein the recombinant NDV comprises a packaged genome comprising a transgene that comprises a nucleotide sequence encoding a SARS-CoV-2 spike protein or portion thereof (e.g., the ectodomain or receptor binding domain of a SARS-CoV-2 spike protein) . See, e.g., Section 5.1.2 and 6-12 for transgenes encoding a SARS-CoV-2 spike protein or portion thereof (e.g., the ectodomain or receptor binding domain of a SARS-CoV-2 spike protein) which the packaged genome may comprise. See also Sections 5.1.3 and 6-12 for examples of recombinant NDV that may be used in the methods. In a specific embodiment, the transgene comprises a codon optimized nucleic acid sequence encoding the SARS-CoV-2 spike protein or portion thereof (e.g., the ectodomain or receptor binding domain of a SARS-CoV-2 spike protein). In another aspect, presented herein are methods for immunizing a subject (e.g., a human subject) against SARS-CoV-2 comprising administering the subject (e.g., a human subject) a recombinant NDV, wherein the recombinant NDV comprises a packaged genome comprising a transgene that comprises a nucleotide sequence encoding a chimeric F protein, wherein the chimeric F protein comprises the ectodomain of a SARS-CoV-2 spike protein and the transmembrane and cytoplasmic domains of NDV F protein. In one embodiment, presented herein are methods for immunizing a subject (e.g., a human subject) against SARS-CoV-2 comprising administering the subject (e.g., a human subject) a recombinant NDV, wherein the recombinant NDV comprises a packaged genome comprising a transgene that comprises a nucleotide sequence encoding a chimeric F protein, wherein the chimeric F protein comprises the ectodomain of a SARS-CoV-2 spike protein and the transmembrane and cytoplasmic domains of NDV F protein, wherein the SARS-CoV-2 spike protein ectodomain lacks a polybasic cleavage site (e.g.,amino acid residues 682 to 685 of the polybasic cleavage site are substituted for a single alanine). In another embodiment, presented herein are methods for immunizing a subject (e.g., a human subject) against SARS-CoV-2 comprising administering the subject (e.g., a human subject) a recombinant NDV, wherein the recombinant NDV comprises a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site (e.g.,amino acid residues 682 to 685 of the polybasic cleavage site are substituted for a single alanine). In specific embodiments, the SARS-CoV-2 spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. See, e.g., Sections 5.1.2 and 6-10 as well as Section 12 for transgenes encoding a chimeric F protein which the packaged genome may comprise. See also Sections 5.1.3 and 6-12 for examples of recombinant NDV that may be used in the methods. In a specific embodiment, the ectodomain of the SARS-CoV-2 spike protein is encoded by a codon optimized nucleic acid sequence. In certain embodiments, the method further comprises administering to the subject a second recombinant NDV, wherein the second recombinant NDV comprises a packaged genome comprising a transgene that comprises a nucleotide sequence encoding a SARS-CoV-2 nucleocapsid protein.

In another aspect, presented herein are methods for immunizing a subject (e.g., a human subject) against SARS-CoV-2 comprising administering the subject (e.g., a human subject) a recombinant NDV, wherein the recombinant NDV comprises a packaged genome comprising a transgene that comprises a nucleotide sequence encoding a SARS-CoV-2 nucleocapsid . See, e.g., Sections 5.1.2 and 6-12 for transgenes encoding a SARS-CoV-2 nucleocapsid protein which the packaged genome may comprise. See also Sections 5.1.3 and 6-12 for examples of recombinant NDV that may be used in the methods. In a specific embodiment, the transgene comprises a codon optimized nucleic acid sequence encoding the SARS-CoV-2 nucleocapsid protein. See also Sections 5.1.3 and 6-10 for examples of recombinant NDV that may be used in the methods.

In another aspect, presented herein are methods for preventing COVID-19 in a subject (e.g., a human subject) comprising administering the subject (e.g., a human subject) a recombinant NDV described herein or a composition comprising a recombinant NDV described herein. See, e.g., Section 5.1 and the Examples for recombinant NDV and Section 5.4 as well as the Examples (e.g., Sections 10 and 11) for compositions. In another aspect, presented herein are methods for preventing COVID-19 in a subject (e.g., a human subject) comprising administering the subject (e.g., a human subject) a recombinant NDV, wherein the recombinant NDV comprises a packaged genome comprising a transgene that comprises a nucleotide sequence encoding a SARS-CoV-2 spike protein or portion thereof (e.g., the ectodomain or receptor binding domain of a SARS-CoV-2 spike protein). See, e.g., Sections 5.1.2 and 6-10 for transgenes encoding a SARS-CoV-2 spike protein or portion thereof (e.g., the ectodomain or receptor binding domain of a SARS-CoV-2 spike protein) which the packaged genome may comprise. See also Sections 5.1.3 and 6-10 for examples of recombinant NDV that may be used in the methods. In a specific embodiment, the transgene comprises a codon optimized nucleic acid sequence encoding the SARS-CoV-2 spike protein or portion thereof (e.g., the ectodomain or receptor binding domain of a SARS-CoV-2 spike protein). In another aspect, presented herein are methods for preventing COVID-19 in a subject (e.g., a human subject) comprising administering the subject (e.g., a human subject) a recombinant NDV, wherein the recombinant NDV comprises a packaged genome comprising a transgene that comprises a nucleotide sequence encoding a chimeric F protein, wherein the chimeric F protein comprises the ectodomain of a SARS-CoV-2 spike protein and the transmembrane and cytoplasmic domains of NDV F protein. In one embodiment, presented herein are methods for preventing COVID-19 in a subject (e.g., a human subject) comprising administering the subject (e.g., a human subject) a recombinant NDV, wherein the recombinant NDV comprises a packaged genome comprising a transgene that comprises a nucleotide sequence encoding a chimeric F protein, wherein the chimeric F protein comprises the ectodomain of a SARS-CoV-2 spike protein and the transmembrane and cytoplasmic domains of NDV F protein, wherein the SARS-CoV-2 spike protein ectodomain lacks a polybasic cleavage site (e.g.,amino acid residues 682 to 685 of the polybasic cleavage site are substituted for a single alanine). In another embodiment, presented herein are methods for preventing COVID-19 a subject (e.g., a human subject) comprising administering the subject (e.g., a human subject) a recombinant NDV, wherein the recombinant NDV comprises a packaged genome, wherein the packaged genome comprises a transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site (e.g., amino acid residues 682 to 685 of the polybasic cleavage site are substituted for a single alanine). In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. See, e.g., Sections 5.1.2 and 6-12 for transgenes encoding a chimeric F protein which the packaged genome may comprise. See also Sections 5.1.3 and 6-12 for examples of recombinant NDV that may be used in the methods. In a specific embodiment, the ectodomain of the SARS-CoV-2 spike protein is encoded by a codon optimized nucleic acid sequence. In certain embodiments, the method further comprises administering to the subject a second recombinant NDV, wherein the second recombinant NDV comprises a packaged genome comprising a transgene that comprises a nucleotide sequence encoding a SARS-CoV-2 nucleocapsid protein.

In another aspect, presented herein are methods for preventing COVID-19 in a subject (e.g., a human subject) comprising administering the subject (e.g., a human subject) a recombinant NDV, wherein the recombinant NDV comprises a packaged genome comprising a transgene that comprises a nucleotide sequence encoding a SARS-CoV-2 nucleocapsid . See, e.g., Sections 5.1.2 and 6-12 for transgenes encoding a SARS-CoV-2 nucleocapsid protein which the packaged genome may comprise. See also Sections 5.1.3 and 6-12 for examples of recombinant NDV that may be used in the methods. In a specific embodiment, the transgene comprises a codon optimized nucleic acid sequence encoding the SARS-CoV-2 nucleocapsid protein. See also Sections 5.1.3 and 6-12 for examples of recombinant NDV that may be used in the methods.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally. See, e.g., Sections 5.1, and 6-12, infra for information regarding recombinant NDV, Section 5.5.3 for information regarding other therapies, and Section 5.4, infra, for information regarding compositions and routes of administration.

The recombinant NDV and one or more additional therapies may be administered concurrently or sequentially to the subject. In certain embodiments, the recombinant NDV and one or more additional therapies are administered in the same composition. In other embodiments, the recombinant NDV and one or more additional therapies are administered in different compositions. The recombinant NDV and one or more other therapies may be administered by the same or different routes of administration to the subject. Any route known to one of skill in the art or described herein may be used to administer the recombinant NDV and one or more other therapies. In a specific embodiment, the recombinant NDV is administered intranasally or intramuscularly and the one or more other therapies are administered by the same or a different route. In a specific embodiment, the recombinant NDV is administered intranasally and the one or more other therapies is administered intravenously.

In some embodiments, a recombinant NDV described herein or a composition thereof, or a combination therapy described herein is administered to a patient to prevent the onset of one, two or more symptoms of COVID-19. In a specific embodiment, the administration of a recombinant NDV described herein or a composition thereof, or a combination therapy described herein to a subject prevents the onset or development of one, two or more symptoms of COVID-19, reduces the severity of one, two or more symptoms of COVID-19, or prevents the onset or development of one, two or more symptoms of COVID-19 and reduces the severity of one, two or more symptoms of COVID-19. Symptoms of COVID-19 include congested or runny nose, cough, fever, sore throat, headache, wheezing, rapid or shallow breathing or difficulty breathing, bluish color the skin due to lack of oxygen, chills, muscle pain, loss of taste and/or smell, nausea, vomiting, and diarrhea.

In a specific embodiment, the administration of a recombinant NDV described herein or a composition thereof, or a combination therapy described herein to a subject prevents the spread of SARS-CoV-2 infection. In another specific embodiment, the administration of a recombinant NDV described herein or a composition thereof, or a combination therapy described herein to a subject prevents hospitalization. In another specific embodiment, the administration of a recombinant NDV described herein or a composition thereof, or a combination therapy described herein to a subject prevents COVID-19. In another embodiment, the administration of a recombinant NDV described herein or a composition thereof, or a combination therapy described herein to a subject reduces the length of hospitalization. In another embodiment, the administration of a recombinant NDV described herein or a composition thereof, or a combination therapy described herein to a subject reduces the likelihood of intubation. In another specific embodiment, the administration of a recombinant NDV described herein or a composition thereof, or a combination therapy described herein to a subject prevents recurring SARS-CoV-2 infections. In another specific embodiment, the administration of a recombinant NDV described herein or a composition thereof, or a combination therapy described herein to a subject prevents asymptomatic SARS-CoV-2 infection.

In another specific embodiment, the administration of a recombinant NDV described herein or a composition thereof induces antibodies to SARS-CoV-2 spike protein or nucleocapsid protein. In another specific embodiment, the administration of a recombinant NDV described herein or a composition thereof induces both mucosal and systemic antibodies to SARS-CoV-2 spike protein or nucleocapsid protein (e.g., neutralizing antibodies). In another specific embodiment, the administration of a recombinant NDV described herein or a composition thereof, or a combination therapy described herein to a subject induces neutralizing IgG antibody to SARS-CoV-2 spike protein. In another specific embodiment, the administration of a recombinant NDV described herein or a composition thereof, or a combination therapy described herein to a subject induces IgG antibody to SARS-CoV-2 spike protein at a level that is considerate moderate to high in an ELISA approved by the FDA for measuring antibody in a patient specimen. In another specific embodiment, the administration of a recombinant NDV described herein or a composition thereof, or a combination therapy described herein to a subject induces neutralizing antibody to SARS-CoV-2 spike protein. In another specific embodiment, the administration of a recombinant NDV described herein or a composition thereof, or a combination therapy described herein to a subject induces robust, long-lived (e.g., 6 months, 1 year, 2 years, 3 years or more), antigen-specific humoral immunity. In another specific embodiment, the administration of a recombinant NDV described herein or a composition thereof, or a combination therapy described herein to a subject induces T cell immunity.

In a specific embodiment, recombinant NDV described herein or a composition thereof, or a combination therapy described herein induces protective immunity in a subject (e.g., a human subject or non-human subject). In a particular, recombinant NDV described herein or a composition thereof, or a combination therapy described herein induces immunity in a subject (e.g., a human subject or non-human subject) that protects (partially or completely) the subject from disease (e.g., COVID-19) due to subsequent infection by SARS-CoV-2.

In some embodiments, a recombinant NDV described herein or a composition thereof, or a combination therapy described herein is administered to a subject predisposed or susceptible to COVID-19.

In certain embodiments, a recombinant NDV described herein or a composition thereof, or a combination therapy described herein is administered to a human. In some embodiments, a recombinant NDV described herein or a composition thereof, or a combination therapy described herein is administered to a human infant. In another specific embodiment, the subject is a human infant six months old or older. In other embodiments, a recombinant NDV described herein or a composition thereof, or a combination therapy described herein is administered to a human toddler. In other embodiments, a recombinant NDV described herein or a composition thereof, or a combination therapy described herein is administered to a human child. In other embodiments, a recombinant NDV described herein or a composition thereof, or a combination therapy described herein is administered to a human adult. In yet other embodiments, a recombinant NDV described herein or a composition thereof, or a combination therapy described herein is administered to an elderly human.

In a specific embodiment, a recombinant NDV described herein or a composition thereof, or a combination therapy described herein is administered a subject (e.g., a human subject) in close contact with an individual with increased risk of COVID-19 or SARS-CoV-2 infection. In some embodiments, a recombinant NDV described herein or a composition thereof, or a combination therapy described herein is administered a subject (e.g., a human subject) with a condition that increases susceptibility to SARS-CoV-2 complications or for which SARS-CoV-2 increases complications associated with the condition. Examples of conitions that increase susceptibility to SARS-CoV-2 complications or for which SARS-CoV-2 increases complications associated with the condition include conditions that affect the lung, such as cystic fibrosis, chronic obstructive pulmonary disease (COPD), emphysema, asthma, or bacterial infections (e.g., infections caused by Haemophilus influenzae, Streptococcus pneumoniae, Legionella pneumophila, and Chlamydia trachomatus); cardiovascular disease (e.g., congenital heart disease, congestive heart failure, and coronary artery disease); and endocrine disorders (e.g., diabetes).

In some embodiments, a recombinant NDV described herein or a composition thereof, or a combination therapy described herein is administered a subject (e.g., a human subject) that resides in a group home, such as a nursing home. In some embodiments, a recombinant NDV described herein or a composition thereof, or a combination therapy described herein is administered a subject (e.g., a human subject) that works in, or spends a significant amount of time in, a group home, e.g., a nursing home. In some embodiments, a recombinant NDV described herein or a composition thereof, or a combination therapy described herein is administered a subject (e.g., a human subject) that is a health care worker (e.g., a doctor or nurse). In some embodiments, a recombinant NDV described herein or a composition thereof, or a combination therapy described herein is administered a subject (e.g., a human subject) that is a smoker.

In some embodiments, a recombinant NDV described herein or a composition thereof, or a combination therapy described herein is administered to (1) a subject (e.g., a human subject) who can transmit SARS-CoV-2 to those at high risk for complications, such as, e.g., members of households with high-risk subjects, including households that will include human infants (e.g., infants younger than 6 months), (2) a subject coming into contact with human infants (e.g., infants less than 6 months of age), (3) a subject who will come into contact with subjects who live in nursing homes or other long-term care facilities, (4) a subject who is or will come into contact with an elderly human, or (5) a subject who will come into contact with subjects with long-term disorders of the lungs, heart, or circulation; individuals with metabolic diseases (e.g., diabetes) or subjects with weakened immune systems (including immunosuppression caused by medications, malignancies such as cancer, organ transplant, or HIV infection).

In specific embodiments, a recombinant NDV described herein or a composition thereof, or a combination therapy described herein is administered to a subject (e.g., human) that fulfills one, two or more, or all of the inclusion criteria described in Section 11, infra. In some embodiments, a recombinant NDV described herein or a composition thereof, or a combination therapy described herein is administered to a subject (e.g., human) that fulfills one, two or more, or all of the inclusion criteria described in Section 11, infra, and meets one, two or more, or all of the exclusion criteria described in Section 11, infra. In certain embodiments, a recombinant NDV described herein or a composition thereof, or a combination therapy described herein is administered to a subject (e.g., human) that fulfills one, two or more, or all of the criteria described in Section 11, infra.

### 5.5.2 Dosage and Frequency

The amount of a recombinant NDV or a composition thereof, which will be effective in the prevention of COVID-19, or immunization against SARS-CoV-2 will depend on the route of administration, the general health of the subject, etc. Standard clinical techniques, such as in vitro assays, may optionally be employed to help identify dosage ranges. However, suitable dosage ranges of a recombinant NDV for administration are generally about 10⁴ to about 10¹², and can be administered to a subject once, twice, three, four or more times with intervals as often as needed. In some embodiments, a recombinant NDV described herein is administered to a subject (e.g., human) at a dose of 10⁴ to about 10¹² EID50. In certain embodiments, a recombinant NDV described herein is administered to a subject (e.g., human) at a dose of 1 to 15 micrograms of SARS-CoV-2 spike protein or a portion or a chimeric F protein. In some embodiments, a recombinant NDV described herein is administered to a subject (e.g., human) at a dose of 1 to 10 micrograms of SARS-CoV-2 spike protein or a portion or a chimeric F protein. In a specific embodiment, a recombinant NDV described herein is administered to a subject (e.g., human) at a dose of 1 microgram, 3 micrograms, or 10 micrograms of SARS-CoV-2 spike protein or a portion or a chimeric F protein. In another specific embodiment, a recombinant NDV described herein is administered to a subject (e.g., human) at a dose of 4 micrograms, 5 micrograms, 6 micrograms, 7 micrograms, 8 micrograms or 9 micrograms of SARS-CoV-2 spike protein or a portion or a chimeric F protein. In certain embodiments, a composition described herein is administered to a subject (e.g., human) at a dose of 1 to 15 micrograms of SARS-CoV-2 spike protein or a portion or a chimeric F protein. In some embodiments, a composition described herein is administered to a subject (e.g., human) at a dose of 1 to 10 micrograms of SARS-CoV-2 spike protein or a portion or a chimeric F protein. In a specific embodiment, a composition NDV described herein is administered to a subject (e.g., human) at a dose of 1 microgram, 3 micrograms, or 10 micrograms of SARS-CoV-2 spike protein or a portion or a chimeric F protein. In another specific embodiment, a composition described herein is administered to a subject (e.g., human) at a dose of 4 micrograms, 5 micrograms, 6 micrograms, 7 micrograms, 8 micrograms or 9 micrograms of SARS-CoV-2 spike protein or a portion or a chimeric F protein. In another specific embodiment, a recombinant NDV described herein is administered to a subject (e.g., human) at a dose described in Section 11, infra. In certain embodiments, dosages similar to those currently being used in clinical trials for NDV are administered to a subject.

In certain embodiments, a recombinant NDV or a composition thereof is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In accordance with these embodiments, booster inoculations may be administered to the subject at 3 to 6 month or 6 to 12 month intervals following the second inoculation. In certain embodiments, a subject is administered one or more boosters. The recombinant NDV used for each booster may be the same or different.

In certain embodiments, administration of the same recombinant NDV or a composition thereof may be repeated and the administrations may be separated by at least 7 days, 10 days, 14 days, 15 days, 21 days, 28 days, 30 days, 45 days, 2 months, 75 days, 3 months, or at least 6 months. In other embodiments, administration of the same recombinant NDV or a composition thereof may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first recombinant NDV or a composition thereof is administered to a subject followed by the administration of a second recombinant NDV or a composition thereof. In some embodiments, the first and second recombinant NDV are different from each other. In certain embodiments, a first pharmaceutical composition is administered to a subject as a priming dose and after a certain period (e.g., 1 month, 2 months, 3 months, 4 monthts, 5 months, 6 months, or 1-6 months) a booster dose of a second pharmaceutical composition is administered. For example, the first recombinant NDV may comprise a packaged genome comprising a transgene that comprises a nucleotide sequence encoding a SARS-CoV-2 spike protein or portion thereof (e.g., ectodomain or receptor binding domain of the SARS-CoV-2 spike protein), and the second recombinant NDV may comprise a package genome comprising a transgene that comprises a nucleotide sequence encoding a chimeric F protein, wherein the chimeric F protein comprises a SARS-CoV-2 spike protein ectodomain and NDV F protein transmembrane and cytoplasmic domains. In another example, the first recombinant NDV may comprise a packaged genome comprising a transgene encoding a SARS-CoV-2 nucleocapsid protein, and the second recombinant NDV may comprise a package genome comprising a transgene encoding a chimeric F protein, wherein the chimeric F protein comprises a SARS-CoV-2 spike protein ectodomain and NDV F protein transmembrane and cytoplasmic domains. In specific embodiments, the SARS-CoV-2 spike protein ectodomain lacks the polybasic cleavage site (e.g., amino acid residues of the polybasic cleavage site (RRAR) are substituted with a single alainine). In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. In another example, the first recombinant NDV may comprise a packaged genome comprising a transgene encoding a SARS-CoV-2 nucleocapsid protein, and the second recombinant NDV may comprise a package genome comprising a transgene encoding a chimeric F protein,, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site (e.g., amino acid residues 682 to 685 of the polybasic cleavage site are substituted for a single alanine). In specific embodiments, the SARS-CoV-2 spike protein ectodomain is fused to the NDV F protein transmembrane and cytoplasmic domains via a linker (e.g, GGGGS (SEQ ID NO:24)). The linker may be any linker that does not interfere with folding of the ectodomain, function of the ectodomain or both. In some embodiments, the linker is an amino acid sequence (e.g, a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ, wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 spike protein ectodomain. See, e.g., Sections 5.1 and 6-12 for examples of recombinant NDVs. In another example, the first recombinant NDV may comprise a packaged genome comprising a transgene that comprises a nucleotide sequence encoding a SARS-CoV-2 nucleocapsid protein, and the second recombinant NDV may comprise a package genome comprising a transgene that comprises a nucleotide sequence encoding a chimeric F protein, wherein the chimeric F protein comprises a SARS-CoV-2 spike protein ectodomain and NDV F protein transmembrane and cytoplasmic domains. In certain embodiments, the first and second recombinant NDVs or compositions thereof may be separated by at least 7 days, 10 days, 14 days, 15 days, 21 days, 28 days, 30 days, 45 days, 2 months, 75 days, 3 months, or at least 6 months. In other embodiments, the first and second recombinant NDVs or compositions thereof may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months.

In certain embodiments, a first dose of a recombinant NDV described herein or composition described herein may be administered to a subject (e.g., a human) and a second dose of the recombinant NDV or composition may be administered to the subject 3 to 6 weeks later. In some embodiments, the subject is administered two or more boosters of the recombinant NDV. In a specific embodiment, a subject (e.g., human) is administered a recombinant NDV described herein using a regimen described in an Example below. In another specific embodiment, a subject (e.g., human) is administered a recombinant NDV described herein or composition thereof using a regimen described in Section 11, infra. In another specific embodiment, a subject (e.g., human) is administered a recombinant NDV described herein or composition thereof described in Section 11, infra, using a regimen described in Section 11, infra.

In certain embodiments, a recombinant NDV or composition thereof is administered to a subject in combination with one or more additional therapies, such as a therapy described in Section 5.5.3, infra. The dosage of the other one or more additional therapies will depend upon various factors including, e.g., the therapy, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. Recommended doses for approved therapies can be found in the Physician’s Desk Reference.

In certain embodiments, a recombinant NDV or composition thereof is administered to a subject concurrently with the administration of one or more additional therapies. In some embodiments, a first pharmaceutical composition comprising recombinant NDV and a second pharmaceutical composition comprising one or more additional therapies may be administered concurrently, or before or after each other. In certain embodiments, the first and second pharmaceutical compositions are administered concurrently to the subject, or within 1 minute, 2 minutes, 5 minutes, 10 minutes, 15 minutes, 20 minutes, 30 minutes, 45 minutes, 60 minutes, 1.5 hours, 2 hours, 3 hours, 4 hours, 5 hours, or 6 hours of each other. In certain embodiments, the first and second pharmaceutical compositions are administered to the subject within 7 days, 2 weeks, 3 weeks, 4 weeks, 5 weeks, 6 weeks, 7 weeks, 8 weeks or 12 weeks of each other. In certain embodiments, the first and second pharmaceutical compositions are administered to the subject within 3-6 months, 6-9 months, 6-12 months, or 3 months, 4 months, 6 months, 9 months, or 12 months of each other.

### 5.5.3 Additional Therapies

Additional therapies that can be used in a combination with a recombinant NDV described herein or a composition thereof include, but are not limited to, acetaminophen, ibuprofen, throat lozenges, cough suppressants, inhalers, antibiotics and oxygen. In a specific embodiment, the additional therapy is a second recombinant NDV described herein. In another specific embodiment, the additional therapy(ies) may include remdesivir, bamlanivimab plus etesevimab (AIIa), casirivimab plus imdevimab (AIIa), dexamethasone, tocilizumab, oxygen, or a combination thereof.

### 5.5.4 Other Uses of Recombinant NDV

In some embodiments, a recombinant NDV described herein is administered to a non-human subject (e.g., a mouse, rat, etc.) and the antibodies generated in response to the polypeptide are isolated. Hybridomas may be made and monoclonal antibodies produced as known to one of skill in the art. The antibodies may also be optimized. In some embodiments, the antibodies produced are humanized or chimerized. In certain embodiments, the non-human subject produces human antibodies. The antibodies produced using a recombinant NDV described herein may be optimized, using techniques known to one of skill in the art. In a specific embodiment, antibodies generated using a recombinant NDV described herein may be used to prevent, treat or prevent and treat COVID-19.

In some embodiments, a recombinant NDV described herein is used in an immunoassay (e.g., an ELISA assay) known to one of skill in the art or described herein to detect antibody specific for SARS-CoV-2 spike protein or nucleocapsid protein. In one embodiment, method for detecting the presence of antibody specific to SARS-CoV-2 spike protein or nucleocapsid, comprising contacting a specimen with the recombinant NDV described herein in an immunoassay (e.g., an ELISA). In some embodiments, the specimen is a biological specimen. In a specific embodiment, the biological specimen is blood, plasma or sera from a subject (e.g., a human subject). In other embodiments, the specimen is an antibody or antisera. See, the Examples, infra, for ELISA assays, which may be used.

### 5.6 Biological Assays

In a specific embodiment, one, two or more of the assays described in Sections 6-12 may be used to characterize a recombinant NDV described herein, or a SARS-CoV-2 spike protein or portion thereof (e.g., the ectodomain or receptor binding domain of the SARS-CoV-2 spike protein), SARS-CoV-2 nucleocapsid protein or a chimeric F protein. In another specific embodiment, one, two or more of the assays described in Sections 6-12 may be used to characterize immunoglobulin samples from a subject (e.g., a human subject) administered a recombinant NDV described herein or a composition described herein, such as, e.g., described in the Examples, infra (e.g., Section 6, 7, 8, 9, 10, 11, or 12). For example, the IgG titer and microneutralization of IgG may be assessed as described in the Examples below (e.g., Section 6, 7, 8, or 10). In some embodiments, a subject administered a recombinant NDV described herein or a composition described herein is assessed for anti-NDV antibodies as well as anti-SARS-CoV-2 spike or nucleocapsid antibodies.

### 5.6.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the recombinant NDVs described herein can be assessed by any method known in the art or described herein (e.g., in cell culture (e.g., cultures of BSTT7 or embryonated chicken cells) (see, e.g., Section 6, 7 or 10). Viral titer may be determined by inoculating serial dilutions of a recombinant NDV described herein into cell cultures (e.g., BSTT7 or embryonated chicken cells), chick embryos (e.g., 9 to 11 day old embryonated eggs), or live non-human animals. After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50). An exemplary method of assessing viral titer is described in Section 6, 7 or 10, below.

Incorporation of nucleotide sequences encoding a heterologous peptide or protein (e.g., a transgene into the genome of a recombinant NDV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art. In a specific embodiment, a method described in Section 6, 7, 9 or 10, infra, is used to assess the incorporation of a transgene into the genome of a recombinant NDV.

Immunofluorescence-based approaches may also be used to detect virus and assess viral growth. Such approaches are well known to those of skill in the art, e.g., fluorescence microscopy and flow cytometry (see, eg., Section 6, 7, or 10, infra). Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO). See, e.g., the assays described in Section 6 or 7, infra.

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY). See also Section 6, 7 or 10, infra, for histology and immunohistochemistry assays that may be used.

### 5.6.2 Interferon Assays

IFN induction and release by a recombinant NDV described herein may be determined using techniques known to one of skill in the art. For example, the amount of IFN induced in cells following infection with a recombinant NDV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. Further, the induction and release of cytokines and/or interferon-stimulated genes may be determined by, e.g., an immunoassay or ELISPOT assay at the protein level and/or quantitative RT-PCR or northern blots at the RNA level.

### 5.6.3 Toxicity Studies

In some embodiments, the recombinant NDVs described herein or compositions thereof, or combination therapies described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N- MC, SK-N-MC, SK-N-DZ, SH-SY5Y, and BE(2)-C. In some embodiments, the ToxLite assay is used to assess cytotoxicity.

Many assays well-known in the art can be used to assess viability of cells or cell lines following infection with a recombinant NDV described herein or composition thereof, and, thus, determine the cytotoxicity of the recombinant NDV or composition thereof. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, a recombinant NDV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The recombinant NDVs described herein or compositions thereof, or combination therapies can be tested for in vivo toxicity in animal models. For example, animals are administered a range of pfu of a recombinant NDV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages. See, e.g., the Examples, infra, for assays that may be used to assess toxicity.

The toxicity, efficacy or both of a recombinant NDV described herein or a composition thereof, or a combination therapy described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from the cell culture assays and animal studies can be used in formulating a range of dosage of the therapies for use in subjects. The dosage of such agents lies preferably within a range of circulating concentrations that include the ED50 with little or no toxicity. The dosage may vary within this range depending upon the dosage form employed and the route of administration utilized. For any therapy described herein, the therapeutically effective dose can be estimated initially from cell culture assays.

### 5.6.4 Biological Activity Assays

The recombinant NDVs described herein or compositions thereof, or combination therapies described herein can be tested for biological activity using animal models for inhibiting COVID-19, antibody response to the recombinant NDVs, etc. (see, e.g., Section 6, 7, 8 or 10). Such animal model systems include, but are not limited to, rats, mice, hamsters, cotton rats, chicken, cows, monkeys (e.g., African green monkey), pigs, dogs, rabbits, etc.

In a specific embodiment, the recombinant NDVs described herein or compositions thereof, or combination therapies described herein may be tested using animal models for the ability to induce a certain geometric mean titer of antibody(ies) that binds to the SARS-CoV-2 spike protein or nucleocapsid protein. An immunoassay, such as an ELISA, described in Section 7 or 10, infra, or known to one of skill in the art may be used to measure antibody titer. In another specific embodiment, the recombinant NDVs described herein or compositions thereof, or combination therapies described herein may be tested using animal models for the ability to induce antibodies that have neutralizing activity against SARS-CoV-2 spike protein or nucleocapsid protein in a microneutralizsation assay. In some embodiments, the recombinant NDVs described herein or compositions thereof, or combination therapies described herein may be tested using animal models for the ability to induce antibodies that neutralize SARS-CoV-2 in a microneutralizsation assay such as described herein (e.g., Section 7 or 10). In some embodiments, the recombinant NDVs described herein or compositions thereof, or combination therapies described herein may be tested using animal models for the ability to induce a certain geometric mean titer of antibody(ies) that binds to the SARS-CoV-2 spike protein or nucleocapsid protein and neutralizes SARS-CoV-2 spike protein or nucleocapsid protein in a microneutralizsation assay. In some embodiments, the recombinant NDVs described herein or compositions thereof, or combination therapies described herein may be tested using animal models for the ability to induce a certain geometric mean titer of antibody(ies) that binds to the SARS-CoV-2 spike protein or nucleocapsid protein and neutralizes SARS-CoV-2 in a microneutralizsation assay such as described herein (e.g., Section 7 or 10). In certain embodiments, the recombinant NDVs described herein or compositions thereof, or combination therapies described herein may be tested using animal models for the ability to induce a protective immune response (see, e.g, Section 10).

In a specific embodiment, a recombinant NDV described herein or a composition thereof, or a combination therapy described herein may be tested in a clinical trial study, such as described in Section 11, infra. In certain embodiments, a recombinant NDV described herein or a composition thereof, or a combination therapy described herein is administered to a human subject as described in Section 11, infra. In some embodiments, a human subject administered a recombinant NDV described herein or a composition thereof, or a combination therapy described herein may be assessed for one, two or more, or all of the things described in Section 11, infra. For example, one, two, or more or all of the following may be assessed following administration of a recombinant NDV described herein or a composition thereof, or a combination therapy described herein: GMT, anti-SARS-CoV-2 spike protein Ig (e.g., IgG, IgA, IgM, etc.), T cell response, NT50 seropositive response, NT80 seropostive response, T cell response, anti-NDV HN antibody, and anti-NDV F antibody. In certain embodiments, a recombinant NDV described herein or a composition thereof, or a combination therapy described herein is administered to a human subject as described in Section 11, infra, and the subject is assessed for one, two or more, or all of the things described in Section 11, infra.

### 5.6.5 Expression of Transgene

Assays for testing the expression of SARS-CoV-2 spike protein or portion thereof (e.g., SARS-CoV-2 ectodomain or receptor binding domain), chimeric F protein, or SARS-CoV-2 nucleocapsid protein in cells infected with a recombinant NDV comprising a packaged genome comprising a transgene that comprises a nucleotide sequence encoding SARS-CoV-2 spike protein or portion thereof (e.g., SARS-CoV-2 ectodomain or receptor binding domain), chimeric F protein, or SARS-CoV-2 nucleocapsid protein, respectively may be conducted using any assay known in the art, such as, e.g., western blot, immunofluorescence, and ELISA, or any assay described herein (see, e.g., Section 6, 7, 8, 9 or 10).

In a specific aspect, ELISA is utilized to detect expression of SARS-CoV-2 spike protein or portion thereof (e.g., SARS-CoV-2 ectodomain or receptor binding domain), chimeric F protein, or SARS-CoV-2 nucleocapsid protein in cells infected with a recombinant NDV comprising a packaged genome comprising a transgene that comprises a nucleotide sequence encoding of SARS-CoV-2 spike protein or portion thereof (e.g., SARS-CoV-2 ectodomain or receptor binding domain), chimeric F protein, or SARS-CoV-2 nucleocapsid protein. In a specific embodiment, an ELISA described in one of the Examples may be used to detect expression of SARS-CoV-2 spike protein or portion thereof (e.g., SARS-CoV-2 ectodomain or receptor binding domain), chimeric F protein, or SARS-CoV-2 nucleocapsid protein in cells infected with a recombinant NDV described herein.

In one embodiment, a SARS-CoV-2 spike protein or portion thereof (e.g., SARS-CoV-2 ectodomain or receptor binding domain), SARS-CoV-2 nucleocapsid protein or chimeric F protein encoded by a packaged genome of a recombinant NDV described herein is assayed for proper folding by testing its ability to bind specifically to an anti-SARS-CoV-2 spike protein or nucleocapsid antibody using any assay for antibody-antigen interaction known in the art. In another embodiment, a SARS-CoV-2 spike protein or portion thereof (e.g., SARS-CoV-2 ectodomain or receptor binding domain), SARS-CoV-2 nucleocapsid protein or chimeric F protein SARS-CoV-2 spike protein or portion thereof (e.g., SARS-CoV-2 ectodomain or receptor binding domain), SARS-CoV-2 nucleocapsid protein or chimeric F protein encoded by a packaged genome of a recombinant NDV described herein is assayed for proper folding by determination of the structure or conformation of the SARS-CoV-2 spike protein or portion thereof (e.g., SARS-CoV-2 ectodomain or receptor binding domain), SARS-CoV-2 nucleocapsid protein or chimeric F protein, respectively using any method known in the art such as, e.g., NMR, X-ray crystallographic methods, or secondary structure prediction methods, e.g., circular dichroism. Additional assays assessing the conformation and antigencity of SARS-CoV-2 spike protein or portion thereof (e.g., SARS-CoV-2 ectodomain or receptor binding domain), SARS-CoV-2 nucleocapsid protein or chimeric F protein may include, e.g., immunofluorescence microscopy, flow cytometry, western blot, and ELISA may be used.

### 5.7 Kits

In one aspect, provided herein is a pharmaceutical pack or kit comprising one or more containers filled with one or more of the ingredients of a composition (e.g., a pharmaceutical compositions) described herein. In a specific embodiment, provided herein is a pharmaceutical pack or kit comprising a container, wherein the container comprises a recombinant NDV described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, provided herein is a kit comprising in one or more containers filled with one or more recombinant NDVs described herein. In another embodiment, provided herein is a kit comprising in one or more containers one or more transgenes described herein. In another embodiment, provided herein is a kit comprising in one or more containers one or more nucleotide sequences comprising the genome of NDV and a transgene described herein. In another embodiment, provided herein is a kit comprising, in a container, a vector comprising a transgene described herein.

In a specific embodiment, provided herin is a kit comprising, in a container, a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

In a specific embodiment, provided herin is a kit comprising, in a container, a vector comprising a nucleotide sequence, wherein the nucleotide sequence comprises a transgene described herein and (1) a NDV F transcription unit, (2) a NDV NP transcription unit, (3) a NDV M transcription unit, (4) a NDV L transcription unit, (5) a NDV P transcription unit, (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

### 5.8 Sequences

TABLE 1

cDNA of genome of NDV Strains

| | | |
|---|---|---|
| cDNA of genomic sequence of NDV strain LaSota | accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgccCttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggcCgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaGgaGgggcctggcagctgctgcccaacgggtctccgaGgaGaccagcagcataGacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactAcgTacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagCtcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcGgacccaaggtccaactctccaagcggcaatcctctctcgcttcctcagccccactgaatgAtcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagAcacaggagatgggaagaagcaaatcgccccgcaatataggatccagcgccttgacttgtggactgatagtaaggaggactcagtattcatcaccacctatggattcatctttcaagttgggaatgaagaagccacCgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaGggatgtctacaagatcccagctgcagtattgaaggtttctggctcgagtctgtacaatcttgcgctcaatgtcactattaatgtggaggtagacccgaggagtcctttggttaaatctCtgtctaagtctgacagcggatactatgctaacctcttcttgcatattggacttatgaccacTgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccAaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaAtaGtctcttgattaagaaaaaatgtaagtggcaatgagatacaaggcaaaacagctcatggtTaaCaatacgggtaggacatggcgagctccggtcctgaaagggcagagcatcagattatcctaccagagTcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaaGaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaGgacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacaggGtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctGgacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagCgacctgacctcagataaagTagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcGaaaactctggtgcagcggcacggtacgctTttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatCatcgctagtcacattgacacagttatccggtctgtgatatatatggaagctgagggtgatctcgctgacacagtatttctatttaccccttacaatctctctactgacgggaaaaagaggacatcacttaAacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtaCtgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt | SEQ ID NO: 1 |
| cDNA of genomic sequence of NDV strain Hitchner B1 | ACCAAACAGAGAATCCGTAAGTTACGATAAAAGGCGAAGGAGCAATTGAAGTCGCACGGGTAGAAGGTGTGAATCTCGAGTGCGAGCCCGAAGCACAAACTCGAGGAAGCCTTCTGCCAACATGTCTTCCGTATTCGACGAGTACGAACAGCTCCTCGCGGCTCAGACTCGCCCCAATGGAGCTCATGGAGGGGGGGAGAAAGGGAGTACCTTAAAAGTAGACGTCCCGGTATTCACTCTTAACAGTGATGACCCAGAAGATAGGTGGAGCTTTGTGGTATTCTGCCTCCGGATTGCTGTTAGCGAAGATGCCAACAAACCACTCAGGCAAGGTGCTCTCATATCTCTTTTATGCTCCCACTCACAGGTAATGAGGAACCATGTTGCCCTTGCAGGGAAACAGAATGAAGCCACATTGGCCGTGCTTGAGATTGATGGCTTTGCCAACGGCACGCCCCAGTTCAACAATAGGAGTGGAGTGTCTGAAGAGAGAGCACAGAGATTTGCGATGATAGCAGGATCTCTCCCTCGGGCATGCAGCAACGGCACCCCGTTCGTCACAGCCGGGGCTGAAGATGATGCACCAGAAGACATCACCGATACCCTGGAGAGGATCCTCTCTATCCAGGCTCAAGTATGGGCACAGTAGCAAAAGCCATGACTGCGTATGAGACTGCAGATGAGTCGGAAACAAGGCGAATCAATAAGTATATGCAGCAAGGCAGGGTCCAAAAGAAATACATCCTCTACCCCGTATGCAGGAGCACAATCCAACTCACGATCAGACAGTCTCTTGCAGTCCGCATCTTTTTGGTTAGCGAGCTCAAGAGAGGCCGCAACACGGCAGGTGGTACCTCTACTTATTATAACCTAGTAGGGGACGTAGACTCATATATCAGGAATACCGGGCTTACTGCATTCTTCTTGACACTCAAGTACGGAATCAACACCAAGACATCAGCCCTTGCACTTAGTAGCCTCTCAGGCGACATCCAGAAGATGAAGCAGCTCATGCGTTTGTATCGGATGAAAGGAGATAATGCGCCGTACATGACATTACTTGGTGATAGTGACCAGATGAGCTTTGCGCCTGCCGAGTATGCACAACTTTACTCCTTTGCCATGGGTATGGCATCAGTCCTAGATAAAGGTACTGGGAAATACCAATTTGCCAGGGACTTTATGAGCACATCATTCTGGAGACTTGGAGTAGAGTACGCTCAGGCTCAGGGAAGTAGCATTAACGAGGATATGGCTGCCGAGCTAAAGCTAACCCCGGCAGCAAGGAGGGGCCTGGCAGCTGCTGCCCAACGAGTCTCCGAGGTGACCAGCAGCATAGACATGCCTACTCAACAAGTCGGAGTCCTCACTGGGCTTAGCGAGGGGGGATCCCAAGCCCTACAAGGCGGATCGAATAGATCGCAAGGGCAACCAGAAGCCGGGGATGGGGAGACCCAATTCCTGGATCTGATGAGAGCGGTAGCAAATAGCATGAGGGAGGCGCCAAACTCTGCACAGGGCACTCCCCAATCGGGGCCTCCCCCAACTCCTGGGCCATCCCAAGATAACGACACCGACTGGGGGTATTGATTGACAAAACCCAGCCTGCTTCTACAAGAACATCCCAATGCTCTCACCCGTAGTCGACCCCTCGATTTGCGGCTCTATATGACCACACCCTCAAACAAACATCCCCCTCTTTCCTCCCTCCCCCTGCTGTACAACTCCGCACGCCCTAGATACCACAGGCACACCGCGGCTCACTAACAATCAAAACAGAGCCGAGGGAATTAGAAAAAAGTACGGGTAGAAGAGGGATATTCAGAGATCAGGGCAAGTCTCCCGAGTCTCTGCTCTCTCCTCTACCTGATAGACCAGGACAAACATGGCCACCTTTACAGATGCAGAGATCGACGAGCTATTTGAGACAAGTGGAACTGTCATTGACAACATAATTACAGCCCAGGGTAAACCAGCAGAGACTGTTGGAAGGAGTGCAATCCCACAGGGCAAGACCAAGGTGCTGAGCGCAGCATGGGAGAAGCATGGGAGCATCCAGCCACCGGCCAGTCAAGACAACCTCGATCGACAGGACAGATCTGACAAACAACCATCCACACCCGAGCAAACGACCCCGCACGACAGCCCGCCGGCCACATCCGCTGACCAGCCCCCCACCCAGGCCACAGACGAAGCCGTCGACACACAGCTCAGGACCGGAGCAAGCAACTCTCTGCTGTTGATGCTTGACAAGCTCAGCAATAAATCGTCCAATGCTAAAAAGGGCCCATGGTCGAGCCCCCAAGAGGGGAATCACCAACGTCCGACTCAACAGCAGGGGAGTCAACCCAGTCGCGGAAACAGCCAGGAAAGACTGCAGAACCAAGTCAAGGCCGCCCCTGGAAACCAGGGCACAGACGTGAACACAGCATATCATGGACAATGGGAGGAGTCACAACTATCAGCTGGTGCAACCCCTCATGCTCTCCGATCAAGGCAGAGCCAAGACAATACCCTTGTATCTGCGGATCATGTCCAGCCACCTGTAGACTTTGTGCAAGCGATGATGTCTATGATGGGGGCGATATCACAGAGAGTAAGTAAGGTTGACTATCAGCTAGATCTTGTCTTGAAACAGACATCCTCCATCCCTATGATGCGGTCCGAAATCCAACAGCTGAAAACATCTGTTGCAGTCATGGAAGCCAACTTGGGAATGATGAAGATTCTGGATCCCGGTTGTGCCAACATTTCATCTCTGAGTGATCTACGGGCAGTTGCCCGATCTCACCCGGTTTTAGTTTCAGGCCCTGGAGACCCATCTCCCTATGTGATACAAGGAGGCGAAATGGCACTTAATAAACTTTCGCAACCAGTGCCACATCCATCTGAATTGATTAAACCCGCCACTGCATGCGGGCCTGATATAGGAGTGGAGAGGGACACTGTCCGTGCATTGATCATGTCACGCCCAATGCACCCGAGTTCTTCAGCCAAGCTCCTAAGCAAGTTAGATGCAGCCGGGTCGATCGAGGAAATCAGGAAAATCAAGCGCCTTGCTCTAAATGGCTAATTACTACTGCCACACGTAGCGGGTCCCTGTCCACTCGGCATCACACGGAATCTGCACCGAGTTCCCCCCCGCAGACCCAAGGTCCAACTCTAGAAGCGGCAATCCTCTCTCGCTTCCTCAGCCCCACTGAATGATCGCGTAACCGTAATTAATCTAGCTACATTAAGGATTAAGAAAAAATACGGGTAGAATTGGAGTGCCCCAATTGTGCCAAGATGGACTCATCTAGGACAATTGGGCTGTACTTTGATTCTGCCCATTCTTCTAGCAACCTGTTAGCATTTCCGATCGTCCTACAAGACACAGGAGATGGGAAGAAGCAAATCGCCCCGCAATATAGGATCCAGCGCCTTGACTCGTGGACTGATAGTAAGGAAGACTCAGTATTCATCACCACCTATGGATTCATCTTTCAAGTTGGGAATGAGGAAGCCACTGTCGGCATGATCGATGATAAACCCAAGCGCGAGTTACTTTCCGCTGCGATGCTCTGCCTAGGAAGCGTCCCAAATACCGGAGACCTTGTTGAGCTGGCAAGGGCCTGTCTCACTATGATGGTCACATGCAAGAAGAGTGCAACTAATACTGAGAGAATGGTTTTCTCAGTAGTGCAGGCACCCCAAGTGCTGCAAAGCTGTAGGGTTGTGGCAAATAAATACTCATCAGTGAATGCAGTCAAGCACGTGAAAGCGCCAGAGAAGATCCCCGGGAGTGGAACCCTAGAATACAAGGTGAACTTTGTCTCCTTGACTGTGGTACCGAAGAAGGATGTCTACAAGATCCCAGCTGCAGTATTGAAGATTTCTGGCTCGAGTCTGTACAATCTTGCGCTCAATGTCACTATTAATGTGGAGGTAGACCCGAGGAGTCCTTTGGTTAAATCTCTGTCTAAGTCTGACAGCGGATACTATGCTAACCTCTTCTTGCATATTGGACTTATGACCACCGTAGATAGGAAGGGGAAGAAAGTGACATTTGACAAGCTGGAAAAGAAAATAAGGAGCCTTGATCTATCTGTCGGGCTCAGTGATGTGCTCGGGCCTTCCGTGTTGGTAAAAGCAAGAGGTGCACGGACTAAGCTTTTGGCACCTTTCTTCTCTAGCAGTGGGACAGCCTGCTATCCCATAGCAAATGCTTCTCCTCAGGTGGCCAAGATACTCTGGAGTCAAACCGCGTGCCTGCGGAGCGTTAAAATCATTATCCAAGCAGGTACCCAACGCGCTGTCGCAGTGACCGCTGACCACGAGGTTACCTCTACTAAGCTGGAGAAGGGGCACACCCTTGCCAAATACAATCCTTTTAAGAAATAAGCTGCGTCTCTGAGATTGCGCTCCGCCCACTCACCCAGATCATCATGACACAAAAAACTAATCTGTCTTGATTATTTACAGTTAGTTTACCTGTCCATCAAGTTAGAAAAAACACGGGTAGAAGATTCTGGATCCCGGTTGGCGCCCTCCAGGTGCAGGATGGGCTCCAGACCTTCTACCAAGAACCCAGCACCTATGATGCTGACTATCCGGGTCGCGCTGGTACTGAGTTGCATCTGCCCGGCAAACTCCATTGATGGCAGGCCTCTTGCAGCTGCAGGAATTGTGGTTACAGGAGACAAAGCAGTCAACATATACACCTCATCCCAGACAGGATCAATCATAGTTAAGCTCCTCCCGAATCTGCCCAAGGATAAGGAGGCATGTGCGAAAGCCCCCTTGGATGCATACAACAGGACATTGACCACTTTGCTCACCCCCCTTGGTGACTCTATCCGTAGGATACAAGAGTCTGTGACTACATCTGGAGGGGGGAGACAGGGGCGCCTTATAGGCGCCATTATTGGCGGTGTGGCTCTTGGGGTTGCAACTGCCGCACAAATAACAGCGGCCGCAGCTCTGATACAAGCCAAACAAAATGCTGCCAACATCCTCCGACTTAAAGAGAGCATTGCCGCAACCAATGAGGCTGTGCATGAGGTCACTGACGGATTATCGCAACTAGCAGTGGCAGTTGGGAAGATGCAGCAGTTTGTTAATGACCAATTTAATAAAACAGCTCAGGAATTAGACTGCATCAAAATTGCACAGCAAGTTGGTGTAGAGCTCAACCTGTACCTAACCGAATTGACTACAGTATTCGGACCACAAATCACTTCACCTGCCTTAAACAAGCTGACTATTCAGGCACTTTACAATCTAGCTGGTGGGAATATGGATTACTTATTGACTAAGTTAGGTATAGGGAACAATCAACTCAGCTCATTAATCGGTAGCGGCTTAATCACCGGTAACCCTATTCTATACGACTCACAGACTCAACTCTTGGGTATACAGGTAACTCTACCTTCAGTCGGGAACCTAAATAATATGCGTGCCACCTACTTGGAAACCTTATCCGTAAGCACAACCAGGGGATTTGCCTCGGCACTTGTCCCAAAAGTGGTGACACAGGTCGGTTCTGTGATAGAAGAACTTGACACCTCATACTGTATAGAAACTGACTTAGATTTATATTGTACAAGAATAGTAACGTTCCCTATGTCCCCTGGTATTTACTCCTGCTTGAGCGGCAATACATCGGCCTGTATGTACTCAAAGACCGAAGGCGCACTTACTACACCATATATGACTATCAAAGGCTCAGTCATCGCTAACTGCAAGATGACAACATGTAGATGTGTAAACCCCCCGGGTATCATATCGCAAAACTATGGAGAAGCCGTGTCTCTAATAGATAAACAATCATGCAATGTTTTATCCTTAGGCGGGATAACTTTAAGGCTCAGTGGGGAATTCGATGTAACTTATCAGAAGAATATCTCAATACAAGATTCTCAAGTAATAATAACAGGCAATCTTGATATCTCAACTGAGCTTGGGAATGTCAACAACTCGATCAGTAATGCTTTGAATAAGTTAGAGGAAAGCAACAGAAAACTAGACAAAGTCAATGTCAAACTGACCAGCACATCTGCTCTCATTACCTATATCGTTTTGACTATCATATCTCTTGTTTTTGGTATACTTAGCCTGATTCTAGCATGCTACCTAATGTACAAGCAAAAGGCGCAACAAAAGACCTTATTATGGCTTGGGAATAATACCCTAGATCAGATGAGAGCCACTACAAAAATGTGAACACAGATGAGGAACGAAGGTTTCCCTAATAGTAATTTGTGTGAAAGTTCTGGTAGTCTGTCAGTTCGGAGAGTTAAGAAAAAACTACCGGTTGTAGATGACCAAAGGACGATATACGGGTAGAACGGTAAGAGAGGCCGCCCCTCAATTGCGAGCCAGACTTCACAACCTCCGTTCTA CCGCTTCACCGACAACAGTCCTCAATCATGGACCGCGCCGTTAGCCAAGTTGCGTTAGAGAATGATGAAAGAGAGGCAAAAAATACATGGCGCTTGATATTCCGGATTGCAATCTTATTCTTAACAGTAGTGACCTTGGCTATATCTGTAGCCTCCCTTTTATATAGCATGGGGGCTAGCACACCTAGCGATCTTGTAGGCATACCGACTAGGATTTCCAGGGCAGAAGAAAAGATTACATCTACACTTGGTTCCAATCAAGATGTAGTAGATAGGATATATAAGCAAGTGGCCCTTGAGTCTCCATTGGCATTGTTAAATACTGAGACCACAATTATGAACGCAATAACATCTCTCTCTTATCAGATTAATGGAGCTGCAAACAACAGCGGGTGGGGGGCACCTATTCATGACCCAGATTATATAGGGGGGATAGGCAAAGAACTCATTGTAGATGATGCTAGTGATGTCACATCATTCTATCCCTCTGCATTTCAAGAACATCTGAATTTTATCCCGGCGCCTACTACAGGATCAGGTTGCACTCGAATACCCTCATTTGACATGAGTGCTACCCATTACTGCTACACCCATAATGTAATATTGTCTGGATGCAGAGATCACTCACACTCACATCAGTATTTAGCACTTGGTGTGCTCCGGACATCTGCAACAGGGAGGGTATTCTTTTCTACTCTGCGTTCCATCAACCTGGACGACACCCAAAATCGGAAGTCTTGCAGTGTGAGTGCAACTCCCCTGGGTTGTGATATGCTGTGCTCGAAAGCCACGGAGACAGAGGAAGAAGATTATAACTCAGCTGTCCCTACGCGGATGGTACATGGGAGGTTAGGGTTCGACGGCCAATATCACGAAAAGGACCTAGATGTCACAACATTATTCGGGGACTGGGTGGCCAACTACCCAGGAGTAGGGGGTGGATCTTTTATTGACAGCCGCGTATGGTTCTCAGTCTACGGAGGGTTAAAACCCAATACACCCAGTGACACTGTACAGGAAGGGAAATATGTGATATACAAGCGATACAATGACACATGCCCAGATGAGCAAGACTACCAGATTCGAATGGCCAAGTCTTCGTATAAGCCTGGACGGTTTGGTGGGAAACGCATACAGCAGGCTATCTTATCTATCAAAGTGTCAACATCCTTAGGCGAAGACCCGGTACTGACTGTACCGCCCAACACAGTCACACTCATGGGGGCCGAAGGCAGAATTCTCACAGTAGGGACATCCCATTTCTTGTATCAGCGAGGGTCATCATACTTCTCTCCCGCGTTATTATATCCTATGACAGTCAGCGACAAAACAGCCACTCTTCATAGTCCTTATACATTCAATGCCTTCACTCGGCCAGGTAGTATCCCTTGCCAGGCTTCAGCAAGATGCCCCAACTCGTGTGTTACTGGAGTCTATACAGATCCATATCCCCTAATCTTCTATAGAAACCACACCTTGCGAGGGGTATTCGGGACAATGCTTGATGGTGAACAAGCAAGACTTAACCCTGCGTCTGCAGTATTCGATAGCACATCCCGCAGTCGCATAACTCGAGTGAGTTCAAGCAGCATCAAAGCAGCATACACAACATCAACTTGTTTTAAAGTGGTCAAGACCAATAAGACCTATTGTCTCAGCATTGCTGAAATATCTAATACTCTCTTCGGAGAATTCAGAATCGTCCCGTTACTAGTTGAGATCCTCAAAGATGACGGGGTTAGAGAAGCCAGGTCTGGCTAGTTGAGTCAACTATGAAAGAGTTGGAAAGATGGCATTGTATCACCTATCTTCTGCGACATCAAGAATCAAACCGAATGCCGGCGCGTGCTCGAATTCCATGTCGCCAGTTGACCACAATCAGCCAGTGCTCATGCGATCAGATTAAGCCTTGTCAATAGTCTCTTGATTAAGAAAAAATGTAAGTGGCAATGAGATACAAGGCAAAACAGCTCACGGTAAATAATACGGGTAGGACATGGCGAGCTCCGGTCCTGAAAGGGCAGAGCATCAGATTATCCTACCAGAGTCACACCTGTCTTCACCATTGGTCAAGCACAAACTACTCTATTATTGGAAATTAACTGGGCTACCGCTTCCTGATGAATGTGACTTCGACCACCTCATTCTCAGCCGACAATGGAAAAAAATACTTGAATCGGCCTCTCCTGATACTGAGAGAATGATAAAACTCGGAAGGGCAGTACACCAAACTCTTAACCACAATTCCAGAATAACCGGAGTACTCCACCCCAGGTGTTTAGAAGAACTGGCTAATATTGAGGTCCCTGATTCAACCAACAAATTTCGGAAGATTGAGAAGAAGATCCAAATTCACAACACGAGATATGGAGAACTGTTCACAAGGCTGTGTACGCATATAGAGAAGAAACTGCTGGGGTCATCTTGGTCTAACAATGTCCCCCGGTCAGAGGAGTTCAGCAGCATTCGTACGGATCCGGCATTCTGGTTTCACTCAAAATGGTCCACAGCCAAGTTTGCATGGCTCCATATAAAACAGATCCAGAGGCATCTGATTGTGGCAGCTAGGACAAGGTCTGCGGCCAACAAATTGGTGATGCTAACCCATAAGGTAGGCCAAGTCTTTGTCACTCCTGAACTTGTTGTTGTGACGCATACGAATGAGAACAAGTTCACATGTCTTACCCAGGAACTTGTATTGATGTATGCAGATATGATGGAGGGCAGAGATATGGTCAACATAATATCAACCACGGCGGTGCATCTCAGAAGCTTATCAGAGAAAATTGATGACATTTTGCGGTTAATAGACGCTCTGGCAAAAGACTTGGGTAATCAAGTCTACGATGTTGTATCACTAATGGAGGGATTTGCATACGGAGCTGTCCAGCTACTCGAGCCGTCAGGTACATTTGCGGGAGATTTCTTCGCATTCAACCTGCAGGAGCTTAAAGACATTCTAATTGGCCTCCTCCCCAATGATATAGCAGAATCCGTGACTCATGCAATCGCTACTGTATTCTCTGGTTTAGAACAGAATCAAGCAGCTGAGATGTTGTGCCTGTTGCGTCTGTGGGGTCACCCACTGCTTGAGTCCCGTATTGCAGCAAAGGCAGTCAGGAGCCAAATGTGCGCACCGAAAATGGTAGACTTTGATATGATCCTTCAGGTACTGTCTTTCTTCAAGGGAACAATCATCAACGGATACAGAAAGAAGAATGCAGGTGTGTGGCCGCGAGTCAAAGTGGATACAATATATGGGAAGGTCATTGGGCAACTACATGCAGATTCAGCAGAGATTTCACACGATATCATGTTGAGAGAGTATAAGAGTTTATCTGCACTTGAATTTGAGCCATGTATAGAATACGACCCTGTCACTAACCTGAGCATGTTCCTAAAAGACAAGGCAATCGCACACCCCAACGATAATTGGCTTGCCTCGTTTAGGCGGAACCTTCTCTCCGAAGACCAGAAGAAACATGTAAAGGAAGCGACTTCGACTAACCGCCTCTTGATAGAGTTTTTAGAGTCAAATGATTTTGATCCATATAAAGAGATGGAATATCTGACGACCCTTGAGTACCTTAGAGATGACAATGTGGCAGTATCATACTCGCTCAAAGAGAAGGAAGTGAAAGTTAATGGACGGATCTTCGCTAAGCTGACAAAGAAGTTAAGGAACTGTCAGGTGATGGCGGAAGGGATCCTAGCCGATCAGATTGCACCTTTCTTTCAGGGAAATGGAGTCATTCAGGATAGCATATCCTTGACCAAGAGTATGCTAGCGATGAGTCAACTGTCTTTTAACAGCAATAAGAAACGTATCACTGACTGTAAAGAAAGAGTATGTTCAAACCGCAATCATGATCCGAAAAGCAAGAACCGTCGGAGAGTTGCAACCTTCATAACAACTGACCTGCAAAAGTACTGTCTTAATTGGAGATATCAGACGATCAAATTGTTCGCTCATGCCATCAATCAGTTGATGGGCCTACCTCATTTCTTCGAGTGGATTCACCTAAGACTGATGGACACTACGATGTTCGTAGGAGACCCTTTCAATCCTCCAAGTGACCCTACTGACTGTGACCTCTCAAGAGTCCCTAATGATGACATATATATTGTCAGTGCCAGAGGGGGTATCGAAGGATTATGCCAGAAGCTATGGACAATGATCTCAATTGCTGCAATCCAACTTGCTGCAGCTAGATCGCATTGTCGTGTTGCCTGTATGGTACAGGGTGATAATCAAGTAATAGCAGTAACGAGAGAGGTAAGATCAGATGACTCTCCGGAGATGGTGTTGACACAGTTGCATCAAGCCAGTGATAATTTCTTCAAGGAATTAATCCATGTCAATCATTTGATTGGCCATAATTTGAAGGATCGTGAAACCATCAGGTCAGACACATTCTTCATATACAGCAAACGAATCTTCAAAGATGGAGCAATCCTCAGTCAAGTCCTCAAAAATTCATCTAAATTAGTGCTAGTGTCAGGTGATCTCAGTGAAAACACCGTAATGTCCTGTGCCAACATTGCCTCTACTGTAGCACGGCTATGCGAGAACGGGCTTCCCAAAGACTTCTGTTACTATTTAAACTATATAATGAGTTGTGTGCAGACATACTTTGACTCTGAGTTCTCCATCACCAACAATTCGCACCCCGATCTTAATCAGTCGTGGATTGAGGACATCTCTTTTGTGCACTCATATGTTCTGACTCCTGCCCAATTAGGGGGACTGAGTAACCTTCAATACTCAAGGCTCTACACTAGAAATATCGGTGACCCGGGGACTACTGCTTTTGCAGAGATCAAGCGACTAGAAGCAGTGGGACTACTGAGTCCTAACATTAGGACTAATATCTTAACTAGGCCGCCTGGGAATGGAGATTGGGCCAGTCTGTGCAACGACCCATACTCTTTCAATTTTGAGACTGTTGCAAGCCCAAACATTGTTCTTAAGAAACATACGCAAAGAGTCCTATTTGAAACTTGTTCAAATCCCTTATTGTCTGGAGTGCACACAGAGGATAATGAGGCAGAAGAGAAGGCATTGGCTGAATTCTTGCTTAATCAAGAGGTGATTCATCCCCGCGTTGCGCATGCCATCATGGAGGCAAGCTCTGTAGGTAGGAGAAAGCAAATTCAAGGGCTTGTTGACACAACAAACACTGTAATTAAGATTGCGCTTACTAGGAGGCCATTAGGCATCAAGAGGCTGATGCGGATAGTCAATTATTCTAGCATGCATGCAATGCTGTTTAGAGACGATGTTTTTTCCTCTAGTAGATCCAACCACCCCTTAGTCTCTTCTAATATGTGTTCTCTGACACTGGCAGACTATGCACGGAATAGAAGCTGGTCACCTTTGACGGGAGGCAGGAAAATACTGGGTGTATCTAATCCTGATACGATAGAACTCGTAGAGGGTGAGATTCTTAGTGTAAGCGGAGGGTGTACAAGATGTGACAGCGGAGATGAACAATTTACTTGGTTCCATCTTCCAAGCAATATAGAATTGACCGATGACACCAGCAAGAATCCTCCGATGAGGGTACCATATCTCGGGTCAAAGACACAGGAGAGGAGAGCTGCCTCACTTGCGAAAATAGCTCATATGTCGCCACATGTGAAGGCTGCCCTAAGGGCATCATCCGTGTTGATCTGGGCTTATGGGGATAATGAAGTAAATTGGACTGCTGCTCTTACGATTGCAAAATCTCGGTGTAATGTAAACTTAGAGTATCTTCGGTTACTGTCCCCTTTACCCACGGCTGGGAATCTTCAACATAGACTAGATGATGGTATAACTCAGATGACATTCACCCCTGCATCTCTCTACAGGGTGTCACCTTACATTCACATATCCAATGATTCTCAAAGGCTGTTCACTGAAGAAGGAGTCAAAGAGGGGAATGTGGTTTACCAACAGATCATGCTCTTGGGTTTATCTCTAATCGAATCGATCTTTCCAATGACAACAACCAGAACATATGATGAGATCACACTGCACCTACATAGTAAATTTAGTTGCTGTATCAGGGAAGCACCTGTTGCGGTTCCTTTCGAGCTACTTGGGGTGGCACCGGAACTGAGGACAGTGACCTCAAATAAGTTTATGTATGATCCTAGCCCTGTATCGGAGGGAGACTTTGCGAGACTTGACTTAGCTATCTTCAAGAGTTATGAGCTTAATCTGGAGTCATATCCCACGATAGAGCTAATGAACATTCTTTCAATATCCAGCGGGAAGTTGATTGGCCAGTCTGTGGTTTCTTATGATGAAGATACCTCCATAAAGAATGATGCCATAATAGTGTATGACAATACCCGAAATTGGATCAGTGAAGCTCAGAATTCAGATGTGGTCCGCCTATTTGAATATGCAGCACTTGAAGTGCTCCTCGACTGTTCTTACCAACTCTATTACCTGAGAGTAAGAGACCTAGACAATATTGTCTTATATATGGGTGATTTATACAAGAATATGCCAGGAATTCTACTTTCCAACATTGCAGCTACAATATCTCATCCTGTCATTCATTCAAGGTTACATGCAGTGGGCCTGGTCAACCATGACGGATCACACCAACTTGCAGATACGGATTTTATCGAAATGTCTGCAAAACTGTTAGTATCTTGCACCCGACGTGTGATCTCCGGCTTATATTCAGGAAATAAGTATGATCTGCTGTTCCCATCTGTCTTAGATGATAACCTGAATGAGAAGATGCTTCAGCTGATATCCCGGTTATGCTGTCTGTACACGGTACTCTTTGCTACAACAAGAGAAATCCCGAAAATAAGAGGCTTAACTGCAGAAGAGAAATGTTCAATACTCACTGAGTATTTACTGTCGGATGCTGTGAAACCATTACTTAGCCCCGATCAAGTGAGCTCTATCATGTCTCCTAACATAATTACATTCCCAGCTAATCTGTACTACATGTCTCGGAAGAGCCTCAATTTGATCAGGGAAAGGGAGGACAGGGATACTATCCTGGCGTTGTTGTTCCCCCAAGAGCCATTATTAGAGTTCCCTTCTGTGCAAGATATTGGTGCTCGAGTGAAAGATCCATTCACCCGACAACCTGCGGCATTTTTGCAAGAGTTAGATTTGAGTGCTCCAGCAAGGTATGACGCATTCACACTTAGTCAGATTCATCCTGAACTCACATCTCCAAATCCGGAGGAAGACTACTTAGTACGATACTTGTTCAGAGGGATAGGGACTGCATCTTCCTCTTGGTATAAGGCATCCCATCTCCTTTCTGTACCCGAGGTAAGATGTGCAAGACACGGGAACTCCTTATACTTGGCTGAAGGAAGCGGAGCCATCATGAGTCTTCTTGAACTGCATGTACCACATGAAACTATCTATTACAATACGCTCTTTTCAAATGAGATGAACCCCCCGCAACGACATTTCGGGCCGACCCCAACTCAGTTTTTGAATTCGGTTGTTTATAGGAATCTACAGGCGGAGGTAACATGCAAGGATGGATTTGTCCAAGAGTTCCGTCCATTATGGAGAGAAAATACAGAGGAAAGTGACCTGACCTCAGATAAAGCAGTGGGGTATATTACATCTGCAGTACCCTACAGATCTGTATCATTGCTGCATTGTGACATTGAAATTCCTCCAGGGTCCAATCAAAGCTTACTAGATCAACTAGCTATCAATTTATCTCTGATTGCCATGCATTCTGTAAGGGAGGGCGGGGTAGTAATCATCAAAGTGTTGTATGCAATGGGATACTACTTTCATCTACTCATGAACTTGTTTGCTCCGTGTTCCACAAAAGGATATATTCTCTCTAATGGTTATGCATGTCGAGGGGATATGGAGTGTTACCTGGTATTTGTCATGGGTTACCTGGGCGGGCCTACATTTGTACATGAGGTGGTGAGGATGGCAAAAACTCTGGTGCAGCGGCACGGTACGCTTTTGTCTAAATCAGATGAGATCACACTGACCAGGTTATTCACCTCACAGCGGCAGCGTGTGACAGACATCCTATCCAGTCCTTTACCAAGATTAATAAAGTACTTGAGGAAGAATATTGACACTGCGCTGATTGAAGCCGGGGGACAGCCCGTCCGTCCATTCTGTGCGGAGAGTCTGGTGAGCACGCTAGCGAACATAACTCAGATAACCCAGATCATCGCTAGTCACATTGACACAGTCATCCGGTCTGTGATATATATGGAAGCTGAGGGTGATCTCGCTGACACAGTATTTCTATTTACCCCTTACAATCTCTCTACTGACGGGAAAAAGAGGACATCACTTAAACAGTGCACGAGACAGATCCTAGAGGTTACAATACTAGGTCTTAGAGTCGAAAATCTCAATAAAATAGGCGATATAATCAGCCTAGTGCTTAAAGGCATGATCTCCATGGAGGACCTTATCCCACTAAGGACATACTTGAAGCATAGTACCTGCCCTAAATATTTGAAGGCTGTCCTAGGTATTACCAAACTCAAAGAAATGTTTACAGACACTTCTGTACTGTACTTGACTCGTGCTCAACAAAAATTCTACATGAAAACTATAGGCAATGCAGTCAAAGGATATTACAGTAACTGTGACTCCTAACGAAAATCACATATTAATAGGCTCCTTTTTTGGCCAATTGTATTCTTGTTGATTTAATTATATTATGTTAGAAAAAAGTTGAACTCTGACTCCTTAGGACTCGAATTCGAACTCAAATAAATGTCTTTAAAAAAGGTTGCGCACAATTATTCTTGAGTGTAGTCTCGTCATTCACCAAATCTTTGTTTGGT | SEQ ID NO: 2 |

TABLE 2

NDV LaSota F protein

| | | |
|---|---|---|
| Amino acid sequence of F protein of NDV strain LaSota (transmembrane domain is underlined and cytoplasmic domain is in bold) | MGSRPSTKNPAPMTLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM | SEQ ID NO: 3 |

TABLE 3

SARS-CoV-2 Spike Protein Sequences

| | | |
|---|---|---|
| Secreted Receptor Binding Domain (RBD) of SARS-CoV-2 Spike Protein (nucleotide sequence) | ATGTTCGTGTTTCTGGTGCTGCTGCCTCTGGTGTCCAGCCAGCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAAGATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGGAAGGCTTCAACTGCTACTTCCCACTGCAGTCCTACGGCTTTCAGCCCACAAATGGCGTGGGCTATCAGCCCTACAGAGTGGTGGTGCTGAGCTTCGAACTGCTGCATGCCCCTGCCACAGTGTGCGGCCCTAAGAAAAGCACCAATCTCGTGAAGAACAAATGCGTGAACTTCTGA | SEQ ID NO: 4 |
| Secreted Receptor Binding Domain (RBD) of SARS-CoV-2 Spike Protein (amino acid sequence) | MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF* | SEQ ID NO: 5 |
| Secreted RBD of SARS-CoV-2 Spike Protein 6×His (nucleotide sequence) | ATGTTCGTGTTTCTGGTGCTGCTGCCTCTGGTGTCCAGCCAGCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAAGATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGGAAGGCTTCAACTGCTACTTCCCACTGCAGTCCTACGGCTTTCAGCCCACAAATGGCGTGGGCTATCAGCCCTACAGAGTGGTGGTGCTGAGCTTCGAACTGCTGCATGCCCCTGCCACAGTGTGCGGCCCTAAGAAAAGCACCAATCTCG TGAAGAACAAATGCGTGAACTTCcaccatcaccatcaccatTGA | SEQ ID NO: 6 |
| Secreted RBD of SARS-CoV-2 Spike Protein 6×His (amino acid sequence) | MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH* | SEQ ID NO: 7 |
| Secreted ectodomain of SARS-CoV-2 spike protein 6× His (nucleotide sequence) | ATGTTCGTGTTTCTGGTGCTGCTGCCTCTGGTGTCCAGCCAGTGTGTGAACCTGACCACAAGAACCCAGCTGCCTCCAGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAGATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAAAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAAGATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAG ATCTATCAGGCCGGCAGCACCCCTTGTAACGGCGTGGAAGGCTTCAACTGCTACTTCCCACTGCAGTCCTACGGCTTTCAGCCCACAAATGGCGTGGGCTATCAGCCCTACAGAGTGGTGGTGCTGAGCTTCGAACTGCTGCATGCCCCTGCCACAGTGTGCGGCCCTAAGAAAAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGACGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGCACGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGCCGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCTTCATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCGCCGCTCTGCAGATCCCCTTTGCTATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAGCAAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACAAGGTGGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCAGCGGCCGCTTGGTCCCACGTGGCTCACCCGGATCTGGATACATCCCGGAGGCCCCTAGGGACGGTCAAGCTTACGTGAGAAAGGACGGCGAATGGGTTCTGCTGTCGACCTTCTTGGGACATCATCATCATCATCACTAA | SEQ ID NO: 8 |
| Secreted ectodomain of SARS-CoV-2 spike protein 6× His (amino acid sequence) | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH* | SEQ ID NO: 9 |
| Full length SARS-CoV-2 spike protein (nucleotide sequence) | ATGTTCGTGTTTCTGGTGCTGCTGCCTCTGGTGTCCAGCCAGTGTGTGAACCTGACCACAAGAACCCAGCTGCCTCCAGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAGATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAAAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAAGATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGGAAGGCTTCAACTGCTACTTCCCACTGCAGTCCTACGGCTTTCAGCCCACAAATGGCGTGGGCTATCAGCCCTACAGAGTGGTGGTGCTGAGCTTCGAACTGCTGCATGCCCCTGCCACAGTGTGCGGCCCTAAGAAAAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGACGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGCACGTGAACAATAGCTACGAGTGCGACATCCCCATCGGCGCTGGCATCTGTGCCAGCTACCAGACACAGACAAACAGCCCCAGACGGGCCAGATCTGTGGCCAGCCAGAGCATCATTGCCTACACAATGTCTCTGGGCGCCGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCTTCATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCGCCGCTCTGCAGATCCCCTTTGCTATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAGCAAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACAAGGTGGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCTGGTACATCTGGCTGGGCTTTATCGCCGGACTGATTGCCATCGTGATGGTCACAATCATGCTGTGTTGCATGACCAGCTGCTGTAGCTGCCTGAAGGGCTGTTGTAGCTGTGGCAGCTGCTGCAAGTTCGACGAGGACGATTCTGAGCCCGTGCTGAAGGGCGTGAAACTGCACTACACCTGA | SEQ ID NO: 10 |
| Full length SARS-CoV-2 spike protein (amino acid sequence) | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT* | SEQ ID NO: 11 |
| Chimeric F protein (SARS-CoV-2 spike protein ectodomain and NDV F protein transmembrane and cytoplasmic domains; nucleotide sequence) | ATGTTCGTGTTTCTGGTGCTGCTGCCTCTGGTGTCCAGCCAGTGTGTGAACCTGACCACAAGAACCCAGCTGCCTCCAGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAGATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAAAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAAGATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGGAAGGCTTCAACTGCTACTTCCCACTGCAGTCCTACGGCTTTCAGCCCACAAATGGCGTGGGCTATCAGCCCTACAGAGTGGTGGTGCTGAGCTTCGAACTGCTGCATGCCCCTGCCACAGTGTGCGGCCCTAAGAAAAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGACGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGCACGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGCCGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCTTCATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCGCCGCTCTGCAGATCCCCTTTGCTATGCAGATGGCCTACCGGTTCAACGGCATCGGAGTGACCCAGAATGTGCTGTACGAGAACCAGAAGCTGATCGCCAACCAGTTCAACAGCGCCATCGGC AAGATCCAGGACAGCCTGAGCAGCACAGCAAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACAAGGTGGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCGGCGGAGGGGGGAGTCTCATTACCTATATCGTTTTGACTATCATATCTCTTGTTTTTGGTATACTTAGCCTGATTCTAGCATGCTACCTAATGTACAAGCAAAAGGCGCAACAAAAGACCTTATTATGGCTTGGGAATAATACTCTAGATCAGATGAGAGCCACTACAAAAATGTGATAA | SEQ ID NO: 12 |
| Chimeric F protein (SARS-CoV-2 spike protein ectodomain and NDV F protein transmembrane and cytoplasmic domains; amino acid sequence) | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNT LDQMRATTKM* | SEQ ID NO: 13 |
| S-F chimera HexaPro (Nucleotide sequence) | ATGTTCGTGTTTCTGGTGCTGCTGCCTCTGGTGTCCAGCCAGTGTGTGAACCTGACCACAAGAACCCAGCTGCCTCCAGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAGATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAAAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAAGATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGGAAGGCTTCAACTGCTACTTCCCACTGCAGTCCTACGGCTTTCAGCCCACAAATGGCGTGGGCTATCAGCCCTACAGAGTGGTGGTGCTGAGCTTCGAACTGCTGCATGCCCCTGCCACAGTGTGCGGCCCTAAGAAAAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGACGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGCACGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGCCGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCcctATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCcctGCTCTGCAGATCCCCTTTccaATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAcccAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACccccctGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCggcggaggtgggtcgCTCATAACATACATCGTCCTGACTATAATCAGCTTGGTATTTGGTATTTTGTCTTTGATTCTTGCATGCTATTTGATGTATAAACAGAAAGCTCAGCAGAAGACTCTCCTGTGGCTCGGTAACAACACACTCGACCAGATGAGAGCAACTACAAAGATGTGATAA | SEQ ID NO: 14 |
| S-F chimera HexaPro (Amino acid sequence) | MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM* | SEQ ID NO: 15 |
| NDV-HXP-S (B.1.351) (Nucleotide sequence) | ATGTTCGTGTTTCTGGTGCTGCTGCCTCTGGTGTCCAGCCAGTGTGTGAACTTCACCACAAGAACCCAGCTGCCTCCAGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGCCAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAGATCTACAGCAAGCACACCCCTATCAACCTCGTGCGGGGCCTGCCTCAGGGCTTCTCTGCTCTGGAACCCCTGGTGGATCTGCCCATCGGCATCAACATCACCCGGTTTCAGACACTGCACATCAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAgAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAACATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGAAGGGCTTCAACTGCTACTTCCCACTGCAGTCCTACGGCTTTCAGCCCACATACGGCGTGGGCTATCAGCCCTACAGAGTGGTGGTGCTGAGCTTCGAACTGCTGCATGCCCCTGCCACAGTGTGCGGCCCTAAGAAgAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGGCGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGCACGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGTGGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCcctATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCcctGCTCTGCAGATCCCCTTTccaATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAcccAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACccccctGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCggcggaggtgggtcgCTCATAACATACATCGTCCTGACTATAATCAGCTTGGTATTTGGTATTTTGTCTTTGATTCTTGCATGCTATTTGATGTATAAACAGAAAGCTCAGCAGAAGACTCTCCTGTGGCTCGGTAACAACACACTCGACCAGATGAGAGCAACTACAAAGATGTGA | SEQ ID NO: 16 |
| NDV-HXP-S (B.1.351) (Amino acid sequence) | MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM* | SEQ ID NO: 17 |
| NDV-HXP-S (P.1) (Nucleotide sequence) | ATGTTCGTGTTTCTGGTGCTGCTGCCTCTGGTGTCCAGCCAGTGTGTGAACttcACCaacAGAACCCAGCTGCCTagcGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACtacCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGagcGAGTTCGTGTTCAAGAACATCGACGGCTACTTCAAGATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAgAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCaccATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGaagGGCTTCAACTGCTACTTCCCACTGCAGTCCTACGGCTTTCAGCCCACAtacGGCGTGGGCTATCAGCCCTACAGAGTGGTGGTGCTGAGCTTCGAACTGCTGCATGCCCCTGCCACAGTGTGCGGCCCTAAGAAgAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGACGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGtacGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGCCGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCcctATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCcctGCTCTGCAGATCCCCTTTccaATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAcccAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACccccctGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCatcAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCggcggaggtgggtcgCTCATAACATACATCGTCCTGACTATAATCAGCTTGGTATTTGGTATTTTGTCTTTGATTCTTGCATGCTATTTGATGTATAAACAGAAAGCTCAGCAGAAGACTCTCCTGTGGCTCGGTAACAACACACTCGACCAGATGAGAGCAACTACAAAGATGTGA | SEQ ID NO: 18 |
| NDV-HXP-S (P.1) (Amino acid sequence) | MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM* | SEQ ID NO: 19 |
| cDNA of genomic sequence of NDV strain LaSota (L289A mutation) | accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtegggaacetaaataatatgcgtgccacetacttggaaacettatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggetcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagetggtcacetttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt | SEQ ID NO:25 |

TABLE 8

Other Sequences

| | | |
|---|---|---|
| SacII Restriction Sequence | CCGCGG | SEQ ID NO:20 |
| NDV Gene End Sequence | TTAGAAAAAA | SEQ ID NO:21 |
| NDV Gene Start Sequence | ACGGGTAGAA | SEQ ID NO:22 |
| Kozak Sequence | CCGCCACC | SEQ ID NO:23 |
| Linker Sequence | GGGGS | SEQ ID NO:24 |

### 6. EXAMPLE 1: RECOMBINANT NDV EXPRESSING SARS-COV-2 SPIKE PROTEIN

This example demonstrates that the expression of full length SARS-CoV-2 spike protein, a protein comprising the SARS-CoV-2 spike ectodomain, a protein comprising the SARS-CoV-2 spike protein receptor binding domain. In particular, this example describes engineering lentogenic Newcastle disease virus (NDV) vectors expressing the receptor-binding domain (RBD), the ectodomain, or the full-length of the spike of SARS-CoV-2. The NDV expressing these proteins may be used as diagnostic reagents or vaccine candidates.

Newcastle disease virus (NDV) belongs to the genus of Avulavirus in the family of Paramyxoviridae. Despite the fact that NDV is an important avian pathogen, it only causes mild flu-like symptoms or conjunctivitis in humans. In the past, the lentogenic NDV strains have been engineered and tested as oncolytic agents or viral vector vaccines expressing foreign antigens. Reverse genetic systems for NDV-LaSota (LS) wild type or L289A mutant strains were used to genetically modify NDV to encode a transgene.

To provide SARS-CoV-2 diagnostic reagents, NDV vectors (wild-type or L289A mutant) expressing 1) the soluble RBD (S_RBD 6 × His) or 2) the ectodomain of the spike (S_Ecto 6 × His) with a purification tag were generated. The two proteins could be expressed and purified from allantoic fluid of embryonated chicken eggs inoculated with NDV_LS_S_RBD 6 × His or NDV_LS_S_Ecto 6 × His viruses. These proteins could be used as substrates in serology tests such as ELISAs to measure SARS-CoV-2 spike-specific antibody titers. The advantage of using the NDV protein expression system is that NDV grows to high titers in embryonated chicken eggs, allowing the protein production to be high yield but low cost.

To meet the urgent need of SARS-CoV-2 vaccines, NDV vectors (WT or L289A mutant) expressing 1) the secreted RBD (S_RBD); 2) full-length spike (S); and 3) a modified chimeric spike (S-F), in which the ectodomain of the spike is fused to the transmembrane domain and cytoplasmic tail of the F protein of NDV were generated. They were designated NDV_LS_S_RBD, NDV_LS_S and NDV_LS_S-F, respectively. All three NDV vectors may be used as live-attenuated vaccines, while 2) and 3) may be used as adjuvanted inactivated vaccines due to the incorporation of the spike protein into the NDV virions. The RNA genome of NDV has the advantage of not being integrated into the human genome. As an avian pathogen that is non-pathogenic in humans, NDV vectors are safe and not be counteracted by any pre-existing immunity in humans.

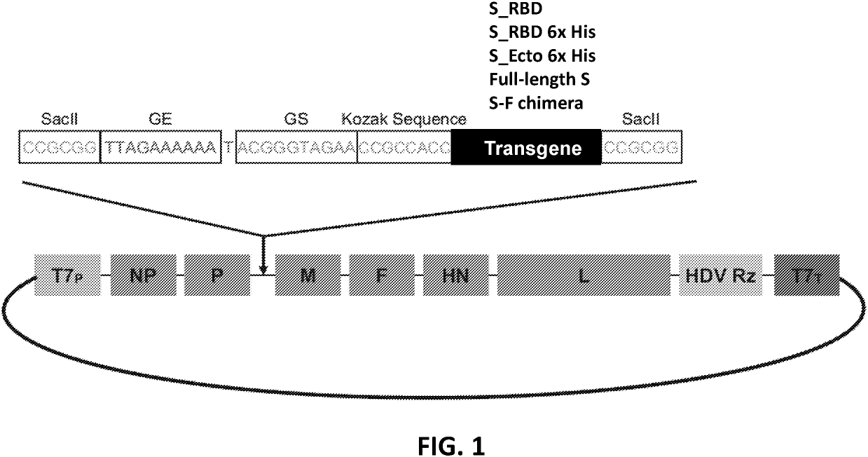

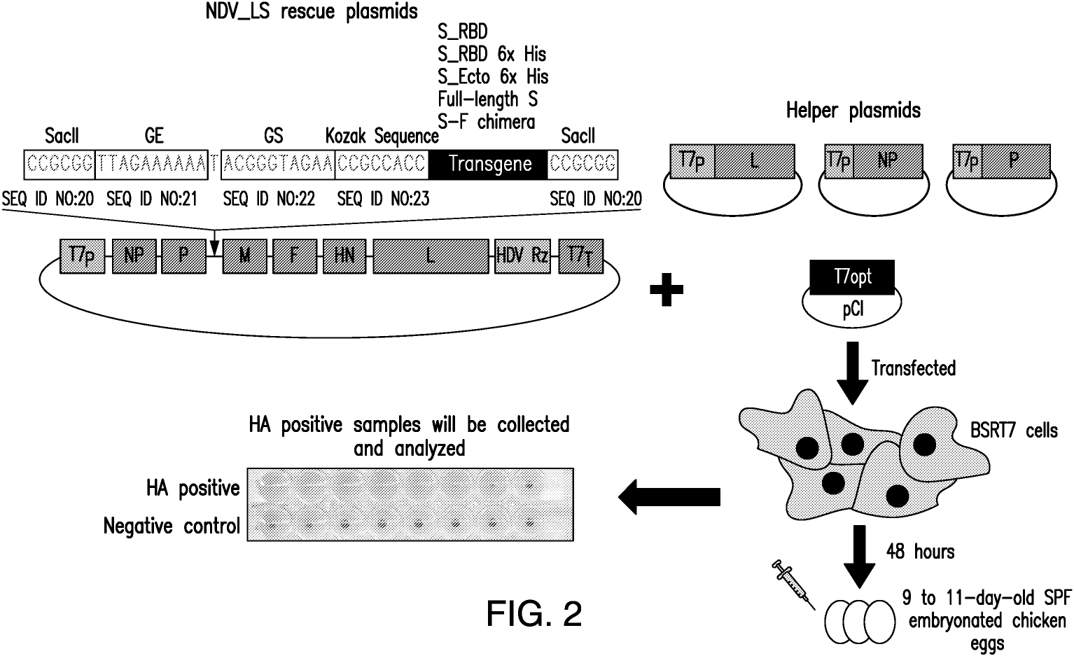

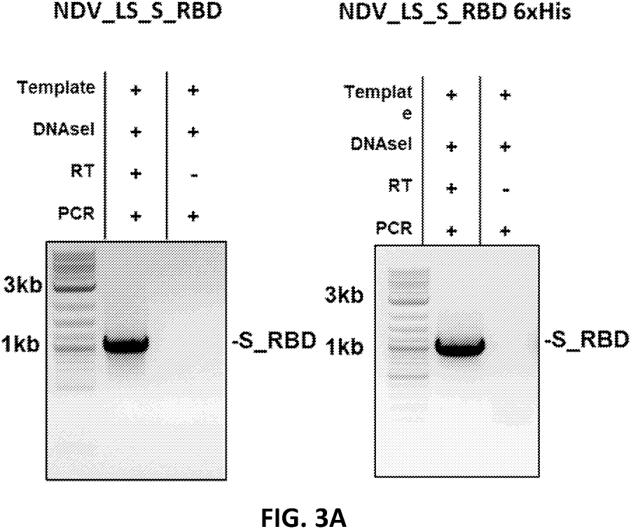

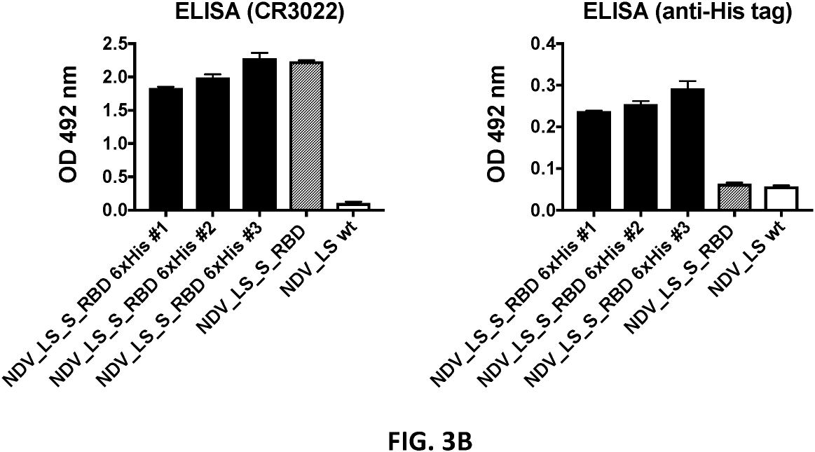

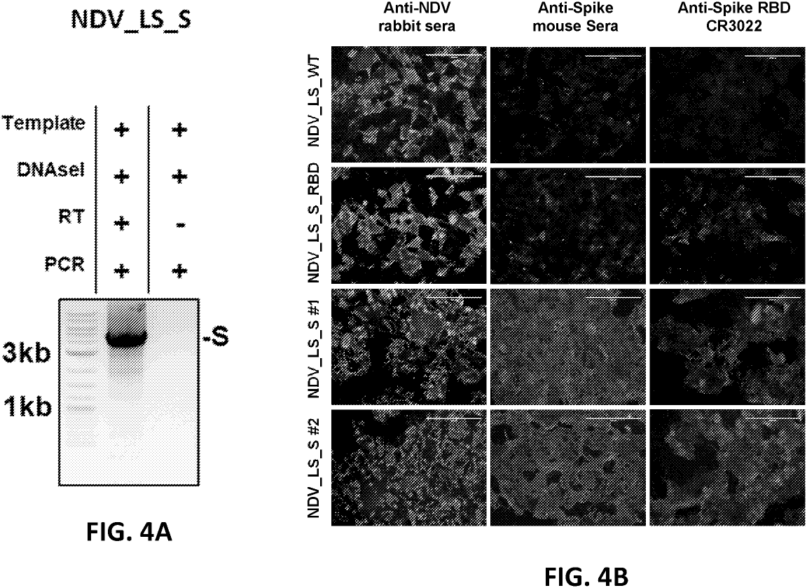

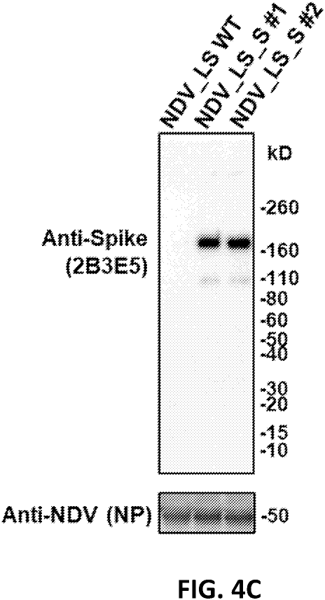

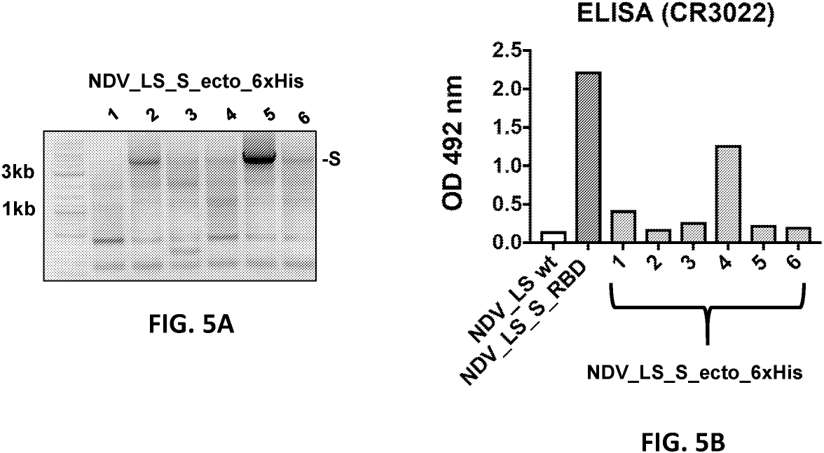

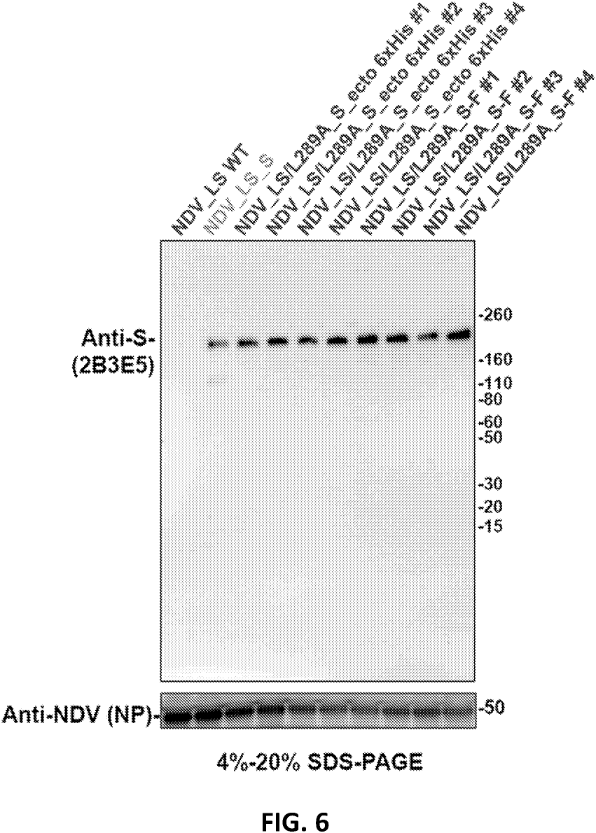

A rescue plasmid containing the antigenomic cDNA of the NDV LaSota strain, downstream of a T7 promoter, was constructed. The DNA sequence of the S_RBD, S_Ecto or full-length S was inserted into the intergenic region between the phosphoprotein (P) and matrix (M) protein genes. See FIG. 1 for a depiction of the construction of NDV LaSota plasmids. To rescue the virus, BSRT7 cells were transfected with the antigenomic cDNA rescue plasmid with helper plasmids expressing the nucleoprotein (N), the P protein and the large polymerase (L) protein and the T7 polymerase. The supernatant of the transfected cells was collected and injected into 9 to 11 day-old embryonated chicken eggs. The eggs were incubated at 37° C. for 48 - 96 hours and then were cooled overnight at 4° C. Allantoic fluids were collected and the rescue of the virus was examined by hemagglutination (HA) assay. See FIG. 2 for a depiction of the methodology used to rescue NDV expressing the 1) the soluble RBD (S_RBD 6 × His), 2) the ectodomain of the spike (S_Ecto 6 × His), 3) the secreted RBD (S_RBD); 4) full-length spike (S); or 5) a modified chimeric spike (S-F), in which the ectodomain of the spike is fused to the transmembrane domain and cytoplasmic tail of the F protein of NDV. RNA of the HA positive samples was extracted and the presence of the transgene were confirmed by RT-PCR. See FIGS. 3A, 4A, and 5A. The transgenes in the viral genome were sequenced by Sanger sequencing. The expression of the S_RBD, S_Ecto and full-length S was confirmed by immunoassays such as ELISAs, immunofluorescent assays, or Western blot using spike-specific monoclonal antibodies or mouse antisera. See FIGS. 3B, 4B, 4C5, 5B, and 6. Viruses with correctly expressed transgenes were further amplified in embryonated chicken eggs to expand large virus stocks. Virus stocks were aliquoted and stored at -80° C. The infectious titer of the virus stocks was determined by immunofluorescent assay on infected Vero cells.

### 7. EXAMPLE 2: NEWCASTLE DISEASE VIRUS (NDV) EXPRESSING THE SPIKE PROTEIN OF SARS-COV-2 AS VACCINE CANDIDATE

Due to the lack of protective immunity of humans towards the newly emerged SARS-CoV-2, this virus has caused a massive pandemic across the world resulting in hundreds of thousands of deaths. Thus, a vaccine is urgently needed to contain the spread of the virus. This example describes Newcastle disease virus (NDV) vector vaccines expressing the spike protein of SARS-CoV-2 in its wild type or a pre-fusion membrane anchored format. All described NDV vector vaccines grow to high titers in embryonated chicken eggs. In a proof of principle mouse study, this example reports that the NDV vector vaccines elicit high levels of antibodies that are neutralizing when the vaccine is given intramuscularly. Importantly, these COVID-19 vaccine candidates protect mice from a mouse-adapted SARS-CoV-2 challenge with no detectable viral titer and viral antigen in the lungs.

The NDV vector vaccine against SARS-CoV-2 described in this study has advantages similar to those of other viral vector vaccines. But the NDV vector can be amplified in embryonated chicken eggs, which allows for high yields and low costs per dose. Also, the NDV vector is not a human pathogen, therefore the delivery of the foreign antigen would not be compromised by any pre-existing immunity in humans. Finally, NDV has a very good safety record in humans, as it has been used in many oncolytic virus trials. This study provides an important option for a cost-effective SARS-CoV-2 vaccine.

This study informs of the value of a viral vector vaccine against SARS-CoV-2. Specifically, for this NDV based SARS-CoV-2 vaccine, the existing egg-based influenza virus vaccine manufactures in the U.S. and worldwide would have the capacity to rapidly produce hundreds of millions of doses to mitigate the consequences of the ongoing COVID-19 pandemic.

### 7.1 Background

The unprecedented coronavirus disease 2019 (COVID-19) pandemic caused by severe acute respiratory syndrome coronavirus 2 (SARS-CoV-2) has resulted in ~16.3 million infections with more than half a million deaths since the end of 2019 as of Jul. 26, 2020, and continues to pose a threat to public health. To mitigate the spread of the virus, social distancing, mask-wearing, and the lockdown of cities, states or even countries were practiced, with a heavy price paid both medically and economically. Unfortunately, due to the relative lack of pre-existing immunity of humans to this virus, no countermeasures will be completely effective without a vaccine. Because of the urgent need for an effective SARS-CoV-2 vaccine, many candidates are being developed using various vaccine platforms, including mRNA vaccines (1, 2), inactivated whole virus vaccines (3), subunit vaccines, DNA vaccines and viral vector vaccines (4). These vaccine candidates are designed to essentially target the spike (S) protein of the SARS-CoV-2 (5), which is the major structural protein displayed on the surface of the SARS-CoV-2. The S protein mediates the entry of the virus via binding to the angiotensin converting enzyme 2 (ACE2) receptor in humans. The S protein is also the most important antigen of the virus that harbors many B cell and T cell epitopes (6-9). Neutralizing antibodies, most of which target the receptor-binding domain (RBD), can be induced by the S protein (9, 10). However, to eventually contain the virus spread worldwide, not only the efficacy, but also the cost and scalability of the vaccine are crucial, especially in low and middle income countries with limited resources.

This example reports the construction and characterization of Newcastle disease virus (NDV) vectors expressing the SARS-CoV-2 S protein. NDV belongs to the genus of Avulavirus in the family of Paramyxoviridae, it is an avian pathogen, typically causing no symptoms in humans although mild influenza-like symptoms or conjunctivitis have been described in rare cases. The lentogenic NDV vaccine strain such as the LaSota strain, in addition to be avirulent in birds, has been used as an oncolytic agent and a vaccine vector (11-15). As a large negative strand RNA virus, NDV is stable and well tolerates transgenes into its genome. NDV vectors have been successfully used to express the spike protein of other coronaviruses (16, 17). The NDV platform is also appealing, because the virus grows to high titers in embryonated chicken eggs, which are also used to produce influenza virus vaccines. Humans typically lack pre-existing immunity toward the NDV, which makes the virus preferable over other viral vectors that are human pathogens, such as human adenovirus, measles virus or Modified Vaccinia Ankara (MVA). The lentogenic NDV vector has proven to be safe in humans as it has been tested extensively in human trials (18-20). Most importantly, at low cost, NDV vector vaccines could be generated in embryonated chicken eggs quickly under biosafety level 2 (BSL-2) conditions to meet the vast demand on a global scale. This study reports successfully rescued NDV vectors expressing two forms of the spike protein of SARS-CoV-2, the wild type (WT) S and a chimeric version containing the ectodomain (with the polybasic cleavage site deleted) of the spike and the transmembrane domain and cytoplasmic domain of the NDV F (pre-fusion S-F chimera). The data provided in this example shows that WT S and S-F were well expressed from the NDV as transgenes in infected cells. While both WT S and S-F were displayed on the surface of the NDV particles, the incorporation of the S-F into NDV particles was substantially improved compared to that of the WT S, as expected. A proof of concept study in mice using three live NDV vectors expressing the spike protein (NDV_LS_S, NDV_LS_S-F and NDV_LS/L289A_S-F) showed that high titers of binding and neutralizing antibodies were induced. All three NDV vector vaccines fully protected mice from challenge with a SARS-CoV-2 mouse-adapted strain, showing no detectable viral titers and viral antigens in the lungs at day four post-challenge. To conclude, promising cost-effective SARS-CoV-2 vaccine candidates have been developed using the NDV LaSota strain as the viral vector, which could be generated to high yield under BSL-2 conditions.

### 7.2 Materials and Methods

Plasmids. The sequence of the wild type S was amplified from pCAGGS plasmid (21) encoding the codon-optimized nucleotide sequence of the spike gene (GenBank: MN908947.3) of a SARS-CoV-2 isolate by PCR, using primers containing the gene end (GE), gene start (GS) and a Kozak sequences at the 5′ end (22). To construct the S-F chimera, the ectodomain of the S without the polybasic cleavage site (CS, ⁶⁸²RRAR⁶⁸⁵ to A) (22) was generated by PCR. A mammalian cell codon-optimized nucleotide sequence of the transmembrane domain (TM) and the cytoplasmic tail (CT) of the NDV LaSota fusion (F) protein was synthesized commercially (gBlock, Integrated DNA technologies). The S ectodomain (no CS) was fused to the TM/CT of F through a GS linker (GGGGS (SEQ ID NO:24)). The sequence was again modified by adding GE, GS and a Kozak sequence at the 5′. Additional nucleotides were added at the 3′ of both inserts to follow the “rule of six”. The transgenes were inserted between the P and M gene of pNDV LaSota (LS) wild type or the L289A (15, 22, 23) mutant (NDV_LS/L289A) antigenomic cDNA by in-Fusion cloning (Clontech). The recombination products were transformed into NEB® Stable Competent E. coli (NEB) to generate NDV_LS_S, NDV_LS_S-F and NDV_LS/L289A_S-F rescue plasmids. The plasmids were purified using QIAprep Spin Miniprep kit (Qiagen) for Sanger sequencing (Macrogen). Maxipreps of rescue plasmids were purified using PureLink™ HiPure Plasmid Maxiprep Kit (Thermo Fisher Scientific).

Cells. BSRT7 cells stably expressing the T7 polymerase were kindly provided by Dr. Benhur Lee at ISMMS. The cells were maintained in Dulbecco’s Modified Eagle’s medium (DMEM; Gibco) containing 10% (vol/vol) fetal bovine serum (FBS) and 100 unit/ml of penicillin/streptomycin (P/S; Gibco) at 37° C. with 5% CO₂. Vero E6 cells were obtained from American Type Culture Collection (ATCC, CRL-1586). Vero E6 cells were also maintained in DMEM containing 10% FBS with 100 unit/ml P/S at 37° C. with 5% CO₂.

Rescue of NDV LaSota expressing the spike protein of SARS-CoV-2. Six-well plates of BSRT7 cells were seeded 3 × 10⁵ cells per well the day before transfection. The next day, a transfection cocktail was prepared consisting of 250 µl of Opti-MEM (Gibco) including 4 µg of pNDV_LS_S or pNDV_LS_S-F or pNDV_LS/L289A_S-F, 2 µg of pTM1-NP, 1 µg of pTM1-P, 1 µg of pTM1-L and 2 µg of pCI-T7opt. Thirty µl of TransIT LT1 (Mirus) were added to the plasmid cocktail and gently mixed by pipetting three times and incubated at room temperature (RT) for 30 min. Toward the end of the incubation, the medium was replaced with 1 ml of Opti-MEM. The transfection complex was added dropwise to each well and the plates were incubated at 37° C. with 5% CO₂. Forty-eight hours post transfection, the supernatant and the cells were harvested and briefly homogenized by several strokes with an insulin syringe. Two hundred microliters of the cell/supernatant mixture were injected into the allantoic cavity of 8- to 10-day old specific pathogen free (SPF) embryonated chicken eggs. The eggs were incubated at 37° C. for 3 days before being cooled at 4° C. overnight. The allantoic fluid was collected and clarified by low-spin centrifugation to remove debris. The presence of the rescued NDV was determined by hemagglutination (HA) assay using 0.5% chicken or turkey red blood cells. The RNA of the positive samples was extracted and treated with DNase I (Thermo Fisher Scientific). Reverse transcriptase-polymerase chain reaction (RT-PCR) was performed to amplify the transgenes. The sequences of the transgenes were confirmed by Sanger Sequencing (Genewiz).

Immunofluorescence assay (IFA). Vero E6 cells were seeded onto 96-well tissue culture plates at 2.5 × 10⁴ cells per well. The next day, cells were washed with 100 µl warm phosphate buffered saline (PBS) and infected with 50 µl of allantoic fluid at 37° C. for 1h. The inocula were removed and replaced with 100 µl of growth medium. The plates were then incubated at 37° C. Sixteen to eighteen hours after infection, the cells were washed with 100 µl of warm PBS and fixed with 4% methanol-free paraformaldehyde (PFA) (Electron Microscopy Sciences) for 15 min at 4° C. The PFA was discarded, cells were washed with PBS and blocked in PBS containing 0.5% bovine serum albumin (BSA) for 1 hour at 4° C. The blocking buffer was discarded and surface proteins were stained with anti-NDV rabbit serum or SARS-CoV-2 spike receptor-binding domain (RBD) specific human monoclonal antibody CR3022 (24, 25) for 2h at RT. The primary antibodies were discarded, cells were then washed 3 times with PBS and incubated with goat anti-rabbit Alexa Fluor 488 or goat anti-human Alexa Fluor 488 secondary antibodies (Thermo Fisher Scientific) for 1h at RT. The secondary antibodies were discarded, cells were washed again 3 times with PBS and images were captured using an EVOS fl inverted fluorescence microscope (AMG).

Virus titration. Stocks of NDV expressing the S or S-F proteins were titered using an immunofluorescence assay (IFA). Briefly, Vero cells were seeded onto 96-well (Denville) tissue culture plates at 2.5 × 10⁴ cells/well the day before infection. The next day, five-fold serial dilutions of each virus stocks were prepared in a separate 96-well plate in Opti-MEM (Gibco). Medium in the 96-well plate was removed and the cells were washed with 100 µL of warm PBS. Fifty µL of the virus dilutions were added to each well. The plates were incubated at 37° C. for one hour and shaken every 15 minutes to ensure the cells were infected evenly. The inoculum was removed and 100 µL of DMEM containing 10% FBS with 100 unit/ml P/S was added. The plates were incubated at 37° C. overnight for 16 to 18 hours. The next day, the media were aspirated off and cells were washed once with 100 µL of warm PBS. IFA was performed as described above to staining NDV surface glycoproteins. Infected fluorescent cells were counted starting from the undiluted wells until a well down the dilution with a countable number of cells was found. The fluorescent cells in the entire well were counted. Titer of the virus (focus forming unit, FFU per ml) was determined by the following formula: Titer (FFU/ml) = No. of fluorescent cells x Dilution factor x (1000 uL/volume of infection)

Virus concentration. Allantoic fluids were clarified by centrifugation at 4,000 rpm using a Sorvall Legend RT Plus Refrigerated Benchtop Centrifuge (Thermo Fisher Scientific) at 4° C. for 30 min. Viruses in the allantoic fluid were pelleted through a 20% sucrose cushion in NTE buffer (100 mM NaCl, 10 mM Tris-HCl, 1 mM ethylenediaminetetraacetic acid (EDTA), pH 7.4) by centrifugation in a Beckman L7-65 ultracentrifuge at 25,000 rpm for 2 h at 4° C. using a Beckman SW28 rotor (Beckman Coulter, Brea, CA, USA). Supernatants were aspirated and the pellets were re-suspended in PBS (pH 7.4). The protein content was determined using the bicinchoninic acid (BCA) assay (Thermo Fisher Scientific).

Western Blot. Concentrated virus samples were mixed with Novex™ Tris-Glycine SDS Sample Buffer (2X) (Thermo Fisher Scientific) with NuPAGE™ Sample Reducing Agent (10X) (Thermo Fisher Scientific). The samples were heated at 95° C. for 5 min. Two microgram of concentrated viruses were resolved on 4-20% sodium dodecyl sulfate-polyacrylamide gel electrophoresis (SDS-PAGE) gels (Biorad) using the Novex™ Sharp Pre-stained Protein Standard (Thermo Fisher Scientific) as the marker. Proteins were transferred onto polyvinylidene difluoride (PVDF) membrane (GE healthcare). The membrane was blocked with 5% dry milk in PBS containing 0.1% v/v Tween 20 (PBST) for 1h at RT. The membrane was washed with PBST on a shaker 3 times (10 min at RT each time) and incubated with primary antibodies diluted in PBST containing 1% BSA overnight at 4° C. To detect the spike protein of SARS-CoV-2, a mouse monoclonal antibody 2B3E5 kindly provided by Dr. Thomas Moran at ISMMS was used, while the HN protein was detected by a mouse monoclonal antibody 8H2 (MCA2822, Bio-Rad). The membranes were then washed with PBST on a shaker 3 times (10 min at RT each time) and incubated with sheep anti-mouse IgG linked with horseradish peroxidase (HRP) diluted (1:2000) in PBST containing 5% dry milk for 1h at RT. The secondary antibody was discarded and the membranes were washed with PBST on a shaker 3 times (10 min at RT each time). Pierce™ ECL Western Blotting Substrate (Thermo Fisher Scientific) was added to the membrane, the blots were imaged using the Bio-Rad Universal Hood Ii Molecular imager (Bio-Rad) and processed by Image Lab Software (Bio-Rad).

Mice immunizations. Ten-week old female BALB/cJ mice (Jackson Laboratories) were used. Experiments were performed in accordance with protocols approved by the Icahn School of Medicine at Mount Sinai Institutional Animal Care and Use Committee (IACUC). Mice were divided into 9 groups (n=5) receiving four different concentrated live viruses at two doses (10 µg and 50 µg) intramuscularly (i.m). Specifically, group 1 (10 µg per mouse) and 2 (50 µg per mouse) were given wild type NDV_LS; group 3 (10 µg per mouse) and 4 (50 µg per mouse) received NDV_LS_S; group 5 (10 µg) and 6 (50 µg) received NDV_LS_S-F and group 7 (10 µg per mouse) and 8 (50 µg per mouse) received NDV_LS/L289A_S-F. Group 9 given PBS was used as the negative controls. A prime-boost immunization regimen was used for all the groups in a 3-week interval.

Enzyme linked immunosorbent assay (ELISA). Immunized mice were bled pre-boost and 8 days after the boost. Sera were isolated by low-speed centrifugation. To perform ELISAs, Immulon 4 HBX 96-well ELISA plates (Thermo Fisher Scientific) were coated with 2 µg/ml of recombinant trimeric S protein (50 µl per well) in coating buffer (SeraCare Life Sciences Inc.) overnight at 4° C. (21). The next day, all plates were washed 3 times with 220 µL PBS containing 0.1% (v/v) Tween-20 (PBST) and 220 µL blocking solution (3% goat serum, 0.5% dry milk, 96.5% PBST) was added to each well and incubated for 1 h at RT. Mouse sera were 3-fold serially diluted in blocking solution starting at 1:30 followed by a 2 h incubation at RT. ELISA plates were washed 3 times with PBST and 50 µL of sheep anti-mouse IgG-horseradish peroxidase (HRP) conjugated antibody (GE Healthcare) was added at a dilution of 1:3,000 in blocking solution. Then, plates were again incubated for one hour at RT. Plates were washed 3 times with PBST and 100 µL of o-phenylenediamine dihydrochloride (SigmaFast OPD, Sigma) substrate was added per well. After 10 min, 50 µL of 3 M hydrochloric acid (HCl) was added to each well to stop the reaction and the optical density (OD) was measured at 492 nm on a Synergy 4 plate reader (BioTek). An average of OD values for blank wells plus three standard deviations was used to set a cutoff for plate blank outliers. A cutoff value was established for each plate that was used for calculating the endpoint titers. The endpoint titers of serum IgG responses was graphed using GraphPad Prism 7.0.

SARS-CoV-2 challenge in mice. The SARS-CoV-2 challenge was performed at the University of North Carolina by Dr. Ralph Baric’s group in a Biosafety Level 3 (BSL-3) facility. Mice were challenged 11 days after the boost using a mouse adapted SARS-CoV-2 strain at 10⁴ plaque forming unit (PFU) intranasally (i.n) under ketamine/xylazine anesthesia as described previously (1, 26).

Lung titers. Lung lobes of mice were collected and homogenized in PBS. A plaque assay was performed to measure viral titer in the lung homogenates as described previously (1, 26). Geometric mean titers of plaque forming units (PFU) per lobe were calculated using GraphPad Prism 7.0.

Micro-neutralization assay. All neutralization assays were performed in the biosafety level 3 (BSL-3) facility following institutional guidelines as described previously (21, 27). Briefly, serum samples were heat-inactivated at 56° C. for 60 minutes prior to use. 2× minimal essential medium (MEM) supplemented with glutamine, sodium biocarbonate, 4-(2-hydroxyethyl)1-piperazineethanesulfonic acid (HEPES), and antibiotics P/S was used for the assay. Vero E6 cells were maintained in culture using DMEM supplemented with 10% fetal bovine serum (FBS). Twenty-thousands cells per well were seeded the night before in a 96-well cell culture plate. 1× MEM was prepared from 2× MEM and supplemented with 2% FBS. Three-fold serial dilutions starting at 1:20 of pooled sera were prepared in a 96-well cell culture plate and each dilution was mixed with 600 times the 50% tissue culture infectious dose (TCID₅₀) of SARS-CoV-2 (USA-WA1/2020, BEI Resources NR-52281). Serum-virus mixture was incubated for 1 h at room temperature. Virus-serum mixture was added to the cells for 1 h and kept in a 37° C. incubator. Next, the virus-serum mixture was removed and the corresponding serum dilution was added to the cells with addition 1× MEM. The cells were incubated for 2 days and fixed with 100 µL 10% formaldehyde per well for 24 h before taken out of the BSL-3 facility. The staining of the cells was performed in a biosafety cabinet (BSL-2). The formaldehyde was carefully removed from the cells. Cells were washed with 200 µL PBS once before being permeabilized with PBS containing 0.1% Triton X-100 for 15 min at RT. Cells were washed with PBS and blocked in PBS containing 3% dry milk for 1 h at RT. Cells were then stained with 100 µL per well of a mouse monoclonal anti-NP antibody (1C7), kindly provided by Dr. Thomas Moran at ISMMS, at 1 µg/ml for 1 h at RT. Cells were washed with PBS and incubated with 100 µL per well Anti-mouse IgG HRP (Rockland, cat. no. 610-4302) secondary antibody at 1:3,000 dilution in PBS containing 1% dry milk for 1 h at RT. Finally, cells were washed twice with PBS and the plates were developed using 100 µL of SigmaFast OPD substrate. Ten minutes later, the reactions were stopped using 50 µL per well of 3 M HCI. The OD 492 nM was measured on a Biotek SynergyH1 Microplate Reader. Non-linear regression curve fit analysis (The top and bottom constraints are set at 100% and 0%) over the dilution curve was performed to calculate 50% of inhibitory dilution (ID₅₀) of the serum using GraphPad Prism 7.0.

Immunohistochemistry (IHC). The lung lobes of mice were perfused and fixed in 10% phosphate buffered formalin for 7 days before transferred out of the BSL-3 facility. The fixed lungs were paraffin embedded, and sectioned at 5 µm for immunohistochemistry (IHC) staining (HistoWiz). IHC was performed using a rabbit SARS-CoV-2 nucleocapsid (N) protein (NB100-56576, Novus Biologicals). Slides were counter stained with hematoxylin. All slides were examined by a board-certified veterinary pathologist (HistoWiz).

Statistics. Statistical analysis was performed using GraphPad Prism 7.0. The statistical difference in viral titers in the lungs was determined using one-way analysis of variance (ANOVA), and corrected for multiple comparisons using Dunnett’s test.

### 7.3 Results

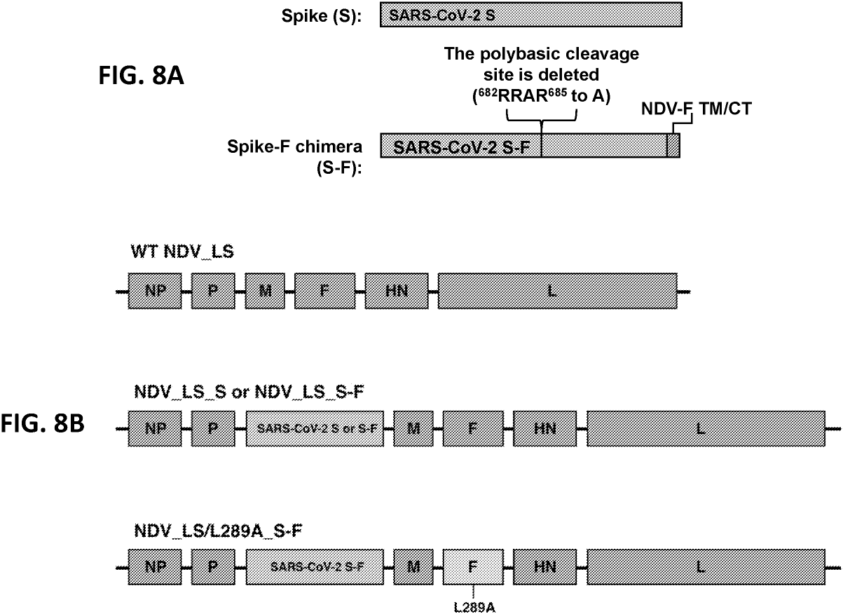

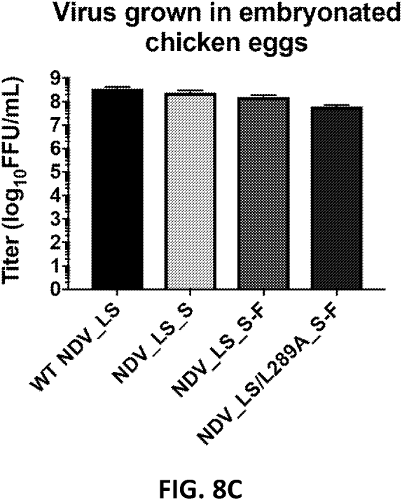

Design and rescue of NDV LaSota expressing the spike protein of SARS-CoV-2. For protective immunity, the S protein is the most important antigen of SARS-CoV-2. To express S antigen by the NDV LaSota vaccine strain, two constructs were designed. One is the wild type spike (S), the other is the spike-F chimera (S-F). The S-F consists of the ectodomain of the S, in which the polybasic cleavage site ⁶⁸²RRAR⁶⁸⁵ is removed by deleting the three arginines to stabilize the protein in its pre-fusion conformation (21). Importantly, to increase membrane-anchoring of the spike on the surface of the NDV virions, the transmembrane domain (TM) and cytoplasmic tail (CT) of the spike were replaced with those from the fusion (F) protein of NDV (FIG. 8A)(28). The nucleotide sequences of each construct were inserted between the P and M genes of the antigenomic cDNA of WT NDV LaSota strain and/or NDV LaSota/L289A mutant strain, in which the mutation L289A in the F protein supports HN independent fusion (23). The latter NDV mutant has been safely used in humans (15) (FIG. 8B). NDV expressing the spike proteins were rescued by transient transfection of BSRT7 cells followed by amplification in embryonated chicken eggs. All the viruses expressing the S or S-F grew to high titers (~10⁸ FFU/ml) in embryonated chicken eggs (FIG. 8C), which is advantageous for the development of a low-cost vaccine.

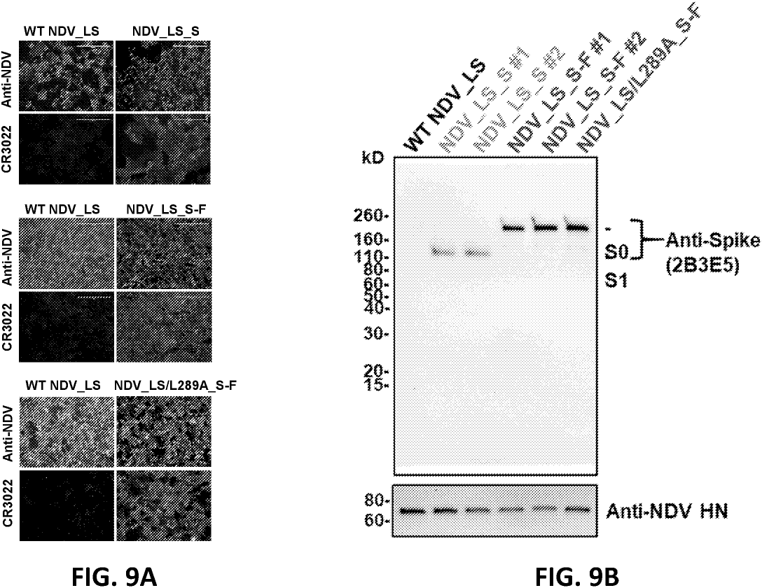

The spike protein is incorporated into NDV particles. To validate the expression of S and S-F as transgenes, Vero E6 cells were infected with WT NDV or NDV expressing the S or S-F. The surface of the cells was stained with anti-NDV rabbit serum or spike-specific monoclonal antibody CR3022 that recognizes the RBD. It was confirmed that only NDV expressing the S or S-F showed robust expression of the spike on the cell surface, while NDV proteins were detected in all virus-infected cells (FIG. 9A). This demonstrates that S and S-F are successfully expressed by the NDV. To examine the incorporation of the S and S-F into the NDV virions, the NDV_LS_S, NDV_LS_S-F and NDV_LS/L289A_S-F were concentrated through a 20% sucrose cushion. The pellets were re-suspended in PBS. The WT NDV_LS was prepared the same way and was used as the negative control. The protein content of each concentrated virus was determined by BCA assay. Two micrograms of each virus was resolved on an SDS-PAGE. A Western blot was performed to examine the abundance of the spike using mouse monoclonal antibody 2B3E5 that binds to a linear epitope of the S1 protein. The expression of the NDV viral hemagglutinin-neuraminidase (HN) protein was also shown as an internal control of the concentrated viruses (FIG. 9B). As expected, both S and S-F incorporated into the NDV particles. Of note, the WT S harboring the polybasic cleavage site (CS) was completely cleaved showing only the S1, while the S-F was maintained at its pre-fusion S0 stage. Importantly, the S-F expressed either by the WT or L289A NDV_LS backbone exhibited superior incorporation into the virions over the WT S shown by much higher abundance of S-F than S1 cleaved from the WT S (FIG. 9B). This confirms that the TM/CT of F in the S-F chimera indeed facilitates the membrane-anchoring of the spike. Since the anti-NDV rabbit sera completely neutralize focus formation of these three NDV vectors vaccines, and for the fact, that the S-F constructs don’t have a polybasic cleavage site, it is unlikely the expression of the transgenes alters the tropism of these viruses.

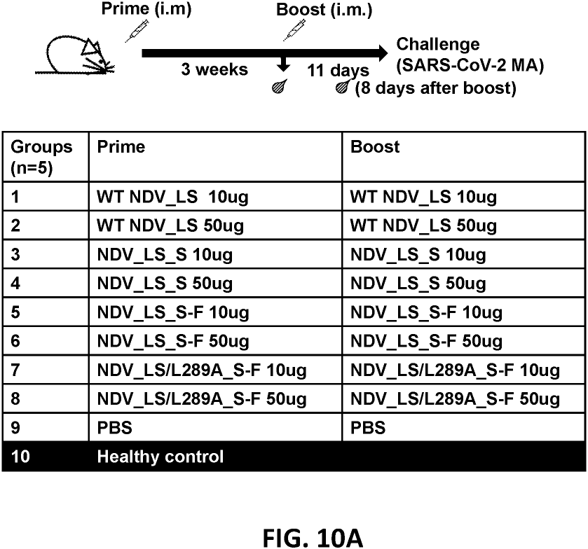

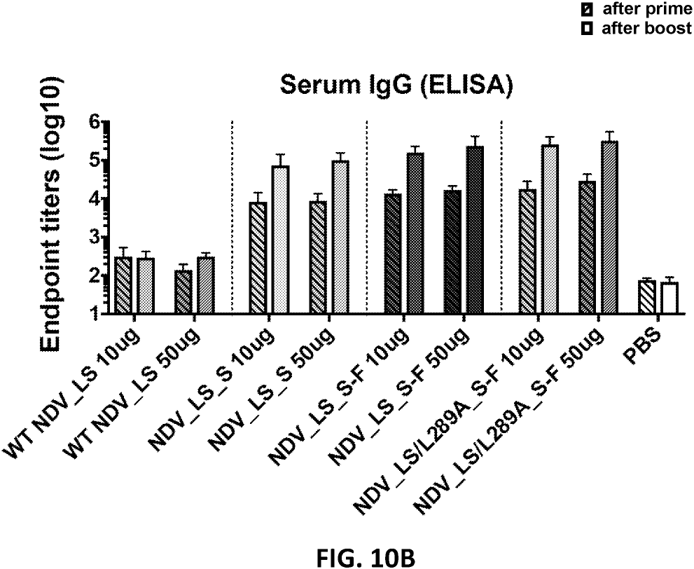

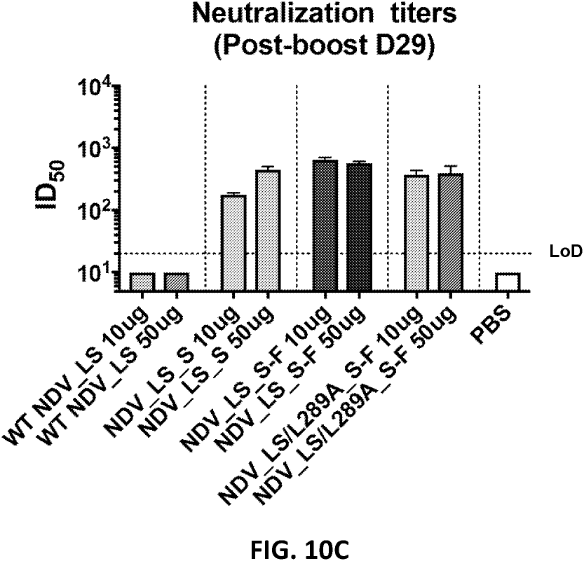

Immunization of mice with NDV LaSota expressing the spike protein elicited potently binding and neutralizing antibodies. To evaluate the immunogenicity of the NDV vectors expressing the S or S-F as vaccine candidates against SARS-CoV-2, a proof of principle study was performed in mice. Specifically, BALB/c mice were immunized with live NDV_LS_S, NDV_LS_S-F and NDV_LS/L289A_S-F intramuscularly, as live NDV barely replicates in the muscle and causes no symptoms in mammals. Here, a prime-boost immunization regimen was used in a three-week interval. Mice were bled pre-boost (after prime) and 8 days after the boost for in vitro serological assays (FIG. 10A). Two doses (10 µg and 50 µg) of each NDV construct including NDV_LS_S (group 3 and 4), NDV_LS_S-F (group 5 and 6) and NDV_LS_S-F (group 7 and 8) were tested as shown in FIG. 10A. Animals vaccinated with WT NDV expressing no transgenes (group 1 and 2) were used as vector-only controls. Mice receiving only the PBS (group 9) were used as negative controls. Mouse sera from the two bleedings were harvested. Serum IgG titers and neutralizing antibody titers were measured by ELISAs and microneutralization assays, respectively. To perform ELISA, full-length trimeric spike protein was coated onto ELISA plates. The endpoint titers of serum IgG were used as the readout (FIG. 10B). After one immunization, all the NDV constructs expressing the spike protein elicited S-binding antibodies, whereas WT NDV constructs and PBS controls show negligible antibody binding signals. The second immunization significantly increased the antibody titers around 1 week after the boost without showing significant difference among the three NDV constructs (FIG. 10B). The neutralizing activity of the antibodies was measured in a microneutralization assay using the USA-WA1/2020 SARS-CoV-2 strain. Pooled sera from each group were tested in a technical duplicate. The ID₅₀ value was calculated as the readout of neutralizing activity of post-boost sera (Day 29). Sera from all vaccinated groups showed neutralizing activity. The neutralization titer of sera from the NDV_LS_S high-dose (50 µg) vaccination group (ID₅₀ ≈ 444) appeared to be slightly higher than that from the low-dose (10 µg) vaccination group (ID₅₀ ≈178). No substantial difference was observed between the low-dose and high-dose groups using the NDV_LS_S-F and NDV_LS/L289A_S-F constructs (FIG. 10C), the neutralization titers of which are comparable to that of the NDV_LS_S high-dose (50 µg) group. To summarize, all the NDV vectors that were engineered to express the S or S-F elicited high titers of binding and neutralizing antibodies in mice. The WT S and S-F constructs appeared to exhibit similar immunogenicity when expressed by live NDV vectors that were given intramuscularly to mice.

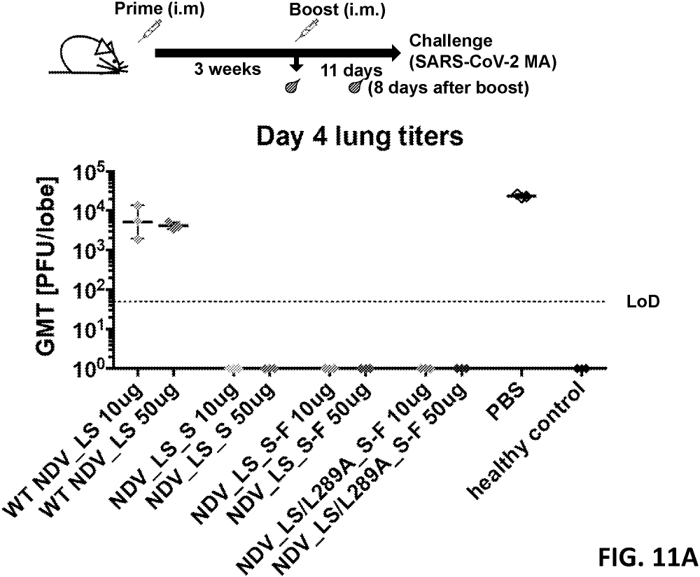

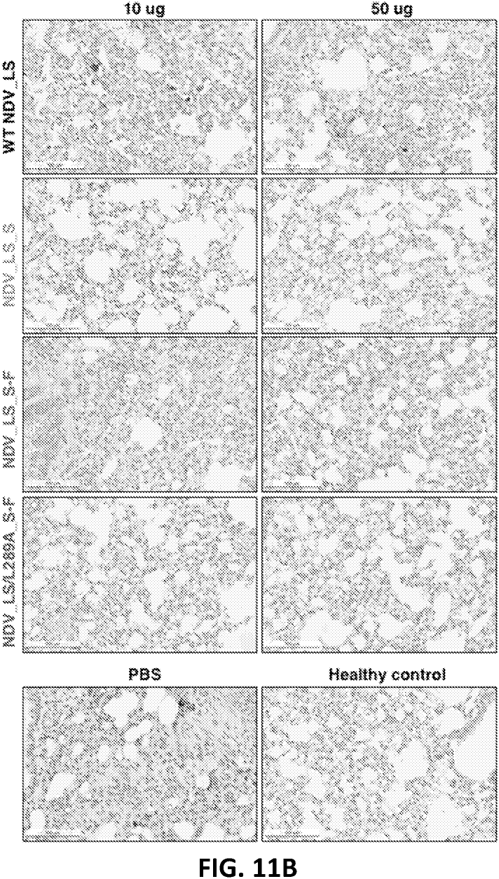

Immunization with NDV LaSota expressing the spike proteins protects mice from challenge with a mouse-adapted SARS-CoV-2. To assess in vivo activity of S-specific antibodies induced by the NDV constructs as well as potential cell-mediated protection, we took advantage of a mouse - adapted SARS-CoV-2 strain that replicates efficiently in BALB/c mice (1, 26). The immunized mice were challenged with 10⁴ PFU of the mouse-adapted SARS-CoV-2 at day 11 after the boost, and viral titers in the lungs at day 4 post -challenge were measured. Mice receiving WT NDV and PBS exhibited high viral titers in the lung, while all the groups given NDV expressing the S or S-F showed no detectible viral load in the lung (FIG. 11A). This showed that vaccination of mice using NDV expressing the S and S-F protected mice against SARS-CoV-2 infections. The lungs of infected mice were fixed in 10% neutral buffered formalin for IHC staining using an anti-SARS-CoV-2 NP antibody. The IHC staining showed that the SARS-CoV-2 NP protein was largely detected in the lungs of mice that received NDV_LS WT or PBS. The SARS-CoV-2 NP was absent in the lungs of mice vaccinated with the three NDV constructs expressing the S or S-F protein (FIG. 11B). These data demonstrated that the all three NDV vector vaccines could efficiently prevent SARS-CoV-2 infection in a mouse model.

### 7.4 Discussion

The consequences of the ongoing COVID-19 pandemic since the end of 2019 are disheartening. With the high transmissibility of the culprit, SARS-CoV-2, and the lack of substantial pre-existing immunity of humans to this virus, many people have succumbed to COVID-19, especially the elderly and people with underlying health conditions. With both therapeutic and prophylactic countermeasures (29-31) still under rapid development, no currently available treatment appears to be effective enough for an over-burdened health care system with limited resources. A vaccine is needed to prevent or at least attenuate the symptoms of COVID-19. As many vaccine candidates are being tested in pre-clinical or clinical studies, a vaccine for cost effective production in low- and middle-income countries has not yet been developed and is still very much in need. Also, the vaccination of small numbers of high-income populations who can afford the vaccine would not efficiently prevent the spreading of the disease in the global population. In this report, a promising viral vector vaccine candidates are described based on NDV expressing the major antigen of SARS-CoV-2. The NDV vectors were engineered to express either the wild type S or a pre-fusion spike with improved membrane anchoring (S-F). These NDV vector vaccines showed robust growth in embryonated chicken eggs despite the fact that a large transgene is inserted into the NDV genome. Importantly, the spike protein is successfully expressed in infected cells, and the S-F construct exhibited superior incorporation into NDV particles, which could potentially be used as an inactivated virus vaccine as well.

In a proof of principle study, mice receiving live NDV vector vaccines twice intramuscularly have developed high levels of spike-specific antibodies that are neutralizing. Mice given the NDV vector expressing S or S-F were protected equally well against the challenge of a mouse-adapted SARS-CoV-2 strain showing no detectable infectious virus or viral antigens in the lungs, while high viral titers were observed in the lungs of mice given the WT NDV expressing no transgenes or PBS. In this study, no significant dose-dependent antibody responses were seen, which was similar to what was observed for different doses (100 µg and 250 µg) of mRNA vaccine in a human trial (2). It could be that the peak antibody responses were not measured due to the problem of having to transfer mice to the University of North Carolina for the challenge study, or an antibody response ceiling was reached with the low dose of 10 µg concentrated virus in mice. In the present study, cellular immunity was not measured, however, this will be of interest to investigate in the future studies. Nevertheless, this study strongly supports that the NDV vector vaccines are promising, as they are expressing immunogenic spike proteins of SARS-CoV-2 inducing high levels of protective antibodies. Unlike other viral vectors that humans might be exposed to, the NDV vector would deliver the spike antigen more efficiently without encountering pre-existing immune responses in humans. Importantly, NDV vector vaccines are not only cost-effective with respect to large scale manufacturing but can also be produced under BSL-2 conditions using influenza virus vaccine production technology. In summary, NDV vector SARS-CoV-2 vaccines are a safe and immunogenic alternative to other SARS-CoV-2 vaccines that can be produced using existing infrastructure in a cost-effective way.

### 7.5 References Cited in Example 2

1. Corbett KS, Edwards D, Leist SR, Abiona OM, Boyoglu-Barnum S, Gillespie RA, Himansu S, Schafer A, Ziwawo CT, DiPiazza AT, Dinnon KH, Elbashir SM, Shaw CA, Woods A, Fritch EJ, Martinez DR, Bock KW, Minai M, Nagata BM, Hutchinson GB, Bahl K, Garcia-Dominguez D, Ma L, Renzi I, Kong WP, Schmidt SD, Wang L, Zhang Y, Stevens LJ, Phung E, Chang LA, Loomis RJ, Altaras NE, Narayanan E, Metkar M, Presnyak V, Liu C, Louder MK, Shi W, Leung K, Yang ES, West A, Gully KL, Wang N, Wrapp D, Doria-Rose NA, Stewart-Jones G, Bennett H, Nason MC, Ruckwardt TJ, et al. 2020. SARS-CoV-2 mRNA Vaccine Development Enabled by Prototype Pathogen Preparedness. bioRxiv doi:10.1101/2020.06.11.145920.

2. Jackson LA, Anderson EJ, Rouphael NG, Roberts PC, Makhene M, Coler RN, McCullough MP, Chappell JD, Denison MR, Stevens LJ, Pruijssers AJ, McDermott A, Flach B, Doria-Rose NA, Corbett KS, Morabito KM, O’Dell S, Schmidt SD, Swanson PA, 2nd, Padilla M, Mascola JR, Neuzil KM, Bennett H, Sun W, Peters E, Makowski M, Albert J, Cross K, Buchanan W, Pikaart-Tautges R, Ledgerwood JE, Graham BS, Beigel JH, m RNASG. 2020. An mRNA Vaccine against SARS-CoV-2 -Preliminary Report. N Engl J Med doi:10.1056/NEJMoa2022483.

3. Gao Q, Bao L, Mao H, Wang L, Xu K, Yang M, Li Y, Zhu L, Wang N, Lv Z, Gao H, Ge X, Kan B, Hu Y, Liu J, Cai F, Jiang D, Yin Y, Qin C, Li J, Gong X, Lou X, Shi W, Wu D, Zhang H, Zhu L, Deng W, Li Y, Lu J, Li C, Wang X, Yin W, Zhang Y, Qin C. 2020. Rapid development of an inactivated vaccine candidate for SARS-CoV-2. Science doi:10.1126/science.abc1932.

4. Zhu FC, Li YH, Guan XH, Hou LH, Wang WJ, Li JX, Wu SP, Wang BS, Wang Z, Wang L, Jia SY, Jiang HD, Wang L, Jiang T, Hu Y, Gou JB, Xu SB, Xu JJ, Wang XW, Wang W, Chen W. 2020. Safety, tolerability, and immunogenicity of a recombinant adenovirus type-5 vectored COVID-19 vaccine: a dose-escalation, open-label, non-randomised, first-in-human trial. Lancet 395:1845-1854.

5. Amanat F, Krammer F. 2020. SARS-CoV-2 Vaccines: Status Report. Immunity 52:583-589.

6. Poh CM, Carissimo G, Wang B, Amrun SN, Lee CY, Chee RS, Fong SW, Yeo NK, Lee WH, Torres-Ruesta A, Leo YS, Chen MI, Tan SY, Chai LYA, Kalimuddin S, Kheng SSG, Thien SY, Young BE, Lye DC, Hanson BJ, Wang CI, Renia L, Ng LFP. 2020. Two linear epitopes on the SARS-CoV-2 spike protein that elicit neutralising antibodies in COVID-19 patients. Nat Commun 11:2806.

7. Grifoni A, Sidney J, Zhang Y, Scheuermann RH, Peters B, Sette A. 2020. A Sequence Homology and Bioinformatic Approach Can Predict Candidate Targets for Immune Responses to SARS-CoV-2. Cell Host Microbe 27:671-680 e2.

8. Ahmed SF, Quadeer AA, McKay MR. 2020. Preliminary Identification of Potential Vaccine Targets for the COVID-19 Coronavirus (SARS-CoV-2) Based on SARS-CoV Immunological Studies. Viruses 12.

9. Shivarov V, Petrov PK, Pashov AD. 2020. Potential SARS-CoV-2 Preimmune IgM Epitopes. Front Immunol 11:932.

10. Pinto D, Park YJ, Beltramello M, Walls AC, Tortorici MA, Bianchi S, Jaconi S, Culap K, Zatta F, De Marco A, Peter A, Guarino B, Spreafico R, Cameroni E, Case JB, Chen RE, Havenar-Daughton C, Snell G, Telenti A, Virgin HW, Lanzavecchia A, Diamond MS, Fink K, Veesler D, Corti D. 2020. Cross-neutralization of SARS-CoV-2 by a human monoclonal SARS-CoV antibody. Nature doi:10.1038/s41586-020-2349-y.

11. Peeters BP, de Leeuw OS, Koch G, Gielkens AL. 1999. Rescue of Newcastle disease virus from cloned cDNA: evidence that cleavability of the fusion protein is a major determinant for virulence. J Virol 73:5001-9.

12. Romer-Oberdorfer A, Mundt E, Mebatsion T, Buchholz UJ, Mettenleiter TC. 1999. Generation of recombinant lentogenic Newcastle disease virus from cDNA. J Gen Virol 80 (Pt 11):2987-95.

13. Zamarin D, Palese P. 2012. Oncolytic Newcastle disease virus for cancer therapy: old challenges and new directions. Future Microbiol 7:347-67.

14. Vigil A, Martinez O, Chua MA, Garcia-Sastre A. 2008. Recombinant Newcastle disease virus as a vaccine vector for cancer therapy. Mol Ther 16:1883-90.

15. Vijayakumar G, Palese P, Goff PH. 2019. Oncolytic Newcastle disease virus expressing a checkpoint inhibitor as a radioenhancing agent for murine melanoma. EBioMedicine 49:96-105.

16. DiNapoli JM, Kotelkin A, Yang L, Elankumaran S, Murphy BR, Samal SK, Collins PL, Bukreyev A. 2007. Newcastle disease virus, a host range-restricted virus, as a vaccine vector for intranasal immunization against emerging pathogens. Proc Natl Acad Sci U S A 104:9788-93.

17. Liu RQ, Ge JY, Wang JL, Shao Y, Zhang HL, Wang JL, Wen ZY, Bu ZG. 2017. Newcastle disease virus-based MERS-CoV candidate vaccine elicits high-level and lasting neutralizing antibodies in Bactrian camels. J Integr Agric 16:2264-2273.

18. Freeman AI, Zakay-Rones Z, Gomori JM, Linetsky E, Rasooly L, Greenbaum E, Rozenman-Yair S, Panet A, Libson E, Irving CS, Galun E, Siegal T. 2006. Phase I/II trial of intravenous NDV-HUJ oncolytic virus in recurrent glioblastoma multiforme. Mol Ther 13:221-8.

19. Pecora AL, Rizvi N, Cohen GI, Meropol NJ, Sterman D, Marshall JL, Goldberg S, Gross P, O’Neil JD, Groene WS, Roberts MS, Rabin H, Bamat MK, Lorence RM. 2002. Phase I trial of intravenous administration of PV701, an oncolytic virus, in patients with advanced solid cancers. J Clin Oncol 20:2251-66.

20. Schirrmacher V. 2016. Fifty Years of Clinical Application of Newcastle Disease Virus: Time to Celebrate! Biomedicines 4.

21. Amanat F, Stadlbauer D, Strohmeier S, Nguyen THO, Chromikova V, McMahon M, Jiang K, Arunkumar GA, Jurczyszak D, Polanco J, Bermudez-Gonzalez M, Kleiner G, Aydillo T, Miorin L, Fierer DS, Lugo LA, Kojic EM, Stoever J, Liu STH, Cunningham-Rundles C, Felgner PL, Moran T, Garcia-Sastre A, Caplivski D, Cheng AC, Kedzierska K, Vapalahti O, Hepojoki JM, Simon V, Krammer F. 2020. A serological assay to detect SARS-CoV-2 seroconversion in humans. Nat Med 26:1033-1036.

22. Vijayakumar G, Zamarin D. 2020. Design and Production of Newcastle Disease Virus for Intratumoral Immunomodulation. Methods Mol Biol 2058:133-154.

23. Li J, Melanson VR, Mirza AM, Iorio RM. 2005. Decreased dependence on receptor recognition for the fusion promotion activity of L289A-mutated newcastle disease virus fusion protein correlates with a monoclonal antibody-detected conformational change. J Virol 79:1180-90.

24. ter Meulen J, van den Brink EN, Poon LL, Marissen WE, Leung CS, Cox F, Cheung CY, Bakker AQ, Bogaards JA, van Deventer E, Preiser W, Doerr HW, Chow VT, de Kruif J, Peiris JS, Goudsmit J. 2006. Human monoclonal antibody combination against SARS coronavirus: synergy and coverage of escape mutants. PLoS Med 3:e237.

25. Yuan M, Wu NC, Zhu X, Lee CD, So RTY, Lv H, Mok CKP, Wilson IA. 2020. A highly conserved cryptic epitope in the receptor binding domains of SARS-CoV-2 and SARS-CoV. Science 368:630-633.

26. Dinnon KH, Leist SR, Schafer A, Edwards CE, Martinez DR, Montgomery SA, West A, Yount BL, Hou YJ, Adams LE, Gully KL, Brown AJ, Huang E, Bryant MD, Choong IC, Glenn JS, Gralinski LE, Sheahan TP, Baric RS. 2020. A mouse-adapted SARS-CoV-2 model for the evaluation of COVID-19 medical countermeasures. bioRxiv doi:10.1101/2020.05.06.081497.

27. Amanat F, White KM, Miorin L, Strohmeier S, McMahon M, Meade P, Liu WC, Albrecht RA, Simon V, Martinez-Sobrido L, Moran T, Garcia-Sastre A, Krammer F. 2020. An In Vitro Microneutralization Assay for SARS-CoV-2 Serology and Drug Screening. Curr Protoc Microbiol 58:e108.

28. Park MS, Steel J, Garcia-Sastre A, Swayne D, Palese P. 2006. Engineered viral vaccine constructs with dual specificity: avian influenza and Newcastle disease. Proc Natl Acad Sci U S A 103:8203-8.

29. Casadevall A, Pirofski LA. 2020. The convalescent sera option for containing COVID-19. J Clin Invest 130:1545-1548.

30. Li L, Zhang W, Hu Y, Tong X, Zheng S, Yang J, Kong Y, Ren L, Wei Q, Mei H, Hu C, Tao C, Yang R, Wang J, Yu Y, Guo Y, Wu X, Xu Z, Zeng L, Xiong N, Chen L, Wang J, Man N, Liu Y, Xu H, Deng E, Zhang X, Li C, Wang C, Su S, Zhang L, Wang J, Wu Y, Liu Z. 2020. Effect of Convalescent Plasma Therapy on Time to Clinical Improvement in Patients With Severe and Life-threatening COVID-19: A Randomized Clinical Trial. JAMA doi:10.1001/jama.2020.10044.

31. Wang Y, Zhang D, Du G, Du R, Zhao J, Jin Y, Fu S, Gao L, Cheng Z, Lu Q, Hu Y, Luo G, Wang K, Lu Y, Li H, Wang S, Ruan S, Yang C, Mei C, Wang Y, Ding D, Wu F, Tang X, Ye X, Ye Y, Liu B, Yang J, Yin W, Wang A, Fan G, Zhou F, Liu Z, Gu X, Xu J, Shang L, Zhang Y, Cao L, Guo T, Wan Y, Qin H, Jiang Y, Jaki T, Hayden FG, Horby PW, Cao B, Wang C. 2020. Remdesivir in adults with severe COVID-19: a randomised, double-blind, placebo-controlled, multicentre trial. Lancet 395:1569-1578.

### 8. EXAMPLE 3: SERUM IGG TITER IN SERUM OF MICE IMMUNIZED WITH NEWCASTLE DISEASE VIRUS (NDV) EXPRESSING SPIKE PROTEIN OF SARS-COV-2

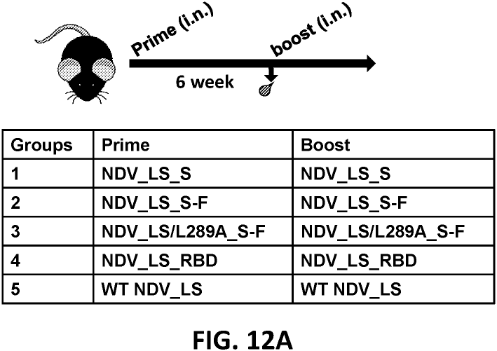

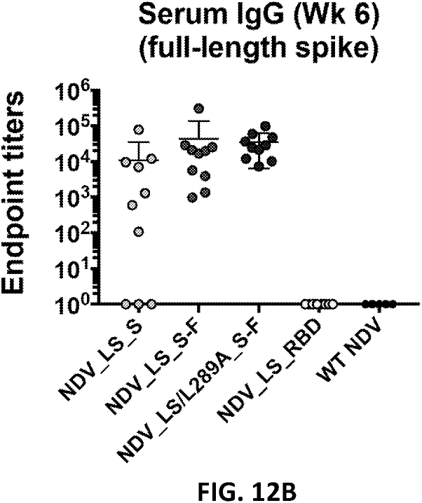

C57BL/6 mice mice were immunized with 10⁵ ffu/mouse of NDV_LS_S, NDV_LS_S-F, NDV _LS/L289A_S-F or NDV_LS_RBD (secreted RBD was expressed as the transgene) intranasally (i.n.). See Example 2 for a description of the NDV_LS_S, NDV_LS_S-F, NDV _LS/L289A_S-F constructs. Wild type NDV_LS was given to a group of mice at 10⁵ ffu/mouse as negative controls. Here, a prime-boost immunization regimen was used. Mice were primed, and six weeks later each group of mice was bled and then boosted with the same virus at the same dose. Mice were bled pre-boost (after prime) for in vitro serological assays (FIG. 12A). Animals vaccinated with WT NDV expressing no transgenes (group 5) were used as vector-only controls. Serum IgG titers were measured by ELISAs. To perform ELISA, full-length trimeric spike protein was coated onto ELISA plates. The endpoint titers of serum IgG were used as the readout (FIG. 12B). After one immunization, all the NDV constructs expressing the spike protein elicited S-binding antibodies, whereas the WT NDV construct (group 5) and NDV_LS_RBD construct (group 4) show negligible antibody binding signals.

### 9. EXAMPLE 4: INCORPORATION OF S-F CHIMERA HEXAPRO PROTEIN EXPRESSED BY NDV_LS/L289A BACKBONE INTO THE VIRION

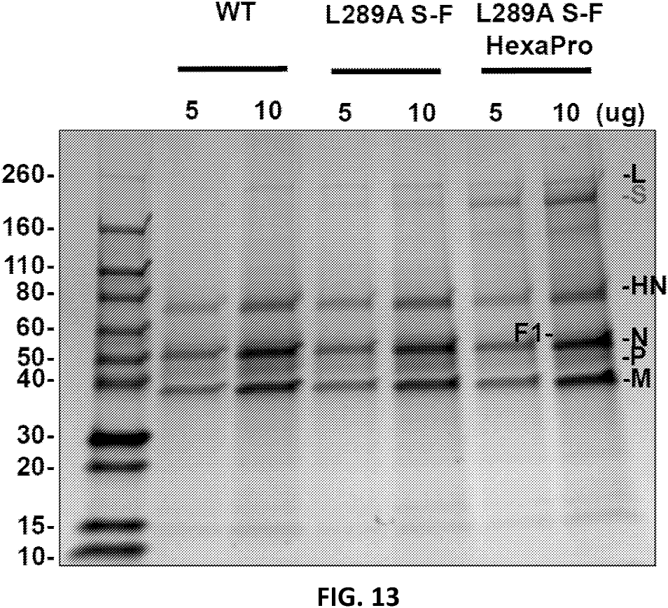

The S-F protein encoded by the NDV construct NDV _LS/L289A_S-F described in Example 2 was modified to include 6 proline substitutions. In particular, amino acid residues corresponding to amino acid residues 817, 892, 899, 942, 986, and 987 of the spike protein found at GenBank Accession No. MN908947 were substituted with prolines. The nucleotide sequence encoding the S-F chimera HexaPro protein and the amino acid sequence of the S-F chimera HexaPro protein are provided in SEQ ID Nos: 14 and 15, respectively. The NDV construct comprising the nucleotide sequence encoding the S-F chimera HexaPro protein is termed “NDV_LS/L289A_S-F HexaPro.” NDV expressing the spike protein of SARS-CoV-2 were rescued as described in Example 2 and the incorporation of S-F transgenes into the virion was confirmed using the techniques described in Example 2. In particular, NDV_LS/L289A_S-F and NDV _LS/L289A_S-F HexaPro were concentrated through a 20% sucrose cushion. The pellets were re-suspended in PBS. The WT NDV_LS was prepared the same way and was used as the negative control. The protein content of each concentrated virus was determined by BCA assay. Five to ten micrograms of each virus was resolved on a 4-20% SDS-PAGE and the gel was stained with Coomassie G-250. As shown in FIG. 13, the S-F HexaPro expressed by the NDV_LS/L289A backbone exhibited superior incorporation into virions over the S-F expressed by the NDV_LS/L289A.

### 10. EXAMPLE 5: A NEWCASTLE DISEASE VIRUS (NDV) EXPRESSING MEMBRANE-ANCHORED SPIKE AS A COST-EFFECTIVE INACTIVATED SARS-COV-2 VACCINE

A successful SARS-CoV-2 vaccine must be not only safe and protective but must also meet the demand on a global scale at low cost. Using the current influenza virus vaccine production capacity to manufacture an egg-based inactivated Newcastle disease virus (NDV)/SARS-CoV-2 vaccine would meet that challenge. This example reports pre-clinical evaluations of an inactivated NDV chimera stably expressing the membrane-anchored form of the spike (NDV-S) as a potent COVID-19 vaccine in mice and hamsters. The inactivated NDV-S vaccine was immunogenic inducing strong binding and/or neutralizing antibodies in both animal models. More importantly, the inactivated NDV-S vaccine protected animals from SARS-CoV-2 infections or significantly attenuated SARS-CoV-2 induced disease. In the presence of an adjuvant, antigen-sparing could be achieved, which would further reduce the cost while maintaining the protective efficacy of the vaccine.

### 10.1 Introduction

A SARS-CoV-2 vaccine is urgently needed to mitigate the current COVID-19 pandemic worldwide. Numerous vaccine approaches are being developed (1-4), however, many of them are not likely to be cost-effective and affordable by low-income countries and under-insured populations. This could be of concern in the long run, as it is crucial to vaccinate a larger population than the high-income minority to effectively contain the spread of the virus. Among all the SARS-CoV-2 vaccine candidates, an inactivated vaccine is attractive as it has a more acceptable safety profile to the public and could be combined with an adjuvant for better protective efficacy and dose-sparing to meet the large global demand. The current platform to produce the inactivated whole virion SARS-CoV-2 vaccine requires the propagation of the virus in cell culture under BSL-3 conditions (3). Excessive inactivation procedures might have to be implemented to ensure the complete inactivation of the virus, at the risk of losing antigenicity of the vaccine. Many viral vector vaccines against coronaviruses have been developed, but they can only be tested as live vaccines (4-9). In addition, the efficacy of certain viral vectors, could be dampened by pre-existing immunity to the viral backbone in the human population.

The construction of Newcastle disease virus (NDV)-based viral vectors expressing a pre-fusion S-F chimera have previously been reported. These NDV vector vaccines have been shown to grow well in embryonated chicken eggs, and that the SARS-CoV-2 spike proteins are abundantly incorporated into the NDV virions. The NDV vector, based on an avian pathogen, overcomes the abovementioned limitation for viral vector vaccines and allows the manufacturing of the vaccine under BSL-2 conditions. In this study, the NDV LaSota L289A mutant expressing the membrane-anchored S-F chimera (NDV-S) was investigated as an inactivated SARS-CoV-2 vaccine candidate with and without an adjuvant in mice and hamsters. The S-F chimera expressed by the NDV chimera was found to be very stable with no antigenicity loss after 3 weeks of 4° C. storage in allantoic fluid. The beta-propiolactone (BPL) inactivated NDV-S vaccine is immunogenic, inducing high titers of S-specific antibodies in both animal models. Furthermore, the effects of a clinical-stage investigational liposomal suspension adjuvant (R-enantiomer of the cationic lipid DOTAP, R-DOTAP)(10-13), as well as an MF-59 like oil-in-water emulsion adjuvant (AddaVax) were also evaluated in mice. Both adjuvants were shown to achieve dose sparing (>10 fold) in mice. The vaccinated animals were protected from SARS-CoV-2 infection or SARS-CoV-2 induced disease. This is encouraging as the existing global egg-based manufacturers of inactivated influenza virus vaccines could be utilized immediately to rapidly produce egg-based NDV-S vaccine with minimal modifications to the production pipelines. Most importantly, this class of products is amenable to large-scale production at low cost and has an excellent safety profile in infants, pregnant women and the elderly (14-16). Alternatively, the NDV-S and other chimeric NDV vaccines can also be produced in tissue culture including Vero cells.

### 10.2 Materials and Methods

Plasmids. The construction of NDV _LS/L289A_S-F rescue plasmid has been described in a previous study. Briefly, the sequence of the ectodomain of the S without the polybasic cleavage site (⁶⁸²RRAR⁶⁸⁵ to A) was amplified from pCAGGS plasmid (17) encoding the codon-optimized nucleotide sequence of the spike gene (GenBank: MN908947.3) of a SARS-CoV-2 isolate by polymerase chain reaction (PCR), using primers containing the gene end (GE), gene start (GS) and a Kozak sequences at the 5′ end (18). The nucleotide sequence of the transmembrane domain (TM) and the cytoplasmic tail (CT) of the NDV_LaSota fusion (F) protein was codon-optimized for mammalian cells and synthesized by IDT (gBlock). The amplified S ectodomain was fused to the TM/CT of F through a GS linker (GGGGS (SEQ ID NO:24)). Additional nucleotides were added at the 3′ end to follow the “rule of six” of paramyxovirus genome. The S-F gene was inserted between the P and M gene of pNDV_LaSota (LS) L289A mutant (NDV_LS/L289A) antigenomic cDNA by in-Fusion cloning (Clontech). The recombination product was transformed into NEB® Stable Competent E. coli (New England Biolabs, Inc.) to generate the NDV_LS/L289A_S-F rescue plasmid. The plasmid was purified using PureLink™ HiPure Plasmid Maxiprep Kit (Thermo Fisher Scientific).

Cells and viruses. BSRT7 cells stably expressing the T7 polymerase were kindly provided by Dr. Benhur Lee at ISMMS. The cells were maintained in Dulbecco’s Modified Eagle’s medium (DMEM; Gibco) containing 10% (vol/vol) fetal bovine serum (FBS) and 100 unit/ml of penicillin and 100 µg/ml of streptomycin (P/S; Gibco) at 37° C. with 5% CO₂. SARS-CoV-2 isolate USA-WA1/2020 (WA-1, BEI Resources NR-52281) used for hamster challenge were propagated in Vero E6 cells (ATCC CRL-1586) in Dulbecco’s Modified Eagle Medium (DMEM), supplemented with 2% fetal bovine serum (FBS), 4.5 g/L D-glucose, 4 mM L-glutamine, 10 mM Non-Essential Amino Acids, 1 mM Sodium Pyruvate, and 10 mM HEPES at 37° C. All experiments with live SARS-CoV-2 were performed in the Centers for Disease Control and Prevention (CDC)/US Department of Agriculture (USDA)-approved biosafety level 3 (BSL-3) biocontainment facility of the Global Health and Emerging Pathogens Institute at the Icahn School of Medicine at Mount Sinai in accordance with institutional biosafety requirements.

Rescue of NDV LaSota expressing the spike of SARS-CoV-2. To rescue NDV_LS/L289A_S-F, six-well plates of BSRT7 cells were seeded 3 × 10⁵ cells per well the day before transfection. The next day, 4 µg of pNDV_LS/L289A_S-F, 2 µg of pTM1-NP, 1 µg of pTM1-P, 1 µg of pTM1-L and 2 µg of pCI-T7opt were re-suspended in 250 µl of Opti-MEM (Gibco). The plasmids cocktail was then gently mixed with 30 µL of TransIT LT1 transfection reagent (Mirus). The mixture was incubated at room temperature (RT) for 30 min. Toward the end of the incubation, the growth medium of each well was replaced with 1 ml of Opti-MEM. The transfection complex was added dropwise to each well and the plates were incubated at 37° C. with 5% CO₂. The supernatant and the cells from transfected wells were harvested at 48 h post-transfection, and briefly homogenized by several strokes using an insulin syringe. Two hundred microliters of the homogenized mixture were injected into the allantoic cavity of 8- to 10-day old specific-pathogen-free (SPF) embryonated chicken eggs. The eggs were incubated at 37° C. for 3 days before cooled at 4° C. overnight. The allantoic fluid was collected and clarified by centrifugation. The rescue of NDV was determined by hemagglutination (HA) assay using 0.5% chicken or turkey red blood cells. The RNA of the positive samples was extracted and treated with DNase I (Thermo Fisher Scientific). Reverse transcriptase-polymerase chain reaction (RT-PCR) was performed to amplify the transgene. The sequences of the transgenes were confirmed by Sanger Sequencing (Genewiz). Recombinant DNA experiments were performed in accordance with protocols approved by the Icahn School of Medicine at Mount Sinai Institutional Biosafety Committee (IBC).

Preparation of concentrated virus. Before concentrating the virus, allantoic fluids were clarified by centrifugation at 4,000 rpm using a Sorvall Legend RT Plus Refrigerated Benchtop Centrifuge (Thermo Fisher Scientific) at 4° C. for 30 min to remove debris. Live virus in the allantoic fluid was pelleted through a 20% sucrose cushion in NTE buffer (100 mM NaCl, 10 mM Tris-HCl, 1 mM EDTA, pH 7.4) by ultra-centrifugation in a Beckman L7-65 ultracentrifuge at 25,000 rpm for two hours at 4° C. using a Beckman SW28 rotor (Beckman Coulter, Brea, CA, USA). Supernatants were aspirated off and the pellets were re-suspended in PBS (pH 7.4). The protein content was determined using the bicinchoninic acid (BCA) assay (Thermo Fisher Scientific). To prepare inactivated concentrated viruses, 1 part of 0.5 M disodium phosphate (DSP) was mixed with 38 parts of the allantoic fluid to stabilize the pH. One part of 2% beta-Propiolactone (BPL) was added dropwise to the mixture during shaking, which gave a final concentration of 0.05% BPL. The treated allantoic fluid was mixed thoroughly and incubated on ice for 30 min. The mixture was then placed in a 37° C. water bath for two hours shaken every 15 min. The inactivated allantoic fluid was clarified by centrifugation at 4,000 rpm for 30 minutes. The inactivation of the virus was confirmed by the lack of growth of the virus from 10-day old embryonated chicken eggs that were inoculated with inactivated virus preparation. The inactivated viruses were concentrated as described above.

Evaluation of stability of the S-F in the allantoic fluid. The allantoic fluid containing the NDV_LS/L289A_S-F virus was harvested and clarified by centrifugation. The clarified allantoic fluid was aliquoted into 15 ml volumes. Week (wk) 0 allantoic fluid was concentrated immediately after centrifugation as described above through a 20% sucrose cushion. The pelleted virus was re-suspended in 300 µL PBS and stored at -80° C. The other three aliquots of the allantoic fluid were maintained at 4° C. to test the stability of the S-F construct. Wk 1, 2 and 3 samples were collected consecutively on a weekly basis, and concentrated virus was prepared in 300 µL PBS using the same method. The protein content of the concentrated virus from wk 0, 1, 2, and 3 was determined using BCA assay after one free-thaw from -80° C. One microgram of each concentrated viruses was resolved on a 4-20% SDS-PAGE (Bio-Rad) and the S-F protein and the HN protein were detected by western blot.

Western Blot. Concentrated live or inactivated virus samples were mixed with Novex™ Tris-Glycine SDS Sample Buffer (2×) (Thermofisher Scientific) with NuPAGE™ Sample Reducing Agent (10×) (Thermofisher Scientific). One or two micrograms of the concentrated viruses were heated at 95° C. for 5 min before being resolved on 4-20% SDS-PAGE (Bio-Rad) using the Novex™ Sharp Pre-stained Protein Standard (ThermoFisher Scientific) as the protein marker. To perform western blot, proteins were transferred onto polyvinylidene difluoride (PVDF) membrane (GE healthcare). The membrane was blocked with 5% non-fat dry milk in PBS containing 0.1% v/v Tween 20 (PBST) for 1 h at RT. The membrane was washed with PBST on a shaker three times (10 min at RT each time) and incubated with an S-specific mouse monoclonal antibody 2B3E5 (provided by Dr. Thomas Moran at ISMMS) or an HN-specific mouse monoclonal antibody 8H2 (MCA2822, Biorad) diluted in PBST containing 1% bovine serum albumin (BSA), overnight at 4° C. The membranes were then washed with PBST on a shaker 3 times (10 min at RT each time) and incubated with secondary sheep anti-mouse IgG linked with horseradish peroxidase (HRP) diluted (1:2,000) in PBST containing 5% non-fat dry milk. The secondary antibody was discarded and the membranes were washed with PBST on a shaker three times (10 min at RT each time). Pierce™ ECL Western Blotting Substrate (Thermo Fisher Scientific) was added to the membrane, the blots were imaged using the Bio-Rad Universal Hood Ii Molecular imager (Bio-Rad) and processed by Image Lab Software (Bio-Rad)

Immunization and challenge study in BALB/c mice. Seven-week old female BALB/cJ mice (Jackson Laboratories) were used in this study. Experiments were performed in accordance with protocols approved by the Icahn School of Medicine at Mount Sinai Institutional Animal Care and Use Committee (IACUC). Mice were divided into 10 groups (n=5) receiving the inactivated virus without or with an adjuvant at three different doses intramuscularly. The vaccination followed a prime-boost regimen in a 2-week interval. Specifically, group 1, group 2 and group 3 received 5 µg, 10 µg and 20 µg inactivated NDV-S vaccine (total protein) without the adjuvant, respectively; Group 4, group 5 and group 6 received low doses of 0.2 µg, 1 µg and 5 µg of inactivated NDV-S vaccine, respectively, combined with 300 µg of R-DOTAP (PDS Biotechnology) per mouse; Group 7, group 8 and group 9 mice received 0.2 µg, 1 µg and 5 µg of inactivated NDV-S vaccine, respectively, with AddaVax (Invivogen) as the adjuvant. Group 10 received 20 µg inactivated WT NDV as the vector-only control. The SARS-CoV-2 challenge was performed at the University of North Carolina by Dr. Ralph Baric’s group in a Biosafety Level 3 (BSL-3) facility. Mice were challenged 19 days after the boost using a mouse-adapted SARS-CoV-2 strain at 7.5 × 10⁴ plaque forming unit (PFU) intranasally (i.n). Weight loss was monitored for 4 days.

Immunization and challenge study in golden Syrian hamsters. Eight-week old female golden Syrian hamsters were used in this study. Experiments were performed in accordance with protocols approved by the Icahn School of Medicine at Mount Sinai Institutional Animal Care and Use Committee (IACUC). Five groups (n=8) of hamsters were included. The inactivated vaccines were given intramuscularly following a prime-boost regimen in a 2-week interval. Group 1 received 10 µg of inactivated NDV-S vaccine; group 2 received 5 µg of inactivated NDV-S vaccine combined with AddaVax; group 3 hamsters received 10 µg of inactivated WT NDV as vector-only control. A healthy control group receiving no vaccines was also included. Twenty-four days after the boost, hamsters were challenged intranasally with 10⁴ PFU of USA-WA1/2020 SARS-CoV-2 strain. Weight loss was monitored for 5 days.

Lung titers. Lung lobes of mice were collected and homogenized in PBS. A plaque assay was performed to measure viral titer in the lung homogenates as described previously (1, 19). Geometric mean titers of plaque forming units (PFU) per lobe were calculated using GraphPad Prism 7.0.

ELISAs. Mice were bled pre-boost and 11 days after the boost. Hamsters were bled pre-boost and 26 days after the boost. Sera were isolated by low-speed centrifugation. ELISAs were performed as described previously (17). Briefly, Immulon 4 HBX 96-well ELISA plates (Thermo Fisher Scientific) were coated with 2 µg/ml of recombinant trimeric S protein (50 µl per well) in coating buffer (SeraCare Life Sciences Inc.) overnight at 4° C. The next day, all plates were washed 3 times with 220 µL PBS containing 0.1% (v/v) Tween-20 (PBST) and blocked in 220 µL blocking solution (3% goat serum, 0.5% non-fat dried milk powder, 96.5% PBST) for 1 h at RT. Both mouse sera and hamster sera were 3-fold serially diluted in blocking solution starting at 1:30 followed by a 2 h incubation at RT. ELISA plates were washed 3 times with PBST and incubated in 50 µL per well of sheep anti-mouse IgG-horseradish peroxidase(HRP) conjugated antibody (GE Healthcare) or goat anti-hamster IgG-HRP conjugated antibody (Invitrogen) diluted (1:3,000) in blocking solution. Plates were washed 3 times with PBST and 100 µL of o-phenylenediamine dihydrochloride (SigmaFast OPD, Sigma) substrate was added per well. After developing the plates for 10 min, 50 µL of 3 M hydrochloric acid (HCL) was added to each well to stop the reactions. The optical density (OD) was measured at 492 nm on a Synergy 4 plate reader (BioTek) or equivalents. An average of OD values for blank wells plus three standard deviations was used to set a cutoff for plate blank outliers. A cutoff value was established for each plate that was used for calculating the endpoint titers. The endpoint titers of serum IgG responses was graphed using GraphPad Prism 7.0.

Micro-neutralization assay. All neutralization assays were performed in the biosafety level 3 (BSL-3) facility following institutional guidelines as described previously (17, 20). Briefly, serum samples were heat-inactivated at 56° C. for 60 minutes prior to use. Vero E6 cells were maintained in culture using DMEM supplemented with 10% fetal bovine serum (FBS). Twenty-thousands cells per well were seeded in a 96-well cell culture plate the night before the assay. Pooled sera in technical duplicates were serially diluted by 3-fold in starting at 1:20 in a 96-well cell culture plate and each dilution was mixed with 600 times the 50% tissue culture infectious dose (TCID₅₀) of SARS-CoV-2 (USA-WA1/2020, BEI Resources NR-52281). Serum-virus mixture was incubated for 1 h at RT before added to the cells for another hour of incubation in a 37° C. incubator. The virus-serum mixture was removed and the corresponding serum dilution was added to the cells. The cells were incubated for 2 days and fixed with 100 µL 10% formaldehyde per well for 24 h before taken out of the BSL-3 facility. The staining of the cells was performed in a biosafety cabinet (BSL-2). The formaldehyde was carefully removed from the cells. Cells were washed with 200 µL PBS once before permeabilized with PBS containing 0.1% Triton X-100 for 15 min at RT. Cells were washed with PBS and blocked in PBS containing 3% dry milk for 1 h at RT. Cells were then stained with 100 µL per well of a mouse monoclonal anti-NP antibody (1C7), kindly provided by Dr. Thomas Moran at ISMMS, at 1 µg/ml for 1 h at RT. Cells were washed with PBS and incubated with 100 µL per well anti-mouse IgG HRP (Rockland) secondary antibody at 1:3,000 dilution in PBS containing 1% dry milk for 1 h at RT. Finally, cells were washed twice with PBS and the plates were developed using 100 µL of SigmaFast OPD substrate. Ten minutes later, the reactions were stopped using 50 µL per well of 3 M HCI. The OD 492 nm was measured on a Biotek SynergyH1 Microplate Reader. Non-linear regression curve fit analysis (The top and bottom constraints are set at 100% and 0%) over the dilution curve was performed to calculate 50% of inhibitory dilution (ID₅₀) of the serum using GraphPad Prism 7.0.

Statistics. The statistical analysis was performed using GraphPad Prism 7.0. The statistical difference in lung viral titers was determined using the Kruskal-Wallis test with Dunn’s correction for multiple comparisons.

### 10.3 Results

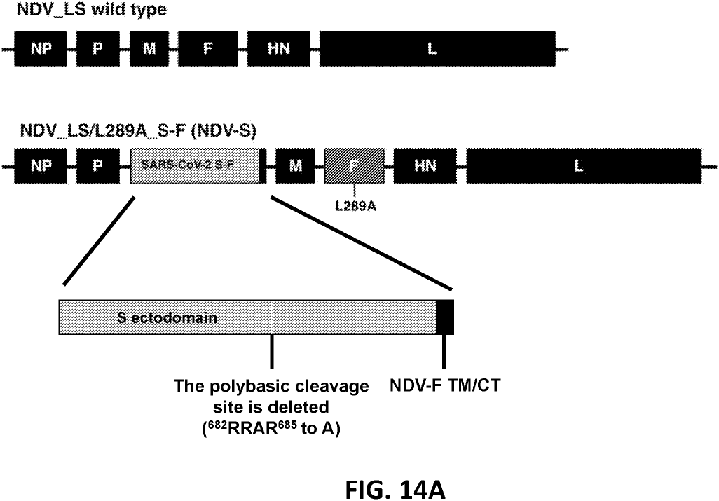

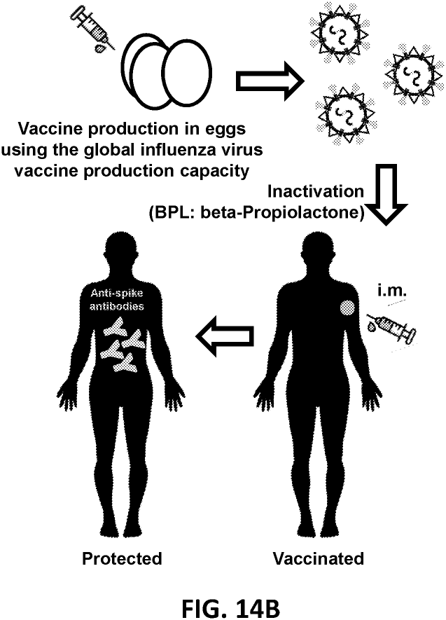

The design and concept of NDV-based inactivated SARS-CoV-2 vaccines. The construction of NDV-based SARS-CoV-2 vaccine candidates were previously reported, among which NDV vectors expressing the spike without the polybasic cleavage site (and the transmembrane region and cytoplasmic tail of NDV F) showed higher abundance of the spike protein in the NDV particles than the NDV vector expressing just the wild type (WT) S protein. The final construct also had a mutation (L289A) in the F protein of NDV which was shown to facilitate HN-independent fusion of the virus (FIG. 14A). To develop an NDV-based inactivated SARS-CoV-2 vaccine, the existing global influenza virus vaccine production capacity could be employed as both influenza virus and NDV grow to high titers in embryonated chicken eggs. With few modifications to the manufacturing process of inactivated influenza virus vaccines, NDV-S vaccine can be purified by zonal sucrose density centrifugation. Instead of formalin inactivation for influenza virus vaccine, beta-Propiolactone (BPL) inactivation can be performed (because of a milder inactivation process). Such inactivated NDV-S vaccine will display large spike proteins, which are likely more immunodominant over the HN and F proteins of NDV, on the surface of the whole inactivated virion. The inactivated NDV-S vaccine could be administered intramuscularly, with an adjuvant for dose sparing. This approach should be suited to safely induce spike-specific protective antibodies (FIG. 14B).

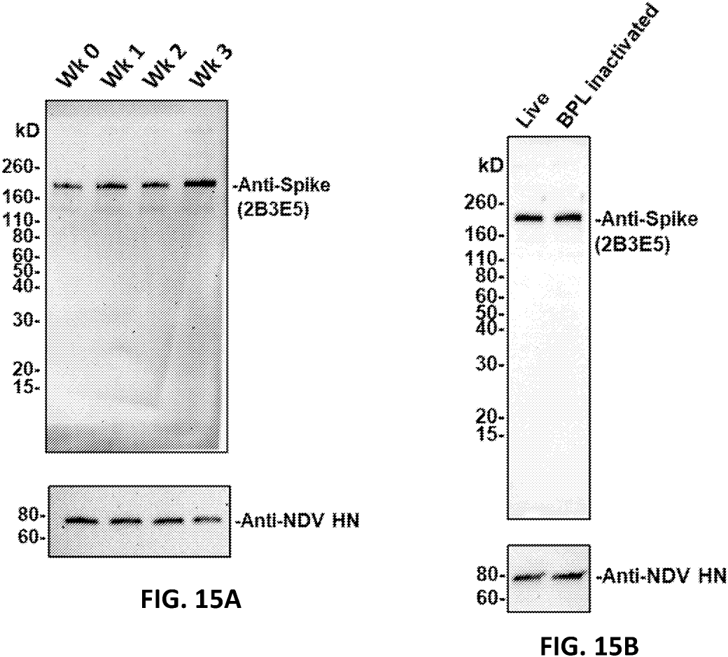

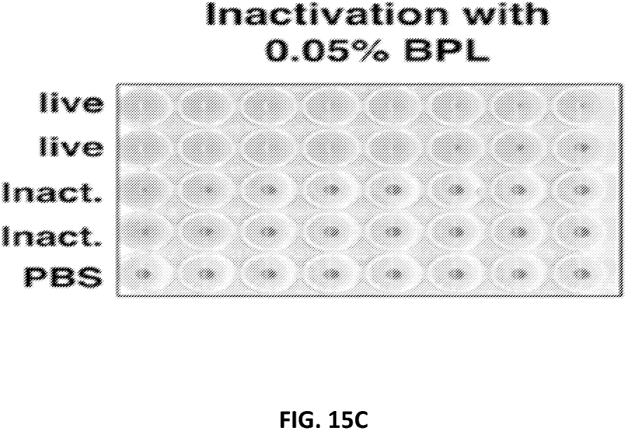

The spike protein expressed by NDV is stable in allantoic fluid. The stability of the antigen could be of concern as the vaccine needs to be purified and inactivated through a temperature-controlled (~4° C.) process. The final product is often formulated and stored in liquid buffer at 4° C. To examine the stability of the S-F protein, allantoic fluid containing the NDV-S live virus was aliquoted into equal volume (15 ml), and stored at 4° C. Samples were collected weekly (wk 0, 1, 2, 3) and concentrated through a 20% sucrose cushion. The concentrated virus was re-suspended in equal amounts of PBS. The total protein content of the 4 aliquots was comparable among the preparations (wk 0: 0.94 mg/ml; wk 1: 1.04 mg/ml; wk 2: 0.9 mg/ml; wk 3: 1.08 mg/ml). The stability of the S-F construct was evaluated by western blot. while the NDV HN protein was used as a control. Interestingly, as HN protein slightly degraded over time, the S-F showed extraordinary stability when kept in allanotic fluid at 4° C. (FIG. 15A). The inactivation by 0.05% BPL was confirmed by the lack of HA activity following inoculation of the inactivated virus into embryonated chicken eggs (FIG. 15C). Moreover, the inactivation procedure using 0.05% BPL did not cause any loss of antigenicity of the S-F, as evaluated by western blot (FIG. 15B). These observations demonstrated the membrane anchored S-F chimera expressed by the NDV vector was very stable with no degradation caused by storage at 4° C. for weeks or treatment with BPL for inactivation.

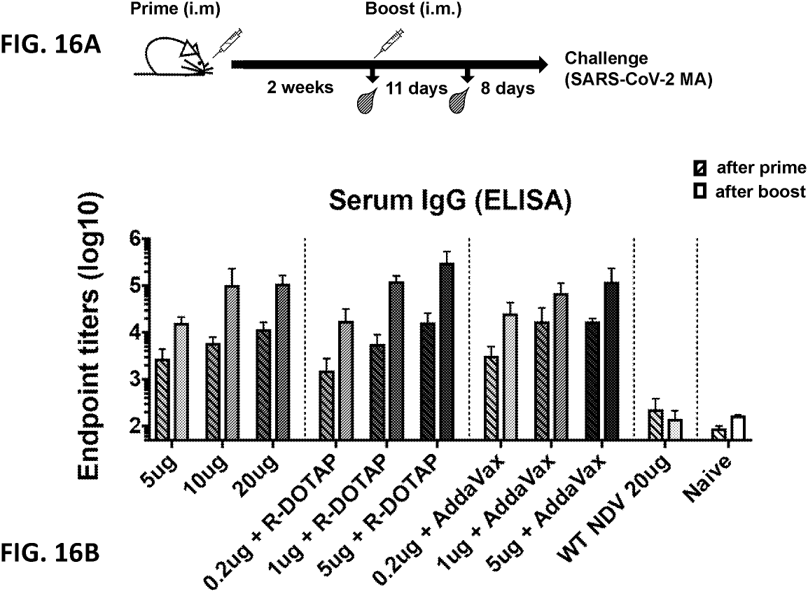

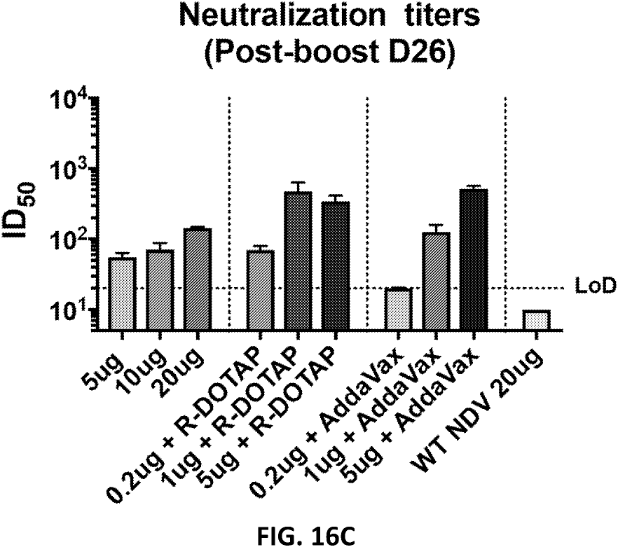

Inactivated NDV-S vaccine induced high titers of binding and neutralizing antibodies in mice. For a pre-clinical evaluation of the inactivated NDV-S vaccine, the immunogenicity of the vaccine as well as the dose sparing ability of the adjuvants were investigated in mice. The vaccines were administered intramuscularly, following a prime-boost regimen in a 2-week interval. Specifically, for the three unadjuvanted groups, mice were immunized with inactivated NDV-S vaccine at 5 µg, 10 µg or 20 µg per mouse intramuscularly. Two adjuvants were tested here, a clinical-stage investigational liposomal suspension of the pure R-enantiomer of the cationic lipid DOTAP (R-DOTAP) and an MF59-like oil-in-water emulsion adjuvant AddaVax. Each adjuvant was combined with low doses of NDV-S vaccines at 0.2 µg, 1 µg and 5 µg. Mice receiving 20 µg of inactivated WT NDV were used as vector-only (negative) controls. Mice were bled pre-boost (2 weeks after prime) and 11 days post-boost to examine antibody responses by ELISAs using a trimeric full-length S protein as the substrate (17), and micro-neutralization assay using the USA-WA1/2020 strain of SARS-CoV-2 (FIG. 16A). After one immunization all the groups developed S-specific antibodies. The boost greatly increased the antibody titers of all NDV-S immunizations. R-DOTAP combined with 5 µg of vaccine showed the highest antibody titer. One microgram of vaccine with R-DOTAP or AddaVax and 5 µg of vaccine with AddaVax induced comparable levels of binding antibody, which is also similar to the titer induced by 20 µg of vaccine without an adjuvant. As expected, the inactivated wild type NDV only induced baseline level of antibody responses (FIG. 16B). Moreover, microneutralization assays were performed to determine the neutralizing activity of serum antibodies collected from vaccinated mice. Sera from all the groups except the WT NDV groups showed neutralizing activity against the SARS-CoV-2 USA-WA1/2020 strain. The neutralization titers of 1 µg of vaccine with R-DOTAP (ID₅₀ of ~476) and 5 µg of vaccine with AddaVax groups (ID₅₀ of ~515) appear to be the highest and comparable to each other. Interestingly, although the group receiving 5 µg of vaccine with R-DOTAP developed the most abundant binding antibodies in ELISA, these sera were not the most neutralizing ones suggesting R-DOTAP might have a different mechanism of action from that of AddaVax. R-DOTAP is an immune modulator, that induces the production of important cytokines and chemokines and enhances cytolytic T cells when combined with proteins.. It is likely that with more antigen, the immune responses were skewed towards CD8+ T-cell responses with R-DOTAP, and non-neutralizing antibodies were induced (FIG. 16C). These results demonstrated that inactivated NDV-S vaccine expressing the membrane anchored S-F was immunogenic inducing potent binding and neutralizing antibodies. Importantly, at least 10-fold dose sparing was achieved with an adjuvant in mice.

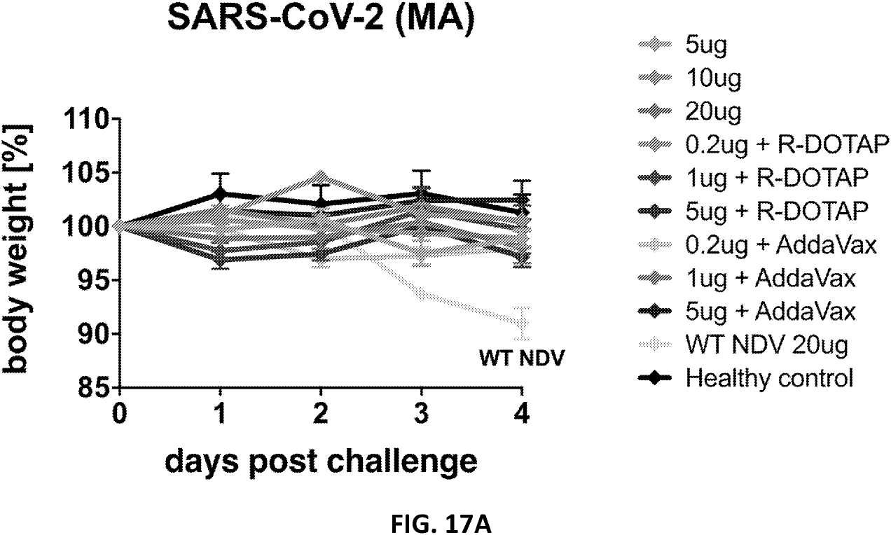

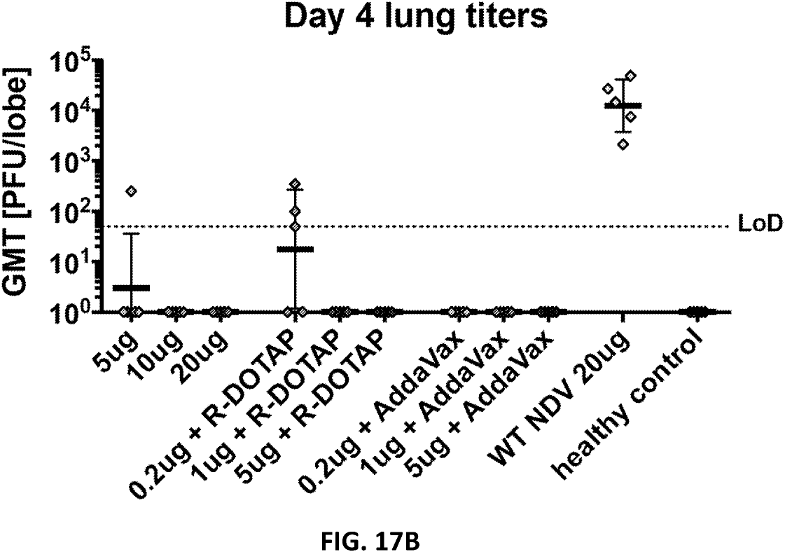

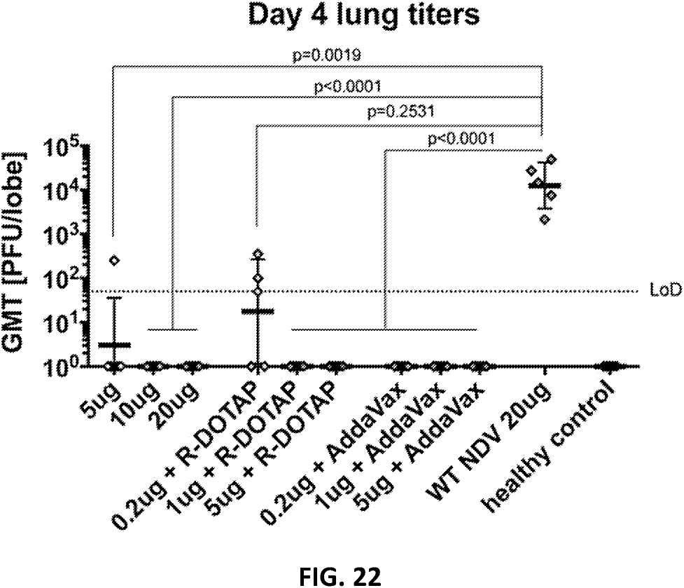

The inactivated NDV-S vaccine protected mice from the challenge of a mouse-adapted SARS-CoV-2. To evaluate vaccine-induced protection, mice were challenged 19 days after boost using a mouse-adapted SARS-CoV-2 virus (FIG. 16A). Weight loss was monitored for 4 days. Only the negative control group receiving the WT NDV was observed to lose notable weight (~10%) by day 4, while all the vaccinated groups showed no weight loss (FIG. 17A). Viral titers in the lung at 4 days post challenge were also measured. As expected, the negative control group given the WT NDV exhibited the highest viral titer of >10⁴ pfu/lobe. Groups receiving 5 µg of unadjuvanted vaccine and 0.2 µg of vaccine with R-DOTAP showed detectable but low viral titers in the lung, while all the other groups were fully protected (FIG. 17B). See also FIG. 22.These results are encouraging as 0.2 µg of vaccine with AddaVax protected as well as 10 µg of vaccine without an adjuvant. Although 0.2 µg of vaccine with R-DOTAP did not induce sterilizing immunity, approximately a 1000-fold reduction of viral titer in the lungs was achieved. To conclude, the inactivated NDV-S exhibits great potentials as a cost-effective vaccine as it induces protective immunity against the SARS-CoV-2 at very low doses with an adjuvant.

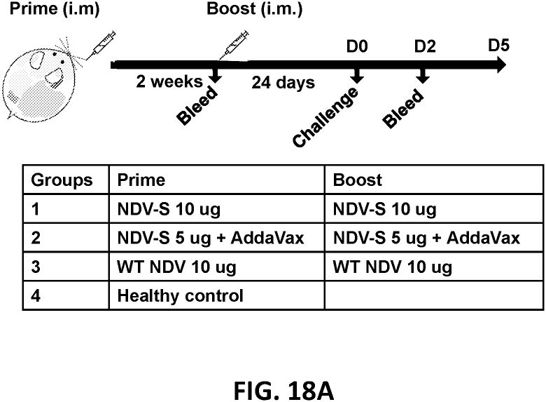

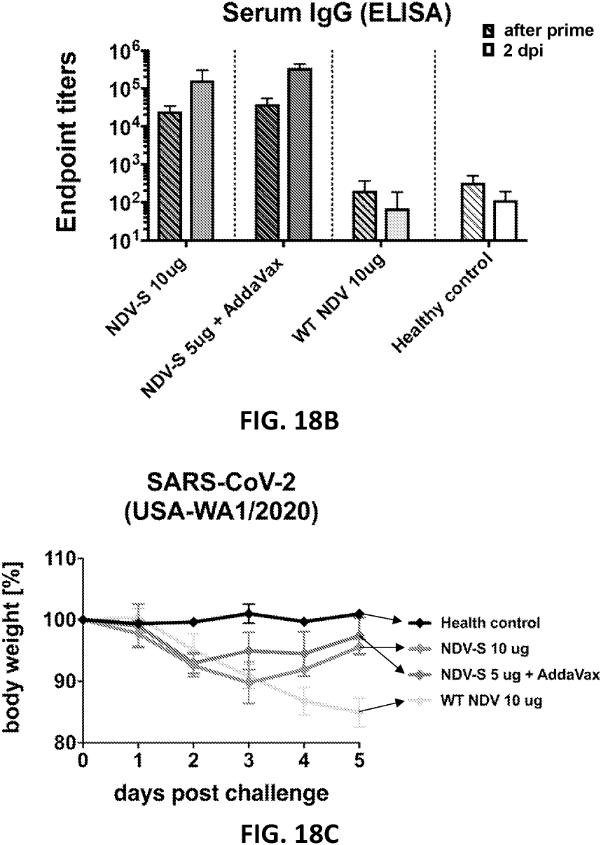

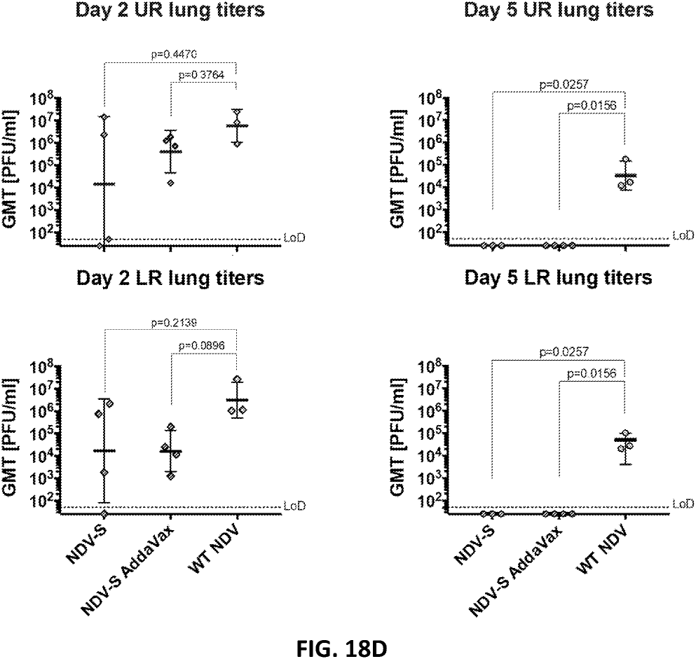

The inactivated NDV-S vaccine confers protection against the SARS-CoV-2 challenge in a hamster model. Golden Syrian hamsters have been characterized as a useful small animal model for COVID-19 as they are susceptible to SARS-CoV-2 infections and manifest SARS-CoV-2 induced diseases (21, 22). Here, a pilot immunogenicity and efficacy study of the inactivated NDV-S vaccine in hamsters was conducted. The vaccinations also followed a prime-boost regimen in a 2-week interval via intramuscular administration route. Twenty-four days after the boost, hamsters were challenged with the SARS-CoV-2 USA-WA1/2020 at 10⁴ pfu per animal intranasally. Four groups of hamsters were included in this study. Group 1 was given 10 µg of inactivated NDV-S vaccine per animal without adjuvants. Group 2 received 5 µg of inactivated NDV-S vaccine with AddaVax as an adjuvant. Group 3 was the vector-only negative control immunized with 10 µg of inactivated WT NDV. Group 4 receiving no vaccine and mock-challenged with PBS was used as healthy controls (FIG. 18A). Serum IgG titers from animals at pre-boost and 2-day post infection (dpi) were measured by ELISAs. One immunization with NDV-S vaccine ± the adjuvant successfully induced spike-specific antibodies. Since there was no seroconversion from infection at 2 dpi indicated by baseline level of the WT NDV sera, the increase in titers at 2 dpi as compared with titers after vaccine priming most likely represented vaccine-induced antibody levels after the boost. As expected, the boost substantially increased the antibody titers in the NDV-S vaccination groups, whereas the WT NDV sera showed negligible binding signals (FIG. 18B). Nevertheless, we cannot exclude a contribution from a rapid production of S antibodies by vaccine-induced memory B cells after exposure to SARS-CoV-2. Hamsters were challenged and weight loss was monitored for 5 days. The WT NDV group lost up to 15% of weight by 5 dpi. Animals receiving 10 µg of inactivated NDV-S vaccine lost ~10% of weight by 3 dpi and started to recover. Animals receiving 5 µg inactivated NDV-S vaccine with AddaVax only lost weight on 2 dpi but quickly recovered (FIG. 18C). Viral titers in the upper right (UR) lung lobes and lower right (LR) lung lobes were also measured. The lung lobes were homogenized in 1 mL of PBS. Viral titers in the lung homogenates were measured by a plaque assay. Animals vaccinated with NDV-S with or without adjuvant displayed a substantial reduction of viral adjuvant displayed a substantial reduction of viral titers at 2 dpi, while the viral titers of these two groups at 5 dpi were below the limit of detection (FIG. 18D). These data suggested inactivated NDV-S vaccine could effectively attenuate the symptoms of SARS-CoV-2 induced diseases in hamsters.

### 10.4 Discussion

To develop viral vector vaccines against SARS-CoV-2, NDV-based SARS-CoV-2 vaccines expressing two forms of spike protein (S and S-F) have previously been reported. Since the S-F showed superior incorporation into NDV particles, its potential of being used as an inactivated vaccine was investigated in this study. The NDV-S was found to be very stable when stored at 4° C. for 3 weeks with no loss of antigenicity of the S-F protein. In mice, here it has been shown a total amount of inactivated NDV-S vaccine as low as 0.2 µg could significantly reduce viral titers in the lung, approximately by a factor of 1000 when combined with R-DOTAP, while the adjuvant AddaVax conferred even better protection. NDV-S vaccine at 1 µg with either adjuvant elicited potent neutralizing antibodies and resulted in undetectable viral titers in the lung. These pre-clinical results demonstrate that antigen-sparing greater than 10-fold can be achieved in a mouse model, providing valuable input for clinical trials in humans. In a pilot hamster experiment, the inactivated NDV-S vaccine is also immunogenic inducing high titers of spike-specific antibodies. Since hamsters are much more susceptible to SARS-CoV-2 infection, the group receiving the WT NDV lost up to 15% of weight by day 5, while both NDV-S vaccinated groups ± the adjuvant greatly attenuated SARS-CoV-2 induced disease determined by the weight loss. The AddaVax adjuvant again enhanced vaccine-induced protection, resulting in weight loss only on 2 dpi of the group. The dosing of the adjuvant R-DOTAP was not well determined for this model by the time of vaccination. Therefore, it was not used in this experiment. However, R-DOTAP as well as additional adjuvants will be evaluated in combination with the inactivated NDV-S vaccine in future studies. In addition, other outcomes of SARS-CoV-2 induced disease in hamsters, such as viral titers in nasal washes or lungs, will be examined.

Promising protection by immunization with inactivated NDV-S in both the mouse and the hamster model has been shown. Even though sterilizing immunity might not always be induced, the trade-off for having an affordable and widely available effective vaccine that reduces the symptoms of COVID-19 should be much preferred over a high-cost vaccine that is limited to high income populations. Most importantly, the egg-based production of NDV-S vaccine only requires few changes of current inactivated influenza virus vaccine manufacturing procedures. The cost of goods should be similar to that of a monovalent inactivated influenza virus vaccine (a fraction of the cost of a quadrivalent seasonal influenza virus vaccine), or even lower due to dose sparing with an adjuvant that is inexpensive to manufacture.

### 10.5 References Cited in Example 5

1. Corbett KS, Edwards D, Leist SR, Abiona OM, Boyoglu-Barnum S, Gillespie RA, Himansu S, Schafer A, Ziwawo CT, DiPiazza AT, Dinnon KH, Elbashir SM, Shaw CA, Woods A, Fritch EJ, Martinez DR, Bock KW, Minai M, Nagata BM, Hutchinson GB, Bahl K, Garcia-Dominguez D, Ma L, Renzi I, Kong WP, Schmidt SD, Wang L, Zhang Y, Stevens LJ, Phung E, Chang LA, Loomis RJ, Altaras NE, Narayanan E, Metkar M, Presnyak V, Liu C, Louder MK, Shi W, Leung K, Yang ES, West A, Gully KL, Wang N, Wrapp D, Doria-Rose NA, Stewart-Jones G, Bennett H, Nason MC, Ruckwardt TJ, et al. 2020. SARS-CoV-2 mRNA Vaccine Development Enabled by Prototype Pathogen Preparedness. bioRxiv doi:10.1101/2020.06.11.145920.

2. Jackson LA, Anderson EJ, Rouphael NG, Roberts PC, Makhene M, Coler RN, McCullough MP, Chappell JD, Denison MR, Stevens LJ, Pruijssers AJ, McDermott A, Flach B, Doria-Rose NA, Corbett KS, Morabito KM, O’Dell S, Schmidt SD, Swanson PA, 2nd, Padilla M, Mascola JR, Neuzil KM, Bennett H, Sun W, Peters E, Makowski M, Albert J, Cross K, Buchanan W, Pikaart-Tautges R, Ledgerwood JE, Graham BS, Beigel JH, m RNASG. 2020. An mRNA Vaccine against SARS-CoV-2 -Preliminary Report. N Engl J Med doi:10.1056/NEJMoa2022483.

3. Gao Q, Bao L, Mao H, Wang L, Xu K, Yang M, Li Y, Zhu L, Wang N, Lv Z, Gao H, Ge X, Kan B, Hu Y, Liu J, Cai F, Jiang D, Yin Y, Qin C, Li J, Gong X, Lou X, Shi W, Wu D, Zhang H, Zhu L, Deng W, Li Y, Lu J, Li C, Wang X, Yin W, Zhang Y, Qin C. 2020. Rapid development of an inactivated vaccine candidate for SARS-CoV-2. Science doi:10.1126/science.abc1932.

4. Zhu FC, Li YH, Guan XH, Hou LH, Wang WJ, Li JX, Wu SP, Wang BS, Wang Z, Wang L, Jia SY, Jiang HD, Wang L, Jiang T, Hu Y, Gou JB, Xu SB, Xu JJ, Wang XW, Wang W, Chen W. 2020. Safety, tolerability, and immunogenicity of a recombinant adenovirus type-5 vectored COVID-19 vaccine: a dose-escalation, open-label, non-randomised, first-in-human trial. Lancet 395:1845-1854.

5. DiNapoli JM, Kotelkin A, Yang L, Elankumaran S, Murphy BR, Samal SK, Collins PL, Bukreyev A. 2007. Newcastle disease virus, a host range-restricted virus, as a vaccine vector for intranasal immunization against emerging pathogens. Proc Natl Acad Sci U S A 104:9788-93.

6. Liu RQ, Ge JY, Wang JL, Shao Y, Zhang HL, Wang JL, Wen ZY, Bu ZG. 2017. Newcastle disease virus-based MERS-CoV candidate vaccine elicits high-level and lasting neutralizing antibodies in Bactrian camels. J Integr Agric 16:2264-2273.

7. Li K, Li Z, Wohlford-Lenane C, Meyerholz DK, Channappanavar R, An D, Perlman S, McCray PB, Jr., He B. 2020. Single-Dose, Intranasal Immunization with Recombinant Parainfluenza Virus 5 Expressing Middle East Respiratory Syndrome Coronavirus (MERS-CoV) Spike Protein Protects Mice from Fatal MERS-CoV Infection. mBio 11.

8. Liniger M, Zuniga A, Tamin A, Azzouz-Morin TN, Knuchel M, Marty RR, Wiegand M, Weibel S, Kelvin D, Rota PA, Naim HY. 2008. Induction of neutralising antibodies and cellular immune responses against SARS coronavirus by recombinant measles viruses. Vaccine 26:2164-74.

9. Koch T, Dahlke C, Fathi A, Kupke A, Krahling V, Okba NMA, Halwe S, Rohde C, Eickmann M, Volz A, Hesterkamp T, Jambrecina A, Borregaard S, Ly ML, Zinser ME, Bartels E, Poetsch JSH, Neumann R, Fux R, Schmiedel S, Lohse AW, Haagmans BL, Sutter G, Becker S, Addo MM. 2020. Safety and immunogenicity of a modified vaccinia virus Ankara vector vaccine candidate for Middle East respiratory syndrome: an open-label, phase 1 trial. Lancet Infect Dis 20:827-838.

10. Vasievich EA, Ramishetti S, Zhang Y, Huang L. 2012. Trp2 peptide vaccine adjuvanted with (R)-DOTAP inhibits tumor growth in an advanced melanoma model. Mol Pharm 9:261-8.

11. Smalley Rumfield C, Pellom ST, Morillon Ii YM, Schlom J, Jochems C. 2020. Immunomodulation to enhance the efficacy of an HPV therapeutic vaccine. J Immunother Cancer 8.

12. Riehl M, Harms M, Hanefeld A, Baleeiro RB, Walden P, Mader K. 2017. Combining R-DOTAP and a particulate antigen delivery platform to trigger dendritic cell activation: Formulation development and in-vitro interaction studies. Int J Pharm 532:37-46.

13. Gandhapudi SK, Ward M, Bush JPC, Bedu-Addo F, Conn G, Woodward JG. 2019. Antigen Priming with Enantiospecific Cationic Lipid Nanoparticles Induces Potent Antitumor CTL Responses through Novel Induction of a Type I IFN Response. J Immunol 202:3524-3536.

14. Langley JM, Wang L, Aggarwal N, Bueso A, Chandrasekaran V, Cousin L, Halperin SA, Li P, Liu A, McNeil S, Mendez LP, Rivera L, Innis BL, Jain VK. 2015. Immunogenicity and Reactogenicity of an Inactivated Quadrivalent Influenza Vaccine Administered Intramuscularly to Children 6 to 35 Months of Age in 2012-2013: A Randomized, Double-Blind, Controlled, Multicenter, Multicountry, Clinical Trial. J Pediatric Infect Dis Soc 4:242-51.

15. Madan A, Ferguson M, Rheault P, Seiden D, Toma A, Friel D, Soni J, Li P, Innis BL, Schuind A. 2017. Immunogenicity and safety of an AS03-adjuvanted H7N1 vaccine in adults 65 years of age and older: A phase II, observer-blind, randomized, controlled trial. Vaccine 35:1865-1872.

16. Wang L, Chandrasekaran V, Domachowske JB, Li P, Innis BL, Jain VK. 2016. Immunogenicity and Safety of an Inactivated Quadrivalent Influenza Vaccine in US Children 6-35 Months of Age During 2013-2014: Results From A Phase II Randomized Trial. J Pediatric Infect Dis Soc 5:170-9.

17. Amanat F, Stadlbauer D, Strohmeier S, Nguyen THO, Chromikova V, McMahon M, Jiang K, Arunkumar GA, Jurczyszak D, Polanco J, Bermudez-Gonzalez M, Kleiner G, Aydillo T, Miorin L, Fierer DS, Lugo LA, Kojic EM, Stoever J, Liu STH, Cunningham-Rundles C, Felgner PL, Moran T, Garcia-Sastre A, Caplivski D, Cheng AC, Kedzierska K, Vapalahti O, Hepojoki JM, Simon V, Krammer F. 2020. A serological assay to detect SARS-CoV-2 seroconversion in humans. Nat Med 26:1033-1036.

18. Vijayakumar G, Zamarin D. 2020. Design and Production of Newcastle Disease Virus for Intratumoral Immunomodulation. Methods Mol Biol 2058:133-154.

19. Dinnon KH, Leist SR, Schafer A, Edwards CE, Martinez DR, Montgomery SA, West A, Yount BL, Hou YJ, Adams LE, Gully KL, Brown AJ, Huang E, Bryant MD, Choong IC, Glenn JS, Gralinski LE, Sheahan TP, Baric RS. 2020. A mouse-adapted SARS-CoV-2 model for the evaluation of COVID-19 medical countermeasures. bioRxiv doi:10.1101/2020.05.06.081497.

20. Amanat F, White KM, Miorin L, Strohmeier S, McMahon M, Meade P, Liu WC, Albrecht RA, Simon V, Martinez-Sobrido L, Moran T, Garcia-Sastre A, Krammer F. 2020. An In Vitro Microneutralization Assay for SARS-CoV-2 Serology and Drug Screening. Curr Protoc Microbiol 58:e108.

21. Imai M, Iwatsuki-Horimoto K, Hatta M, Loeber S, Halfmann PJ, Nakajima N, Watanabe T, Ujie M, Takahashi K, Ito M, Yamada S, Fan S, Chiba S, Kuroda M, Guan L, Takada K, Armbrust T, Balogh A, Furusawa Y, Okuda M, Ueki H, Yasuhara A, Sakai-Tagawa Y, Lopes TJS, Kiso M, Yamayoshi S, Kinoshita N, Ohmagari N, Hattori SI, Takeda M, Mitsuya H, Krammer F, Suzuki T, Kawaoka Y. 2020. Syrian hamsters as a small animal model for SARS-CoV-2 infection and countermeasure development. Proc Natl Acad Sci U S A doi:10.1073/pnas.2009799117.

22. Chan JF, Zhang AJ, Yuan S, Poon VK, Chan CC, Lee AC, Chan WM, Fan Z, Tsoi HW, Wen L, Liang R, Cao J, Chen Y, Tang K, Luo C, Cai JP, Kok KH, Chu H, Chan KH, Sridhar S, Chen Z, Chen H, To KK, Yuen KY. 2020. Simulation of the clinical and pathological manifestations of Coronavirus Disease 2019 (COVID-19) in golden Syrian hamster model: implications for disease pathogenesis and transmissibility. Clin Infect Dis doi:10.1093/cid/ciaa325.

### 11. EXAMPLE 6: VACCINATION WITH NDV-HXP-S

Live and inactivated NDV-HXP-S viruses described in Section 10 are currently in clinical trials. NDV-HXP-S is an egg-based, inactivated or live, whole chimeric Newcastle disease virus (NDV) expressing membrane-anchored pre-fusion-stabilized trimeric SARS-CoV-2 spike protein carrying the 6-proline stabilized, cleavage-site deleted spike (Hexapro). Manufacturers in Latin America (Butantan Institute in Brazil; AviMex in Mexico) and South East Asia (the Government Pharmaceutical Organization [GPO] in Thailand and the Institute of Vaccines and Medical Biologicals [IVAC] in Vietnam) have started phase ½ studies. Several hundred subjects have been administered the inactivated NDV-HXP-S vaccine without any reported side effects.

### 11.1 NDV-HXP-S Clinical Trial

One clinical trial is conducted in 2 phases. Phase 1 assesses the safety, tolerability and immunogenicity of the NDV-HXP-S vaccine administered at different doses levels (1, 3, and 10 µg) without adjuvant, and at two different dose levels (1 and 3 µg) with the adjuvant CpG 1018 among healthy adults, (age 18-59 years) (approximately 210 subjects). Subjects receive 2 doses of assigned investigational product (IP) on D1 and D29 (V1 and V3), and are assessed in the clinic for safety and reactogenicity at 7 days after each vaccination (day 1 as day vaccination). NDV-HXP-S or placebo (0.9% normal saline for injection) is administered intramuscularly (IM) according to a repeat vaccination schedule (given 28 days apart). In addition, a total of 36 subjects are randomly selected (1:1:1 ratio) from placebo and two high-dose groups i.e. NDV-HXP-S 10 µg and NDV-HXP-S 3 µg + CpG 1018, to provide additional blood at V1, V5 and V7 for assessment of T-cell-mediated immunity (CMI). See Table 4 for the arms and interventions for the Phase 1 clinical study. An interim analysis of Phase 1 data is conducted as the basis for decisions about advancement to Phase 2 of the clinical study and about treatment group down selection.

TABLE 4

| | |
|---|---|
| Arm | Intervention Treatment |
| Placebo Comparator: Placebo 0.9% Normal Saline for injection | Biological: Normal Saline 0.9% normal saline for injection |
| Active Comparator: NDV-HXP-S 1 µg 35 subjects age 18-59 will receive NDV-HXP-S 1 µg study vaccine administered 0.5 mL IM | Biological: NDV-HXP-S vaccine Vaccine NDV-HXP-S, manufactured by GPO with or without adjuvant CpG1018 |
| Active Comparator: NDV-HXP-S 3 µg 35 subjects age 18-59 will receive NDV-HXP-S 3 µg study vaccine administered 0.5 mL IM | Biological: NDV-HXP-S vaccine Vaccine NDV-HXP-S, manufactured by GPO with or without adjuvant CpG1018 |
| Active Comparator: NDV-HXP-S 10 µg 35 subjects age 18-59 will receive NDV-HXP-S 10 µg study vaccine administered 0.5 mL IM | Biological: NDV-HXP-S vaccine Vaccine NDV-HXP-S, manufactured by GPO with or without adjuvant CpG1018 |
| Active Comparator: NDV-HXP-S 1 µg + CpG1018 1.5 mg 35 subjects age 18-59 will receive NDV-HXP-S 1 µg + CpG1018 1.5 mg study vacine administered 0.5 mL IM | Biological: NDV-HXP-S vaccine Vaccine NDV-HXP-S, manufactured by GPO with or without adjuvant CpG1018 |
| Active Comparator: NDV-HXP-S 3 µg + CpG1018 1.5 mg 35 subjects age 18-59 will receive NDV-HXP-S 3 µg + CpG1018 1.5 mg study vacine administered 0.5 mL IM | Biological: NDV-HXP-S vaccine Vaccine NDV-HXP-S, manufactured by GPO with or without adjuvant CpG1018 |

In the Phase 2 study, approximately 250 subjects aged 18-75 years are randomized (1:2:2) to placebo (0.9% normal saline for injection), or one of two selected formulations of NDV HXP S being evaluated in Phase 1 are enrolled to Phase 2 study. Approximately twelve subjects in each of the three Phase 2 groups (distributed among the two age strata) are randomized to provide additional blood at V1, V5 and V7 for assessment of T-cell-mediated immunity (CMI).

The criteria for inclusion in the clinical trial may include:
Phase 1 Only:
   Adult 18 through 59 years of age, inclusive, at screening
   Healthy, as defined by absence of clinically significant medical condition, either acute or chronic, as determined by medical history, physical examination, screening laboratory test results, and clinical assessment of the investigator.
Phase 2 Only:
   Adult 18 through 75 years of age, inclusive, at screening.
   Having no clinically significant acute medical condition, and no chronic medical condition that has not been controlled within 90 days of randomization, as determined by medical history, physical examination, screening laboratory test results, and clinical assessment of the investigator.
Both Phase 1 and Phase 2:
   Has a body mass index (BMI) of 17 to 40 kg/m2, inclusive, at screening.
   If a woman is of childbearing potential, must not be breastfeeding or be pregnant (based on a negative serum pregnancy test at screening and a negative urine pregnancy test during the 24 hours prior to receipt of the first dose of IP), must plan to avoid pregnancy for at least 28 days after the last dose of IP, and be willing to use an adequate method of contraception consistently and have a repeated pregnancy test prior to the second (last) dose of IP.

The criteria for exclusion from the clinical trial may include:
Phase 1 Only:
   A positive serologic test for SARS-CoV-2 IgG test.
Both Phase 1 and Phase 2:
   History of administration of any non-study vaccine within 28 days prior to administration of study vaccine or planned vaccination during the course of study participation Receipt of any COVID-19 vaccine that is licensed or granted Emergency Use Authorization in Thailand during the course of study participation may not exclusionary if administered after Visit 5
   Previous receipt of investigational vaccine for SARS or MERS, or any investigational or licensed vaccine that may have an impact on interpretation of the trial results.
   History of hypersensitivity reaction to any prior vaccination or known hypersensitivity to any component of the study vaccine
   History of egg or chicken allergy
   History of angioedema
   History of anaphylaxis
   Acute illness (moderate or severe) and/or fever (body temperature measured orally ≥38° C.)
   Any abnormal vital sign deemed clinically relevant by the PI.
   Abnormality in screening laboratory test deemed exclusionary by the PI.
   A positive serologic test for SARS-CoV-2 IgM test, human immunodeficiency virus (HIV ½ Ab), hepatitis B (HBsAg) or hepatitis C (HCV Ab)
   History of laboratory-confirmed COVID-19 (RT-PCR positive to SAR-CoV-2)
   History of malignancy, which may exclude non-melanoma skin and cervical carcinoma in situ.
   Any confirmed or suspected immunosuppressive or immunodeficient state
   Administration of immunoglobulin or any blood product within 90 days prior to first study injection or planned administration during the study period.
   Administration of any long-acting immune-modifying drugs (e.g., infliximab or rituximab) or the chronic administration (defined as more than 14 days) of immunosuppressants within six months prior to first study injection, or planned administration during the study period (includes systemic corticosteroids at doses equivalent to ≥ 0.5 mg/kg/day of prednisone; the use of topical steroids including inhaled and intranasal steroids is permitted).
   History of known disturbance of coagulation or blood disorder that could cause anemia or excess bleeding. (e.g, thalassemia, coagulation factor deficiencies).
   Recent history (within the past year) or signs of alcohol or substance abuse.
   Any medical, psychiatric or behavior condition that in the opinion of the PI may interfere with the study objectives, pose a risk to the subject, or prevent the subject from completing the study follow-up.

The subjects enrolled in the clinical study may be assessed for primary, secondary and other outcomes. For example, subjects enrolled in the clinical study may be assessed for the adverse effects and changes in certain blood tests. In particular, subjects enrolled in the clinical study may be assessed for one, two or more, or all of the following primary outcome measures:
Frequency of solicited reportable local adverse event after first vaccination [ Time Frame: Day 1 up to Day 7 ]
Frequency of solicited reportable local adverse events (pain or tenderness, erythema, swelling or induration) of first vaccination
Frequency of solicited reportable local adverse event after second vaccination [ Time Frame: Day 1 up to Day 7 ]
Frequency of solicited reportable local adverse events (pain or tenderness, erythema, swelling or induration) of second vaccination
Frequency of solicited reportable systemic adverse event after first vaccination [ Time Frame: Day 1 up to Day 7 ]
Frequency of solicited reportable systemic adverse events (fever, headache,fatigue or malaise, myalgia, arthralgia,nausea or vomitting) of first vaccination
Frequency of solicited reportable systemic adverse event after second vaccination [ Time Frame: Day 1 up to Day 7 ]
Frequency of solicited reportable systemic adverse events (fever, headache,fatigue or malaise, myalgia, arthralgia,nausea or vomitting) of second vaccination
Measurement of hemoglobin changed from baseline at 7 days after first vaccination [ Time Frame: Day 8 ]
Measurement of hemoglobin (g/dl) changed from baseline at 7 days after first vaccination
Measurement of hemoglobin changed from baseline at 7 days after second vaccination [ Time Frame: Day 36 ]
Measurement of hemoglobin (g/dl) changed from baseline at 7 days after the second vaccination
Measurement of white blood cells changed from baseline at 7 days after first vaccination [ Time Frame: Day 8 ]
Measurement of white blood cells (10^3 cells/ul) changed from baseline at 7 days after first vaccination
Measurement of white blood cells changed from baseline at 7 days after second vaccination [ Time Frame: Day 36 ]
Measurement of white blood cells (10^3 cells/ul) changed from baseline at 7 days after second vaccination
Measurement of platelet count changed from baseline at 7 days after first vaccination [ Time Frame: Day 8 ]
Measurement of platelet count (10^3 cells/ul) changed from baseline at 7 days after first vaccination
Measurement of platelet count changed from baseline at 7 days after second vaccination [ Time Frame: Day 36 ]
Measurement of platelet count (10^3 cells/ul) changed from baseline at 7 days after second vaccination
Measurement of creatinine changed from baseline at 7 days after first vaccination [ Time Frame: Day 8 ]
Measurement of creatinine (mg/dl) changed from baseline at 7 days after first vaccination
Measurement of creatinine changed from baseline at 7 days after second vaccination [ Time Frame: Day 36 ]
Measurement of creatinine (mg/dl) changed from baseline at 7 days after second vaccination
Measurement of AST changed from baseline at 7 days after first vaccination [ Time Frame: Day 8 ]
Measurement of AST (U/L) changed from baseline at 7 days after first vaccination
Measurement of AST changed from baseline at 7 days after second vaccination [ Time Frame: Day 36 ]
Measurement of AST (U/L) changed from baseline at 7 days after second vaccination
Measurement of ALT change from baseline at 7 days after first vaccination [ Time Frame: Day 8 ]
Measurement of ALT (U/L) change from baseline at 7 days after first vaccination
Measurement of ALT change from baseline at 7 days after second vaccination [ Time Frame: Day 36 ]
Measurement of ALT (U/L) change from baseline at 7 days after second vaccination
Measurement of total bilirubin changed from baseline at 7 days after first vaccination [ Time Frame: Day 8 ]
Measurement of total bilirubin (mg/dl) change from baseline at 7 days after first vaccination
Measurement of total bilirubin changed from baseline at 7 days after second vaccination [ Time Frame: Day 36 ]
Measurement of total bilirubin (mg/dl) change from baseline at 7 days after second vaccination
Frequency of all unsolicited AEs [ Time Frame: Day 56 ]
Frequency of all unsolicited AEs
Frequency of SAEs [ Time Frame: Day 365 ]\
Frequency of SAEs throughout the entire study period
Frequency of medically-attended adverse event (MAAEs) [Time Frame: Day 365]
Frequency of medically-attended adverse event (MAAEs) throughout the entire study period
Frequency of AESI [ Time Frame: Day 365 ]
Frequency of AESI throughout the entire study period, including AESI relevant to COVID-19, and potential immune-mediated medical conditions (PIMMC) presented as number and percentage

Subjects enrolled in the clinical study may be assessed for the neutralizing antibody and seroresponses. In particular, subjects enrolled in the clinical study may be assessed for one, two or more, or all of the following secondary outcomes measures:
GMT Neutralizing antibody titer 50 changed from baseline at 28 days after the first vaccination [ Time Frame: Day 29 ]
GMT Neutralizing antibody titer 50 changed from baseline at 28 days after the first vaccination
GMT Neutralizing antibody titer 50 changed from baseline at 14 days after the second vaccination [ Time Frame: Day 43 ]
GMT Neutralizing antibody titer 50 changed from baseline at 14 days after the second vaccination
GMT Neutralizing antibody titer 50 changed from baseline at 6 months after the second vaccination [ Time Frame: Day 197 ]
GMT Neutralizing antibody titer 50 changed from baseline at 6 months after the second vaccination
GMT Neutralizing antibody titer 50 changed from baseline at 12 months after the second vaccination [ Time Frame: Day 365 ]
GMT Neutralizing antibody titer 50 changed from baseline at 12 months after the second vaccination
GMT Neutralizing antibody titer 80 changed from baseline at 28 days after the first vaccination [ Time Frame: Day 29 ]
GMT Neutralizing antibody titer 80 changed from baseline at 28 days after the first vaccination
GMT Neutralizing antibody titer 80 changed from baseline at 14 days after the second vaccination [ Time Frame: Day 43 ]
GMT Neutralizing antibody titer 80 changed from baseline at 14 days after the second vaccination
GMT Neutralizing antibody titer 80 changed from baseline at 6 months after the second vaccination [ Time Frame: Day 197 ]
GMT Neutralizing antibody titer 80 changed from baseline at 6 months after the second vaccination
GMT Neutralizing antibody titer 80 changed from baseline at 12 months after the second vaccination [ Time Frame: Day 365 ]
GMT Neutralizing antibody titer 80 changed from baseline at 12 months after the second vaccination
NT50 seroresponses changed from baseline at 28 days after the first vacccination [ Time Frame: Day 29 ]
Frequency of subjects with NT50 seroresponses against SARS-CoV-2 pseudovirus as defined by (1) a ≥ 4-fold increase from baseline, and (2) a ≥ 10-fold increase from baseline at 28 days after the first vacccination compare to baseline
NT50 seroresponses changed from baseline at 14 days after the second vaccination [ Time Frame: Day 43 ]
Frequency of subjects with NT50 seroresponses against SARS-CoV-2 pseudovirus as defined by (1) a ≥ 4-fold increase from baseline, and (2) a ≥ 10-fold increase from baseline at 14 days after the second vaccination compare to baseline
NT50 seroresponses changed from baseline at 6 months after the second vaccination [ Time Frame: Day 197 ]
Frequency of subjects with NT50 seroresponses against SARS-CoV-2 pseudovirus as defined by (1) a ≥ 4-fold increase from baseline, and (2) a ≥ 10-fold increase from baseline at 6 months after the second vaccination compare to baseline
NT50 seroresponses changed from baseline at 12 months after the second vaccination [ Time Frame: Day 365 ]
Frequency of subjects with NT50 seroresponses against SARS-CoV-2 pseudovirus as defined by (1) a ≥ 4-fold increase from baseline, and (2) a ≥ 10-fold increase from baseline at12 months after the second vaccination compare to baseline
NT80 seroresponses changed from baseline at 28 days after the first vaccination [ Time Frame: Day 29 ]
Frequency of subjects with NT80 seroresponses against SARS-CoV-2 pseudovirus as defined by (1) a ≥ 4-fold increase from baseline, and (2) a ≥ 10-fold increase from baseline at 28 days after first vaccination compare to baseline
NT80 seroresponses changed from baseline at 14 days after the second vaccination [ Time Frame: Day 43 ]
Frequency of subjects with NT80 seroresponses against SARS-CoV-2 pseudovirus as defined by (1) a ≥ 4-fold increase from baseline, and (2) a ≥ 10-fold increase from baseline at 14 after the second vaccination compare to baseline
NT80 seroresponses changed from baseline at 6 months after the second vaccination [ Time Frame: Day 197 ]
Frequency of subjects with NT80 seroresponses against SARS-CoV-2 pseudovirus as defined by (1) a ≥ 4-fold increase from baseline, and (2) a ≥ 10-fold increase from baseline at 6 months after the second vaccination compare to baseline
NT80 seroresponses changed from baseline at 12 months after the second vaccination [ Time Frame: Day 365 ]
Frequency of subjects with NT80 seroresponses against SARS-CoV-2 pseudovirus as defined by (1) a ≥ 4-fold increase from baseline, and (2) a ≥ 10-fold increase from baseline at 12 months after the second vaccination compare to baseline
GMT Anti-S IgG at 28 days after the first vaccination [ Time Frame: Day 29 ]
GMT Anti-S IgG at 28 days after the first vaccination in subjects who are anti-S IgG seronegative at baseline
GMT Anti-S IgG at 14 days after the second vaccination [ Time Frame: Day 43 ]
GMT Anti-S IgG at 14 days after the second vaccination in subjects who are anti-S IgG seronegative at baseline
GMT Anti-S IgG at 6 months after the second vaccination [ Time Frame: Day 197]
GMT Anti-S IgG at 6 months after the second vaccination in subjects who are anti-S IgG seronegative at baseline
GMT Anti-S IgG at 12 months after the second vaccination [ Time Frame: Day 365 ]
GMT Anti-S IgG at 12 months after the second vaccination in subjects who are anti-S IgG seronegative at baseline
GMFR changed from baseline in anti-S IgG GMT at 28 days after the first vaccination [ Time Frame: Day 29 ]
GMFR changed from baseline in anti-S IgG GMT at 28 days after the first vaccination
GMFR changed from baseline in anti-S IgG GMT at 14 days after the second vaccination [ Time Frame: Day 43 ]
GMFR changed from baseline in anti-S IgG GMT 14 days after second vaccination
GMFR changed from baseline in anti-S IgG GMT at 6 months after the second vaccination [ Time Frame: Day 197 ]
GMFR changed from baseline in anti-S IgG GMT at 6 months after the second vaccination
GMFR changed from baseline in anti-S IgG GMT at 12 months after the second vaccination [ Time Frame: Day 365 ]
GMFR changed from baseline in anti-S IgG GMT at 12 months after the second vaccination
Anti-S IgG Seroresponses changed from baseline at 28 days after the first vaccination [ Time Frame: Day 29 ]
Frequency of subjects with seroresponses in anti-S IgG titer as defined by (1) a ≥ 4-fold increase from baseline, and (2) a ≥ 10-fold increase from baseline, at 28 days after the first vaccination
Anti-S IgG Seroresponses changed from baseline at 14 days after the second vaccination [ Time Frame: Day 43 ]
Frequency of subjects with seroresponses in anti-S IgG titer as defined by (1) a ≥ 4-fold increase from baseline, and (2) a ≥ 10-fold increase from baseline, at 14 days after the second vaccination
Anti-S IgG Seroresponses changed from baseline at 6 months after the second vaccination [ Time Frame: Day 197 ]
Frequency of subjects with seroresponses in anti-S IgG titer as defined by (1) a ≥ 4-fold increase from baseline, and (2) a ≥ 10-fold increase from baseline, at 6 months after the second vaccination
Anti-S IgG Seroresponses changed from baseline at 12 months after the second vaccination [ Time Frame: Day 365 ]
Frequency of subjects with seroresponses in anti-S IgG titer as defined by (1) a ≥ 4-fold increase from baseline, and (2) a ≥ 10-fold increase from baseline, at 12 months after the second vaccination

Subjects enrolled in the clinical study may be assessed for the T cell responses and anti-NDV antibody. In particular, subjects enrolled in the clinical study may be assessed for one, two or more, or all of the following outcome measures:
S protein-specific T cells response changed from baseline at 14 days after the second vaccination [ Time Frame: Day 43 ]
Frequency of S protein-specific T cells relative to baseline at 14 days after the second vaccination
S protein-specific T cells response changed from baseline at 6 months after the second vaccination [ Time Frame: Day 197 ]
Frequency of S protein-specific T cells relative to baseline at 6 months after the second vaccination
Anti-NDV HN GMT changed from baseline at 28 days after the first vaccination [ Time Frame: Day 29 ]
Anti-NDV HN GMT changed from baseline at 28 days after the first vaccination
Anti-NDV HN IgG GMT changed from baseline at 14 days after the second vaccination [ Time Frame: Day 43 ]
Anti-NDV HN IgG GMT changed from baseline at 14 days after the second vaccination
Anti-NDV HN IgG GMT changed from baseline at 6 months after the second vaccination [ Time Frame: Day 197 ]
Anti-NDV HN IgG GMT changed from baseline at 6 months after the second vaccination
Anti-NDV HN IgG GMT changed from baseline at 12 months after the second vaccination [ Time Frame: Day 365 ]
Anti-NDV HN IgG GMT changed from baseline at 12 months after the second vaccination

### 12. EXAMPLE 7: NEWCASTLE DISEASE VIRUS (NDV) EXPRESSING THE SPIKE PROTEIN OF SARS-COV-2 VARIANTS AS NEW GENERATIONS OF VACCINES

This example describes the production of Newcastle disease virus (NDV) vectors expressing the spikeprotin of SARS-CoV-2 variants.

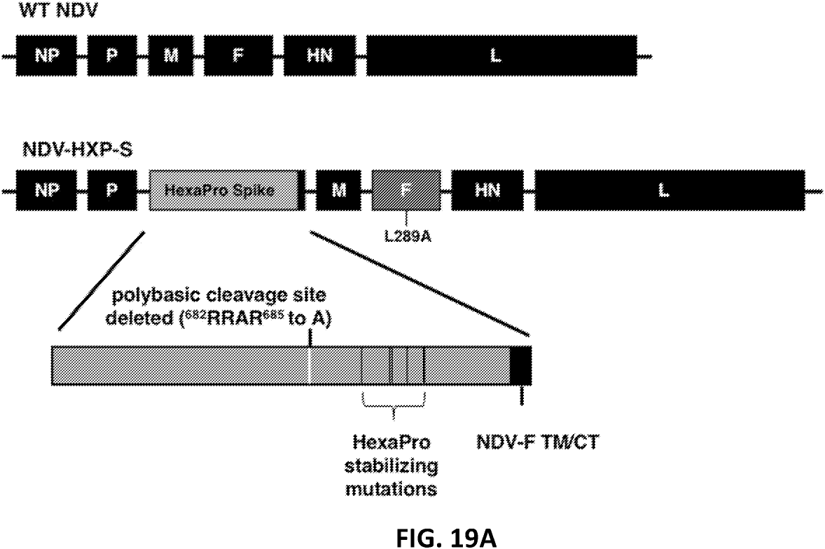

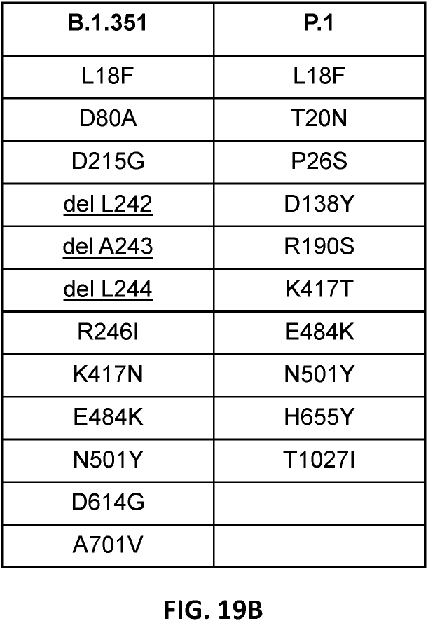

The emergence of SARS-CoV-2 variants, in particular B.1.351 (South Africa), P.1 (Brazil), and B.1.1.7 (UK) with an additional E484K mutation, has raised concerns about the efficacy of existing vaccines under emergency use authorization in the United States (1-5). These variants appeared to exhibit increased resistance to the neutralizing antibodies elicited by the Wuhan prototype SARS-CoV-2 spike. This example describes the contruction of NDV vectors expressing the prefusion-stabilized spike protein of SARS-CoV-2 variants, in which the polybasic cleavage site was removed, the hexa pro (HXP) stabilizing mutations (6) were introduced, and the transmembrane domain/cytoplasmic tail were replaced with those from NDV fusion (F) protein (7, 8). See FIG. 19A for a schematic illustration of the design of NDV-HXP-S vectors, which may be used as vaccines. In particular, NDV-vectors expressing the HXP-S of the B.1.351 and P.1 variants (see Table 5) were generated. These NDV vectors may be used as a monovalent NDV-HXP-S variant vaccine or a bivalent NDV-HXP-S vaccine to be use in countries where the variants are predominately circulating.

### 12.1 Materials & Methods

Design and Expression of the NDV-HXP-S variants. FIG. 19A provides a schematic illustration of the design of NDV-HXP-S (B.1.351) and NDV-HXP-S (P.1). The mutations introduced into NDV-HXP-S (B.1.351) and NDV-HXP-S (P.1) are shown in FIG. 19B and the sequences are provided in Table 5. The viruses were purified by limiting dilutions in chicken embryonated eggs via two passages, which are used as the pre-master virus seeds (MVSs) for GMP MVS production. To examine the expression of the spike protein in the NDV-HXP-S variants, the pre-MVS of the NDV-HXP-S (B.1.351) and NDV-HXP-S (P.1) were passaged in egg and concentrated the viruses in the harvested allantoic fluid through a 20% sucrose cushion via ultracentrifugation. The concentrated viruses were resolved on SDS-PAGE with the prototype NDV-HXP-S followed by Coomassie blue staining.

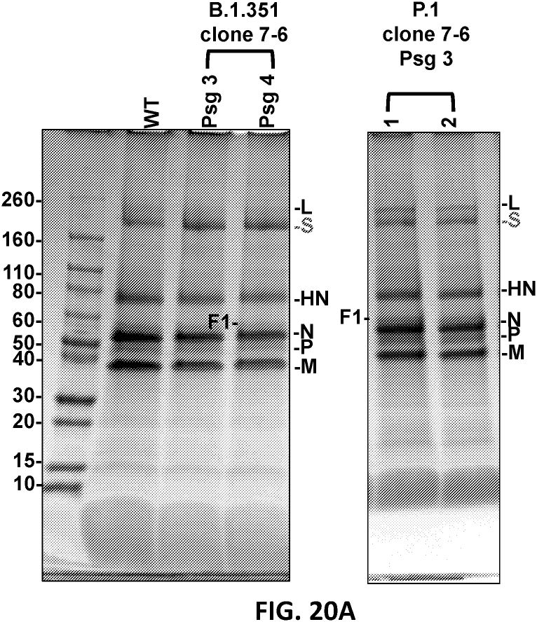

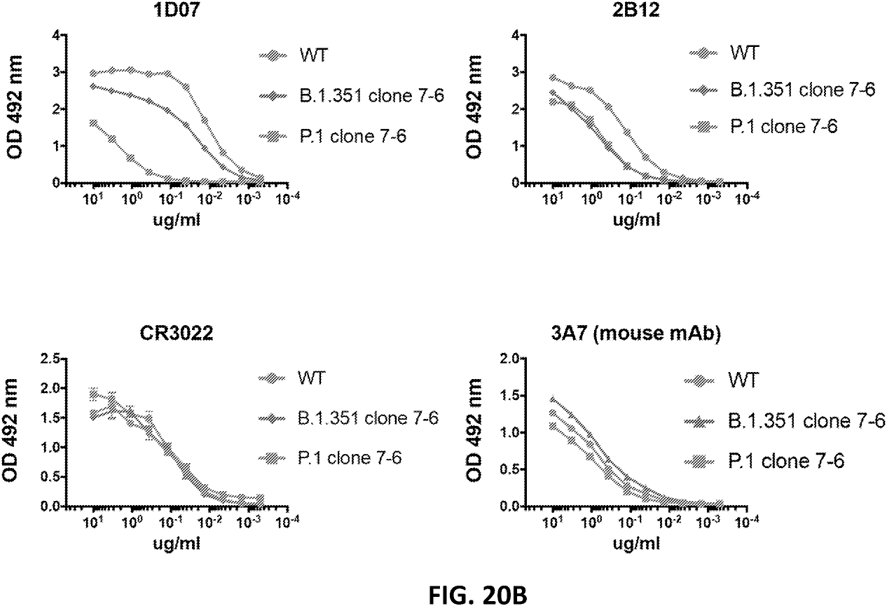

Characterization of the NDV-HXP-S variants. Human or mouse monoclonal antibodies that cross-react with both the prototype (Wuhan) or the B.1.351 spike proteins were previously isolated and characterized. The binding of the spike protein expressed by the NDV-HXP-S variants to those cross-reactive antibodies including human monoclonal antibodies (mAbs) 1D07 (RBD), 2B12 (NTD) and CR3022 (RBD) and a mouse monoclonal antibody (mAb) 3A7 (RBD) were determined by ELISA using the concentrated virus preparations shown in FIG. 20A.

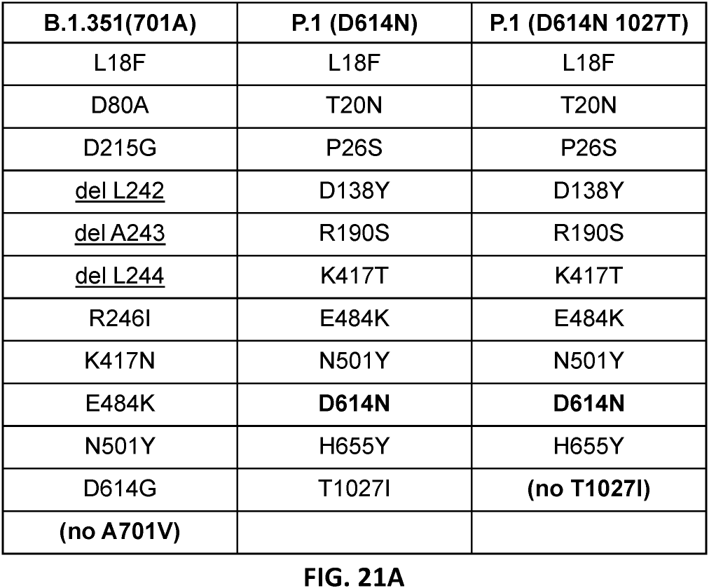

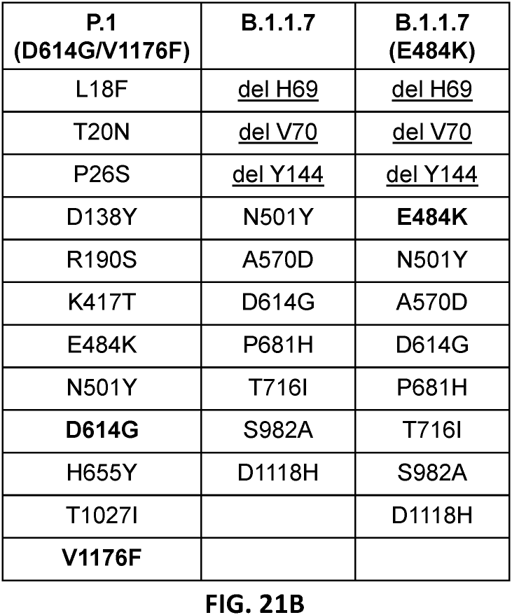

Mutagenesis profile of the NDV-HXP-S variants B.1.351 and P.1. Specific mutations were introduced into the NDV-HXP-S variants B.1.351 and P.1 . For example, the A701V mutation was reverted back to the original A701 in the NDV-HXP-S variant B.1.351. For the rescued P.1 variant in FIG. 19B D614 was not mutated. A D614N mutation was introduced into P.1 spike as it is reported to stabilize spike trimer, which was slightly more effective than the D614G (12). D614N was introduced in the absence or presence of the S2 mutation (T1027I). The mutations performed in this example are shown in FIGS. 21A-21B.

### 12.2 Results

Two NDV vectors (NDV-HXP-S (B1.351) and NDV-HXP-S (P.1) were designed as shown in FIG. 19A. FIG. 19B provides the mutations in the spike proteins of the two vectors. The two vectors were successfully resecued and as shown in FIG. 20A, the expression of spike protein by NDV-HXP-S (B.1.351) and NDV-HXP-S (P.1) was detected by SDS-PAGE The ability of the spike proteins expressed by the two variants to bind to particular monoclonal antibodies was examined. When 5 micrograms/mL of each virus was coated onto the ELISA plate (possibly not containing the same amount of spike), the B.1.351 spike showed slightly reduced binding to human mAbs 1D07 and 2B12, whereas P.1 showed a significantly reduced binding to mAb 1D07 and similar binding to mAb 2B12 as that of B.1351 (FIG. 20B). Both variants P.1 and B.1.351 and the NDV-HXP-S described in Section 10 (Wild-type or WT) bound comparably to human mAb CR3022 and mouse mAb 3A7 (FIG. 20B). These demonstrate that B.1.351 spike expressed by the NDV-HXP-S (B.1.351) maintains the epitopes that are recognized by these cross-reactive antibodies. In addition, binding information of P.1 spike expressed by NDV-HXP-S (P.1) to these antibodies were obtained, and it was observed that mAbs 2B12, CR3022 and 3A7 appear to cross-react with P.1 spike (FIG. 20B).

Mutagenesis profiles of the NDV-HXP-S variants B.1.351 and P.1 are explored to ensure the optimal expression, stability, and integrity of the spikes expressed by the NDV without changing the strain-specific antigenicity of the spike. In the B.1.351 spike, the A 701V mutation appeared to be close to the second furin cleavage site (11), which may impact the cleavage of the protein (although the major polybasic furin cleavage site is removed. Therefore, the A701V mutation was reverted back to the original A701. For the rescued P.1 varint in FIG. 19B D614 was not mutated. The D614N mutation, which is reported to stabilize spike trimer, was introduced into the P.1 spike, which was slightly more effective than the D614G (12). Three more viruses shown in FIG. 21A were rescued. The expression, stability and immunogenicity of these viruses are compared to those shown in FIG. 19B. The mutation profile later identified for P.1 spike also included D614G and V1176F. NDV-HXP-S (P.1) with these mutations together with NDV-HXP-S expressing spikes of the B.1.17 strain with or without the E48K mutation are rescued (FIG. 21B).

TABLE 5

Sequences for the NDV-vectors Expressing the HXP-S of the B.1.351 and P.1 Variants

| | | |
|---|---|---|
| NDV-HXP-S (B.1.351) (nucleotide sequence) | ATGTTCGTGTTTCTGGTGCTGCTGCCTCTGGTGTCCAGCCAGTGTGTGAACTTCACCACAAGAACCCAGCTGCCTCCAGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGCCAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACGACCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGCGCGAGTTCGTGTTCAAGAACATCGACGGCTACTTCAAGATCTACAGCAAGCACACCCCTATCAACCTCGTGCGGGGCCTGCCTCAGGGCTTCTCTGCTCTGGAACCCCTGGTGGATCTGCCCATCGGCATCAACATCACCCGGTTTCAGACACTGCACATCAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAgAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCAACATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGAAGGGCTTCAACTGCTACTTCCCACTGCAGTCCTACGGCTTTCAGCCCACATACGGCGTGGGCTATCAGCCCTACAGAGTGGTGGTGCTGAGCTTCGAACTGCTGCATGCCCCTGCCACAGTGTGCGGCCCTAAGAAgAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGGCGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGCACGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGTGGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCcctATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCcctGCTCTGCAGATCCCCTTTccaATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAcccAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACccccctGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCACCAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCggcggaggtgggtcgCTCATAACATACATCGTCCTGACTATAATCAGCTTGGTATTTGGTATTTTGTCTTTGATTCTTGCATGCTATTTGATGTATAAACAGAAAGCTCAGCAGAAGACTCTCCTGTGGCTCGGTAACAACACACTCGACCAGATGAGAGCAACTACAAAGATGTGA | 16 |
| NDV-HXP-S (B.1.351) (amino acid sequence) | MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM* | 17 |
| NDV-HXP-S (P.1) (nucleotide sequence) | ATGTTCGTGTTTCTGGTGCTGCTGCCTCTGGTGTCCAGCCAGTGTGTGAACttcACCaacAGAACCCAGCTGCCTagcGCCTACACCAACAGCTTTACCAGAGGCGTGTACTACCCCGACAAGGTGTTCAGATCCAGCGTGCTGCACTCTACCCAGGACCTGTTCCTGCCTTTCTTCAGCAACGTGACCTGGTTCCACGCCATCCACGTGTCCGGCACCAATGGCACCAAGAGATTCGACAACCCCGTGCTGCCCTTCAACGACGGGGTGTACTTTGCCAGCACCGAGAAGTCCAACATCATCAGAGGCTGGATCTTCGGCACCACACTGGACAGCAAGACCCAGAGCCTGCTGATCGTGAACAACGCCACCAACGTGGTCATCAAAGTGTGCGAGTTCCAGTTCTGCAACtacCCCTTCCTGGGCGTCTACTATCACAAGAACAACAAGAGCTGGATGGAAAGCGAGTTCCGGGTGTACAGCAGCGCCAACAACTGCACCTTCGAGTACGTGTCCCAGCCTTTCCTGATGGACCTGGAAGGCAAGCAGGGCAACTTCAAGAACCTGagcGAGTTCGTGTTCAAGAACATCGACGGCTACTTCAAGATCTACAGCAAGCACACCCCTATCAACCTCGTGCGGGATCTGCCTCAGGGCTTCTCTGCTCTGGAACCCCTGGTGGATCTGCCCATCGGCATCAACATCACCCGGTTTCAGACACTGCTGGCCCTGCACAGAAGCTACCTGACACCTGGCGATAGCAGCAGCGGATGGACAGCTGGTGCCGCCGCTTACTATGTGGGCTACCTGCAGCCTAGAACCTTTCTGCTGAAGTACAACGAGAACGGCACCATCACCGACGCCGTGGATTGTGCTCTGGATCCTCTGAGCGAGACAAAGTGCACCCTGAAGTCCTTCACCGTGGAgAAGGGCATCTACCAGACCAGCAACTTCCGGGTGCAGCCCACCGAATCCATCGTGCGGTTCCCCAATATCACCAATCTGTGCCCCTTCGGCGAGGTGTTCAATGCCACCAGATTCGCCTCTGTGTACGCCTGGAACCGGAAGCGGATCAGCAATTGCGTGGCCGACTACTCCGTGCTGTACAACTCCGCCAGCTTCAGCACCTTCAAGTGCTACGGCGTGTCCCCTACCAAGCTGAACGACCTGTGCTTCACAAACGTGTACGCCGACAGCTTCGTGATCCGGGGAGATGAAGTGCGGCAGATTGCCCCTGGACAGACAGGCaccATCGCCGACTACAACTACAAGCTGCCCGACGACTTCACCGGCTGTGTGATTGCCTGGAACAGCAACAACCTGGACTCCAAAGTCGGCGGCAACTACAATTACCTGTACCGGCTGTTCCGGAAGTCCAATCTGAAGCCCTTCGAGCGGGACATCTCCACCGAGATCTATCAGGCCGGCAGCACCCCTTGTAACGGCGTGaagGGCTTCAACTGCTACTTCCCACTGCAGTCCTACGGCTTTCAGCCCACAtacGGCGTGGGCTATCAGCCCTACAGAGTGGTGGTGCTGAGCTTCGAACTGCTGCATGCCCCTGCCACAGTGTGCGGCCCTAAGAAgAGCACCAATCTCGTGAAGAACAAATGCGTGAACTTCAACTTCAACGGCCTGACCGGCACCGGCGTGCTGACAGAGAGCAACAAGAAGTTCCTGCCATTCCAGCAGTTTGGCCGGGATATCGCCGATACCACAGACGCCGTTAGAGATCCCCAGACACTGGAAATCCTGGACATCACCCCTTGCAGCTTCGGCGGAGTGTCTGTGATCACCCCTGGCACCAACACCAGCAATCAGGTGGCAGTGCTGTACCAGGACGTGAACTGTACCGAAGTGCCCGTGGCCATTCACGCCGATCAGCTGACACCTACATGGCGGGTGTACTCCACCGGCAGCAATGTGTTTCAGACCAGAGCCGGCTGTCTGATCGGAGCCGAGtacGTGAACAATAGCTACGAGTGCGACATCCCCATCGGCGCTGGCATCTGTGCCAGCTACCAGACACAGACAAACAGCCCCGCCTCTGTGGCCAGCCAGAGCATCATTGCCTACACAATGTCTCTGGGCGCCGAGAACAGCGTGGCCTACTCCAACAACTCTATCGCTATCCCCACCAACTTCACCATCAGCGTGACCACAGAGATCCTGCCTGTGTCCATGACCAAGACCAGCGTGGACTGCACCATGTACATCTGCGGCGATTCCACCGAGTGCTCCAACCTGCTGCTGCAGTACGGCAGCTTCTGCACCCAGCTGAATAGAGCCCTGACAGGGATCGCCGTGGAACAGGACAAGAACACCCAAGAGGTGTTCGCCCAAGTGAAGCAGATCTACAAGACCCCTCCTATCAAGGACTTCGGCGGCTTCAATTTCAGCCAGATTCTGCCCGATCCTAGCAAGCCCAGCAAGCGGAGCcctATCGAGGACCTGCTGTTCAACAAAGTGACACTGGCCGACGCCGGCTTCATCAAGCAGTATGGCGATTGTCTGGGCGACATTGCCGCCAGGGATCTGATTTGCGCCCAGAAGTTTAACGGACTGACAGTGCTGCCTCCTCTGCTGACCGATGAGATGATCGCCCAGTACACATCTGCCCTGCTGGCCGGCACAATCACAAGCGGCTGGACATTTGGAGCTGGCcctGCTCTGCAGATCCCCTTTccaATGCAGATGGCCTACCGGTTCAACGGCATCGGAGTGACCCAGAATGTGCTGTACGAGAACCAGAAGCTGATCGCCAACCAGTTCAACAGCGCCATCGGCAAGATCCAGGACAGCCTGAGCAGCACAcccAGCGCCCTGGGAAAGCTGCAGGACGTGGTCAACCAGAATGCCCAGGCACTGAACACCCTGGTCAAGCAGCTGTCCTCCAACTTCGGCGCCATCAGCTCTGTGCTGAACGATATCCTGAGCAGACTGGACccccctGAAGCCGAGGTGCAGATCGACAGACTGATCACCGGAAGGCTGCAGTCCCTGCAGACCTACGTTACCCAGCAGCTGATCAGAGCCGCCGAGATTAGAGCCTCTGCCAATCTGGCCGCCatcAAGATGTCTGAGTGTGTGCTGGGCCAGAGCAAGAGAGTGGACTTTTGCGGCAAGGGCTACCACCTGATGAGCTTCCCTCAGTCTGCCCCTCACGGCGTGGTGTTTCTGCACGTGACATACGTGCCCGCTCAAGAGAAGAATTTCACCACCGCTCCAGCCATCTGCCACGACGGCAAAGCCCACTTTCCTAGAGAAGGCGTGTTCGTGTCCAACGGCACCCATTGGTTCGTGACCCAGCGGAACTTCTACGAGCCCCAGATCATCACCACCGACAACACCTTCGTGTCTGGCAACTGCGACGTCGTGATCGGCATTGTGAACAATACCGTGTACGACCCTCTGCAGCCCGAGCTGGACAGCTTCAAAGAGGAACTGGATAAGTACTTTAAGAACCACACAAGCCCCGACGTGGACCTGGGCGATATCAGCGGAATCAATGCCAGCGTCGTGAACATCCAGAAAGAGATCGACCGGCTGAACGAGGTGGCCAAGAATCTGAACGAGAGCCTGATCGACCTGCAAGAACTGGGGAAGTACGAGCAGTACATCAAGTGGCCCggcggaggtgggtcgCTCATAACATACATCGTCCTGACTATAATCAGCTTGGTATTTGGTATTTTGTCTTTGATTCTTGCATGCTATTTGATGTATAAACAGAAAGCTCAGCAGAAGACTCTCCTGTGGCTCGGTAACAACACACTCGACCAGATGAGAGCAACTACAAAGATGTGA | 18 |
| NDV-HXP-S (P.1) (amino acid sequence) | MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM* | 19 |

### 12.3 References Cited in Example 6

1. Diamond M, Chen R, Xie X, Case J, Zhang X, VanBlargan L, Liu Y, Liu J, Errico J, Winkler E, Suryadevara N, Tahan S, Turner J, Kim W, Schmitz A, Thapa M, Wang D, Boon A, Pinto D, Presti R, O’Halloran J, Kim A, Deepak P, Fremont D, Corti D, Virgin H, Crowe J, Droit L, Ellebedy A, Shi PY, Gilchuk P. 2021. SARS-CoV-2 variants show resistance to neutralization by many monoclonal and serum-derived polyclonal antibodies. Res Sq doi: 10.21203/rs.3.rs-228079/v1.

2. Shen X, Tang H, Pajon R, Smith G, Glenn GM, Shi W, Korber B, Montefiori DC. 2021. Neutralization of SARS-CoV-2 Variants B.1.429 and B.1.351. N Engl J Med doi: 10. 1056/NEJMc2103740.

3. Garcia-Beltran WF, Lam EC, St Denis K, Nitido AD, Garcia ZH, Hauser BM, Feldman J, Pavlovic MN, Gregory DJ, Poznansky MC, Sigal A, Schmidt AG, Iafrate AJ, Naranbhai V, Balazs AB. 2021. Multiple SARS-CoV-2 variants escape neutralization by vaccine-induced humoral immunity. Cell doi: 10.1016/j.cell.2021.03.013.

4. Wang P, Nair MS, Liu L, Iketani S, Luo Y, Guo Y, Wang M, Yu J, Zhang B, Kwong PD, Graham BS, Mascola JR, Chang JY, Yin MT, Sobieszczyk M, Kyratsous CA, Shapiro L, Sheng Z, Huang Y, Ho DD. 2021. Antibody resistance of SARS-CoV-2 variants B.1.351 and B.1.1.7. Nature doi:10.1038/s41586-021-03398-2.

5. Wang Z, Schmidt F, Weisblum Y, Muecksch F, Barnes CO, Finkin S, Schaefer-Babajew D, Cipolla M, Gaebler C, Lieberman JA, Oliveira TY, Yang Z, Abernathy ME, Huey-Tubman KE, Hurley A, Turroja M, West KA, Gordon K, Millard KG, Ramos V, Da Silva J, Xu J, Colbert RA, Patel R, Dizon J, Unson-O′Brien C, Shimeliovich I, Gazumyan A, Caskey M, Bjorkman PJ, Casellas R, Hatziioannou T, Bieniasz PD, Nussenzweig MC. 2021. mRNA vaccine-elicited antibodies to SARS-CoV-2 and circulating variants. Nature doi:10.1038/s41586-021-03324-6.

6. Hsieh CL, Goldsmith JA, Schaub JM, DiVenere AM, Kuo HC, Javanmardi K, Le KC, Wrapp D, Lee AG, Liu Y, Chou CW, Byrne PO, Hjorth CK, Johnson NV, Ludes-Meyers J, Nguyen AW, Park J, Wang N, Amengor D, Lavinder JJ, Ippolito GC, Maynard JA, Finkelstein IJ, McLellan JS. 2020. Structure-based design of prefusion-stabilized SARS-CoV-2 spikes. Science 369:1501-1505.

7. Sun W, McCroskery S, Liu WC, Leist SR, Liu Y, Albrecht RA, Slamanig S, Oliva J, Amanat F, Schafer A, Dinnon KH, 3rd, Innis BL, Garcia-Sastre A, Krammer F, Baric RS, Palese P. 2020. A Newcastle Disease Virus (NDV) Expressing a Membrane-Anchored Spike as a Cost-Effective Inactivated SARS-CoV-2 Vaccine. Vaccines (Basel) 8.

8. Sun W, Leist SR, McCroskery S, Liu Y, Slamanig S, Oliva J, Amanat F, Schaefer A, Dinnon K, Garcia-Sastre A, Krammer F, Baric RS, Palese P. 2020. Newcastle disease virus (NDV) expressing the spike protein of SARS-CoV-2 as vaccine candidate. bioRxiv doi:10.1101/2020.07.26.221861.

9. Nuno R. Farial, 3, Ingra Morales Claro3,4, Darlan Candido2,3, Lucas A. Moyses Franco3,4, Pamela S. Andrade3,4, Thais M. Coletti3,4, Camila A. M. Silva3,4, Flavia C. Sales3,4, Erika R. Manuli3,4, Renato S. Aguiar5, Nelson Gaburo6, Cecilia da C. Camilo7, Nelson A. Fraiji8, Myuki A. Esashika Crispim8, Maria do Perpétuo S. S. Carvalho8, Andrew Rambaut9, Nick Loman10, Oliver G. Pybus2, Ester C. Sabino3,4, on behalf of CADDE Genomic Network11, MRC Centre for Global Infectious Disease Analysis J-I, Imperial College London, London, United Kingdom., Department of Zoology UoO, Oxford, United Kingdom., Institute of Tropical Medicine UoSP, São Paulo, Brazil., Department of Infectious Disease SoM, University of São Paulo, São Paulo, Brazil., Departamento de Genética EeE, Instituto de Ciências Biológicas, Universidade Federal de Minas Gerais, Belo Horizonte, Brazil., DB Diagnósticos do Brasil SP, Brazil., CDL Laboratório Santos e Vidal Ltda. M, Brazil., HEMOAM FdHeHdA, Manaus, Brazil., Institute of Evolutionary Biology UoE, Edinburgh, UK., Institute for Microbiology and Infection UoB, Birmingham, UK., 287 hwco. 2021. Genomic characterisation of an emergent SARS-CoV-2 lineage in Manaus: preliminary findings.

10. Wu K, Werner AP, Moliva JI, Koch M, Choi A, Stewart-Jones GBE, Bennett H, Boyoglu-Barnum S, Shi W, Graham BS, Carfi A, Corbett KS, Seder RA, Edwards DK. 2021. mRNA-1273 vaccine induces neutralizing antibodies against spike mutants from global SARS-CoV-2 variants. bioRxiv doi:10.1101/2021.01.25.427948.

11. Xia X. 2021. Domains and Functions of Spike Protein in Sars-Cov-2 in the Context of Vaccine Design. Viruses 13.

12. Juraszek J, Rutten L, Blokland S, Bouchier P, Voorzaat R, Ritschel T, Bakkers MJG, Renault LLR, Langedijk JPM. 2021. Stabilizing the closed SARS-CoV-2 spike trimer. Nat Commun 12:244.

### EMBODIMENTS

The following are exemplary embodiments:
1. A recombinant Newcastle disease virus (NDV) comprising a packaged genome comprising a transgene that comprises a nucleotide sequence encoding a SARS-CoV-2 spike protein.
2. A recombinant Newcastle disease virus (NDV) comprising a packaged genome comprising a transgene that comprises a nucleotide sequence encoding a secreted protein comprising the receptor binding domain of a SARS-CoV-2 spike protein.
3. The recombinant NDV of embodiment 2, wherein the protein further comprises a tag.
4. The recombinant NDV of embodiment 3, wherein the tag is a histidine or flag tag.
5. A recombinant Newcastle disease virus (NDV) comprising a packaged genome comprising a transgene that comprises a nucleotide sequence encoding a secreted protein comprising the ectodomain of a SARS-CoV-2 spike protein.
6. The recombinant NDV of embodiment 5, wherein the protein further comprises a tag.
7. The recombinant NDV of embodiment 6, wherein the tag is a histidine or flag tag.
8. A recombinant Newcastle disease virus (NDV) comprising a packaged genome, wherein the package genome comprises a transgene, wherein the transgene comprises a nucleotide sequence encoding a SARS-CoV-2 spike protein, and wherein the transgene comprises an RNA sequence corresponding to the negative sense of the cDNA sequence of SEQ ID NO:4, 6, 8 or 10.
9. A recombinant Newcastle disease virus (NDV) comprising a packaged genome, wherein the packaged genome comprises a transgene, wherein the transgene comprises a nucleotide sequence encoding a SARS-CoV-2 spike protein, and wherein the transgene comprises an RNA sequence encoding the amino acid sequence set forth in SEQ ID NO:5, 7, 9 or 11.
10. A recombinant NDV comprising a packaged genome, wherein the packaged genome comprises a transgene, wherein the transgene comprises a nucleotide sequence encoding a chimeric F protein, and wherein the chimeric F protein comprises a SARS-CoV-2 spike protein ectodomain and NDV F protein transmembrane and cytoplasmic domains.
11. A recombinant NDV comprising a packaged genome, wherein the packaged genome comprises a transgene, wherein the transgenecomprises a nucleotide sequence encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site.
12. The recombinant NDV of embodiment 11, wherein amino acid residues corresponding to amino acid residues 682 to 685 of the polybasic cleavage site of the the spike protein found at GenBank Accession No. MN908947 are substituted with a single alanine.
13. The recombinant NDV of any one of embodiments 10 to 12, wherein the SARS-CoV-2 spike protein ectodomain is linked via a linker (e.g., SEQ ID NO:24) to the NDV F protein transmembrane and cytoplasmic domains.
14. A recombinant NDV comprising a packaged genome, wherein the packaged genome comprises a transgene, wherein transgene comprises a nucleotide sequence encoding a chimeric F protein, wherein the transgene comprises an RNA sequence corresponding to the negative sense of the cDNA sequence of SEQ ID NO: 12.
15. A recombinant NDV comprising a packaged genome, wherein the packaged genome comprises a transgene, wherein the transgene comprises a nucleotide sequence encoding a chimeric F protein, wherein the chimeric F protein comprises the amino acid sequence set forth in SEQ ID NO: 13.
16. A recombinant NDV comprising a packaged genome, wherein the packaged genome comprises a transgene, wherein the transgene comprises a nucleotide sequence encoding a chimeric F protein, wherein the transgene comprises an RNA sequence corresponding to the negative sense of the cDNA sequence of SEQ ID NO: 14.
17. A recombinant NDV comprising a packaged genome, wherein the packaged genome comprises a transgene, wherein the transagene comprises a nucleotide sequence encoding a chimeric F protein, wherein the chimeric F protein comprises the amino acid sequence set forth in SEQ ID NO: 15.
18. A recombinant NDV comprising a packaged genome, wherein the packaged genome comprises a transgene, wherein the transgene comprises a nucleotide sequence encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.
19. The recombinant NDV of embodiment 18, wherein amino acid residues corresponding to amino acid residues 682 to 685 of the the spike protein found at GenBank Accession No. MN908947 are substituted with a single alanine.
20. The recombinant NDV of embodiment 18 or 19, wherein the SARS-CoV-2 spike protein ectodomain is linked via a linker (e.g., SEQ ID NO:24) to the NDV F protein transmembrane and cytoplasmic domains.
21. A recombinant NDV comprising a packaged genome, wherein the packaged genome comprises a transgene, wherein the transgene comprises an RNA sequence corresponding to the negative sense of the cDNA sequence of SEQ ID NO: 16.
22. A recombinant NDV comprising a packaged genome comprising a transgene, wherein the transgene comprises a nucleotide sequence encoding a chimeric F protein, wherein the chimeric F protein comprises the amino acid sequence set forth in SEQ ID NO: 17.
23. A recombinant NDV comprising a packaged genome, wherein the packaged genome comprises a transgene, wherein the transgene comprises an RNA sequence corresponding to the negative sense of the cDNA sequence of SEQ ID NO: 18.
24. A recombinant NDV comprising a packaged genome, wherein the packaged genome comprises a transgene, wherein the transgene comprises a nucleotide sequence encoding a chimeric F protein, wherein the chimeric F protein comprises the amino acid sequence set forth in SEQ ID NO: 19.
25. The recombinant NDV of any one of embodiments 10 to 24, wherein the NDV virion comprises the chimeric F protein.
26. The recombinant NDV of any one of embodiments 1 to 25, wherein the genome comprises a NDV F transcription unit, a NDV NP transcription unit, a NDV M transcription unit, a NDV L transcription unit, a NDV P transcription unit, and a NDV HN transcription unit.
27. The recombinant NDV of embodiment 26, wherein the NDV F transcription unit encodes a NDV F protein comprising a leucine to alanine amino acid substitution at the amino residue corresponding to amino acid residue 289 of the LaSota NDV strain.
28. The recombinant NDV of any one of embodiments 1 to 27, wherein the transgene is between two NDV transcription units of the packaged genome.
29. The recombinant NDV of embodiment 28, wherein the two transcription units of the packaged genome are the transcription units for the NDV P gene and the NDV M gene.
30. The recombinant NDV of any one of embodiments 1 to 29, wherein the genome further comprises a transgene comprising a nucleotide sequence encoding a SARS-CoV-2 nucleocapsid protein.
31. The recombinant NDV of any one of embodiments 1 to 30 which comprises an NDV backbone which is lentogenic.
32. The recombinant NDV of any one of embodiments 1 to 30 which comprises an NDV backbone of LaSota strain (e.g., SEQ ID NO:1 or 25).
33. The recombinant NDV of any one of embodiments 1 to 30 which comprises an NDV backbone of Hitchner B1 strain (e.g., SEQ ID NO:2).
34. A recombinant Newcastle disease virus (NDV) comprising a packaged genome comprising a transgene encoding a SARS-CoV-2 nucleocapsid protein.
35. A recombinant NDV virion comprising a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.
36. The recombinant NDV virion of embodiment 35, wherein amino acid residues corresponding to amino acid residues 682 to 685 of the the spike protein found at GenBank Accession No. MN908947 are substituted with a single alanine.
37. The recombinant NDV virion of embodiment 35 or 36, wherein the SARS-CoV-2 spike protein ectodomain is linked via a linker (e.g., SEQ ID NO:24) to the NDV F protein transmembrane and cytoplasmic domains.
38. A recombinant NDV virion comprising a chimeric F protein, wherein the chimeric F protein comprises the amino acid sequence of SEQ ID NO: 15, 17, or 19.
39. A composition comprising the recombinant NDV of any one of embodiments 1 to 38.
40. An immunogenic composition comprising the recombinant NDV of any one of embodiments 1 to 38.
41. The immunogenic composition of embodiment 36, wherein the recombinant NDV is inactivated.
42. The immunogenic composition of embodiment 40 or 41 further comprising an adjuvant.
43. A method for inducing an immune response to SARS-CoV-2 spike protein or nucleocapsid, comprising administering the immunogenic composition of any one of embodiments 40 to 42 to a subject.
44. A method for preventing COVID-19, comprising administering the immunogenic composition of any one of embodiments 40 to 42 to a subject.
45. A method for immunizing a subject against SARS-CoV-2, comprising administering the immunogenic composition of any one of embodiments 40 to 42 to a subject.
46. The method of any one of embodiments 43 to 45, wherein the composition is administered to the subject intranasally or intramuscularly.
47. The method of any one of embodiments 43 to 46, wherein the subject is a human.
48. A kit comprising the recombinant NDV of any one of embodiments 1 to 38.
49. A cell line or chicken embryonated egg comprising the propagating the recombinant NDV of any one of embodiments 1 to 34.
50. A method for propagating the recombinant NDV of any one of embodiments 1 to 34, the method comprising culturing the cell or embryonated egg of embodiment 49.
51. The method of embodiment 50, wherein the method further comprises isolating the recombinant NDV from the egg or embryonated egg.
52. A method for detecting the presence of antibody specific to SARS-CoV-2 spike protein or nucleocapsid, comprising contacting a specimen with the recombinant NDV of any one of embodiments 1 to 38 in an immunoassay.
53. The method of embodiment 52, wherein the specimen is a biological specimen.
54. The method of embodiment 52, wherein the biological specimen is blood, plasma or sera from a subject.
55. The method of embodiment 54, wherein the subject is human.
56. The method of embodiment 53, wherein the specimen is an antibody or antisera
57. A transgene comprising a nucleotide sequence encoding a chimeric F protein, wherein the chimeric F protein comprises a SARS-CoV-2 spike protein ectodomain and an NDV F protein transmembrane and cytoplasmic domains.
58. A transgene comprising a nucleotide sequence encoding a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site.
59. A transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.
60. The transgene of embodiment 58 or 59, wherein amino acid residues corresponding to amino acid residues 682 to 685 of the the spike protein found at GenBank Accession No. MN908947 are substituted with a single alanine.
61. A transgene comprising a nucleotide sequence encoding a chimeric F protein, wherein the chimeric F protein comprises the amino acid sequence set forth in SEQ ID NO: 13, 15, 17, or 19.
62. A transgene comprising a nucleotide sequence set forth in SEQ ID NO:12, 14, 16 or 18.
63. A vector comprising the transgene of any one of embodiments 57 to 62.
64. A nucleotide sequence comprising the transgene of any one of embodiments 57 to 62 and (1) a NDV F transcription unit, (2) a NDV NP transcription unit, (3) a NDV M transcription unit, (4) a NDV L transcription unit, (5) a NDV P transcription unit, and (6) a NDV HN transcription unit.
65. The nucleotide sequence of embodiment 64, wherein the NDV F transcription unit encodes a NDV F protein comprising a leucine to alanine amino acid substitution at the amino residue corresponding to amino acid residue 289 of the LaSota NDV strain.
66. A vector comprising the nucleotide sequence of embodiment 64 or 65.
67. A kit comprising the nucleotide sequence of embodiment 64 or 65, the transgene of any one of embodiments 57 to 62, or the vector of embodiment 63 or 66.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed:

1. A recombinant Newcastle disease virus (NDV) comprising a packaged genome comprising a transgene that comprises a nucleotide sequence encoding a SARS-CoV-2 spike protein.

2. A recombinant Newcastle disease virus (NDV) comprising a packaged genome comprising a transgene that comprises a nucleotide sequence encoding a secreted protein comprising the receptor binding domain of a SARS-CoV-2 spike protein.

3. The recombinant NDV of claim 2, wherein the protein further comprises a tag.

4. The recombinant NDV of claim 3, wherein the tag is a histidine or flag tag.

5. A recombinant Newcastle disease virus (NDV) comprising a packaged genome comprising a transgene that comprises a nucleotide sequence encoding a secreted protein comprising the ectodomain of a SARS-CoV-2 spike protein.

6. The recombinant NDV of claim 5, wherein the protein further comprises a tag.

7. The recombinant NDV of claim 6, wherein the tag is a histidine or flag tag.

8. A recombinant Newcastle disease virus (NDV) comprising a packaged genome, wherein the package genome comprises a transgene, wherein the transgene comprises a nucleotide sequence encoding a SARS-CoV-2 spike protein, and wherein the transgene comprises an RNA sequence corresponding to the negative sense of the cDNA sequence of SEQ ID NO:4, 6, 8 or 10.

9. A recombinant Newcastle disease virus (NDV) comprising a packaged genome, wherein the packaged genome comprises a transgene, wherein the transgene comprises a nucleotide sequence encoding a SARS-CoV-2 spike protein, and wherein the transgene comprises an RNA sequence encoding the amino acid sequence set forth in SEQ ID NO:5, 7, 9 or 11.

10. A recombinant NDV comprising a packaged genome, wherein the packaged genome comprises a transgene, wherein the transgene comprises a nucleotide sequence encoding a chimeric F protein, and wherein the chimeric F protein comprises a SARS-CoV-2 spike protein ectodomain and NDV F protein transmembrane and cytoplasmic domains.

11. A recombinant NDV comprising a packaged genome, wherein the packaged genome comprises a transgene, wherein the transgenecomprises a nucleotide sequence encoding a chimeric F protein, wherein the chimeric F protein comprises a SARS-CoV-2 spike protein ectodomain and NDV F protein transmembrane and cytoplasmic domains, and wherein the SARS-CoV-2 spike protein ectodomain lacks a polybasic cleavage site.

12. The recombinant NDV of claim 11, wherein amino acid residues corresponding to amino acid residues 682 to 685 of the polybasic cleavage site of the the spike protein found at GenBank Accession No. MN908947 are substituted with a single alanine.

13. The recombinant NDV of any one of claims 10 to 12, wherein the SARS-CoV-2 spike protein ectodomain is linked via a linker to the NDV F protein transmembrane and cytoplasmic domains.

14. A recombinant NDV comprising a packaged genome, wherein the packaged genome comprises a transgene, wherein transgene comprises a nucleotide sequence encoding a chimeric F protein, wherein the transgene comprises an RNA sequence corresponding to the negative sense of the cDNA sequence of SEQ ID NO: 12.

15. A recombinant NDV comprising a packaged genome, wherein the packaged genome comprises a transgene, wherein the transgene comprises a nucleotide sequence encoding a chimeric F protein, wherein the chimeric F protein comprises the amino acid sequence set forth in SEQ ID NO:13.

16. A recombinant NDV comprising a packaged genome, wherein the packaged genome comprises a transgene, wherein the transgene comprises a nucleotide sequence encoding a chimeric F protein, wherein the transgene comprises an RNA sequence corresponding to the negative sense of the cDNA sequence of SEQ ID NO: 14.

17. A recombinant NDV comprising a packaged genome, wherein the packaged genome comprises a transgene, wherein the transagene comprises a nucleotide sequence encoding a chimeric F protein, wherein the chimeric F protein comprises the amino acid sequence set forth in SEQ ID NO: 15.

18. A recombinant NDV comprising a packaged genome, wherein the packaged genome comprises a transgene, wherein the transgene comprises a nucleotide sequence encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.

19. The recombinant NDV of claim 18, wherein amino acid residues corresponding to amino acid residues 682 to 685 of the the spike protein found at GenBank Accession No. MN908947 are substituted with a single alanine.

20. The recombinant NDV of claim 18 or 19, wherein the SARS-CoV-2 spike protein ectodomain is linked via a linker to the NDV F protein transmembrane and cytoplasmic domains.

21. A recombinant NDV comprising a packaged genome, wherein the packaged genome comprises a transgene, wherein the transgene comprises an RNA sequence corresponding to the negative sense of the cDNA sequence of SEQ ID NO: 16.

22. A recombinant NDV comprising a packaged genome comprising a transgene, wherein the transgene comprises a nucleotide sequence encoding a chimeric F protein, wherein the chimeric F protein comprises the amino acid sequence set forth in SEQ ID NO: 17.

23. A recombinant NDV comprising a packaged genome, wherein the packaged genome comprises a transgene, wherein the transgene comprises an RNA sequence corresponding to the negative sense of the cDNA sequence of SEQ ID NO:18.

24. A recombinant NDV comprising a packaged genome, wherein the packaged genome comprises a transgene, wherein the transgene comprises a nucleotide sequence encoding a chimeric F protein, wherein the chimeric F protein comprises the amino acid sequence set forth in SEQ ID NO: 19.

25. The recombinant NDV of any one of claims 10 to 24, wherein the NDV virion comprises the chimeric F protein.

26. The recombinant NDV of any one of claims 1 to 25, wherein the genome comprises a NDV F transcription unit, a NDV NP transcription unit, a NDV M transcription unit, a NDV L transcription unit, a NDV P transcription unit, and a NDV HN transcription unit.

27. The recombinant NDV of claim 26, wherein the NDV F transcription unit encodes a NDV F protein comprising a leucine to alanine amino acid substitution at the amino residue corresponding to amino acid residue 289 of the LaSota NDV strain.

28. The recombinant NDV of any one of claims 1 to 27, wherein the transgene is between two NDV transcription units of the packaged genome.

29. The recombinant NDV of claim 28, wherein the two transcription units of the packaged genome are the transcription units for the NDV P gene and the NDV M gene.

30. The recombinant NDV of any one of claims 1 to 29, wherein the genome further comprises a transgene comprising a nucleotide sequence encoding a SARS-CoV-2 nucleocapsid protein.

31. The recombinant NDV of any one of claims 1 to 30 which comprises an NDV backbone which is lentogenic.

32. The recombinant NDV of any one of claims 1 to 30 which comprises an NDV backbone of LaSota strain.

33. The recombinant NDV of any one of claims 1 to 30 which comprises an NDV backbone of Hitchner B1 strain.

34. A recombinant Newcastle disease virus (NDV) comprising a packaged genome comprising a transgene encoding a SARS-CoV-2 nucleocapsid protein.

35. A recombinant NDV virion comprising a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.

36. The recombinant NDV virion of claim 35, wherein amino acid residues corresponding to amino acid residues 682 to 685 of the the spike protein found at GenBank Accession No. MN908947 are substituted with a single alanine.

37. The recombinant NDV virion of claim 35 or 36, wherein the SARS-CoV-2 spike protein ectodomain is linked via a linker to the NDV F protein transmembrane and cytoplasmic domains.

38. A recombinant NDV virion comprising a chimeric F protein, wherein the chimeric F protein comprises the amino acid sequence of SEQ ID NO:15, 17, or 19.

39. A composition comprising the recombinant NDV of any one of claims 1 to 38.

40. An immunogenic composition comprising the recombinant NDV of any one of claims 1 to 38.

41. The immunogenic composition of claim 36, wherein the recombinant NDV is inactivated.

42. The immunogenic composition of claim 40 or 41 further comprising an adjuvant.

43. A method for inducing an immune response to SARS-CoV-2 spike protein or nucleocapsid, comprising administering the immunogenic composition of any one of claims 40 to 42 to a subject.

44. A method for preventing COVID-19, comprising administering the immunogenic composition of any one of claims 40 to 42 to a subject.

45. A method for immunizing a subject against SARS-CoV-2, comprising administering the immunogenic composition of any one of claims 40 to 42 to a subject.

46. The method of any one of claims 43 to 45, wherein the composition is administered to the subject intranasally or intramuscularly.

47. The method of any one of claims 43 to 46, wherein the subject is a human.

48. A kit comprising the recombinant NDV of any one of claims 1 to 38.

49. A cell line or chicken embryonated egg comprising the propagating the recombinant NDV of any one of claims 1 to 34.

50. A method for propagating the recombinant NDV of any one of claims 1 to 34, the method comprising culturing the cell or embryonated egg of claim 49.

51. The method of claim 50, wherein the method further comprises isolating the recombinant NDV from the egg or embryonated egg.

52. A method for detecting the presence of antibody specific to SARS-CoV-2 spike protein or nucleocapsid, comprising contacting a specimen with the recombinant NDV of any one of claims 1 to 38 in an immunoassay.

53. The method of claim 52, wherein the specimen is a biological specimen.

54. The method of claim 52, wherein the biological specimen is blood, plasma or sera from a subject.

55. The method of claim 54, wherein the subject is human.

56. The method of claim 53, wherein the specimen is an antibody or antisera.

57. A transgene comprising a nucleotide sequence encoding a chimeric F protein, wherein the chimeric F protein comprises a SARS-CoV-2 spike protein ectodomain and an NDV F protein transmembrane and cytoplasmic domains.

58. A transgene comprising a nucleotide sequence encoding a chimeric F protein, wherein the chimeric F protein comprises a SARS-CoV-2 spike protein ectodomain and an NDV F protein transmembrane and cytoplasmic domains, and wherein the SARS-CoV-2 spike protein ectodomain lacks a polybasic cleavage site.

59. A transgene encoding a chimeric F protein, wherein the chimeric F protein comprises a SARS-CoV-2 spike protein ectodomain and NDV F protein transmembrane and cytoplasmic domains, wherein amino acid residues corresponding to amino acid residues 817, 892, 899, 942, 986, and 987 of the spike protein found at GenBank Accession No. MN908947 are substituted with prolines, and wherein the ectodomain of the SARS-CoV-2 spike protein lacks a polybasic cleavage site.

60. The transgene of claim 58 or 59, wherein amino acid residues corresponding to amino acid residues 682 to 685 of the the spike protein found at GenBank Accession No. MN908947 are substituted with a single alanine.

61. A transgene comprising a nucleotide sequence encoding a chimeric F protein, wherein the chimeric F protein comprises the amino acid sequence set forth in SEQ ID NO: 13, 15, 17, or 19.

62. A transgene comprising a nucleotide sequence set forth in SEQ ID NO:12, 14, 16 or 18.

63. A vector comprising the transgene of any one of claims 57 to 62.

64. A nucleotide sequence comprising the transgene of any one of claims 57 to 62 and (1) a NDV F transcription unit, (2) a NDV NP transcription unit, (3) a NDV M transcription unit, (4) a NDV L transcription unit, (5) a NDV P transcription unit, and (6) a NDV HN transcription unit.

65. The nucleotide sequence of claim 64, wherein the NDV F transcription unit encodes a NDV F protein comprising a leucine to alanine amino acid substitution at the amino residue corresponding to amino acid residue 289 of the LaSota NDV strain.

66. A vector comprising the nucleotide sequence of claim 64 or 65.

67. A kit comprising the nucleotide sequence of claim 64 or 65, the transgene of any one of claims 57 to 62, or the vector of claim 63 or 66.
